(19) United States
(12) Patent Application Publication
(10) Pub. No.: US 2023/0285539 A1
(43) Pub. Date: Sep. 14, 2023

(54) VACCINES AGAINST SARS-COV-2 AND OTHER CORONAVIRUSES

(71) Applicants: The Henry M. Jackson Foundation for the Advancement of Military Medicine, Inc., Bethesda, MD (US); The Government of the United States, as Represented by the Secretary of the Army, Fort Detrick, MD (US)

(72) Inventors: Michael Gordon JOYCE, Silver Spring, MD (US); Kayvon MODJARRAD, Bethesda, MD (US)

(73) Assignees: Henry M. Jackson Foundation for the Advancement of Military Medicine, Inc., Bethesda, MD (US); The Government of the United States, as Represented by the Secretary of the Army, Fort Detrick, MD (US)

(21) Appl. No.: 17/905,614

(22) PCT Filed: Mar. 8, 2021
(86) PCT No.: PCT/US2021/021405

§ 371 Date: Sep. 2, 2022

Related U.S. Application Data
(60) Provisional application No. 62/986,522, filed on Mar. 6, 2020.
(60) Provisional application No. 63/038,600, filed on Jun. 12, 2020.

(51) Int. Cl. A61K 39/215; A61K 9/51; A61P 31/14

(52) U.S. Cl. CPC A61K 39/215; A61K 9/5169; A61P 31/14; A61K 2039/55505

(57) ABSTRACT

The present disclosure relates to the field of vaccines and binding molecules, as well as preparations and methods of their use in the treatment and/or prevention of disease. Described are vaccines and binding molecules, compositions containing the same, and uses thereof for treating or preventing coronavirus infections, in particular, β-coronaviruses such as SARS- CoV-2, the causative agent of COVID-19, as well as SARS-CoV-1 and other coronaviruses.

### FIELD OF INVENTION

The present disclosure relates to the field of vaccines, as well as preparations and methods of their use in the treatment and/or prevention of disease. Described are vaccines, pharmaceutical compositions containing the same, and uses thereof for treating or preventing coronavirus infections, including β-coronaviruses such as SARS-CoV-2, the causative agent of COVID-19.

### CROSS REFERENCE STATEMENT

This application claims priority under 35 U.S.C. § 119(e) to U.S. Provisional Application 62/986,522 filed Mar. 6, 2020, and to U.S. Provisional Application 63/038,600 filed Jun. 12, 2020. The entire contents of both provisional applications are incorporated herein by reference.

### GOVERNMENT SUPPORT CLAUSE

This invention was made with government support under W81XWH1820040 awarded by the Defense Health Agency. The government has certain rights in the invention.

### BACKGROUND

The following discussion is merely provided to aid the reader in understanding the disclosure and is not admitted to describe or constitute prior art thereto.

The emergence of SARS-CoV-2—also named COVID-19—marks the seventh coronavirus to be isolated from humans, and the third to cause a severe disease after severe acute respiratory syndrome (SARS) and Middle East respiratory syndrome (MERS). The rapid spread of SARS-CoV-2, and the grave risk it poses to global health, prompted the World Health Organization to declare, on 30 Jan. 2020, the COVID-19 outbreak to be a public health emergency of international concern and on 11 Mar. 2020 to be a pandemic. The rapidly evolving epidemiology of the pandemic has accelerated the need to elucidate the molecular biology of this novel coronavirus.

The present disclosure provides nanoparticle vaccines that can be used to treat or prevent coronavirus infection, such as infections caused by SARS-CoV-2 (i.e., COVID-19).

### SUMMARY

Described herein are vaccines for the treatment and/or prevention of infections caused by coronaviruses, such as SARS-CoV-2 (i.e., COVID-19), and methods and uses of the same.

In a first aspect, the present disclosure provides nanoparticles comprising a fusion protein comprising a nanoparticle-forming peptide and at least one antigenic coronavirus peptide selected from: a receptor-binding domain (RBD) of a coronavirus, or a fragment or variant thereof, an N-terminal domain (NTD) of a coronavirus, or a fragment or variant thereof, an S1 domain of a coronavirus, or a fragment or variant thereof, a stabilized extracellular spike S-2P domain of a coronavirus, or a fragment or variant thereof, a stabilized extracellular spike S domain of a coronavirus, or a fragment or variant thereof, or a stabilized extracellular spike S-trimer of a coronavirus, or a fragment or variant thereof.

The nanoparticle-forming peptide may comprise or be a ferritin protein or a fragment or variant thereof. The nanoparticle-forming peptide may comprise or be Helicobacter pylori ferritin (Hpf) or a fragment or variant thereof. The nanoparticle-forming peptide may comprise an amino acid sequence selected from:

ESQVRQQFSKDIEKLLNEQVNKEMQSSNLYMSMSSWCYTHSLDGAGLFLF
DHAAEEYEHAKKLIIFLNENNVPVQLTSISAPEHKFEGLTQIFQKAYEHE
QHISESINNIVDHAIKSKDHATFNFLQWYVAEQHEEEVLFKDILDKIELI
GNENHGLYLADQYVKGIAKSRKSGS

or a fragment or variant thereof,

DIIKLLNEQVNKEMQSSNLYMSMSSWCYTHSLDGAGLFLFDHAAEEYEHA
KKLIIFLNENNVPVQLTSISAPEHKFEGLTQIFQKAYEHEQHISESINNI
VDHAIKSKDHATFNFLQWYVAEQHEEEVLFKDILDKIELIGNENHGLYLA
DQYVKGIAKSRKSGS

or a fragment or variant thereof, or

SKDIIKLLNEQVNKEMQSSNLYMSMSSWCYTHSLDGAGLFLFDHAAEEYE
HAKKLIIFLNENNVPVQLTSISAPEHKFEGLTQIFQKAYEHEQHISESIN
NIVDHAIKSKDHATFNFLQWYVAEQHEEEVLFKDILDKIELIGNENHGLY
LADQYVKGIAKSRKSGS

or a fragment or variant thereof.

The nanoparticle may possess a 4-fold axis or a 3-fold axis.

The antigenic coronavirus peptide may be connected to the nanoparticle-forming peptide via a linker. The linker may comprise an amino acid sequence selected from: GSGGGG, GGGG, GSGG, GGG, and SGG.

The fusion protein may comprise 2-10 (e.g., 2, 3, 4, 5, 6, 7, 8, 9, or 10) antigenic coronavirus peptides connected in series, optionally via peptide linkers, which linkers may comprise an amino acid sequence selected from GSGGGG, GGGG, GSGG, GGG, and SGG.

The antigenic coronavirus peptide may be isolated or derived from a coronavirus selected from SARS-CoV-2, human coronavirus OC43 (hCoV-OC43), Middle East respiratory syndrome-related coronavirus (MERS-CoV), severe acute respiratory syndrome-related coronavirus (SARS-CoV-1), HKU-1, 229E, or NL63.

The nanoparticle may comprise one or more of an Hpf or a fragment or variant thereof connected via a peptide linker to an RBD or a fragment or variant thereof; an Hpf or a fragment or variant thereof connected via a peptide linker to an NTD or a fragment or variant thereof; an Hpf or a fragment or variant thereof connected via a peptide linker to an S1 or a fragment or variant thereof; an Hpf or a fragment or variant thereof connected via a peptide linker to a stabilized extracellular spike domain (S-2P) or a fragment or variant thereof; a sequence of any fusion protein disclosed in Table 3, and a sequence of any fusion protein disclosed in Table 18.

In some embodiments, the nanoparticle can bind to a human ACE-2 receptor, while in some embodiments, the nanoparticle cannot bind to a human ACE-2 receptor. In some embodiments, the nanoparticle can bind to anti-coronavirus antibody CR3022, or an ACE2 receptor.

In a second aspect, the present disclosure provides vaccines comprising any of the nanoparticles of the first aspect or otherwise disclosed herein. The vaccines may further comprise one or more adjuvants, such as one or more selected from ALFQ, alhydrogel, and combinations thereof.

In a third aspect, the present disclosure provides messenger RNA (mRNA) encoding any of the nanoparticles of the first aspect or otherwise disclosed herein.

In a fourth aspect, the present disclosure provides methods of treating or preventing a coronavirus infection in a subject in need thereof, comprising administering to a subject in need thereof any of the nanoparticles of the first aspect or otherwise disclosed herein, any of the vaccines of the second aspect or otherwise disclosed herein, or any of the mRNA of the third aspect or otherwise disclosed herein.

The subject may be at risk of contracting a coronavirus infection, or the subj ect may already have contracted a coronavirus infection.

The coronavirus may be SARS-CoV-2 or a variant thereof, such as B.1.1.7, B.1.351, and P1. Additionally or alternatively, the coronavirus may be SARS-CoV-1 or a variant thereof.

In a fifth aspect, the present disclosure provides any of the nanoparticles of the first aspect or otherwise disclosed herein, any of the vaccines of the second aspect or otherwise disclosed herein, or any of the mRNA of the third aspect or otherwise disclosed herein for use in treating or preventing a coronavirus infection in a subject in need thereof.

The subject may be at risk of contracting a coronavirus infection, or the subj ect may already have contracted a coronavirus infection.

The coronavirus may be SARS-CoV-2 or a variant thereof, such as B.1.1.7, B.1.351, and P1. Additionally or alternatively, the coronavirus can be SARS-CoV-1 or a variant thereof.

In a sixth aspect, the present disclosure provides uses of any of the nanoparticles of the first aspect or otherwise disclosed herein, any of the vaccines of the second aspect or otherwise disclosed herein, or any of the mRNA of the third aspect or otherwise disclosed herein in the preparation of a medicament for treating or preventing a coronavirus infection in a subject in need thereof.

Prior to being administered a nanoparticle or vaccine as disclosed herein, the subject may be administered a priming dose of a DNA sequence encoding a receptor-binding domain (RBD) of a coronavirus, or a fragment or variant thereof. The RBD may be a SARS-CoV-2 RBD. The DNA sequence may comprise SEQ ID NO: 282. The DNA sequence may encode a protein comprising SEQ ID NO: 283.

In a seventh aspect, the present disclosure provides methods of screening for binding molecules that are capable of binding to coronavirus, comprising using the nanoparticles listed in Table 18 to identify binding molecules that bind to the peptides with sequences listed in Table 18.

In an eighth aspect, the present disclosure provides DNA molecules comprising a sequence encoding any of the nanoparticles of the first aspect or otherwise disclosed herein. In alternative embodiments of the eighth aspect, the present disclosure provides DNA molecules comprising a sequence encoding a receptor-binding domain (RBD) of a coronavirus, or a fragment or variant thereof. The RBD may be from SARS-CoV-2. The DNA sequence may comprise SEQ ID NO: 282. The DNA sequence may encode a protein comprising SEQ ID NO: 283.

In a ninth aspect, the present disclosure provides plasmids comprising any DNA molecule of the eighth aspect or otherwise disclosed herein, wherein the plasmid can express the DNA molecule in vivo.

In a tenth aspect, the present disclosure provides methods of priming an immune response in a subject, comprising administering to a subject any DNA molecule of the eighth aspect or otherwise disclosed herein or any plasmid of the ninth aspect or otherwise disclosed herein, prior to administering to the subject any of the nanoparticles of the first aspect or otherwise disclosed herein, any of the vaccines of the second aspect or otherwise disclosed herein, or any of the mRNA of the third aspect or otherwise disclosed herein.

In an eleventh aspect, the present disclosure provides any DNA molecule of the eighth aspect or otherwise disclosed herein or any plasmid of the ninth aspect or otherwise disclosed herein for use in priming an immune response in a subject prior to administering to the subject any of the nanoparticles of the first aspect or otherwise disclosed herein, any of the vaccines of the second aspect or otherwise disclosed herein, or any of the mRNA of the third aspect or otherwise disclosed herein.

In a twelfth aspect, the present disclosure provides uses of any DNA molecule of the eighth aspect or otherwise disclosed herein or any plasmid of the ninth aspect or otherwise disclosed herein in the preparation of a medicament for in priming an immune response in a subject prior to administering to the subject any of the nanoparticles of the first aspect or otherwise disclosed herein, any of the vaccines of the second aspect or otherwise disclosed herein, or any of the mRNA of the third aspect or otherwise disclosed herein.

In a thirteenth aspect, the present disclosure provides methods of treating or preventing a coronavirus infection in a subject in need thereof, comprising administering to the subject an anti-coronavirus antibody obtained from or cloned from an immunized subject that was administered any of the nanoparticles of the first aspect or otherwise disclosed herein, any of the vaccines of the second aspect or otherwise disclosed herein, or any of the mRNA of the third aspect or otherwise disclosed herein.

In a fourteenth aspect, the present disclosure provides anti-coronavirus antibodies obtained from or cloned from an immunized subject that was administered any of the nanoparticles of the first aspect or otherwise disclosed herein, any of the vaccines of the second aspect or otherwise disclosed herein, or any of the mRNA of the third aspect or otherwise disclosed herein, for use in treating or preventing a coronavirus infection in a subject in need thereof.

In a fifteenth aspect, the present disclosure provides uses of an anti-coronavirus antibody obtained from or cloned from an immunized subject that was administered any of the nanoparticles of the first aspect or otherwise disclosed herein, any of the vaccines of the second aspect or otherwise disclosed herein, or any of the mRNA of the third aspect or otherwise disclosed herein, for use in the preparation of a medicament for treating or preventing a coronavirus infection in a subject in need thereof.

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### BRIEF DESCRIPTION OF THE DRAWINGS

FIG. 1 shows the design of SARS-CoV-2 Spike Domain-Ferritin Nanoparticles. A) Full length SARS-CoV-2 spike primary and three-dimensional structure. Molecular hinges identified by molecular dynamics simulations and electron cryotomography are labeled on the three-dimensional model. A single chain of the structured trimeric ectodomain is shaded according to the simple schematic diagram (top) with the N-terminal domain (NTD) and Receptor-Binding Domain (RBD) of the S1 polypeptide and the C-terminal coiled coil N-terminal to hinge 1. Remaining portions of the S1 and S2 polypeptides are shaded, with regions after the knee hinge colored in white. The transmembrane domain of all chains is depicted inside a patch of membrane. Truncation and optimization of the Spike C-terminal heptad repeat. Residues 1140 to 1161 between Hinge 1 and 2 are shown aligned to the ideal heptad repeat sequence. Residues in the native spike sequence which break this pattern are highlighted. These residues are also labeled and highlighted on the three-dimensional structure which are shaded according to the primary structure diagram. Two engineered sequences are aligned indicating the residue at which they were truncated and mutations made to enforce the heptad repeat are indicated. B) Primary structure and three-dimensional model of a Spike Trimer-Ferritin nanoparticle. Differences between the native spike sequence and the engineered nanoparticle are on the primary schematic (top). A three-dimensional model of a nanoparticles displaying eight trimeric spikes using PDB ID 6VXX and 3EGM is shaded accordingly with ferritin shown in alternating grey and white for clarity. The nanoparticle is depicted along one of the 4-fold symmetry axis of ferritin and one of the 3-fold symmetry axes of both the spike and ferritin. C) Identification of regions hindering assembly and Expression of RBD-Ferritin nanoparticles. The RBD of SARS-CoV-2 (PDB ID:6MOJ) is shown in isolation with the footprint of the ACE2 binding site outlined in dashed lines. Three hydrophobic surfaces are shown in light gray surface, with the corresponding residues shown underneath. A hydrophobic patch near the C-terminus of the RBD is buried by S2 and part of S1 in the trimeric context. Two other strips of hydrophobic residues occur near the ACE2 binding site with some residues contributing to ACE2 binding. D) Primary structure and three-dimensional model of an RBD-Ferritin nanoparticle. A modeled 24-mer nanoparticle display RBD epitopes is depicted along one of the 3-fold symmetry axis of ferritin and colored according to the primary structure of the RBD-ferritin fusion. Truncation points, linkers, and alterations made to the native spike sequence are indicated on the primary structure. E) Primary structure and three-dimensional model of a RBD-NTD-Ferritin nanoparticle. A modeled nanoparticle displaying RBD and NTD epitopes is depicted and colored according to the primary structure of the RBD-NTD-ferritin fusion. Truncation points, linkers, and alterations made to the native spike sequence are indicated on the primary structure. F) S1 forms a hydrophobic collar around the N-terminal beta sheet of S2. The C-terminus of S1 forms natively after furin cleavage. In order to express S1 without S2 in a monomeric context the sequence was first truncated prior to the furin site. However, to express soluble protein and S1-ferritin, the N-terminal portion of S2 was required and could be attached by a linker. The structured regions flanking the S1-S2 cleavage site are shown on PBD ID 6VXX with S1 colored in dark gray and S2 in light gray. A dashed line indicates the unmodelled loop which contains the furin site, and terminal residues of the structured portions of S1 and S2 are labeled. G) Primary structure and three-dimensional model of an S1-Ferritin nanoparticle. A modeled nanoparticle displaying RBD and NTD epitopes and perhaps epitopes comprising both domains is depicted and colored according to the primary structure of the S1-ferritin fusion. Truncation points and placement of linkers are indicated on the primary structure.

FIG. 2 shows the design of SARS-CoV-2 Spike-Ferritin Nanoparticles with extended helical coiled coil regions and/or incorporation of additional stabilization mutations in the S2 domain. Exemplary examples 1B-08, pCoV186, and pCoV187 are shown as examples with linear schematics, and models of the extended coiled-coil regions.

FIG. 3 shows details of select S Trimer-Ferritin nanoparticles including sequence information.

FIG. 4 shows the high-resolution structure of SARS-CoV-2 receptor-binding domain (RBD) in ribbon representation with specific residues labeled and shown in sphere representation. The hydrophobic surface that can be modified for improved production, stability, and yield of the RBD or RBD-Ferritin constructs.

FIG. 5 shows models of the SARS-CoV-2 RBD-Ferritin variants with increased nanoparticle formation, stability, and yield. Panel (A) shows the crystal structure of SARS-CoV-2 RBD and Panels (B-G) show variants comprising select amino acid modifications. Alterations to less hydrophobic residues or introduction of glycans at these residues will serve to increase nanoparticle yield, formation and stability. Panels (H-N) show variants comprising select amino acid modifications. Alterations to less hydrophobic residues or introduction of glycans at these residues will serve to increase nanoparticle yield, formation, and stability. Native residues shown in sphere representation.

FIG. 6 shows biochemical and biophysical characterization of exemplary Spike-Ferritin nanoparticles. A) Size-exclusion chromatography, B) protein expression yields from 1 L 293F, C) SDS-PAGE of representative Spike-Ferritin nanoparticles, D) dynamic light scattering analysis of the representative SpFN particles, E) negative-stain EM images of pCoV-1B-05 and SpFN_1B-06-PL nanoparticles, and representative 2D class average. Fusion proteins and the nanoparticles formed by the fusion proteins: a RBD and ferritin, a NTD and ferritin, S1 and ferritin, RBD-NTD and ferritin, and a stabilized prefusion S trimer and ferritin.

FIG. 7 shows biochemical and biophysical characterization of exemplary RBD-Ferritin nanoparticles. A,B) Size-exclusion chromatography, C) SDS-PAGE of representative RBD-Ferritin nanoparticles, D) dynamic light scattering analysis of the representative RBD-FN particles, E) protein expression yields from 1 L 293F, F) negative-stain EM images of RBD-Ferritin-pCoV131 nanoparticles, and representative 2D class average.

FIG. 8 shows biochemical and biophysical characterization of exemplary NTD-Ferritin nanoparticles. A) Size-exclusion chromatography, B) protein expression yields from 1 L 293F,C) SDS-PAGE of representative NTD-Ferritin nanoparticles, D) dynamic light scattering analysis of the representative NTD-Ferritin particles, F) negative-stain EM images of NTD-Ferritin-pCoV65 nanoparticles, and representative 2D class average.

FIG. 9 shows biochemical and biophysical characterization of exemplary S1-Ferritin nanoparticles. A) Size-exclusion chromatography, B) protein expression yields from 1 L 293F,C) SDS-PAGE of representative S1-Ferritin nanoparticles, D) dynamic light scattering analysis of the representative S1-Ferritin particles, F) negative-stain EM images of S1-Ferritin-pCoV111 nanoparticles, and representative 2D class average.

FIG. 10 shows biochemical and biophysical characterization of exemplary RBD-NTD-Ferritin nanoparticles. A) Size-exclusion chromatography, B) protein expression yields from 1 L 293F,C) SDS-PAGE of representative RBD-NTD-Ferritin nanoparticles, D) dynamic light scattering analysis of the representative RBD-NTD -Ferritin particles, F) negative-stain EM images of RBD-NTD-Ferritin-pCoV146 nanoparticles, and representative 2D class average.

FIG. 11 shows the negative-Stain Electron Microscopy 3D Reconstructions of SARS-CoV-2 Spike Domain-Ferritin Nanoparticles. A) Changes to native sequence made in the SpFN_1B-06-PL construct are depicted along with a negative stain 3D reconstruction with applied octahedral symmetry. An asymmetric unit of non-ferritin density is highlighted in dark gray. A trimeric model of SpFN_1B-06 is docked into the neg-stain map (shown in the inset). (B) RBD-Ferritin_pCoV131 (RFN-131) schematic (top) with the reconstructed 3D negative stain EM map shown with the RBD domain indicated in dark gray. C) RBD-NTD-Ferritin construct pCoV146 schematic (top) with the reconstructed 3D negative stain EM map shown with the RBD and NTD domains indicated in dark gray. D) S1-Ferritin construct pCoV111 schematic (top) with the reconstructed 3D negative stain EM map shown with the S1 domain indicated in dark gray. An asymmetric unit of non-ferritin density is highlighted in the inset. A model of the SARS-CoV-2 S1 molecule is docked into the neg-stain map (shown in the inset).

FIG. 12 shows the correlation between ID50 neutralization values for animals immunized with 8 Antigens and 2 Adjuvants (right hand side) plotted against Octet binding response (nm) at 180 sec at a 1:100 serum dilution. Samples were taken at week 2, week 5, and week 8.

FIG. 13 shows immunogenicity in C57BL/6 and Balb/c mice of SARS-CoV-2 SpFN_1B-06-PL adjuvanted with ALFQ or Alhydrogel elicited RBD-responses measured by Octet Biolayer Interferometry.

FIG. 14 shows antigenicity in C57BL/6 and Balb/c mice of SARS-CoV-2 SpFN_1B-06-PL adjuvanted with ALFQ or Alhydrogel induced RBD or S responses measured by ELISA.

FIG. 15 shows serum blocking of ACE2 interaction with SARS-CoV-2 RBD measured by Octet Biolayer Interferometry.

FIG. 16 shows SpFN_1B-06-PL adjuvanted with ALFQ or Alhydrogel in C57BL/6 and Balb/c mice pseudovirus SARS-CoV-2 neutralization.

FIG. 17 shows SpFN_1B-06-PL adjuvanted with ALFQ in C57BL/6 and Balb/c mice live-virus SARS-CoV-2 neutralization.

FIG. 18 shows antigenicity in C57BL/6 and Balb/c mice of SARS-CoV-2 SpFN_1B-06-PL (0.08 µg dose) adjuvanted with ALFQ measured by Octet Biolayer Interferometry.

FIG. 19 shows spike and RBD Antigenicity in C57BL/6 and Balb/c mice of SARS-CoV-2 SpFN_1B-06-PL (0.08 µg dose) adjuvanted with ALFQ measured by ELISA.

FIG. 20 shows SpFN_1B-06-PL (0.08 µg dose) adjuvanted with ALFQ in C57BL/6 and Balb/c mice pseudovirus SARS-CoV-2 neutralization.

FIG. 21 shows SpFN_1B-06-PL (0.08 µg dose) adjuvanted with ALFQ in C57BL/6 and Balb/c mice live-virus SARS-CoV-2 neutralization.

FIG. 22 Analysis of cellular response following immunization with SpFN + ALFQ. (A) Sera collected on day 10 from immunized mice were added in quadruplicate serial dilutions to ELISA plates coated with S-2P protein. Duplicated wells were probed with anti-mouse-IgG1-HRP. Additional duplicates were probed with either anti-mouse-IgG2c-HRP or anti-mouse IgG2a-HRP for C57BL/6 and BALB/c mice, respectively. Data was interpolated to obtain the dilution factor at OD450 of 1, and plotted as ratios of IgG2/IgG1. (B) Splenocytes were collected 10 day after immunization and prepared for surface and intracellular staining and flow cytometry for analysis. Initial gating identified CD4+ and CD8+ T cell population, and further analysis of the frequency of CD4+ and CD8+ cells producing Th1-specific cytokines IL-2, IFN-g and TNF-a, and Th2-specific cytokine IL-4.

FIG. 23 shows frequency of SARS-CoV-2 Spike specific cytokine secreting (A) CD4⁺ T-cells and (B) CD8+ T cells in splenocytes of C57BL/6 mice vaccinated with SpFN + AH (Group 1) or SpFN + ALFQ (Group 2) at Days 3, 5, 7, and 10.

FIG. 24 shows the vaccine elicited serum from SpFN and RBD-Ferritin vaccinated mice provides protective immunity in K18-ACE2 transgenic mice against SARS-CoV-2. A) Polyclonal Ig from immunized C57BL/6 mice was purified and administered intraperitoneally to recipient mice prior to infection with SARS-CoV-2 virus. Three antibody amounts (high, medium and low) were provided to animal groups from either the SpFN-vaccinated mice, or the RBD-Ferritin immunized mice. Mouse IgG was purified from pooled naive sera and given to 10 mice as a control group, and an additional control group received PBS. B) Schematic of the mouse transfusion and challenge study timeline. C) Mouse serum samples were taken just prior to challenge and measured for SARS-CoV-2 pseudovirus neutralization. D) Percentage change in mouse body weight. Groups are defined based on ID50 GMT shown in panel C. E) Percentage survival of K18-ACE2 mice. Each group is defined by the Immune sera type and the group GMT values from panel C.

FIG. 25 shows the Octet Biolayer Interferometry measurement of vaccinated mouse sera (week 10) reactivity to RBD molecules. Immunogens used to vaccinate mice are indicated at the top of the plots, mouse strain (legend) and the average binding value for each group of mice is indicated at the base of the plot. A) Mouse sera binding to SARS-CoV-2 or SARS-CoV-1 RBD molecules. B) SpFN_1B-06-PL-, C) pCoV131, D) pCoV111-vaccine-elicited sera binding to SARS-CoV-2 and variant RBD molecules. The RBD mutations are indicated on the x-axis of the graph.

FIG. 26 shows that immunization with SARS-CoV-2 immunogens (SpFN_1B-06-PL or RBD-Ferritin_pCoV131) elicits potent neutralizing immune responses against both SARS-CoV-2 and SARS-CoV-1.

FIG. 27 shows that immunization in rhesus macaques with SpFN_1B-06-PL or RFN_pCoV131 induced robust IgG binding and neutralization responses. Antibody responses in serum were assessed every 2 weeks following vaccination by MSD binding to Spike protein (A) or pseudovirus neutralization assay (B) Thick lines indicate geometric means within each group. Responses were compared between vaccination groups at week 8 - either Spike binding by MSD (C), pseudovirus neutralization assay (D), inhibition of ACE2 binding as measured by MSD (E) and live virus neutralization (F). Significance was assessed using a Kruskal-Wallis test followed by a Dunn’s post-test.

FIG. 28 shows that vaccination with SpFN_1B-06-PL and RFN_pCoV131 elicited antibody responses to SARS-1. Binding responses were measured at week 6 by Biolayer Interferometry (A). Circles indicate binding responses to SARS-CoV-2 RBD, and squares indicate binding to SARS-CoV-1 RBD. (B) Pseudovirus neutralization measured against SARS-CoV-1 at week 8. Significance was assessed using a Kruskal-Wallis test followed by a Dunn’s post-test.

FIG. 29 shows the CD4⁺ memory T cell responses to Spike assessed at week 8 by intracellular cytokine staining. Responses shown are the summed responses from cells stimulated with Spike 1 and Spike 2 peptide pools. Closed circles indicate animals with a positive response at week 8 (defined as greater than 3 times the background of the total group measured at baseline). Open circles indicate animals with non-positive responses. Summary of positive responses is shown below each graph. Th1 responses (summed IFNg, TNF and IL-2) are shown in A, and Th2 responses (summed IL-4 and IL-13) are shown in B. Individual cytokine responses to CD40L (C) and IL-21 (D) are also shown. Significance was assessed using a Kruskal-Wallis test followed by a Dunn’s post-test.

FIG. 30 shows the viral replication in the lower and upper airways after SpFN_1B-06-PL or RFN_pCoV131 vaccination and subsequent SARS-CoV-2 respiratory challenge. Subgenomic messenger RNA (sgmRNA) copies per milliliter were measured in the nasopharyngeal swabs (Top Panel), bronchoalveolar lavage fluid (Middle Panel), and saliva (Lower panel) of vaccinated and control animals for two weeks following intranasal and intratracheal SARS-CoV-2 (USA-WA1/2020) challenge of vaccinated and control animals. Specimens were collected on 1, 2, 4, 7, 10, and 14 days post-challenge. Dotted lines demarcate the assay lower limit of linear performance range (corresponding to 450 copies/ml). In the box plots, horizontal lines indicate the mean and the top and bottom reflect the minimum and maximum.

FIG. 31 shows the Histopathological Analysis after SARS-CoV-2 Challenge in Unvaccinated and SpFN-Vaccinated Rhesus Macaques. A-C Histopathology of representative hematoxylin-and-eosin-stained, paraffin-embedded lung parenchyma at 7 dpi. Significant interstitial pneumonia is present only in the unvaccinated animals (A), characterized by inflammatory necrotic debris (white star), type II pneumocyte hyperplasia (black arrow), edema (triangle), and vasculitis of small- to medium- calliber blood vessels (white arrows). Interstitial pneumonia was not observed in the vaccinated animals (B, C). Scale bars, 50 µm.D-F. Immunohistochemical analysis of paraffin-embedded lung parenchyma at 7 dpi. SARS-CoV-2 viral antigen was detected in the lungs of unvaccinated animals (D.) Scale bar, 100 µm. Inset: SARS-CoV-2 viral antigen was detected in alveolar pneumocytes (thick arrow), pulmonary macrophages (arrowhead), and, rarely, endothelial cells (thin arrow). Scale bar, 20 µm. Viral antigen was not detected in vaccinated animals (E, F). Scale bars, 100 µm.

FIG. 32 shows the immunogenicity of SpFN or RFN in rhesus macaques measured by MSD. IgG binding responses were measured to RBD (A). Inhibition of ACE2 binding to either the full spike protein (B) or RBD (C) are shown. Antibody responses in serum were assessed every 2 weeks following immunization and challenge. Thick lines indicate geometric means within each group.

FIG. 33 shows the immunogenicity of SpFN_1B-06-PL or RBD-FN_pCoV131 in rhesus macaques measured by Biolayer Interferometry. SARS-CoV-2 RBD-specific binding antibody responses in serum were assessed every 2 weeks following immunization and challenge.

FIG. 34 shows the immunogenicity of SpFN or RBD-FN in rhesus macaques measured by SARS-CoV-2 live virus neutralization. A live-virus neutralization assay for SARS-CoV-2 assessed responses in serum 4 weeks following each immunization. Thick lines indicate geometric means within each group.

FIG. 35 shows the SpFN_1B-06-PL and RBD-Ferritin_pCoV131 vaccinated rhesus macaque sera neutralizes multiple strains of SARS-CoV-2 including WA1/2020, and emergent strains B.1.1.7 and B.1.351 in a live-virus neutralization assay.

FIG. 36 shows the CD8+ memory T cell responses to Spike assessed at week 8 by intracellular cytokine staining. Responses shown are the summed responses from cells stimulated with Spike 1 and Spike 2 peptide pools. Th1 include summed IFNg, TNF and IL-2. Significance was assessed using a Kruskal-Wallis test followed by a Dunn’s post-test.

FIG. 37 shows the CD4⁺ (A-D) and CD8+ (E) memory T cell responses to Spike were assessed at week 8 by intracellular cytokine staining. Responses shown are the summed responses from cells stimulated with Spike 1 and Spike 2 peptide pools. Responses were measured at weeks 6 and 8 (2 and 4 weeks following the second vaccination) and weeks 9/10 (½ weeks following challenge). CD4+ Th1 responses (summed IFNg, TNF and IL-2) are shown in A, and CD4+ Th2 responses (summed IL-4 and IL-13) are shown in B. Individual CD4+ cytokine responses to CD40L (C) and IL-21 (D) are also shown. CD8+ Th1 responses (summed IFNg, TNF and IL-2) are shown in E.

FIG. 38 shows the Individual IFNg, TNF and IL-2 CD4+ memory T cell responses to Spike were assessed at week 8 by intracellular cytokine staining. For A and B responses shown are the summed responses from cells stimulated with Spike 1 and Spike 2 peptide pools. Significance was assessed using a Kruskal-Wallis test followed by a Dunn’s post-test.

FIG. 39 shows the ratio of Th1 to Th2 cells determined at week 8 in animals with positive Th2 responses. The dashed line indicates an equal proportion of Th1:Th2 cells.

FIG. 40 shows the Antibody effector responses as measured in plasma following immunization with SpFN or RFN.

FIG. 41 shows the viral RNA measured in NP swabs (A), BAL (B) and Saliva (C) following IN/IT SARS-CoV-2 challenge of vaccinated and control animals. SARS-CoV-2 total RNA is shown for days 1, 2, 4, 7, 10, and 14 post-challenge. Dotted line indicates the assay lower limit of linear performance range (corresponding to 450 copies/ml). Values that fall on the line represent samples in which viral load was detected, but values are less than 450 copies/mL.

FIG. 42 shows the histopathological analysis after SARS-CoV-2 Challenge in RBD_pCoV131- and SpFN_1B-06-PL-vaccinated Rhesus Macaques. Interstitial pneumonia was not observed in the vaccinated animals (A-C). Scale bars, 50 µm. Immunohistochemical analysis of paraffin-embedded lung parenchyma at 7 dpi. Viral antigen was not detected in vaccinated animals (D-F). Scale bars, 100 µm.

FIG. 43 shows the Histopathological Analysis after SARS-CoV-2 Challenge in RBD and SpFN-Vaccinated Rhesus Macaques(A) Minimal to mild foci of cellular infiltrates centered around small- to- medium- caliber pulmonary arteries were occasionally noted in some of the animals of all of the vaccine groups. Scale bar, 50 µm. (B) Type II pneumocyte hyperplasia (TIIPH) in an unvaccinated animal. Scale bar, 20 µm.

FIG. 44 shows that immunization with a mixture of SARS-CoV-2 SpFN and SARS-CoV-1 SpFN immunogens elicits potent binding antibodies against both SARS-CoV-2 and SARS-CoV-1.

FIG. 45 shows that immunization with a mixture of SARS-CoV-2 SpFN and SARS-CoV-1 SpFN immunogens elicits potent neutralizing antibodies against both SARS-CoV-2 and SARS-CoV-1 as shown by the ID50 (top 4 panels) and ID80 (lower 4 panels) pseudovirus neutralization titers.

FIG. 46 shows the negative-stain EM characterization of Spike-Ferritin nanoparticles for SARS-CoV-1, HKU-1 and 229E coronaviruses. Proteins were produced in 293F cells, purified by GNA-lectin and size-exclusion chromatography. Purified nanoparticles were visualized on copper grids (top) using a TEM, with 2D class averages (middle), and 3D models (lower) of the nanoparticles shown.

FIG. 47 shows the serum blocking of ACE2 interaction with SARS-CoV-2 RBD as measured by Octet Biolayer Interferometry. PBS and mouse sera prior to immunization was used to show the specific inhibitory effect following vaccination.

FIG. 48 shows the immunization of C57BL/6 and Balb/c mice with SARS-CoV-2 RBD DNA prime followed by RBD or RBD-Ferritin boost elicited SARS-COV-2 RBD responses measured by ELISA.

FIG. 49 shows the schematic of the Spike-Ferritin -- RBD-Ferritin heterologous prime-boost experiment, and the OCTET binding responses to the SARS-CoV-2 RBD.

FIG. 50 shows the electrostatic potential of the SARS-CoV-2 RBD in surface representation. A view of the RBD from the side is shown on the left, and a view of the RBD from the “top” with the ACE-2 receptor site indicated is shown on the right. Lighter regions indicate a hydrophobic surface that can be modified for improved production, stability and yield of the RBD or RBD-Ferritin constructs.

FIG. 51 shows space-filled representations of exemplary nanoparticles that comprise a 4-fold axis or a 3-fold axis.

FIG. 52 shows exemplary fusion proteins and the nanoparticles formed by the fusion proteins: a RBD and ferritin, a NTD and ferritin, S1 and ferritin, RBD-NTD and ferritin, and a stabilized prefusion S trimer and ferritin.

FIG. 53 shows TEM images of select nanoparticles.

FIG. 54 shows linear and modular schematics of a vaccine particle comprising multiple RBDs in a “beads on a string” format.

### DETAILED DESCRIPTION

The present disclosure provides nanoparticle vaccines for treating or preventing coronavirus infections and coronavirus infectious diseases, such as but not limited to COVID-19, which is caused by SARS-CoV-2. The disclosed nanoparticles are made up of fusion proteins that comprise a nanoparticle-forming peptide and an antigenic coronavirus peptide, which may be optionally joined together via a linker. The fusion proteins are capable of self-assembling into nanoparticles that are stable in solution and able to generate a protective neutralizing immune response (i.e., the production of neutralizing antibodies and/or defensive cytokines) when administered to a subject. In addition to the nanoparticles, the disclosed vaccines may also comprise an adjuvant.

### I. Definitions

It is to be understood that the terminology used herein is for the purpose of describing particular embodiments only, and is not intended to be limiting.

Technical and scientific terms used herein have the meanings commonly understood by one of ordinary skill in the art, unless otherwise defined. Unless otherwise specified, materials and/or methodologies known to those of ordinary skill in the art can be utilized in carrying out the methods described herein, based on the guidance provided herein.

As used herein, the singular terms “a,” “an,” and “the” include plural referents unless the context clearly dictates otherwise. Reference to an object in the singular is not intended to mean “one and only one” unless explicitly so stated, but rather “one or more.”

As used herein, “about” when used with a numerical value means the numerical value stated as well as plus or minus 10% of the numerical value. For example, “about 10” should be understood as both “10” and “9-11.”

As used herein, a phrase in the form “A/B” or in the form “A and/or B” means (A), (B), or (A and B); a phrase in the form “at least one of A, B, and C” means (A), (B), (C), (A and B), (A and C), (B and C), or (A, B, and C).

As used herein, the term “comprising” is intended to mean that the compositions and methods include the recited elements, but does not exclude others.

As used herein, a “variant” when used in the context of referring to a peptide means a peptide sequence that is derived from a parent sequence by incorporating one or more amino acid changes, which can include substitutions, deletions, or insertions. For the purposes of this disclosure, a variant may comprise an amino acid sequence that shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or up to about 100% sequence identity or homology with the reference (or “parent”) sequence. For purposes of this disclosure, the terms “variant” and “derivative” when used in the context of referring to a peptide are used interchangeably.

As used herein, a “variant” when used in the context of referring to a virus (e.g., SARS-CoV-2) means a virus that is a progeny of a reference (or “parent”) virus that possesses one or more changes in its genome (e.g., a RNA genome), or a virus that is genetically engineered to have one or more changes in its genome, relative to a reference (or “parent”) virus, which may or may not result in changes to the proteins encoded by the RNA sequence (e.g., one or more proteins of a variant virus may include substitutions, deletions, or insertions compared to a parent strain). For example, known variants of SARS-CoV-2 include, but are not limited to, B.1.1.7 (first identified in the United Kingdom), B.1.351 (first identified in South Africa), and P.1 (first identified in Brazil). For the purposes of this disclosure, a variant of a virus may comprise a genome sequence that shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or up to about 100% sequence identity or homology with the reference (or “parent”) genome sequence.

As used herein, the phrases “effective amount,” “therapeutically effective amount,” and “therapeutic level” mean the dosage or concentration of a disclosed vaccine that provides the specific pharmacological effect for which the vaccine is administered in a subject in need of such treatment, i.e. to treat or prevent a coronavirus infection (e.g., MERS, SARS, or COVID-19). It is emphasized that a therapeutically effective amount or therapeutic level of a vaccine will not always be effective in treating or preventing the infections described herein, even though such dosage is deemed to be a therapeutically effective amount by those of skill in the art. For convenience only, exemplary dosages, drug delivery amounts, therapeutically effective amounts, and therapeutic levels are provided herein. The therapeutically effective amount may vary based on the route of administration and dosage form, the age and weight of the subject, and/or the subject’s condition, including the type and severity of the coronavirus infection.

The terms “treat,” “treatment” or “treating” as used herein with reference to a coronavirus infection refer to reducing or eliminating viral load or eliminating histopathology or virus presence in the airways or lungs.

The terms “prevent,” “preventing” or “prevention” as used herein with reference to a coronavirus infections refer to precluding or reducing the risk of an infection from developing in a subject exposed to a coronavirus, or to precluding or reducing the risk of developing a high viral load of coronavirus or reducing or eliminating histopathology or virus presence in the airways or lungs. Prevention may also refer to the prevention of a subsequent infection once an initial infection has been treated or cured. Prevention may also refer to the prevention of or reduction of risk of transmission of virus from one subject host to another subject host.

The terms “individual,” “subject,” and “patient” are used interchangeably herein, and refer to any individual mammalian subject, e.g., bovine, canine, feline, equine, or human. In specific embodiments, the subject, individual, or patient is a human.

### II. Coronaviruses

Coronaviruses are a family of viruses (i.e., the coronaviridae family) that cause respiratory infections in mammals and that comprise a genome that is roughly 30 kilobases in length. The coronaviridae family is divided into four genera and the genome encodes 28 proteins across multiple open reading frames, including 16 non-structural proteins (nsp) that are post-translationally cleaved from a polyprotein.

The coronaviridae family includes both α-coronaviruses or β-coronaviruses, which both mainly infect bats, but can also infect other mammals like humans, camels, and rabbits. β-coronaviruses have, to date, been of greater clinical importance, having caused epidemics of diseases with high mortality such as severe acute respiratory syndrome (SARS), Middle East respiratory syndrome (MERS), and COVID-19. Other disease-causing β-coronaviruses include OC44, and HKU1. Non-limiting examples of disease-causing α-coronaviruses include, but are not limited to, 229E and NL63.

Although SARS-CoV-2 is a newly identified virus, it shares genetic and morphologic features with others in the Coronaviridae family, particularly those from the β-coronavirus genus. The genome of the recently isolated SARS-CoV-2 shares 82% nucleotide identity with human SARS-CoV (SARS-CoV-1) and 89% with bat SARS-like-CoVZXC21 (Lu et al., 2020). The spike (S) glycoprotein, in particular, bears significant structural homology with SARS-CoV-1 compared to other coronaviruses such as MERS-CoV. Like SARS-CoV-1, the surface Spike (S) glycoprotein of SARS-CoV-2 binds the same host receptor, ACE-2, to mediate cell entry (Letko et al., 2020; Yan et al., 2020a). S—a class I fusion protein—is also a critical determinant of viral host range and tissue tropism and the primary target of the host immune response (Li, 2016). As such, most coronavirus vaccine candidates are based on S or one of its sub-components. Coronavirus S glycoproteins contain three segments: a large ectodomain, a single-pass transmembrane anchor and a short intracellular tail. The ectodomain consists of a receptor-binding subunit, S1, which contains two sub-domains: one at the N-terminus and the other at the C-terminus. The latter comprises the receptor-binding domain (RBD), which serves the vital function of attaching the virus to the host receptor and triggering a conformational change in the protein that results in fusion with the host cell membrane through the S2 subunit.

Multiple technology platforms are currently advancing SARS-CoV-2 vaccine development, including nucleic acid vaccines, whole virus vaccines, recombinant protein subunit vaccines and nanoparticle vaccines. Of these vaccine platform types, nanoparticle technologies have previously been shown to improve antigen structure and stability, as well as vaccine targeted delivery, immunogenicity, and safety.

In some embodiments, the coronavirus that is treated or prevented by the disclosed vaccines is a β-coronavirus. In some embodiments, the β-coronavirus is selected from the group consisting of severe acute respiratory syndrome coronavirus 2 (SARS-CoV-2) (also known by the provisional name 2019 novel coronavirus, or 2019-nCoV or COVID-19), human coronavirus OC43 (hCoV-OC43), Middle East respiratory syndrome-related coronavirus (MERS-CoV, also known by the provisional name 2012 novel coronavirus, or 2012-nCoV), severe acute respiratory syndrome-related coronavirus (SARS-CoV, also known as SARS-CoV-1), HKU-1, 229E, and NL63. In some embodiments, the β-coronaviruses is SARS-CoV-2, the causative agent of COVID-19. In some embodiments, the disclosed vaccines may provide a broad spectrum treatment and/or prevention for multiple different types of coronavirus, such as MERS-CoV, SARS-CoV-1, and/or SARS-CoV-2.

### III. Nanoparticle Vaccines and Binding Agents

Disclosed herein are vaccines that can be used to treat or prevent coronavirus infections such as but not limited to COVID-19, which is caused by SARS-CoV-2. In particular, the disclosed vaccines can comprise a fusion protein comprising a nanoparticle-forming peptide and an antigenic coronavirus peptide, which may optionally be connected by a linker (i.e., a “linker domain”). The antigenic coronavirus peptide may comprise one or more fragments or full-length proteins derived from a coronavirus (e.g., SARS-CoV-2 or SARS-CoV-1).

### A. Nanoparticle-Forming Peptide

The nanoparticle-forming peptide of a vaccine as disclosed herein may be any suitable nanoparticle-forming peptide. H. pylori ferritin and fragments and variants thereof are particularly suitable to serve as a nanoparticle-forming peptides for vaccines as disclosed herein. Thus, the nanoparticle-forming peptide of a vaccine as disclosed herein may comprise a Helicobacter pylori ferritin protein (HpF) or fragment or variant thereof. For instance, the nanoparticle component may comprise the following amino acid sequence derived from H. pylori ferritin:

ESQVRQQFSKDIEKLLNEQVNKEMQSSNLYMSMSSWCYTHSLDGAGLFLF
DHAAEEYEHAKKLIIFLNENNVPVQLTSISAPEHKFEGLTQIFQKAYEHE
QHISESINNIVDHAIKSKDHATFNFLQWYVAEQHEEEVLFKDILDKIELI
GNENHGLYLADQYVKGIAKSRKSGS (SEQID NO: 1).

Thus, the nanoparticle-forming peptide of the vaccine may comprise the foregoing H. pylori ferritin sequence (SEQ ID NO: 1) or a variant thereof, which may comprise 1, 2, 3, 4, 5, 6, 7, 8, 9, 10, 11, 12, 13, 14, or 15 or more substitution, deletion, or insertion mutations. For example, the nanoparticle-forming peptide may comprise a variant of SEQ ID NO: 1 that may comprise a deletion of 1, 2, 3, 4, 5, 6, 7, 8, 9, or 10 or more amino acids from the N-terminal domain of SEQ ID NO: 1. In some embodiments, that nanoparticle-forming peptide may comprise a substitution of the glutamic acid residue (E) at position 13 of SEQ ID NO: 1. In some embodiments, that nanoparticle-forming peptide may comprise a substitution of the glutamic acid residue (E) at position 13 of SEQ ID NO: 1 and a deletion of 1, 2, 3, 4, 5, 6, 7, 8, 9, or 10 or more amino acids from the N-terminal domain of SEQ ID NO: 1, such as in the following sequences:

DIIKLLNEQVNKEMQSSNLYMSMSSWCYTHSLDGAGLFLFDHAAEEYEHA
KKLIIFLNENNVPVQLTSISAPEHKFEGLTQIFQKAYEHEQHISESINNI
VDHAIKSKDHATFNFLQWYVAEQHEEEVLFKDILDKIELIGNENHGLYLA
DQYVKGIAKSRKSGS (SEQ ID NO: 2); or

SKDIIKLLNEQVNKEMQSSNLYMSMSSWCYTHSLDGAGLFLFDHAAEEYE
HAKKLIIFLNENNVPVQLTSISAPEHKFEGLTQIFQKAYEHEQHISESIN
NIVDHAIKSKDHATFNFLQWYVAEQHEEEVLFKDIlDKIELIGNENHGLY
LADQYVKGIAKSRKSGS (SEQ ID NO: 3).

In some embodiments, the nanoparticle-forming peptide may comprise a variant of any of SEQ ID NO: 1, SEQ ID NO: 2, or SEQ ID NO: 3, which may comprise an amino acid sequence that shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or up to 100% sequence identity or homology with any of SEQ ID NO: 1, SEQ ID NO: 2, or SEQ ID NO: 3.

As noted above, in some embodiments, the nanoparticle-forming peptide may be a non-ferritin-based peptide, such as a peptide that comprises the following sequence or a fragment or variant thereof:

MQIYEGKLTAEGLRFGIVASRFNHALVDRLVEGAIDAIVRHGGREEDITL
VRVPGSWEIPVAAGELARKEDIDAVIAIGVLIRGATPHFDYIASEVSKGL
ADLSLELRKPITFGVITADTLEQAIERAGTKHGNKGWEAALSAIEMANLF
KSLR (SEQ ID NO: 4).

In some embodiments, the nanoparticle-forming peptide may comprise a variant of SEQ ID NO: 4, which may comprise 1, 2, 3, 4, 5, 6, 7, 8, 9, 10, 11, 12, 13, 14, or 15 or more substitution, deletion, or insertion mutations in SEQ ID NO: 4. In some embodiments, the nanoparticle-forming peptide may comprise a variant of SEQ ID NO: 4 that may comprise an amino acid sequence that shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or up to 100% sequence identity or homology with SEQ ID NO: 4.

### B. Linker Domain

The disclosed fusion proteins generally comprise a flexible amino acid linker; however, the linker domain (i.e. linker) is optional and in some embodiments the nanoparticle-forming peptide may be directly joined with the antigenic coronavirus peptide. The linker may be about 3 to about 50 amino acids in length, or more particularly about 4 to about 42 amino acids in length. In some embodiments, the linker may be 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 amino acids in length. The linker domain may comprise glycine (G) repeats and or a combination of glycine (G) and serine (S) residues. Several exemplary linker sequences are disclosed in Table 1 below.

TABLE 1

Exemplary Linker Sequences

| Linker Sequence | SEQ ID NO |
|---|---|
| GSGG | 5 |
| GSGGSG | 6 |
| GSGGEMKQIEDKIEEILSKIYHIENEIARIKKLIGRGSGGSG | 7 |
| LDSIKEELDKIHKNGSGGSG | 8 |
| IDSIKEEIDKIHKNGSGGSG | 9 |
| MKQIEDKIEEILSKIYHIENEIARIKKLIGRGSGGSG | 10 |
| GSGGGG | 11 |
| GSG | 12 |
| GSGGSGGSGGSGGG | 13 |
| GGGSGGGSGG | 14 |
| GGGG | 15 |
| GGG | 16 |
| SGG | 17 |

The linker domain may comprise 1, 2, or 3 repeats of any one of SEQ ID NOs: 5-17. In some embodiments, the linker domain comprises a variant of any one of SEQ ID NOs: 5-17 that may comprise an amino acid sequence that shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or up to 100% sequence identity or homology with any one of SEQ ID NOs: 5-17.

The foregoing linker sequences are not intended to be limiting, and those of skill in the art will understand that other flexible peptide linkers may also be suitable for connecting the nanoparticle-forming peptide and the antigenic coronavirus peptide, based on the guidance provided herein.

### C. Antigenic Coronavirus Peptide

In general, the antigenic coronavirus peptide of the disclosed fusion proteins comprises a coronavirus spike protein (also known as “S protein” or “glycoprotein S”), which is generally responsible for viral entry into a host cell, or a fragment or a variant thereof. In some embodiments, the antigenic coronavirus peptide may comprise 1, 2, or 3 or more distinct domains of a coronavirus spike protein connected together in sequence, and in such embodiments, a linker may optionally separate the distinct domains.

The spike protein is selected as an antigenic coronavirus peptide of vaccines as disclosed herein, because antibodies that develop against this peptide are likely to be neutralizing. The spike protein comprises two functional subunits responsible for binding to the host cell receptor (Si subunit) and fusion of the viral and cellular membranes (S₂ subunit). A fusion protein of the present disclosure may comprise the entire spike protein, only the S₁ subunit, only the S₂ subunit, or any antigenic/immunogenic fragment or variant thereof. In some embodiments, the fusion protein comprises full length coronavirus spike protein sequence. In some embodiments, the fusion protein comprises a variant that comprises about 20%, about 25%, about 30%, about 35%, about 40%, about 45%, about 50%, about 55%, about 60%, about 65%, about 70%, about 75%, about 80%, about 85%, about 90%, 91%, 92%, 93%, 94%, 95%, 96%, 97%, 98%, or 99% of a coronavirus spike protein (e.g., SEQ ID NO: 18), so long as the fragment is able to elicit an immune response (i.e., it is an antigenic fragment).

While not wanting to be bound by theory, it is understood that the spike protein of SARS-CoV-2 attaches to human angiotensin converting enzyme (ACE)-2 cell surface receptors facilitating human infection. Thus, antibodies that can bind to the spike glycoprotein and prevent interaction with the ACE2 receptor can facilitate protection from infection. The SARS-CoV-2 spike protein (NCBI Reference Sequence: YP_009724390.1) comprises 1273 amino acids and consists of a signal peptide (amino acids 1-13) located at the N-terminus, the S1 subunit (14-685 residues), and the S2 subunit (686-1273 residues); the last two regions are responsible for receptor binding and membrane fusion, respectively. The amino acid sequence is shown below.

MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT (SEQ ID NO: 18)

Specific domains of the coronavirus spike protein that are particularly useful as an antigenic coronavirus peptide in the disclosed fusion proteins are:
a receptor-binding domain (RBD) of a coronavirus, or a fragment or variant thereof,
an N-terminal domain (NTD) of a coronavirus, or a fragment or variant thereof,
a receptor-binding domain (RBD)-N-terminal domain chimera of a coronavirus, or a fragment or variant thereof,
an S1 domain of a coronavirus, or a fragment or variant thereof,
a stabilized extracellular spike S-2P domain of a coronavirus, or a fragment or variant thereof,
a stabilized extracellular spike S domain of a coronavirus, or a fragment or variant thereof, or
a stabilized extracellular spike S-trimer of a coronavirus, or a fragment or variant thereof.

Thus, the antigenic coronavirus peptide may comprise an RBD. An RBD may comprise the SARS-CoV-2 RBD amino acid sequence set forth below:

NITNLCPFGEVFNATRFASVYAWNRKRISNCVADYSVLYNSASFSTFKCY
GVSPTKLNDLCFTNVYADSFVIRGDEVRQIAPGQTGKIADYNYKLPDDFT
GCVIAWNSNNLDSKVGGNYNYLYRLFRKSNLKPFERDISTEIYQAGSTPC
NGVEGFNCYFPLQSYGFQPTNGVGYQPYRVVVLSFELLHAPATVCGP (S
EQ ID NO: 19).

In some embodiments, the antigenic coronavirus peptide comprises a variant of SEQ ID NO: 19 that may comprise 1, 2, 3, 4, 5, 6, 7, 8, 9, 10, 11, 12, 13, 14, or 15 or more substitution, deletion, or insertion mutations in SEQ ID NO: 19. In some embodiments, the antigenic coronavirus peptide comprises a variant of SEQ ID NO: 19 that may comprise an amino acid sequence that shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or up to 100% sequence identity or homology with SEQ ID NO: 19. In some embodiments, the antigenic coronavirus peptide comprises a fragment of RBD that may be a fragment of SEQ ID NO: 19 that comprises about 20%, about 25%, about 30%, about 35%, about 40%, about 45%, about 50%, about 55%, about 60%, about 65%, about 70%, about 75%, about 80%, about 85%, about 90%, 91%, 92%, 93%, 94%, 95%, 96%, 97%, 98%, or 99% of the length of SEQ ID NO: 19, so long as the fragment is able to elicit an immune response (i.e., it is an antigenic fragment).

The antigenic coronavirus peptide may comprise a variant of an RBD (e.g., SEQ ID NO: 19) with one or more specific modifications made to reduce “sticky” hydrophobic regions, which may increase expression and/or the ability to form nanoparticles, for example, one of more of the following modifications.

TABLE 2

Exemplary Amino Acid Modifications in SARS-CoV-2 RBD

| Mutation (Location corresponds to location in full length spike; SEQ ID NO: 18) | Corresponding mutation location in SEQ ID NO: 19 | Location and Concept |
|---|---|---|
| F456N/K458T | 126/128 | glycan-ACE2BS |
| L455R+Y449K+F490R | 125+119+160 | ACE2BS |
| L455R | 125 | ACE2BS |
| I468R | 138 | ACE2BS |
| Y453R | 123 | ACE2BS |
| L452R | 122 | ACE2BS |
| L492R | 162 | ACE2BS |
| F490R | 160 | ACE2BS |
| F490A | 160 | ACE2BS |
| 517LLH to 517NKS | 187 | glycan- at the bottom hydrophobic patch |
| L518R | 188 | bottom hydrophobic patch |
| V367T+L335N | 37+5 | bottom greasy areas |
| T385N/L387T | 55/57 | glycan-lower hydrophobic patch (not the one already covered by a glycan) |
| V382R | 52 | lower hydrophobic patch (not the one already covered by a glycan) |
| F377R | 47 | lower hydrophobic patch (not the one already covered by a glycan) |
| K417N | 87 | RBD mutation alters antigenicity |
| E484K | 154 | RBD mutation alters antigenicity |
| N501Y | 171 | RBD mutation alters antigenicity |
| K417T/E484K/N501Y | 87/154/171 | Match to variant B.1.351 |

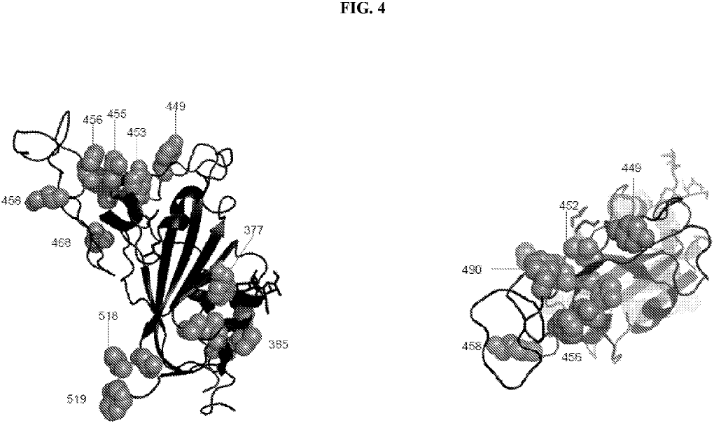

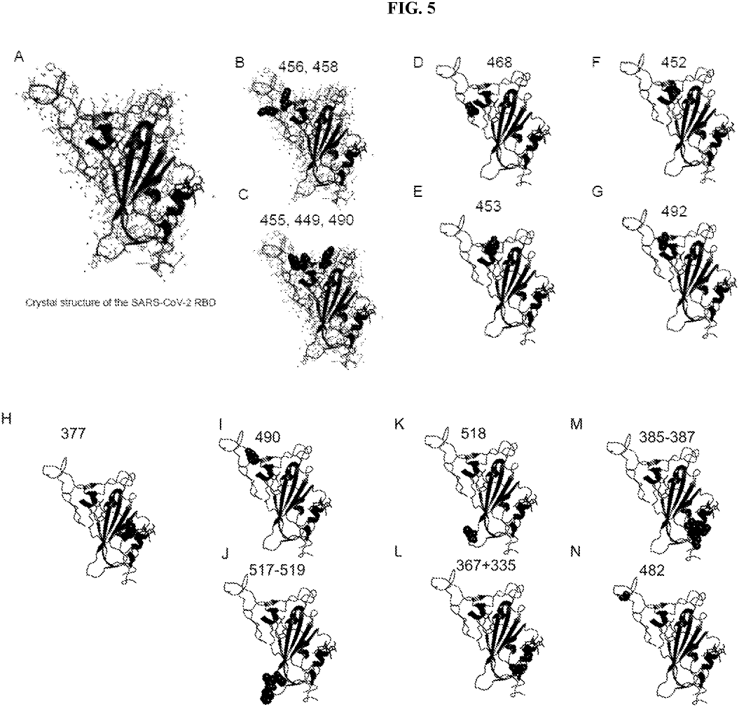

The foregoing modifications may increase the expression and/or nanoparticle formation of fusion proteins comprising an RBD with these modifications. The structure of the SARS-CoV-2 RBD is shown in a ribbon representation with specific residues that may be modified labeled in FIG. 4. The electrostatic potential of SARS-CoV-2 with a hydrophobic can be modified for improved production, stability and yield of the RBD or RBD-Ferritin constructs (see FIG. 50 for a space-filled model showing hydrophobic regions). FIG. 5 further shows variant mutations in the crystal structure of the RBD used to design exemplary ferritin variants with the foregoing modifications.

Additionally or alternatively, with respect to the modifications above, SEQ ID NOs: 308-312, which are also disclosed in Table 20 at the end of the specification, are examples of RBD with mutations at positions that are present in SARS-CopV-2 variants of concern (VOC), including strains B.1.351, B.1.1.7 and P.1, and these sequences include mutations at positions 417, 484, and/or 501 of the SARS-CoV-2 Spike protein. DNA sequences (e.g., plasmids) encoding these VOCs (and/or other coronavirus RBDs, such as SEQ ID NO: 19) can also be used to prime the immune response in a subject prior to administration of a nanoparticle vaccine disclosed herein.

Additionally or alternatively, the antigenic coronavirus peptide may comprise an NTD. An NTD may comprise the SARS-CoV-2 NTD amino acid sequence

QCVNLTTRTQLPPAYTNSFTRGVYYPDKVFRSSVLHSTQDLFLPFFSNVT
WFHAIHVSGTNGTKRFDNPVLPFNDGVYFASTEKSNIIRGWIFGTTLDSK
TQSLLIVNNATNVVIKVCEFQFCNDPFLGVYYHKNNKSWMESEFRVYSSA
NNCTFEYVSQPFLMDLEGKQGNFKNLREFVFKNIDGYFKIYSKHTPINLV
RDLPQGFSALEPLVDLPIGINITRFQTLLALHRSYLTPGDSSSGWTAGAA
AYYVGYLQPRTFLLKYNENGTITDAVDCALDPLSETKCTL

(SEQ ID NO:20). In some embodiments, the antigenic coronavirus peptide comprises a variant of SEQ ID NO: 20 that may comprise 1, 2, 3, 4, 5, 6, 7, 8, 9, 10, 11, 12, 13, 14, or 15 or more substitution, deletion, or insertion mutations in SEQ ID NO: 20. In some embodiments, the antigenic coronavirus peptide comprises a variant of SEQ ID NO: 20 that may comprise an amino acid sequence that shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or up to 100% sequence identity or homology with SEQ ID NO: 20. In some embodiments, the antigenic coronavirus peptide comprises a fragment of NTD that may be a fragment of SEQ ID NO: 20 that comprises about 20%, about 25%, about 30%, about 35%, about 40%, about 45%, about 50%, about 55%, about 60%, about 65%, about 70%, about 75%, about 80%, about 85%, about 90%, 91%, 92%, 93%, 94%, 95%, 96%, 97%, 98%, or 99% of the length of SEQ ID NO: 20, so long as the fragment is able to elicit an immune response (i.e., it is an antigenic fragment).

Additionally or alternatively, the antigenic coronavirus peptide may comprise an S1 protein sequence. An S1 protein sequence may comprise a SARS-CoV-2 S1 protein amino acid sequence

VNLTTRTQLPPAYTNSFTRGVYYPDKVFRSSVLHSTQDLFLPFFSNVTWF
HAIHVSGTNGTKRFDNPVLPFNDGVYFASTEKSNIIRGWIFGTTLDSKTQ
SLLIVNNATNVVIKVCEFQFCNDPFLGVYYHKNNKSWMESEFRVYSSANN
CTFEYVSQPFLMDLEGKQGNFKNLREFVFKNIDGYFKIYSKHTPINLVRD
LPQGFSALEPLVDLPIGINITRFQTLLALHRSYLTPGDSSSGWTAGAAAY
YVGYLQPRTFLLKYNENGTITDAVDCALDPLSETKCTLKSFTVEKGIYQT
SNFRVQPTESIVRFPNITNLCPFGEVFNATRFASVYAWNRKRISNCVADY
SVLYNSASFSTFKCYGVSPTKLNDLCFTNVYADSFVIRGDEVRQIAPGQT
GKIADYNYKLPDDFTGCVIAWNSNNLDSKVGGNYNYLYRLFRKSNLKPFE
RDISTEIYQAGSTPCNGVEGFNCYFPLQSYGFQPTNGVGYQPYRVVVLSF
ELLHAPATVCGPKKSTNLVKNKCVNFNFNGLTGTGVLTESNKKFLPFQQF
GRDIADTTDAVRDPQTLEILDITPCSFGGVSVITPGTNTSNQVAVLYQDV
NCTEVPVAIHADQLTPTWRVYSTGSNVFQTRAGCLIGAEHVNNSYECDIP
IGAGICASYQTQT (SEQ ID NO: 21)

or

QCVNLTTRTQLPPAYTNSFTRGVYYPDKVFRSSVLHSTQDLFLPFFSNVT
WFHAIHVSGTNGTKRFDNPVLPFNDGVYFASTEKSNIIRGWIFGTTLDSK
TQSLLIVNNATNVVIKVCEFQFCNDPFLGVYYHKNNKSWMESEFRVYSSA
NNCTFEYVSQPFLMDLEGKQGNFKNLREFVFKNIDGYFKIYSKHTPINLV
RDLPQGFSALEPLVDLPIGINITRFQTLLALHRSYLTPGDSSSGWTAGAA
AYYVGYLQPRTFLLKYNENGTITDAVDCALDPLSETKCTLKSFTVEKGIY
QTSNFRVQPTESIVRFPNITNLCPFGEVFNATRFASVYAWNRKRISNCVA
DYSVLYNSASFSTFKCYGVSPTKLNDLCFTNVYADSFVIRGDEVRQIAPG
QTGKIADYNYKLPDDFTGCVIAWNSNNLDSKVGGNYNYLYRLFRKSNLKP
FERDISTEIYQAGSTPCNGVEGFNCYFPLQSYGFQPTNGVGYQPYRVVVL
SFELLHAPATVCGPKKSTNLVKNKCVNFNFNGLTGTGVLTESNKKFLPFQ
QFGRDIADTTDAVRDPQTLEILDITPCSFGGVSVITPGTNTSNQVAVLYQ
DVNCTEVPVAIHADQLTPTWRVYSTGSNVFQTRAGCLIGAEHVNNSYECD
IPIGAGICASYQTQTNSPRRAR (SEQ ID NO: 22)

or

SSQCVNLTTRTQLPPAYTNSFTRGVYYPDKVFRSSVLHSTQDLFLPFFSN
VTWFHAIHVSGTNGTKRFDNPVLPFNDGVYFASTEKSNIIRGWIFGTTLD
SKTQSLLIVNNATNVVIKVCEFQFCNDPFLGVYYHKNNKSWMESEFRVYS
SANNCTFEYVSQPFLMDLEGKQGNFKNLREFVFKNIDGYFKIYSKHTPIN
LVRDLPQGFSALEPLVDLPIGINITRFQTLLALHRSYLTPGDSSSGWTAG
AAAYYVGYLQPRTFLLKYNENGTITDAVDCALDPLSETKCTLKSFTVEKG
IYQTSNFRVQPTESIVRFPNITNLCPFGEVFNATRFASVYAWNRKRISNC
VADYSVLYNSASFSTFKCYGVSPTKLNDLCFTNVYADSFVIRGDEVRQIA
PGQTGKIADYNYKLPDDFTGCVIAWNSNNLDSKVGGNYNYLYRLFRKSNL
KPFERDISTEIYQAGSTPCNGVEGFNCYFPLQSYGFQPTNGVGYQPYRVV
VLSFELLHAPATVCGPKKSTNLVKNKCVNFNFNGLTGTGVLTESNKKFLP
FQQFGRDIADTTDAVRDPQTLEILDITPCSFGGVSVITPGTNTSNQVAVL
YQDVNCTEVPVAIHADQLTPTWRVYSTGSNVFQTRAGCLIGAEHVNNSYE
CDIPIGAGICASYQTGGSQSIIAYT (SEQ ID NO: 313)

In some embodiments, the antigenic coronavirus peptide may comprise a variant of SEQ ID NO: 21, SEQ ID NO: 22, or SEQ ID NO: 313 that may comprise 1, 2, 3, 4, 5, 6, 7, 8, 9, 10, 11, 12, 13, 14, or 15 or more substitution, deletion, or insertion mutations in SEQ ID NO: 21, SEQ ID NO: 22, or SEQ ID NO: 313. In some embodiments, the antigenic coronavirus peptide may comprise a variant of SEQ ID NO: 21, SEQ ID NO: 22, or SEQ ID NO: 313 that may comprise an amino acid sequence that shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or up to 100% sequence identity or homology with SEQ ID NO: 21, SEQ ID NO: 22, or SEQ ID NO: 313. In some embodiments, the antigenic coronavirus peptide may comprise a fragment of S1 that may be a fragment of SEQ ID NO: 21, SEQ ID NO: 22, or SEQ ID NO: 313 that comprises about 20%, about 25%, about 30%, about 35%, about 40%, about 45%, about 50%, about 55%, about 60%, about 65%, about 70%, about 75%, about 80%, about 85%, about 90%, 91%, 92%, 93%, 94%, 95%, 96%, 97%, 98%, or 99% of the length of SEQ ID NO: 21, SEQ ID NO: 22, or SEQ ID NO: 313, so long as the fragment is able to elicit an immune response (i.e., it is an antigenic fragment).

Additionally or alternatively, the antigenic coronavirus peptide may comprise an S-2P sequence or a fragment or variant thereof. An S-2P sequence is a stabilized version of the spike ectodomain that includes two proline substitutions and stabilizes the prefusion conformation. Specifically, S-2P comprises proline modifications K986P and V987P, as well as the removal of the Furin cleavage site (RRAS to GSAS). In some embodiments, the antigenic coronavirus peptide may comprise a variant of the S-2P sequence that may comprise 1, 2, 3, 4, 5, 6, 7, 8, 9, 10, 11, 12, 13, 14, or 15 or more substitution, deletion, or insertion mutations in the S-2P sequence. In some embodiments, the antigenic coronavirus peptide may comprise a variant of the S-2P sequence that may comprise an amino acid sequence that shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or up to 100% sequence identity or homology with a stabilized S-2P. In some embodiments, the antigenic coronavirus peptide may comprise a fragment of S-2P that comprises about 20%, about 25%, about 30%, about 35%, about 40%, about 45%, about 50%, about 55%, about 60%, about 65%, about 70%, about 75%, about 80%, about 85%, about 90%, 91%, 92%, 93%, 94%, 95%, 96%, 97%, 98%, or 99% of the stabilized S-2P, so long as the fragment is able to elicit an immune response (i.e., it is an antigenic fragment).

Additionally or alternatively, the antigenic coronavirus peptide may comprise an extracellular spike S domain (e.g., a stabilized extracellular spike S domain) or a fragment or variant thereof. A stabilized extracellular spike S domain may comprise one or more modifications to stabilize the refusion conformation of the extracellular domain. In some embodiments, the antigenic coronavirus peptide may comprise a stabilized extracellular spike S domain that comprises 1, 2, 3, 4, 5, 6, 7, 8, 9, 10, 11, 12, 13, 14, or 15 or more substitution, deletion, or insertion mutations in an extracellular spike S domain. In some embodiments, the antigenic coronavirus peptide may comprise a stabilized extracellular spike S domain that comprises an amino acid sequence that shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or up to 100% sequence identity or homology with an extracellular spike S domain. In some embodiments, the antigenic coronavirus peptide may comprise a fragment of the extracellular spike S domain (e.g., a fragment of a stabilized extracellular spike S domain) that comprises about 20%, about 25%, about 30%, about 35%, about 40%, about 45%, about 50%, about 55%, about 60%, about 65%, about 70%, about 75%, about 80%, about 85%, about 90%, 91%, 92%, 93%, 94%, 95%, 96%, 97%, 98%, or 99% of extracellular spike S domain (e.g., a stabilized extracellular spike S domain), so long as the fragment is able to elicit an immune response (i.e., it is an antigenic fragment).

Additionally or alternatively, the antigenic coronavirus peptide may comprise an extracellular spike S trimer (e.g., a stabilized extracellular spike S trimer) or a fragment or variant thereof. A stabilized extracellular spike S trimer may comprise one or more modifications to stabilize the refusion conformation of the extracellular trimer. In some embodiments, the antigenic coronavirus peptide may comprise a stabilized extracellular spike S trimer that comprises 1, 2, 3, 4, 5, 6, 7, 8, 9, 10, 11, 12, 13, 14, or 15 or more substitution, deletion, or insertion mutations in an extracellular spike S trimer. In some embodiments, the antigenic coronavirus peptide may comprise a stabilized extracellular spike S trimer that comprises an amino acid sequence that shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or up to 100% sequence identity or homology with an extracellular spike S trimer. In some embodiments, the antigenic coronavirus peptide may comprise a fragment of the extracellular spike S trimer (e.g., a fragment of a stabilized extracellular spike S trimer) that comprises about 20%, about 25%, about 30%, about 35%, about 40%, about 45%, about 50%, about 55%, about 60%, about 65%, about 70%, about 75%, about 80%, about 85%, about 90%, 91%, 92%, 93%, 94%, 95%, 96%, 97%, 98%, or 99% of extracellular spike S trimer (e.g., a stabilized extracellular spike S trimer), so long as the fragment is able to elicit an immune response (i.e., it is an antigenic fragment).

Additionally or alternatively, the antigenic coronavirus peptide may comprise a stabilized variant with six prolines (i.e., “Hexapro”), which is another variant of the spike protein that comprises F817P, A892P, A899P, and A942P substitutions in addition to the two proline substitutions of S-2P. In some embodiments, the antigenic coronavirus peptide may comprise a variant of Hexapro that may comprise 1, 2, 3, 4, 5, 6, 7, 8, 9, 10, 11, 12, 13, 14, or 15 or more substitution, deletion, or insertion mutations in a Hexapro. In some embodiments, the antigenic coronavirus peptide may comprise a variant of Hexapro that may comprise an amino acid sequence that shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or up to 100% sequence identity or homology with a Hexapro. In some embodiments, the antigenic coronavirus peptide may comprise a fragment of Hexapro that comprises about 20%, about 25%, about 30%, about 35%, about 40%, about 45%, about 50%, about 55%, about 60%, about 65%, about 70%, about 75%, about 80%, about 85%, about 90%, 91%, 92%, 93%, 94%, 95%, 96%, 97%, 98%, or 99% of the Hexapro, so long as the fragment is able to elicit an immune response (i.e., it is an antigenic fragment).

Additionally or alternatively, the antigenic coronavirus peptide may comprise a SARS-CoV-1 spike protein (S protein) or a fragment or variant thereof. The SARS-CoV-1 spike protein may comprise the amino acid sequence set forth below:

SDLDRCTTFDDVQAPNYTQHTSSMRGVYYPDEIFRSDTLYLTQDLFLPFY
SNVTGFHTINHTFGNPVIPFKDGIYFAATEKSNVVRGWVFGSTMNNKSQS
VIIINNSTNVVIRACNFELCDNPFFAVSKPMGTQTHTMIFDNAFNCTFEY
ISDAFSLDVSEKSGNFKHLREFVFKNKDGFLYVYKGYQPIDVVRDLPSGF
NTLKPIFKLPLGINITNFRAILTAFSPAQDIWGTSAAAYFVGYLKPTTFM
LKYDENGTITDAVDCSQNPLAELKCSVKSFEIDKGIYQTSNFRVVPSGDV
VRFPNITNLCPFGEVFNATKFPSVYAWERKKISNCVADYSVLYNSTFFST
FKCYGVSATKLNDLCFSNVYADSFVVKGDDVRQIAPGQTGVIADYNYKLP
DDFMGCVLAWNTRNIDATSTGNYNYKYRYLRHGKLRPFERDISNVPFSPD
GKPCTPPALNCYWPLNDYGFYTTTGIGYQPYRVVVLSFELLNAPATVCGP
KLSTDLIKNQCVNFNFNGLTGTGVLTPSSKRFQPFQQFGRDVSDFTDSVR
DPKTSEILDISPCAFGGVSVITPGTNASSEVAVLYQDVNCTDVSTAIHAD
QLTPAWRIYSTGNNVFQTQAGCLIGAEHVDTSYECDIPIGAGICASYHTV
SLLRSTSQKSIVAYTMSLGADSSIAYSNNTIAIPTNFSISITTEVMPVSM
AKTSVDCNMYICGDSTECANLLLQYGSFCTQLNRALSGIAAEQDRNTREV
FAQVKQMYKTPTLKYFGGFNFSQILPDPLKPTKRSFIEDLLFNKVTLADA
GFMKQYGECLGDINARDLICAQKFNGLTVLPPLLTDDMIAAYTAALVSGT
ATAGWTFGAGAALQIPFAMQMAYRFNGIGVTQNVLYENQKQIANQFNKAI
SQIQESLTTTSTALGKLQDVVNQNAQALNTLVKQLSSNFGAISSVLNDIL
SRLDPPEAEVQIDRLITGRLQSLQTYVTQQLIRAAEIRASANLAATKMSE
CVLGQSKRVDFCGKGYHLMSFPQAAPHGVVFLHVTYVPSQERNFTTAPAI
CHEGKAYFPREGVFVFNGTSWFITQRNFFSPQIITTDNTFVSGNCDVVIG
IINNTVYDPLQSELDSIKEELDKIHKN

(SEQ ID NO: 314). In some embodiments, the antigenic coronavirus peptide comprises a variant of SEQ ID NO: 314 that may comprise 1, 2, 3, 4, 5, 6, 7, 8, 9, 10, 11, 12, 13, 14, or 15 or more substitution, deletion, or insertion mutations in SEQ ID NO: 314. In some embodiments, the antigenic coronavirus peptide comprises a variant of SEQ ID NO: 314 that may comprise an amino acid sequence that shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or up to 100% sequence identity or homology with SEQ ID NO: 314. In some embodiments, the antigenic coronavirus peptide comprises a fragment of a SARS-CoV-1 spike protein that may be a fragment of SEQ ID NO: 314 that comprises about 20%, about 25%, about 30%, about 35%, about 40%, about 45%, about 50%, about 55%, about 60%, about 65%, about 70%, about 75%, about 80%, about 85%, about 90%, 91%, 92%, 93%, 94%, 95%, 96%, 97%, 98%, or 99% of the length of SEQ ID NO: 314, so long as the fragment is able to elicit an immune response (i.e., it is an antigenic fragment).

### D. Fusion Proteins and Vaccine Nanoparticles

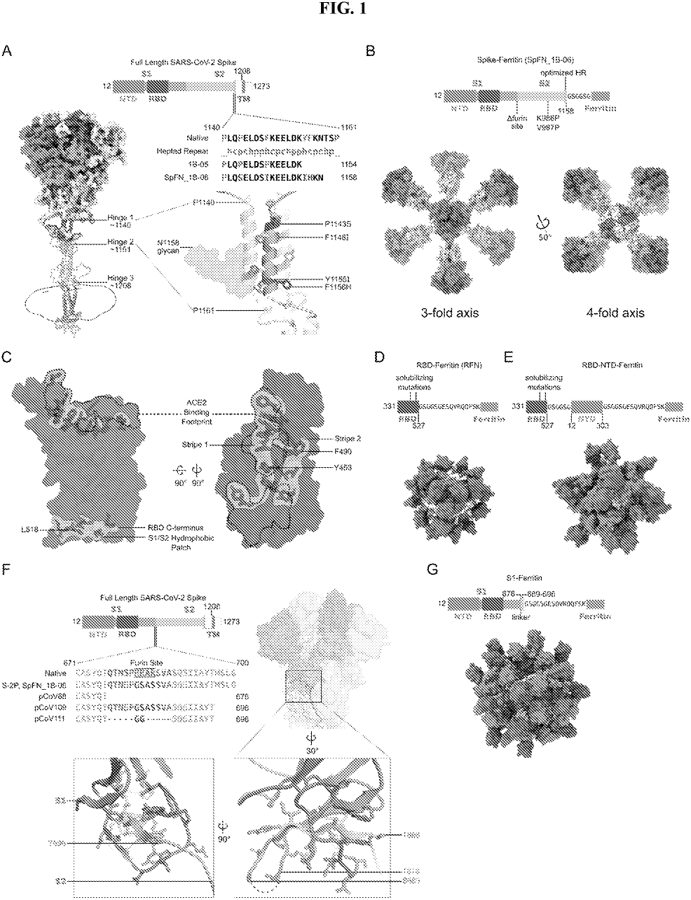

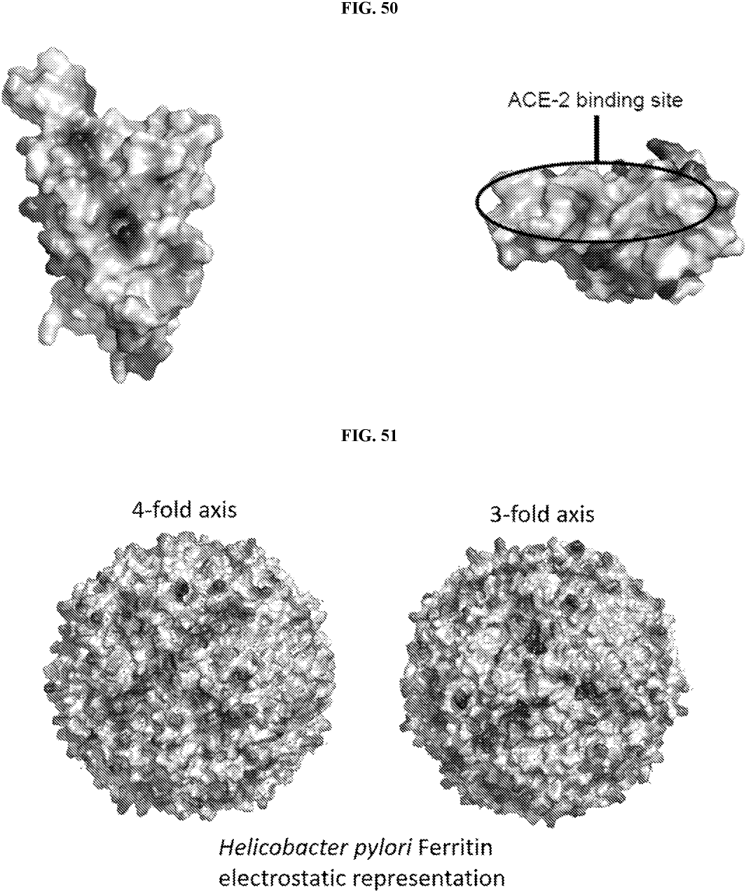

The disclosed vaccine nanoparticles are made up of a plurality of fusion proteins that self-assemble into a nanoparticle. As noted above, the fusion proteins comprise a nanoparticle-forming peptide, which may be an H. pylori ferritin protein or a fragment or variant thereof. Ferritin is a naturally occurring protein that self-assembles into a 24-member spherical particle, made up of multiple three-fold, four-fold, and/or two-fold axes. Thus, the nanoparticle may comprise a 3-fold axis, a 4-fold axis, or a 2-fold axis. Using the 3-fold axes, 8 antigenic trimeric coronavirus peptides can be presented on the surface of the self-assembling protein nanoparticle surface. In the case of monomeric antigens such as the RBD, 24 coronavirus peptides can be presented on the surface of the self-assembling protein nanoparticle surface. Space-filling models of exemplary Spike Ferritin nanoparticles comprising a 4-fold axis and a 3-fold axis are shown in FIG. 1B (see also FIG. 51), and other SARS-CoV-2 Ferritin nanoparticles are shown in FIG. 1.

The antigenic coronavirus peptide component of the disclosed fusion proteins may comprise 1, 2, or 3 or more distinct domains or parts, which may be selected from the exemplary antigenic peptides discussed above. Typically, but not exclusively, a vaccine against a given coronavirus will include antigenic peptides of that coronavirus. For example, typically, but not exclusively, a vaccine against SARS-CoV-2 will include antigenic peptides from SARS-CoV-2, and typically, but not exclusively, a vaccine against SARS-CoV-1 will include antigenic peptides from SARS-CoV-1 (etc.). For example, in some embodiments the antigenic coronavirus peptide my comprise a single domain selected from a RBD, a NTD, a full spike protein, an S1 subunit, an S2 subunit, a stabilized extracellular spike S-2P domain, a stabilized extracellular spike S domain, a stabilized extracellular spike S-trimer, and variants or fragments thereof. Alternatively, the antigenic coronavirus peptide my comprise a combination of two domains, such as two domains selected from a RBD, a NTD, a full spike protein, an S1 subunit, an S2 subunit, a stabilized extracellular spike S-2P domain, a stabilized extracellular spike S domain, a stabilized extracellular spike S-trimer, a Hexapro, and variants or fragments thereof. Alternatively, the antigenic coronavirus peptide my comprise a combination of three domains, such as three domains selected from a RBD, a NTD, a full spike protein, an S1 subunit, an S2 subunit, a stabilized extracellular spike S-2P domain, a stabilized extracellular spike S domain, a stabilized extracellular spike S-trimer, a Hexapro, and variants or fragments thereof.

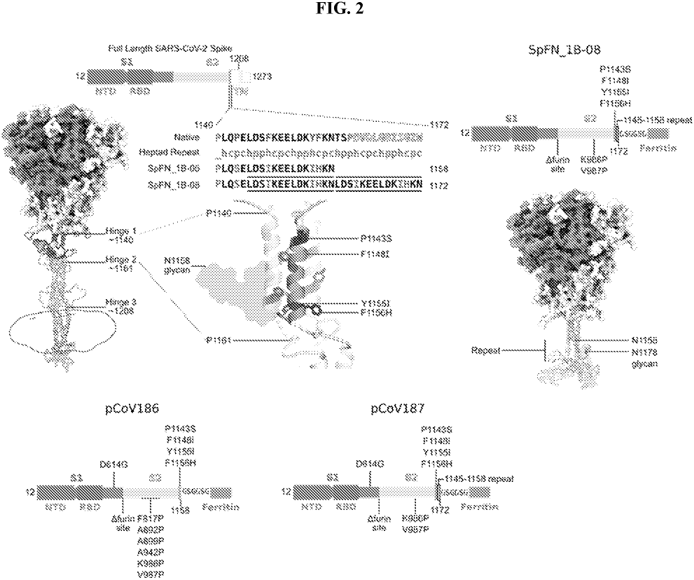

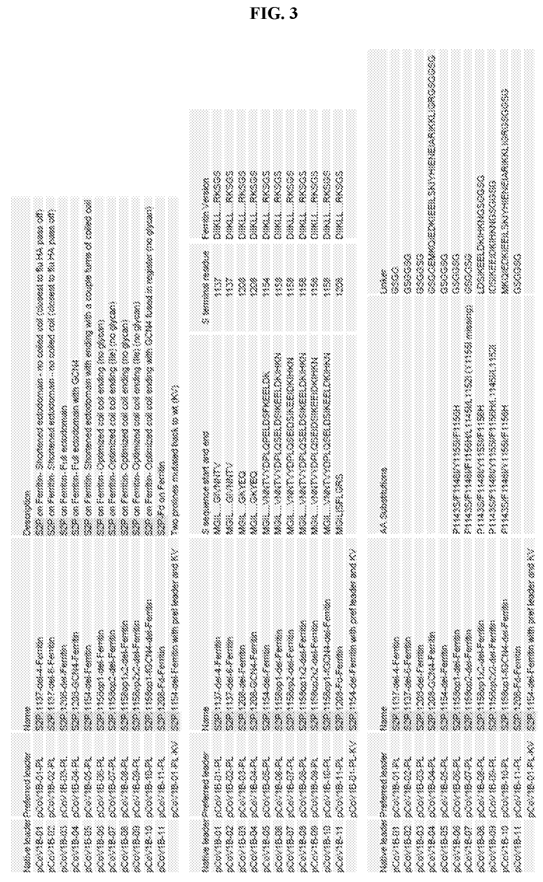

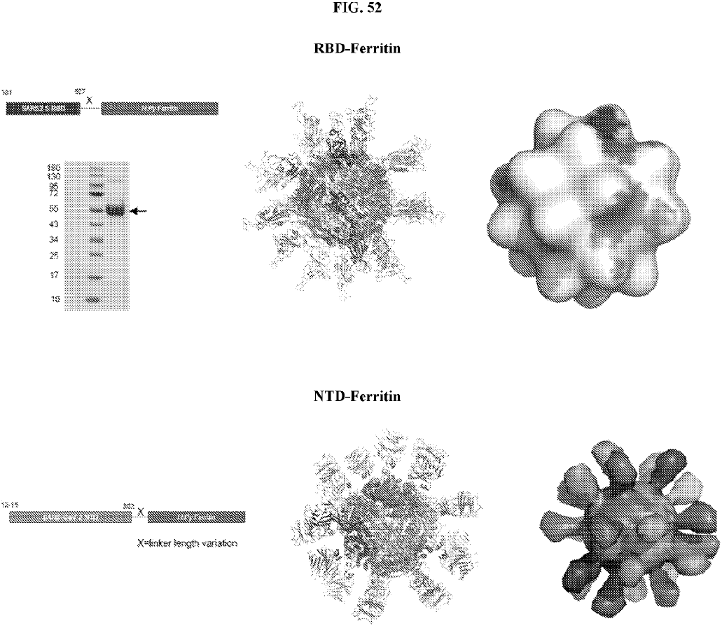

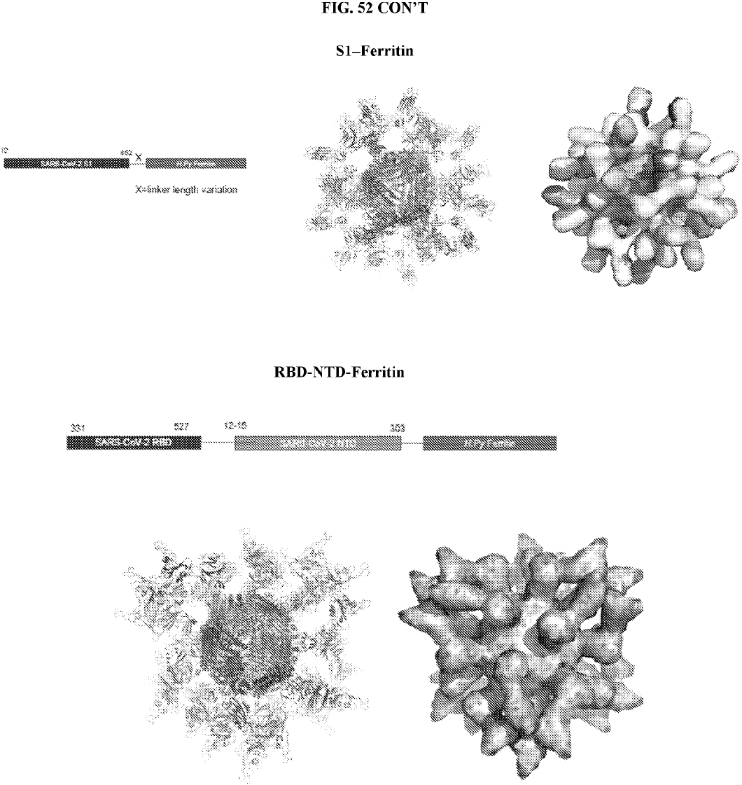

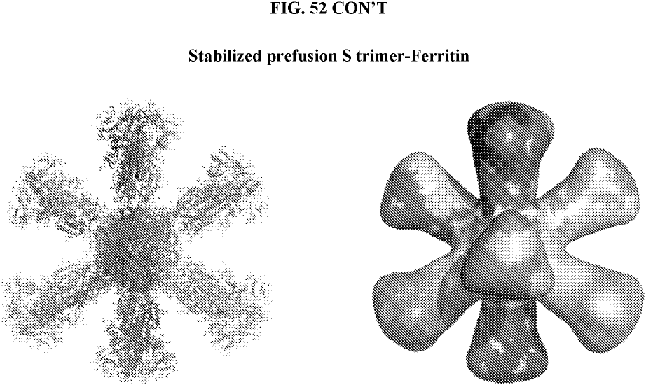

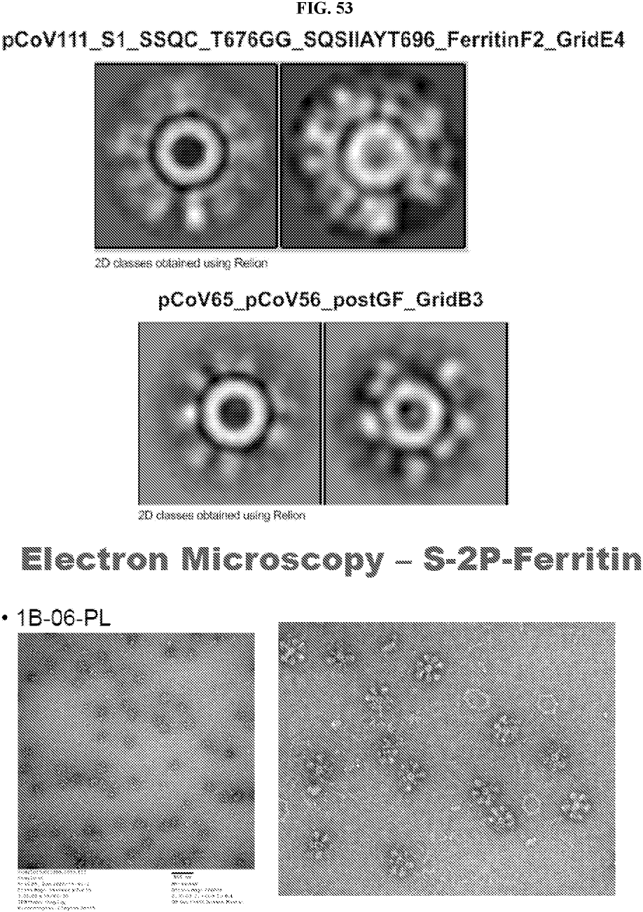

Exemplary fusion proteins include, but are not limited to, a fusion protein comprising (1) a RBD and ferritin, (2) a NTD and ferritin, (3) S1 and ferritin, (4) RBD-NTD and ferritin, and (5) a stabilized prefusion S trimer and ferritin. Ribbon and space-filled representations of these exemplary fusion proteins and the particles that they form are provided in FIGS. 1 and 2 (see also FIG. 52). Sequence information related to the stabilized coiled-coil region and linker sequence for select stabilized prefusion S trimer-Ferritin constructs are provided in FIG. 3 The following Table 3 discloses exemplary vaccine particles that fall into each of the foregoing five categories, and the sequences of exemplary fusion proteins making up each of these particles and others are provided in Table 18 at the end of the specification.

TABLE 3

Exemplary Nanoparticles

| | | | | | |
|---|---|---|---|---|---|
| RBD-Ferritin | NTD-Ferritin | S1-Ferritin | RBD-NTD-Ferritin | S Trimer-Ferritin | |
| pCoV50 | pCoV65 | pCoV111 | pCoV122 | pCoV1B-01 | pCoV186 |
| pCoV58 | pCoV23 | pCoV109 | pCoV125 | pCoV1B-02 | pCoV187 |
| pCoV59 | | pCoV110 | pCoV146 | pCoV1B-03 | pCoV1B-08 |
| pCoV127 | | pCoV112 | pCoV147 | pCoV1B-04 | pCoV1B-09 |
| pCoV129 | | | | pCoV1B-05 | pCoV1B-10 |
| pCoV130 | | | | pCoV1B-06 (SpFN_1B-06) | pCoV1B-11 |
| pCoV131 | | | | pCoV1B-07 | pCoV1B-01-PL-KV |

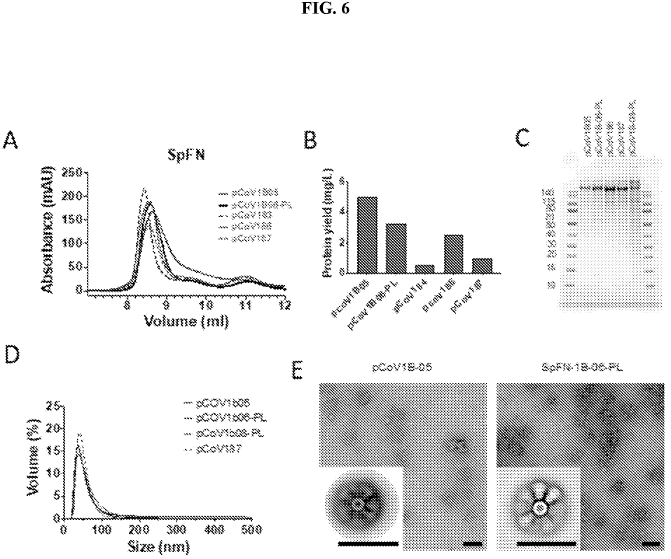

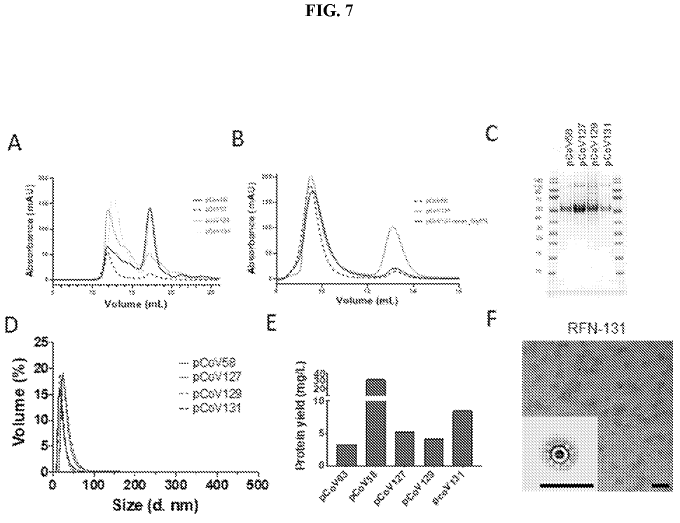

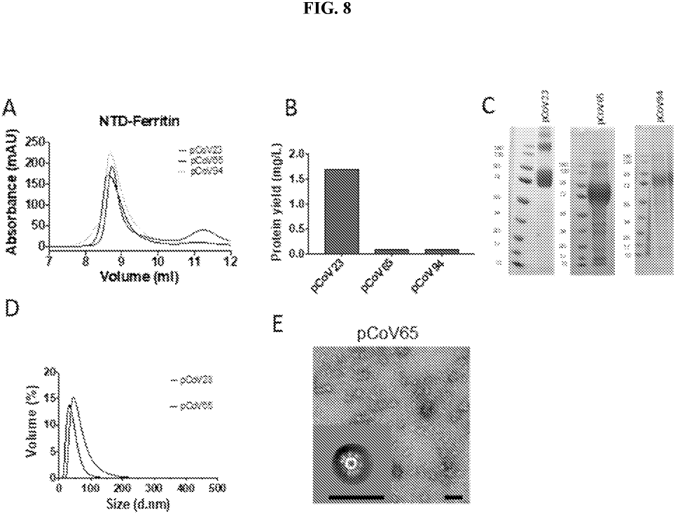

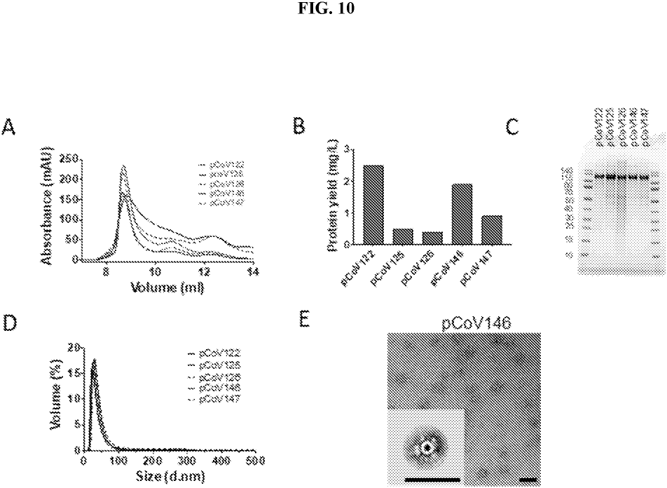

Biochemical and biophysical characterization of select nanoparticles are shown in FIGS. 6-10 including size-exclusion profiles, expression levels, SDS-PAGE, dynamic light scattering and negative-stain transmission electron microscopy.

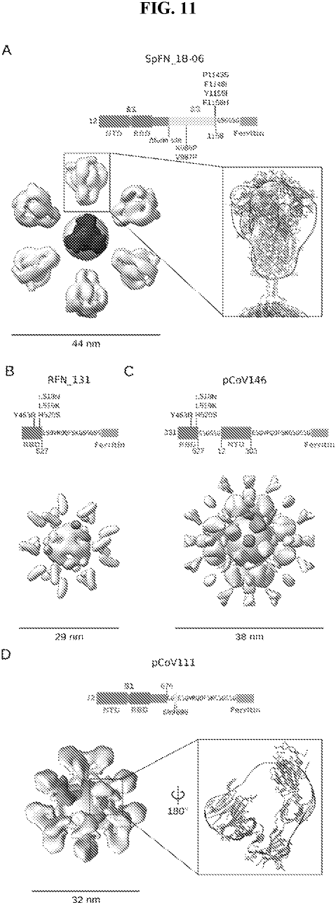

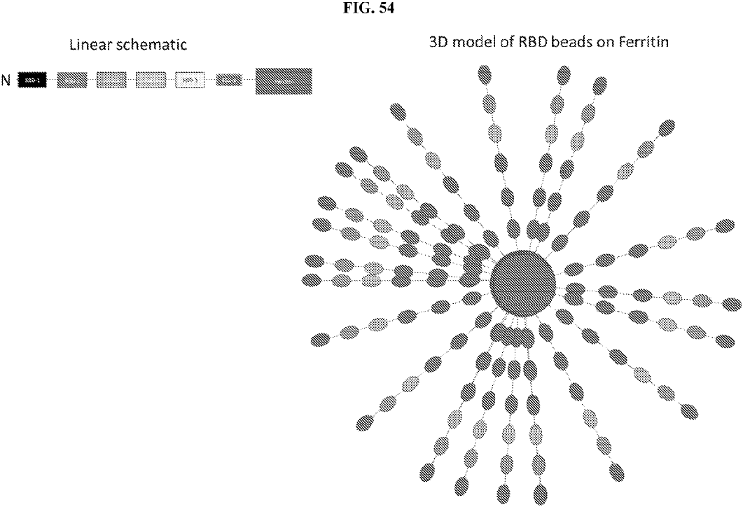

Negative-stain electron microscopy 3-dimernsional reconstructions for select nanoparticles are shown in FIG. 11. TEM images of select nanoparticles are shown in FIG. 53.

Nanoparticles as disclosed herein may bind to a human ACE-2 receptor. Nanoparticles as disclosed herein may bind to anti-coronavirus spike protein antibodies including, but not limited to, CR3022.

The disclosed fusion proteins that self-assemble into the disclosed nanoparticles, including the nanoparticles described in Table 3 above and the fusion protein disclosed in Table 18 below, can be expressed alone or co-expressed (e.g., on two different plasmids) in suitable expression systems, which may include mammalian or eukaryotic expression systems. Some of the fusion proteins disclosed in Table 18 may comprise a histidine tag (i.e., His tag), which comprises a repeat of 5-10 histidine (H) residues or other tag sequences that may be useful in processing or purifying the protein, but which may ultimately be cleaved from the active protein before nanoparticle assembly. For example, pCoV223 (SEQ ID NO: 301) encodes a sequence with a N-terminal His-tag to allow purification of the Spike-Ferritin molecule.

All of the proteins disclosed in Table 18 are exemplary nanoparticle-forming proteins that can form Spike-Ferritin nanoparticles. With respect to SEQ ID NOs: 284-301, which are disclosed in Table 18, these sequences contain a set of alternate sequences to improve the stability and immunogenicity of the Spike-Ferritin constructs. This includes a stabilizing disulfide bond, a D614G mutation, a mutation to remove a glycan in the Spike at N165 to enable the RBD greater freedom of motion and allow the RBD to sit in the “up” and more exposed conformation, and a N234Q mutation to remove a glycan at 234 in the Spike to allow the RBD to sit in a more closed conformation. Additionally or alternatively a glycan at N146 or N77 in the Ferritin sequence will improve and stabilize the Ferritin molecule.

SEQ ID NOs: 302 -307, which are also disclosed in Table 18, are examples of nanoparticles that comprise multiple RBDs connected to a single ferritin molecule contained within a single construct (see, e.g., FIG. 54). The RBDs are arranged analogously to “beads on a string,” which allows multiple antigenic components to be assembled using a single gene insert for production. This concept builds on the results seen with the RBD-NTD-Ferritin constructs (e.g., FIG. 52) such as pCoV146 (SEQ ID NO: 136) where a RBD and NTD are attached sequentially in tandem to a ferritin molecule to allow simple expression of both components.

The “beads on a string” concept can be used to create a nanoparticle with antigenic components from multiple coronaviruses such as SARS-CoV-2, SARS-CoV-1, HKU-1, MERS-CoV, 229E, NL63, OC43, or related coronaviruses including those identified from bats, camels, or pangolins. These embodiments can be utilized to create a pan-β-coronavirus vaccine, or pan-coronavirus vaccine. For example, multiple RBD “beads” comprised of different antigenic sequences can be provided together on a single “string” (i.e., in a single construct) to elicit broad immune responses against coronaviruses. For example, a “string” of antigens such as SARS-CoV-2-RBD-SARS-CoV-1-RBD-HKU-1-RBD-MERS-CoV-RBD-229E-RBD-NL63-RBD could be used with a “string” of antigens such as SARS-CoV-2-RBD-pangolinSARS-CoV-1-RBD-OC43-RBD-camelMERS-CoV-RBD-229E-RBD-NL63-RBD to increase or focus the immune response to a specific pan-reactive or pan-protective immunity. The “beads on a string” may comprise, for example, 2-10 RBD sequences in series, or, in other words, may comprise 2, 3, 4, 5, 6, 7, 8, 9, or 10 RBDs. In accordance with these embodiments, RBD-Ferritin sequences, e.g., pCoV127 (SEQ ID NO: 125 or 194) or pCOV131 (SEQ ID NO: 129 or 198), may serve as a base sequence and additional RBD sequences can be added to the N-terminus. Alternatively, RBD-NTD-Ferritin sequences, e.g., pCoV146 (SEQ ID NO: 136), may serve as a base sequence and additional RBD sequences can be added to the N-terminus. A linker sequence, including but not limited to the linker sequences disclosed in Table 1, may link one or more or each of the RBD sequences in series.

Any of the fusion proteins, nanoparticles, and vaccines disclosed herein can be used for treating or preventing a coronavirus infection, such as SARS-CoV-2 infection (e.g., COVID-19) or SARS-CoV-1 infection, for example. Optimal doses and routes of administration may vary.

The disclosed fusion proteins and nanoparticles can be combined with an adjuvant to improve immune responses and promote protective responses, as discussed in more detail in the following section.

### E. Vaccine Adjuvant

An adjuvant is an ingredient used in some vaccines that helps create a stronger immune response in people receiving the vaccine. Adjuvants help the body to produce an immune response strong enough to protect the person from the disease he or she is being vaccinated against.

The present disclosure provides vaccine formulations that contain any of (or a combination of) the disclosed nanoparticles and at least one adjuvant selected from the group consisting of ALFQ, alhydrogel, and combination thereof.

The adjuvant ALFQ was developed by the U.S. Army, and is an Army-Liposome-Formulation (ALF) containing high amounts of cholesterol together with the QS21 saponin (ALFQ). ALFQ has been used in numerous animal studies and with a variety of immunogens, and has shown effectiveness in eliciting robust immune responses. In contrast to some adjuvants, ALFQ tends to elicit a balanced Th1/Th2 immune response avoiding a skewed immune response that has been implicated in vaccine associated enhanced respiratory disease (VAERD). In some embodiments, the ALFQ adjuvant is a liposomal formulation containing monophosphoryl lipid A (MPLA) and QS-21 saponin. In some embodiments, the ALFQ liposomes may contain about 600 µg/mL monophosphoryl 3-deacyl lipid A (3D-PHAD) and about 300 µg/mL QS-21. To make the ALFQ, in one exemplary embodiment, 14.7 mL of ALF55 (containing 1.236 mg/mL 3D-PHAD) may be diluted with 6.5 mL of isotonic Sorensen’s PBS pH 6.15 in a sterile glass vial and adding 9.08 mL of QS-21 (1 mg/mL) to the diluted ALF55 while slowly stirring.

Alhydrogel refers to a range of aluminum hydroxide gel products which have been specifically developed for use as an adjuvant in human and veterinary vaccines. The gel is a suspension of boehmite-like (aluminium oxyhydroxide) hydrated nano/micron size crystals in loose aggregates. The products have very low conductivity due to the absence of buffering ions. They have a positive charge at neutral pH and effectively adsorb negatively charged antigens. The primary purpose of the adjuvant in vaccines is to boost the antibody-mediated (Th2) immune response to the antigens. Alhydrogel products can be combined with other adjuvant types (such as monophosphoryl lipids) to achieve a balanced Th1/Th2 immune response. For the purposes of formulating the disclosed vaccines, an alhydrogel stock may be diluted before combining with the disclosed nanoparticles such that the concentration of the aluminum is about 500 µg/ml, about 550 µg/ml, about 600 µg/ml, about 650 µg/ml, about 700 µg/ml, about 750 µg/ml, about 800 µg/ml, about 850 µg/ml, about 900 µg/ml, about 950 µg/ml, about 1000 µg/ml, about 1050 µg/ml, about 1100 µg/ml, about 1150 µg/ml, about 1200 µg/ml, about 1250 µg/ml, about 1300 µg/ml, about 1350 µg/ml, about 1400 µg/ml, about 1450 µg/ml, or about 1500 µg/ml, or more.

Other vaccine adjuvants are known in the art, and based on the results reported herein with respect to ALFQ, and Alhydrogel, those of skill in the art will understand that other adjuvants also could be used with and complement the function of the disclosed nanoparticles. Other adjuvants that are suitable for use with the disclosed nanoparticles include, but are not limited to, monophosphoryl lipid A (MPLA), oil in water emulsions, ADJUPLEX™ (a lecithin and carbomer homopolymer), ADDAVAX™ (a squalene-based oil-in-water nano-emulsion), CARBOPOL® polymers (crosslinked polyacrylic acid polymers), Poly IC:LC (a synthetic complex of carboxymethylcellulose, polyinosinic-polycytidylic acid, and poly-L-lysine double-stranded RNA), PolyI:C (polyinosinic:polycytidylic acid), CpG oligodeoxynucleotides, Flagellin, Iscomatrix (comprised of saponin, cholesterol, and dipalmitoylphosphatidylcholine), virosomes, MF59 (a squalene-based oil-in-water emulsion), AS03 (a squalene-based oil-in-water emulsion), and AS04 (alum-absorbed 3-0-desacyl-4′-monophosphoryl lipid A), among others.

### F. Pharmaceutical Compositions

Pharmaceutical compositions of the present disclosure include vaccines comprising nanoparticles as disclosed herein. In general, the pharmaceutical compositions will also comprise an adjuvant (e.g., ALFQ, alhydrogel, or a combination thereof). The nanoparticle(s), alone or in combination with one or more adjuvants, may be formulated into a suitable carrier to form a pharmaceutical composition suitable for the intended route of administration.

In some embodiments, the pharmaceutical composition is formulated for systemic administration via parenteral delivery. Parenteral administration includes intravenous, intra-arterial, subcutaneous, intradermal, intraperitoneal, or intramuscular injection or infusion. Formulations for parenteral administration may include sterile aqueous solutions, which may also contain buffers, diluents and other pharmaceutically acceptable additives known to the skilled artisan. For intravenous use, the total concentration of solutes may be controlled to render the preparation isotonic. Intravenous, intra-arterial, subcutaneous, or intramuscular injection are preferred routes of administration. Additionally or alternatively, the disclosed vaccines can be formulated for intranasal administration or administration via contact with another mucosa membrane.

Pharmaceutical compositions for injection may be presented in unit dosage form, e.g., in ampules, or in multi-dose containers, optionally with an added preservative. The compositions may take such forms as suspensions, solutions or emulsions in oily or aqueous vehicles, and may contain formulatory agents such as suspending, stabilizing and/or dispersing agents. The disclosed vaccines may formulated using any suitable pharmaceutically acceptable excipients.

Pharmaceutical compositions for intranasal administration may take the form of liquid dispersions, suspensions, solutions, or emulsions and may be incorporated into a nasal aerosol or nasal spray. Such compositions may contain formulatory agents such as suspending, stabilizing and/or dispersing agents, and may formulated using any suitable pharmaceutically acceptable excipients. Intranasal administration includes administration via the nose, either with or without concomitant inhalation during administration. Such administration is typically through contact of a disclosed vaccine with the nasal mucosa, nasal turbinates, or sinus cavity. Administration by inhalation may comprise intranasal administration, or may include oral inhalation. Such administration may also include contact with the oral mucosa, bronchial mucosa, and other epithelia.

The disclosed vaccines may be formulated to be administered concurrently with another therapeutic agent. The vaccines may be formulated to be administered in sequence with another therapeutic agent. For example, the vaccine may be administered either before or after the subject has received a regimen of an anti-viral therapy.

Any of the pharmaceutical compositions disclosed herein can be used for treating or preventing a coronavirus infection, such as SARS-CoV-2 infection (e.g., COVID-19) or SARS-CoV-1 infection, for example. A pharmaceutical composition for use against a specific coronavirus infection (such as SARS-CoV-2), typically will include antigenic peptides of the target coronavirus (e.g., SARS-CoV-2), but optionally additionally or alternatively may include antigenic peptides of a closely related coronavirus (such as SARS-CoV-1). Optimal doses and routes of administration may vary.

### IV. Treatment and Prevention of Coronavirus Infection

The present disclosure provides methods of treatment and prevention of coronavirus infections, such as but not limited to SARS-CoV-2 infections (e.g., COVID-19) by administering a vaccine comprising one or more of the nanoparticles disclosed herein. The present disclosure also provides uses of the disclosed vaccines and pharmaceutical compositions for treating or preventing coronavirus infections, such as SARS-CoV-2 infections (e.g., COVID-19). In accordance with any methods and uses disclosed herein, the subject may be at risk of a coronavirus infection or may already be infected with a coronavirus. Methods targeting a specific coronavirus infection (such as SARS-CoV-2), typically will use a vaccine or pharmaceutical composition that includes antigenic peptides of the target coronavirus (e.g., SARS-CoV-2), but optionally additionally or alternatively may include antigenic peptides of a closely related coronavirus (such as SARS-CoV-1).

The disclosed methods comprise administering to a subject an effective amount of one or more of the vaccines or pharmaceutical compositions disclosed herein. Administration may be performed via intravenous, intra-arterial, intramuscular, subcutaneous, or intradermal injection. In some embodiments, the subject may be at risk of exposure to a coronavirus, such as SARS-CoV-2 or SARS-CoV-1, for example. In some embodiments, the subject may have previously been exposed to a coronavirus, such as SARS-CoV-2 or SARS-CoV-1. In some embodiments, the subject may have an active infection (e.g., COVID-19) which may be treated as a result of the administration. In some embodiments, the administration of the vaccine prevents the subject from developing a coronavirus infection (e.g., COVID-19). The methods can further include administration of a priming agent (i.e., “primer”) for the nanoparticle vaccine. The primer can be administered prior to the administration of the nanoparticle vaccine (e.g., 1 week, 2 weeks, 3 weeks, 4 weeks, 5 weeks, or 6 or more weeks prior) and the primer may comprise a nucleic acid (i.e., DNA or mRNA) that encodes a fusion protein or all, a fragment, or a variant of the RBD of a coronavirus S protein (e.g., the S protein of SARS-CoV-2 or SARS-CoV-1).

For the purposes of the disclosed methods and uses, treatment and/or prevention of infection by all strains and variants of SAR-CoV-2 are specifically contemplated, including treatment and/or prevention of B.1.1.7, B.1.351, and P1. Also contemplated are methods and uses for treatment and/or prevention of infection by all strains and variants of SARS-CoV-1, and all strains and variants of other coronaviruses disclosed herein.

Dosage regimens can be adjusted to provide the optimum desired response (e.g., production of antibodies and/or cytokines against a coronavirus such as SAR-CoV-2 or SARS-CoV-1, for example). For example, in some embodiments, a single bolus of vaccine may be administered, while in some embodiments, several doses may be administered over time, or the dose may be proportionally reduced or increased as indicated by the situation. For example, in some embodiments the disclosed vaccines may be administered once or twice weekly, once or twice monthly, once every week, once every other week, once every three weeks, once every four weeks, once every other month, once every three months, once every four months, once every five months, once every six months, once every seven weeks, once every eight weeks, once every three months, once every four months, once every five months, once every six months, or once a year. In some embodiments, a subject may be administered an initial dose and then receive one or more booster doses with a predefined span of time in between each dose (e.g., 1, 2, 3, or 4 weeks, or 1, 2, 3, 4, 5, 6, 9, or 12 months). In some embodiments, a subject may receive only a single dose. In some embodiments, a subject may receive an initial dose followed by one or more subsequent doses of an equal or lesser concentration at a set time after this initial dose, such as 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, or 20 or more weeks, such as 24 weeks, 52 weeks, 104 weeks, 260 weeks, or 520 weeks.

Doses may likewise by adjusted to provide the optimum desired response. For example, in some embodiments, a dose of the disclosed vaccines may comprise 1 µg to 50 mg of vaccine. A single does may comprise about 1 µg, about 5 µg, about 10 µg, about 15 µg, about 20 µg, about 25 µg, about 30 µg, about 35 µg, about 40 µg, about 45 µg, about 50 µg, about 55 µg, about 60 µg, about 65 µg about 70 µg, about 75 µg, about 80 µg, about 85 µg, about 90 µg, about 95 µg, about 100 µg, about 125 µg, about 150 µg, about 175 µg, about 200 µg, about 225 µg, about 250 µg, about 275 µg, about 300 µg, about 325 µg, about 350 µg, about 375 µg, about 400 µg, about 425 µg, about 450 µg, about 475 µg, about 500 µg, about 525 µg, about 550 µg, about 575 µg, about 600 µg, about 625 µg, about 650 µg, about 675 µg, about 700 µg, about 725 µg, about 750 µg, about 775 µg, about 100 µg, about 825 µg, about 850 µg, about 875 µg, about 900 µg, about 925 µg, about 950 µg, about 975 µg, about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 5.75 mg, about 5 mg, about 10 mg, about 15 mg, about 20 mg, about 25 mg, about 30 mg, about 35 mg, about 45 mg, or about 50 mg. In some embodiments, a single dose may comprise 4 mg or less of the vaccine or nanoparticle.

Alternatively, dosing may be based on the number of nanoparticles administered to a subject. For example, in some embodiments, a dose of the disclosed vaccines may comprise 1.0 × 10⁸ to 1.0 × 10¹² nanoparticles. For example, a single dose may comprise 1.0 × 10⁸, 1.5 × 10⁸, 2.0 × 10⁸, 2.5 × 10⁸, 3.0 × 10⁸, 3.5 × 10⁸, 4.0 × 10⁸, 4.5 × 10⁸, 5.0 × 10⁸, 5.5 × 10⁸, 6.0 × 10⁸, 6.5 × 10⁸, 7.0 × 10⁸, 7.5 × 10⁸, 8.0 × 10⁸, 8.5 × 10⁸, 9.0 × 10⁸, 9.5 × 10⁸, 1.0 × 10⁹, 1.5 × 10⁹, 2.0 × 10⁹, 2.5 × 10⁹, 3.0 × 10⁹, 3.5 × 10⁹, 4.0 × 10⁹, 4.5 × 10⁹, 5.0 × 10⁹, 5.5 × 10⁹, 6.0 × 10⁹, 6.5 × 10⁹, 7.0 × 10⁹, 7.5 × 10⁹, 8.0 × 10⁹, 8.5 × 10⁹, 9.0 × 10⁹, 9.5 × 10⁹, 1.0 × 10¹⁰, 1.5 × 10¹⁰, 2.0 × 10¹⁰, 2.5 × 10¹⁰, 3.0 × 10¹⁰, 3.5 × 10¹⁰, 4.0 × 10¹⁰, 4.5 × 10¹⁰, 5.0 × 10¹⁰, 5.5 × 10¹⁰, 6.0 × 10¹⁰, 6.5 × 10¹⁰, 7.0 × 10¹⁰, 7.5 × 10¹⁰, 8.0 × 10¹⁰, 8.5 × 10¹⁰, 9.0 × 10¹⁰, 9.5 × 10¹⁰, 10 × 10¹⁰, 1.0 × 10¹¹ 1.5 × 10¹¹, 2.0 × 10¹¹, 2.5 × 10¹¹, 3.0 × 10¹¹, 3.5 × 10¹¹, 4.0 × 10¹¹, 4.5 × 10¹¹, 5.0 × 10¹¹, 5.5 × 10¹¹, 6.0 × 10¹¹, 6.5 × 10¹¹, 7.0 × 10¹¹, 7.5 × 10¹¹, 8.0 × 10¹¹, 8.5 × 10¹¹, 9.0 × 10¹¹, 9.5 × 10¹¹, or 1.0 × 10¹² nanoparticles. In some embodiments, the dose may be about 9.5 × 10⁸, about 9.75 × 10⁸, about 9.85 × 10⁸, about 9.95 × 10⁸, about 1.0 × 10⁹, about 1.1 × 10⁹, about 1.15 × 10⁹, about 1.2 × 10⁹, about 1.25 × 10⁹, about 1.3 × 10⁹, about 1.35 × 10⁹, about 1.4 × 10⁹, about 1.45 × 10⁹, or about 1.5 × 10⁹ nanoparticles

In some embodiments, the subject is a mammal. In some embodiments, the subject is a human. In preferred embodiments in which the subject is a human, the subject may be at least 18 years old, 40 years old, at least 45 years old, at least 50 years old, at least 55 years old, at least 60 years old, at least 65 years old, at least 70 years old, at least 75 years old, or at least 80 years old or older. In some embodiments, the subject is a pediatric subject (i.e., less than 18 years old).

### V. Nucleic Acids Encoding Nanoparticles and Coronavirus Proteins

Additionally disclosed herein are nucleic acid-based vaccines, priming agents (i.e., vaccine primers), and boosters that can be used to treat or prevent coronavirus infections such as COVID-19, which is caused by SARS-CoV-2, or to treat or prevent SARS-CoV-1 infection. For example, the disclosed nucleic acids can comprise DNA or mRNA that encodes a receptor binding domain (RBD) or other antigenic peptide of a coronavirus (e.g., SARS-CoV-2 or SARS-CoV-1) or any fusion protein described herein (i.e., a fusion protein comprising a nanoparticle-forming peptide and an antigenic coronavirus peptide, which may optionally be connected by a linker). The antigenic coronavirus peptide encoded by the nucleic acid may comprise one or more fragments or full-length proteins derived from a coronavirus (e.g., SARS-CoV-2 or SARS-CoV-1), such as the S protein and, in particular, the RBD of the S protein.

### A. DNA Vaccines, Primers, and Boosters

DNA encoding a fusion protein disclosed herein or a coronavirus S protein or fragment or variant thereof may be used as a vaccine, as a primer that can be administered prior to the administration of a nanoparticle vaccine disclosed herein, or as a booster after the administration of a nanoparticle vaccine disclosed herein. For example, the DNA can encode all, a fragment, or a variant of the RBD (or other antigenic peptide) of a coronavirus S protein (e.g., the S protein of SARS-CoV-2 or SARS-CoV-1). The DNA may be incorporated into a plasmid, which may comprise the necessary components (e.g., promoter) to express the DNA in vivo after administration to a subject, and the plasmid can be operably organized for expression in a mammal, such as a human.

For example, a sequence-optimized DNA encoding SARS-CoV-2 SpFN_1B-06-PL protein or other sequence described herein, can be synthesized in vitro using any method know in the art. Example 8 details the production of an exemplary DNA, which comprises SEQ ID NO: 282 and encodes a protein comprising SEQ ID NO: 283. Both SEQ ID NO: 282 and 283 are shown below. Parallel methodology can be used to practice other embodiments of DNA vaccines, primers, and boosters contemplated herein.

atggactctaagggcagctcccagaagggcagcaggctgctgctgctgct
ggtggtgagcaacctgctgctgcctcagggcgtggtgggcaacatcacca
atctgtgcccattcggcgaggtgtttaatgccacacgcttcgcctccgtg
tatgcctggaaccggaagagaatcagcaattgcgtggccgactattccgt
gctgtacaactctgccagcttctccacctttaagtgctatggcgtgagcc
ctaccaagctgaacgacctgtgcttcacaaacgtgtacgccgactccttt
gtgatccggggcgatgaggtgagacagatcgcaccaggacagaccggcaa
gatcgcagactacaactataagctgcctgacgacttcaccggctgcgtga
tcgcctggaattccaacaatctggattctaaagtgggcggcaactacaat
tatctgtacaggctgttccgcaagagcaacctgaagccatttgagcggga
tatctccaccgagatctaccaggccggctctacaccctgcaacggcgtgg
agggcttcaattgttattttcctctgcagtcctacggcttccagccaacc
aatggcgtgggctatcagccctaccgggtggtggtgctgtcttttgagct
gctgcacgcaccagcaaccgtgtgcggacctctggaggtgctgttccagg
gaccatctgcctggagccacccacagtttgagaagggaggaggctctgga
ggaggctccggaggctctgcctggagccacccccagttcgagaagggcag
ccatcatcatcaccaccaccaccactgatga (SEQ ID NO: 282).

NITNLCPFGEVFNATRFASVYAWNRKRISNCVADYSVLYNSASFSTFKCY
GVSPTKLNDLCFTNVYADSFVIRGDEVRQIAPGQTGKIADYNYKLPDDFT
GCVIAWNSNNLDSKVGGNYNYLYRLFRKSNLKPFERDISTEIYQAGSTPC
NGVEGFNCYFPLQSYGFQPTNGVGYQPYRVVVLSFELLHAPATVCGPLEV
LFQGPSAWSHPQFEKGGGSGGGSGGSAWSHPQFEKGSHHHHHHHH (SEQ
ID NO: 283).

### B. mRNA Vaccines

A mRNA vaccine can be prepared by preparing an mRNA molecule that encodes any one of the fusion proteins disclosed herein. As a result of the self-assembling nature of the disclosed nanoparticle, expression of such an mRNA after administration to a subject will result in the formation of nanoparticles in vivo, and such a nanoparticle can elicit an immunogenic response from the subject, such as the subject will produce coronavirus-specific antibodies. Accordingly the present disclosure provides mRNA, which can be used as vaccines, that encode any fusion protein disclosed herein.

For example, an mRNA vaccine can comprise a mRNA sequence encoding a fusion protein comprising a nanoparticle-forming peptide and an antigenic coronavirus peptide as disclosed herein (e.g., a fusion protein as disclosed herein). For example,the antigenic coronavirus peptide can comprise one or more of the following antigenic coronavirus peptides:
a. a receptor-binding domain (RBD) of a coronavirus, or a fragment or variant thereof,
b. an N-terminal domain (NTD) of a coronavirus, or a fragment or variant thereof,
c. an S1 domain of a coronavirus, or a fragment or variant thereof,
d. a stabilized extracellular spike S-2P domain of a coronavirus, or a fragment or variant thereof,
e. a stabilized extracellular spike S domain of a coronavirus, or a fragment or variant thereof, and
f. a stabilized extracellular spike S-trimer of a coronavirus, or a fragment or variant thereof;.
The nanoparticle-forming peptide can be any nanoparticle-forming peptide described herein, and may be or comprise a ferritin protein or a fragment or variant thereof, which optionally can be or comprise Helicobacter pylori ferritin (Hpf) or a fragment or variant thereof.

The mRNA vaccine can optionally comprise a linker, as disclosed herein, that connects the antigenic coronavirus peptide to the nanoparticle-forming peptide. An mRNA vaccine can encode any protein listed in Table 18.

A sequence-optimized mRNA encoding SARS-CoV-2 SpFN_1B-06-PLprotein or other sequence described herein, can be synthesized in vitro using an optimized T7 RNA polymerase-mediated transcription reaction with complete replacement of uridine by N1-methyl-pseudouridine. The reaction can include a DNA template containing the immunogen open reading frame flanked by 5′ untranslated region (UTR) and 3′ UTR sequences and can be terminated by an encoded polyA tail. After transcription, the Cap 1 structure can be added to the 5′ end using vaccinia capping enzyme (New England Biolabs) and Vaccinia 2′ O-methyltransferase (New England Biolabs). The mRNA can be purified by oligo-dT affinity purification, buffer exchanged by tangential flow filtration into sodium acetate, pH 5.0, sterile filtered, and kept frozen at -20° C. until use.

The mRNA can be encapsulated in a lipid nanoparticle (LNP) through a modified ethanol-drop nanoprecipitation process. In brief, ionizable, structural, helper and polyethylene glycol lipids can be mixed with mRNA in acetate buffer, pH 5.0, at a given ratio of lipids:mRNA. The mixture can be neutralized with Tris-Cl pH 7.5, sucrose added as a cryoprotectant, sterile filtered and stored frozen at -70° C. until further use. The mRNA and LNP can be as follows: The lipid nanoparticle contains RNA, an ionizable lipid, ((4-hydroxybutyl)azanediyl)bis(hexane-6,1-diyl)bis(2-hexyldecanoate)), a PEGylated lipid, 2-[(polyethylene glycol)-2000J-N,N-ditetradecylacetamide and two structural lipids (1,2-distearoyl-sn-glycero-3-phosphocholine (DSPC])and cholesterol). Those skilled in the art will understand that this is merely one exemplary way of formulating mRNA and that other methods and formulating agents (e.g., other lipids) used in the art may be suitable as well. Parallel methodology can be used to practice other embodiments of mRNA vaccines contemplated herein.

The present disclosure provides methods of treating or preventing coronavirus infections, such as COVID-19 or SARS-CoV-1 infections (for example), with the disclosed mRNA vaccines, as well as uses of the disclosed mRNA vaccines for treating or preventing coronavirus infections, such as COVID-19 or other coronavirus infections.

### C. Nucleic Acid Formulations and Adjuvants

The nucleic acid vaccines, primers, and boosters disclosed herein may be formulated for systemic administration via parenteral delivery. Parenteral administration includes intravenous, intra-arterial, subcutaneous, intradermal, intraperitoneal, or intramuscular injection or infusion. Formulations for parenteral administration may include sterile aqueous solutions, which may also contain buffers, diluents and other pharmaceutically acceptable additives known to the skilled artisan. For intravenous use, the total concentration of solutes may be controlled to render the preparation isotonic. Intravenous, intra-arterial, subcutaneous, or intramuscular injection are preferred routes of administration. Additionally or alternatively, the disclosed vaccines can be formulated for intranasal administration or contact with other mucosa membranes.

Formulations of the nucleic acids for injection may be presented in unit dosage form, e.g., in ampules, or in multi-dose containers, optionally with an added preservative. The formulations may take such forms as suspensions, solutions or emulsions in oily or aqueous vehicles, and may contain formulatory agents such as suspending, stabilizing and/or dispersing agents. The formulations may comprise any suitable pharmaceutically acceptable excipients.

Commonly, nucleic acids that are administered to a subject are formulated in a lipid composition, such as a lipid nanoparticle. Such LNPs and other lipid-based carriers are known in the art.

Formulations comprising a disclosed nucleic acid vaccine, primer, or booster may also comprise a suitable adjuvant, such as one or more of ALFQ and Alhydrogel and other adjuvants, such as monophosphoryl lipid A (MPLA), oil in water emulsions, ADJUPLEX™, X, CARBOPOL® polymers, Poly IC:LC, PolyI:C, CpG, Flagellin, Iscomatrix, Virosome, MF59, AS03, and AS04, among others.

### VI. Screening for Binding Molecules

In addition to being used for treatment, the disclosed nanoparticles and fusion proteins can be used to screen binding molecules, such as antibodies, for ability to bind to and neutralize a coronavirus (e.g., SARS-CoV-1 or SARS-CoV-2). Any of the fusion proteins disclosed in Table 18 or nanoparticles comprising the fusion proteins in Table 18 can be contacted with a putative coronavirus binding molecule, such as a putative anti-coronavirus antibody, and assessed for binding to the fusion protein or nanoparticle. Antibodies (or other binding molecules) that bind to the fusion proteins disclosed in Table 18 or nanoparticles comprising the fusion proteins in Table 18 are expected to be neutralizing.

### VII. Passive Immunotherapy and Treatment With Binding Molecules

Binding molecules (e.g., antibodies that bind to SARS-CoV-2 or another coronavirus as disclosed herein) can be used for passive immunotherapy to prevent the development of a coronavirus infection or for the treatment of a subject that already has a coronavirus infection. In general, coronavirus-specific antibodies can be obtained from a subject that was administered a vaccine disclosed herein or coronavirus-specific antibodies can be identified from a subject that recovered from a coronavirus infection (e.g., COVID-19) using the disclosed fusion proteins and nanoparticles as bait for a screening assay. These antibodies can be administered to a subject that has been exposed to or is at risk of exposure to a coronavirus in order to prevent the development of a coronavirus infection such as COVID-19 or SARS-CoV-1 infection, for example (i.e., the antibodies can serve as a “passive immunotherapy”). Additionally or alternatively, these antibodies can be administered to a subject that has been infected with a coronavirus, such as SARS-CoV-1 or SARS-CoV-2, to treat the infection by, for example, reducing or eliminating viral load.

The disclosed binding proteins may be or be derived from a human IgG1 antibody, a human IgG2 antibody, a human IgG3 antibody, or a human IgG4 antibody. In some embodiments, the binding protein may be or be derived from a class of antibody selected from IgG, IgM, IgA, IgE, and IgD. That is, the disclosed binding proteins may comprise all or part of the constant regions, framework regions, or a combination thereof of an IgG, IgM, IgA, IgE, or IgD antibody. For instance, a disclosed binding protein comprising an IgG1 immunoglobulin structure may be modified to replace (or “switch”) the IgG1 structure with the corresponding structure of another IgG-class immunoglobulin or an IgM, IgA, IgE, or IgD immunoglobulin. This type of modification or switching may be performed in order to augment the neutralization functions of the peptide, such as antibody dependent cell cytotoxicity (ADCC) and complement fixation (CDC). A person of ordinary skill in the art will understand that, for example, a recombinant IgG1 immunoglobulin structure can be “switched” to the corresponding regions of immunoglobulin structures from other immunoglobulin classes, such as recombinant secretory IgA1 or recombinant secretory IgA2, such as may be useful for topical application onto mucosal surfaces. For example, immunoglobulin IgA structures are known to have applications in protective immune surveillance directed against invasion of infectious diseases, which makes such structures suitable for methods of using the disclosed binding proteins in such contexts, e.g., treating or preventing coronavirus infection (e.g., COVID-19 or SARS-CoV-1 infection) or the spread of coronavirus from one individual to another.

Any of the coronavirus-specific binding proteins or antibodies obtained from a subject inoculated with a disclosed vaccine or screened/selected using the disclosed fusion proteins can be used for treating and/or preventing a coronavirus infection, such as COVID-19 or SARS-CoV-1 infection, for example. Optimal doses and routes of administration may vary, such as based on the route of administration and dosage form, the age and weight of the subject, and/or the subject’s condition, including the type and severity of the coronavirus infection, and can be determined by the skilled practitioner. The binding proteins can be formulated in a pharmaceutical composition suitable for administration to a subject by any intended route of administration.

The following examples are given to illustrate the present disclosure. It should be understood that the invention is not to be limited to the specific conditions or details described in these examples.

### EXAMPLES

### Example 1 - Design and Testing of Fusion Proteins and Nanoparticles

Recently, the molecular structure of recombinant full-length SARS-CoV-2 Spike protein was solved in a stabilized pre-fusion state, by single particle cryo-Electron Microscopy (cryo-EM), at a resolution of 3.8 Å (Wrapp et al., 2020). Despite the comprehensive structural characterization of the spike protein as a whole, movement of the RBD between “up” and “down” conformational states prevented complete modeling of the RBD domains. Subsequent cryo-EM investigations of SARS-CoV-2 provided more detail of RBD, particularly at sites that contact the human ACE-2 receptor (Yan et al., 2020a). Here, the first high resolution—less than 2 Å—SARS-CoV-2 RBD is reported. Additionally, the antigenicity of this recombinant RBD is reported and it is particularly of interest given the equipoise in the literature regarding the binding affinities of SARS-CoV antibodies for SARS-CoV-2 RBD. Early reports, have described that the human SARS-CoV antibody, CR3022, is able to bind to the SARS-CoV-2 RBD. In the present example, binding was verified, and subsequently solved the structure of SARS-CoV-2 RBD in complex with CR3022 with a novel “cryptic” epitope.

### Protocols

### Production of Recombinant Proteins

The Shanghai Public Health Clinical Center & School of Public Health, in collaboration with the Central Hospital of Wuhan, Huazhong University of Science and Technology, the Wuhan Center for Disease Control and Prevention, the National Institute for Communicable Disease Control and Prevention, Chinese Center for Disease Control, and the University of Sydney, Sydney, Australia released the sequence of a coronavirus genome from a case of a respiratory disease from Wuhan on January 10^{th} 2020 available at recombinomics.co/topic/4351-wuhan-coronavirus-2019-ncov-sequences/. The sequence was also deposited in GenBank (accession MN908947) and GISAID (>EPI_ISL_402125). DNA encoding the SARS-Cov-2 RBD (residues 331-527) was synthesized (Genscript) with a C-terminal His6 purification tag and cloned into a CMVR plasmid, and protein was expressed by transient transfection in 293F cells for six days. The SARS-CoV-2 RBD-His protein was purified from cell culture supernatant using a Ni-NTA (Qiagen) affinity column. DNA encoding the S protein ectodomains (residues 1-1194) from bat SARS-related CoV isolates Rs4231 and Rs4874 (ref.(Hu et al., 2017)) were synthesized (Genscript) with a C-terminal T4-Foldon domain or C-terminal GCN domain, respectively, followed by factor xA cleavage sites and Strep-Tactin purification tags. Bat SARSr-CoV S genes were cloned into a modified pcDNA3.1 expression plasmid (Chan et al., 2009). Protein was initially expressed by transient transfection in 293F cells for six days, then serial cloned to select stably expressing cell lines (Yan L., in submission). The Rs4231-T4 and Rs4874-GCN S proteins were purified from cell culture supernatant using a Strep-Tactin affinity column. The oligomeric structure of these S proteins was selected by size exclusion chromatography (GE/AKTA) and trimeric S proteins were confirmed by Native-PAGE. SARS S-2P was produced as previously described, with Strep-Tactin affinity chromatography followed by gel filtration using a 16/60 Superdex-200 purification column. Purification purity for all S glycoproteins was assessed by SDS-PAGE.

The sequences of the CR3022 variable regions of the heavy and light chains are available in GenBank under accession numbers DQ168569 and DQ168570, respectively (ter Meulen et al., 2006). These sequences were synthesized (Genscript) and cloned into CMVR expression vectors (NIH AIDS reagent program) between a murine Ig leader (GenBank DQ407610) and the constant regions of human IgG1 (GenBank AAA02914), Igκ (GenBank AKL91145). Plasmids encoding heavy and light chains were co-transfected into Expi293F cells (ThermoFisher) according to the manufacturer’s instructions. After 5 days, antibodies were purified from cleared culture supernatants with Protein A agarose (ThermoFisher) using standard procedures, buffer exchanged into Phosphate-Buffered Saline (PBS), and quantified using calculated E and A280 measurements.

The Fab fragment of antibody CR3022 was prepared by digestion of the full-length IgG using enzyme Lys-C (Roche). The digestion reaction was allowed to proceed for 2.5 hours at 37° C. Digestion was assessed by SDS-PAGE and upon completion, the reaction mixture was passed through protein-G beads (0.5-1 ml beads), 3 times and the final flow through was assessed by SDS-PAGE for purity. The Fab fragment was mixed with purified SARS-CoV-2 RBD, and the complex was allowed to form for 1 hour at room temperature.

Cell lines: Expi293F (ThermoFisher Scientific #A14527), and 293F cell lines were utilized in this study.

### X-Ray Crystallography

Crystallization - SARS-CoV-2 RBD at 10 mg/ml and 5 mg/ml in PBS buffer was screened for crystallization conditions using an Art Robbins Gryphon crystallization robot, 0.2 µl drops, and a set of 1200 crystallization conditions. Crystal drops were observed using a Jan Scientific UVEX-PS with automated UV and brightfield drop imaging robot. Crystals of the SARS-CoV-2 RBD grew after 24 hours in multiple conditions from the Molecular Dimensions MIDAS crystal screen, with diffraction-quality crystals seen in conditions B1, G1, F6, and H10. CR3022 Fab was screened for crystallization at 10.0 mg/ml and 5.0 mg/ml concentrations in PBS. Diffraction quality crystals grew after 48 hours in 0.1 M Imidazole pH 6.5, 40% 2-propanol and 15% PEG 8,000. For the complex, CR3022 Fab and SARS-CoV-2 RBD were mixed in 1:1 molar ratio and crystallization drops were set-up at 8.0 and 4.0 mg/ml concentrations in PBS buffer as described above. Crystals grew in a crystallization condition containing 1 M Succinic acid, 0.1 M HEPES pH 7.0 and 2% PEG MME2000. Both, RBD alone and CR3022 Fab-RBD complex, crystals were harvested and cryo-cooled in their respective crystallization conditions plus 25% glycerol.

Diffraction data collection and processing - Single crystals were transferred to mother liquor containing 22% glycerol, and cryo-cooled in liquid nitrogen prior to data collection. Diffraction data for SARS-CoV-2 RBD were collected at Advanced Photon Source (APS), Argonne National Laboratory, NE-CAT ID24-C beamline, and measured using a Dectris Eiger 16 M PIXEL detector. Crystals grown in MIDAS condition B1 (20% Jeffamine D2000, 10% Jeffamine M2005, 0.2 M NaCl, 0.1 M MES pH 5.5) provided the highest resolution diffraction with spots visible to 1.8 Å. A complete dataset could be processed to 1.95 Å in space group P41212. CR3022 Fab crystals diffracted to 3.3 Å on NE-CAT ID24-C beamline. Diffraction data could be scaled in P21 space group with 99.9% completeness. Diffraction data for CR3022 and SARS-CoV-2 RBD complex were collected on NE-CAT ID24-C beamline at Advanced Photon Source (APS), and measured using a Dectris Eiger 16 M PIXEL detector. Diffraction data from multiple crystals were merged and scaled together to achieve a final resolution of 4.2 Å with overall completeness of 82.2%. Data collection statistics are reported in Table 19 at the end of the specification.

Structure solution and refinement - Phenix xtriage was used to analyze the scaled diffraction data produced from HKL2000 and XDS. Data was analyzed for completeness, Matthew’s coefficient, twinning or pseudo-translational pathology. The structure of the SARS-CoV-2 RBD was determined by molecular replacement using Phaser and a search model of the SARS RBD (PDB ID: 2AJF, molecule C). CR3022 Fab crystal structure was determined by molecular replacement using Coxsackievirus A6 neutralizing antibody 1D5 (PDB ID: 5XS7) as a search model. The CR3022-RBD complex structure was determined by molecular replacement using the refined CR3022 and SARS-CoV-2 RBD structures as search models. Refinement was carried out using Phenix refine with positional, global isotropic B factor refinement, and defined TLS groups, with iterative cycles of manual model building using COOT. Structure quality was assessed with MolProbity. The final refinement statistics for all the structures are reported in Table 18. All structure figures were generated using PyMOL (The PyMOL Molecular Graphics System [DeLano Scientific]).

### Structure Comparisons

Weighing epitope sites based on antigen-antibody interactions - Epitope sites correspond to antigen sites that are in contact with the antibody in the antigen-antibody complex (i.e. all sites that have non-hydrogen atoms within 4 Å of the antibody). For a given epitope site, the weight, which characterizes the interaction between the epitope site and the antibody (improved based on (Bai et al., 2019)), was defined as:

$$w = \frac{1}{2} \left(\frac{{n}_{c}}{\left〈{n}_{c}\right〉} + \frac{{n}_{n b}}{\left〈{n}_{n b}\right〉}\right)$$

in which, n_{c} is the number of contacts with the antibody (i.e. the number of non-hydrogen antibody atoms within 4 Å of the site) ; n_{nb} is the number of neighboring antibody residues; 〈n_{c}〉 is the mean number of contacts n, and 〈n_{nb}〉 is the mean number of neighboring antibody residues n_{nb} across all epitope sites. A weight of 1.0 is attributed to the average interaction across all epitope sites. Neighboring residue pairs were identified by Delaunay tetrahedralization of side-chain centers of residues (C_{α} is counted as a side chain atom, pairs further than 8.5 Å were excluded). Quickhull(Barber, 1996) was used for the tetrahedralization and Biopython PDB (Hamelryck and Manderick, 2003) to handle the protein structure.

In the SARS-CoV-2 and SARS-CoV-1 RBD comparison, residues were considered similar for the following residues pairs: RK, RQ, KQ, QE, QN, ED, DN, TS, SA, VI, IL, LM and FY.

Biolayer interferometry - Affinity kinetic interactions between SARS-CoV-2 RBD proteins and antibodies were monitored on an Octet RED96 instrument (ForteBio). After reference subtraction, binding kinetic constants were determined, from at least 4 concentrations of Fab, by fitting the curves to a 1:1 Langmuir binding model using the Data analysis software 9.0 (FortéBio). Antibodies were loaded at 30 µg/ml onto a AHC probe for 120 s followed by baseline incubation for 30-60 s.

To assess antibody competition, either 240CD or CR3022 or a non-specific control antibody CR1-07 was incubated with the SARS-CoV-2 RBD prior to assessment of binding to CR3022 or 240CD. Antibody concentration was 30 µg/ml. To assess binding of human ACE-2 receptor in the presence or absence of antibodies CR3022, or 240CD, RBD was loaded onto a HIS probe. The RBD was then sequentially incubated with either CR3022, 240CD or control antibody CR1-07 prior to incubation with human ACE-2 receptor.

CR3022 was loaded onto an AHC probe for 120s prior to incubation with SARS-CoV S glycoproteins (15 µg/ml) alone or pre-incubated with ACE2 protein. SARS S-2P protein was treated with 0.1% bovine pancreas trypsin for 10 minutes prior to binding to binding measurements. SARS Spike protein was provided by BEI resources, Lot 768P152. Binding of CR3022 was also carried out against a series of concentrations of SARS S-2P which had been treated with 0.1% w/w bovine pancreatic trypsin.

TABLE 4

Crystallographic Data Collection and Refinement Statistics

| | SARS-CoV-2 RBD | CR3022 Fab | SARS-CoV-2 RBD + CR3022 Fab |
|---|---|---|---|
| PDB Code | | 6W7Y | |
| Data collection | | | |
| Space group | P4₁2₁2 | P2₁ | P4₁22 |
| Cell dimensions | | | |
| a, b, c (Å) | 80.5.80.5,161.7 | 52.1, 201.0, 57.0 | 1151.17, 151.17,192.9 |
| α, β, γ (°) | 90.0,90.0,90.0 | 90.0, 109.4.0, 90.0 | 90.0,90.0,90.0 |
| Resolution(Å) | 50.0-1.95 (2.02-1.95) | 50.00-3.3 (3.42-3.30) | 50.0-4.2 (4.35-4.20) |
| Reflection (uni/tot) | 38,164/107,541 | 16,019/30,025 | 13,814/84,711 |
| R_{sym} or R_{merge} | 4.7 (79.3) | 8.9 (28.0) | 24.6 (108.8) |
| Rₚᵢₘ | 3.1 (59.8) | 6.3 (19.8) | 9.4 (57.0) |
| CC½ | 98.9 (70.6) | 199.1 (93.5) | 198.2 (47.6) |
| I/σI | 18.9 (1.1) | 10.2 (1.6) | 5.57 (1.0) |
| Completeness (%) | 96.8 (90.0) | 96.5 (95.4) | 82.2 (48.8) |
| Redundancy | 2.8 (2.4) | 1.9 (1.9) | 6.1(3.4) |
| Refinement | | | |
| Resolution (Å) | 20.0-1.95 | 20.0-3.3 | 30.0-4.2 |
| Reflections | 29,582 | 15,999 | 11,120 |
| R_{work} / R_{free}* | 16.5/20.0 | 25.4/27.5 | 24.2/29.2 |
| Protein | 1,596 | 6,579 | 4,928 |
| Ligand/ion | 97 | – | 28 |
| Water | 79 | – | n/a |
| Protein | 28.8 | 66.7 | 145.6 |
| Ligand/ion | 56.2 | – | 190.4 |
| Water | 45.3 | – | n/a |
| Ramachandran | | | |
| Favored/Allowed/Outliers | 94.5/5.5/0.0 | 90.8/8.0/1.2 | 92.0/8.0/0.0 |
| Bond lengths (Å) | 0.007 | 0.015 | 0.003 |
| Bond angles (°) | 0.874 | 1.52 | 0.621 |

Values in parentheses are for highest-resolution shells.
* Rfree was calculated using ~5% randomly selected reflections.

### Additional Methods

Mouse Immunizations: C57BL/6 or balb/c mice were immunized typically with 10 µg of immunogen mixed with adjuvant, either ALFQ or Alhydrogel (preparation described below) in a final volume of 50 µl. In other instances, 0.08 µg of immunogen was mixed with adjuvant, either ALFQ or Alhydrogel (preparation described below) in a final volume of 50 µl. In other instances, the dose of immunogen was either 2 µg or 0.4 µg or 0.016 µg or 0.0032 µg, which was mixed with adjuvant, either ALFQ or Alhydrogel (preparation described below) in a final volume of 50 µl.

A single injection site was used at a given immunization time point. Mice were immunized at week 0, 3, and 6. Mice were bled prior to the immunization study start (pre-bleed) and at week 2, 5, 8, 10, 12. Mice were 6-10 weeks of age at time of first immunization.

Adjuvants: ALFQ (1.5X) (Lot# 05042020-ALFQ) liposomes contain 600 µg/mL 3D-PHAD and 300 µg/mL QS-21. 14.7 mL of ALF55 (Lot#02282020-ALF55, containing 1.236 mg/mL 3D-PHAD) was diluted with 6.5 mL of isotonic Sorensen’s PBS pH 6.15 in a sterile glass vial. ALFQ was created by adding 9.08 mL of QS-21 (1 mg/mL) to the diluted ALF55 while slowly stirring. The vial was sealed and incubated on a roller for 1 hour at room temperature. ALFQ was stored at 4° C. until use. ALFQ was gently mixed by slow speed vortex prior to use.

Vaccine Formulation: Aliquot 250 µL of ALFQ (1.5X, 600 µg/mL 3D-PHAD) to a sterile glass vial. Add 125 µL of Antigen (600 µg/mL) to the ALFQ and mix with pipetting 10 times. Seal the vial. Vortex the vial with slow speed for 1 min and put it on a roller for 15 mins. Store the vial at 4° C. prior to immunization. Prepare 1 hour before immunization. Inject 50 µL/mouse IM.

Alhydrogel: Alhydrogel Stock contains 10 mg/ml aluminum (GMP grade; Brenntag).

Antigen: All reagents were equilibrated to room temperature before use. Antigens were diluted to be 600 µg/mL by adding filter sterilized dPBS (Lot#723188, Quality Biological) to the tubes. Tubes were mixed by pipetting ten times.

Vaccine Formulation: Dilute Alhydrogel to 900 µg/mL (1.5X) by mixing 43.2 µL of Alhydrogel stock (10 mg/mL) with 436.8 µL of DPBS in a sterile glass vial. Add 240 µL of Antigen (600 µg/mL) to the vial containing the diluted Alhydrogel and seal the vial. Vortex with slow speed for 5 min and store at 4° C. for at least 2 hours prior to immunization. Inject 50 µL/mouse.

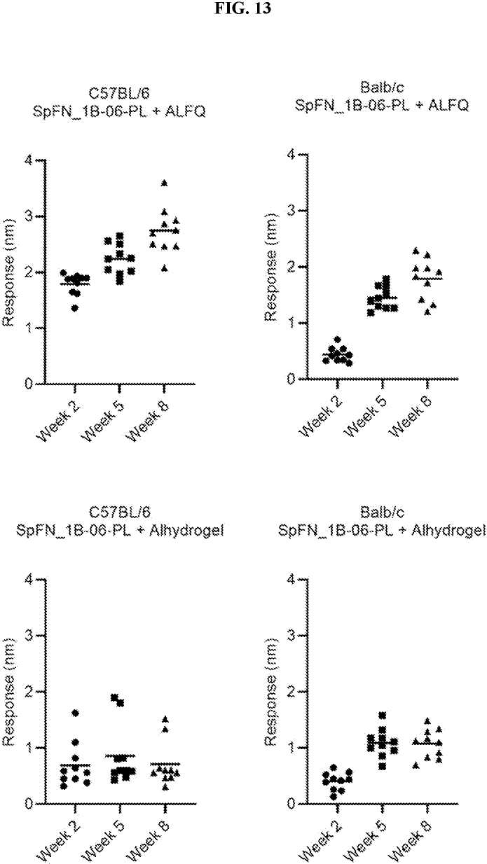

Octet Binding studies: SARS-CoV-2 RBD and mouse sera binding were monitored using an Octet RED96 instrument (FortéBio). Mouse sera was typically diluted 1:100 in BioForte Kinetics Buffer (some samples from the week 5 time point were also assessed at 1:200 or 1:400 dilution). A His1K probe was pre-equilibrated in Kinetics buffer. SARS-CoV-2 RBD-His protein was diluted to 30 µg/ml in PBS and allowed to interact with the His1K probe for 120 s, with typical response levels of 1 nm observed. The probe was briefly equilibrated in Kinetics buffer, and then allowed to interact with the diluted mouse sera for 120-180s. Binding response levels after 180 s were noted and are shown in FIGS. 13, 18, 24 and 25.

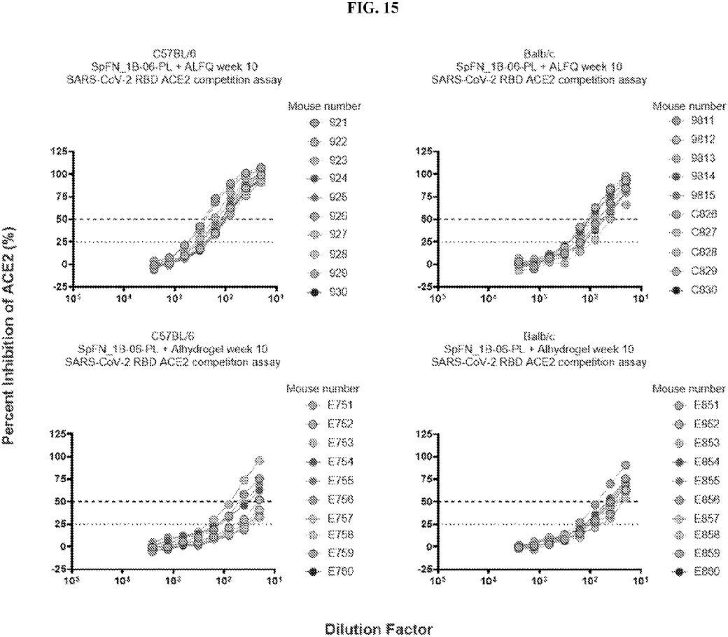

Octet ACE-2 receptor inhibition studies: As described above, Mouse sera at a 1:50 dilution in Kinetics buffer was prepared. Two-fold serial dilutions were prepared. SARS-CoV-2 was bound to a HIS1K probe and incubated with mouse sera for 180 s, and then assessed for binding to human ACE-2 receptor as shown in FIG. 15. Mouse sera from pre-bleed samples were also incubated with RBD and showed no binding to the RBD, or no resulting inhibition of ACE-2 binding.

Characterization of Immunogens by Octet Biolayer Interferometry: A set of monoclonal antibodies were used to assess reactivity to immunogens. These include RBD-targeting antibodies that are non-neutralizing or poorly-neutralizing and include CR3022, CV1, and S625-109, and neutralizing antibodies H14, 441, CVH1, and CVH5, or NTD-targeting antibodies S625-118 and P22_7. Antigens and antibodies were monitored using an Octet RED96 instrument (ForteBio). Antibodies (40 µg/ml) were loaded onto AHC probes, equilibrated in Kinetics buffer, prior to interaction with antigens of interest for 100 s. Antibodies were allowed to be dissociated for 40 s.

Characterization of SARS-CoV-2-Ferritin immunogens by size-exclusion chromatography: Immunogens were initially purified from cell supernatant by affinity chromatography using either NiNTA or GNA-lectin resin. Samples were then loaded onto a Superdex-200 column (20 ml or 120 ml column volume). Nanoparticle formation and uniformity were judged from the resulting chromatogram. S-Ferritin nanoparticles would be expected to elute at 10 ml (20 ml column) or 40 ml (120 ml column). In some immunogens, multiple peaks were observed. The eluted protein corresponding to the expected molecular weight of the S-nanoparticle as shown in FIGS. 6-10 was used for further characterization including the mouse immunization studies.

TABLE 5

Summary of mouse immunization studies (102 groups of mice)
Control group constructs

| pCOV no. | Immunogen design category, Study design | C57BL/6 ALFQ | Balb/c ALFQ | C57BL/6 Alhydrogel | Balb/c Alhydrogel |
|---|---|---|---|---|---|
| 3 | RBD monomer (control) | X | X | X | X |
| 47 | S-2P Trimer (control) | X | X | X | X |
| 71 | NTD monomer (control) | X | X | | |
| DNA prime and Protein Boost | | | | | |
| 3 + 3 | RBD DNA prime, RBD Protein Boost – GS adjuvant | X | X | | |
| 3+1 | RBD DNA prime, RBD-Ferritin Protein Boost – GS adjuvant | X | X | | |
| Spike-Ferritin constructs | | | | | |
| 1B-05 | S-Trimer-Ferritin (x 2 groups) | XX | XX | | |
| 1B-05 | S-Trimer-Ferritin (50 ug) | X | X | | |
| 1B-06-PL | S-Trimer-Ferritin | X | X | X | X |
| 1B-06-PL | S-Trimer-Ferritin 7 doses 10 µg - 0.0032 µg | XXXX XXX | XXXX XXX | | |
| 1B-06-PL | S-Trimer-Ferritin Development Grade cGMP material 3 doses 10 µg - 0.08 µg | XXX | XXX | | |
| 186 | S-Trimer-Ferritin (1B-06-PL with HexaPro + D614G) | X | X | | |
| 187 | S-Trimer-Ferritin (1B-08-PL with D614G) | X | X | | |
| RBD-Ferritin constructs | | | | | |
| pCOV no. | Immunogen design category, Study design | C57BL/6 ALFQ | Balb/c ALFQ | C57BL/6 Alhydrogel | Balb/c Alhydrogel |
| 50 | RBD-Ferritin | | X | | |
| 58 | RBD-Ferritin | X | X | X | X |
| 59 | RBD-Ferritin | | X | | |
| 127 | RBD(57+58)-Ferritin | X | X | X | X |
| 129 | RBD(50+58)-Ferritin | X | X | X | X |
| 130 | RBD(50+59)-Ferritin | | X | | |
| 131 | RBD(53+58)-Ferritin | X | X | X | X |
| Spike-Ferritin constructs Boosted with RBD-Ferritin construct | | | | | |
| 1B-06-PL + 131 boosts | S-Trimer-Ferritin prime with RBD-Ferritin boost | X | X | | |
| 187 + 131 boosts | S-Trimer-Ferritin prime RBD-Ferritin 131 boost | X | X | | |
| NTD-Ferritin constructs | | | | | |
| 23 | NTD-Ferritin | X | X | X | X |
| 65 | NTD-Ferritin (x 2 groups) | XX | XX | | |
| S1–Ferritin constructs | | | | | |
| pCOV no. | Immunogen design category, Study design | C57BL/6 ALFQ | Balb/c ALFQ | C57BL/6 Alhydrogel | Balb/c Alhydrogel |
| 111 | S1-Ferritin | X | X | | |
| RBD-NTD-Ferritin constructs | | | | | |
| pCOV no. | Immunogen design category, Study design | C57BL/6 ALFQ | Balb/c ALFQ | C57BL/6 Alhydrogel | Balb/c Alhydrogel |
| 122 | RBD-NTD-Ferritin | X | X | | |
| 125 | RBD(58)-NTD-Ferritin | | X | | X |
| 146 | RBD(53+58)-NTD-Ferritin | X | X | X | X |
| 147 | RBD(57+58)-NTD-Ferritin | X | | | |
| Co-expression of RBD-Ferritin and NTD-Ferritin constructs | | | | | |
| pCOV no. | Immunogen design category, Study design | C57BL/6 ALFQ | Balb/c ALFQ | C57BL/6 Alhydrogel | Balb/c Alhydrogel |
| 50+65 | Co-express RBD-Ferritin and NTD-Ferritin | X | X | | |
| 56+65 | Co-express RBD-Ferritin and NTD-Ferritin | X | X | | |
| 58+65 | Co-express RBD-Ferritin and NTD-Ferritin | X | X | | |
| 59+65 | Co-express RBD-Ferritin and NTD-Ferritin | X | X | | |
| Mixture of SARS-Cov-2 Spike-Ferritin and SARS-CoV-1 Spike-Ferritin constructs | | | | | |
| pCOV no. | Immunogen design category, Study design | C57BL/6 ALFQ | Balb/c ALFQ | C57BL/6 Alhydrogel | Balb/c Alhydrogel |
| 1B-06-PL + pCoVS01 | SARS-1 SpFN_1B-06-PL and SpFN_1B-06-PL 2 doses: 10 µg and 2 µg | XX | XX | | |
| | Total number of groups | 40 | 43 | 9 | 10 |

### Results

High resolution structure of the SARS-CoV-2 RBD: The SARS-CoV-2 RBD (residues 313-532), with a C-terminal His-tag, was expressed in 293F cells, and purified by NiNTA affinity, and size-exclusion chromatography. Crystallization condition screening identified 20% Jeffamine D2000, 10% Jeffamine M2005, 0.2 M NaCl, 0.1 M MES pH 5.5 for diffraction quality crystal growth. Crystals diffracted to <1.8 Å in group P 41 21 2 and to a complete dataset to 1.95 Å that could be scaled and processed (Table 4). The structure was refined to an Rfree of 20% and Rwork of 22% with no Ramachandran outliers. S residues 313-532 were clearly interpretable from the electron density map, with a dual conformation of a loop containing residues 484 to 487 clearly visible in the electron density map. Structure comparison of the unliganded RBD structure presented here, with the stabilized prefusion SARS-CoV-2 Spike (S-2P) molecule structure determined by Cryo-EM (PDB ID: 6VSB) (Wrapp et al., 2020) shows high structural similarity, with an RMSD of 0.68, 0.68, and 0.71 for each of the spike protomers. In the structure of the S-2P molecule (S-2P) (Wrapp et al., 2020) 25, 29 or 49 amino acids (aa) within each protomer RBD are not modeled, including 40% of the ACE-2 receptor binding site as measured by buried surface area (BSA)(Yan et al., 2020b). The SARS-CoV RBD-2 compared to liganded (PDB ID: 2AJF) and unliganded (PDB ID: 2GHV) SARS-CoV RBD structures shows high structural similarity, except for residues 473-488. A chimeric SARS-CoV-2 RBD structure (PDB ID: 6VW1) with 23 amino acid differences compared to SARS-CoV-2, in complex with human ACE-2 was recently released in the PDB.

Identification of a set of cross-reactive SARS-CoV-2 antibodies: In an effort to identify antibodies that could bind to SARS-CoV-2, a set of SARS-CoV,(Tripp et al., 2005) and MERS CoV(Wang et al., 2018; Wang et al., 2015) RBD-reactive antibodies were screened for binding to the SARS-CoV-2 RBD. It was demonstrated that the SARS-CoV mouse antibody 240CD (Tripp et al., 2005) had nanomolar (nM) affinity for the SARS-CoV-2 RBD and did not significantly block ACE-2 receptor binding. CR3022—a SARS-CoV neutralizing antibody (Tian et al., 2020) identified from a human phage-display library (ter Meulen et al., 2006)—also bound to SARS-CoV-2 RBD with nM affinity. Competition binding was assessed between 240CD and CR3022, and showed that these antibodies cross-compete with each other for binding to the SARS-CoV-2 RBD.

SARS-CoV-2 has a likely zoonotic origin and horseshoe bats have been implicated as natural reservoirs of both SARS-CoV and SARS-CoV-2 (Menachery et al., 2015; Zhou et al., 2020). As such, antibody cross-reactivity was explored with the S glycoproteins of two bat SARS-related CoVs: SARSr-CoV Rs4874 (Ge et al., 2013; Yang et al., 2015) and Rs4231 (Hu et al., 2017), which are closely related to the progenitor of SARS-CoV and retain the ability to utilize human ACE-2. CR3022 was able to recognize a recombinant Spike glycoprotein generated from bat SARSr-CoV Rs4874, while 240CD, and other mouse generated monoclonal antibodies have a mixed recognition phenotype.

Crystal structure of antibody CR3022 in complex with SARS-CoV-2 RBD: The antigenic cross-reactivity of this set of antibodies (240CD and CR3022) precipitated an investigation into their molecular recognition determinants. The potential relevance of a human antibody motivated the investigation to prioritize studies of CR3022, for which a sequence was available (ter Meulen et al., 2006). The CR3022 heavy chain is encoded by IGHV5-51*03, contains a 12-aa CDR H3 with 8 V gene-encoded residues altered by somatic hypermutation. CR3022 light chain is encoded by IGKV4-1*01 with 1 V gene-encoded residue, altered by somatic hypermutation, and a 9-aa CDR L3. To provide an atomic-level understanding of the structure of the CR3022 antibody, the antigen-binding fragment (Fab) of CR3022 was crystalized. Crystals diffracted to 3.2 Å resolution in space group P 2₁. Overall the structure of the CR3022 Fab revealed a relatively flat antigen-combining site, with the exception of an extended protruding 12-aa CDR L1 loop.

To determine the structure of CR3022 in complex with the SARS-CoV-2 RBD, crystallization conditions screening was carried out with crystals of the CR3022-RBD complex forming in 1 M Succinic acid, 0.1 M Hepes pH 7, 2% PEG MME2000 and determined the crystal structure by X-ray diffraction to 4.25 Å. The complex structure was solved by molecular replacement using the refined CR3022 and SARS-CoV-2 RBD structures as search models and was refined to an R_{work}/R_{free} of 0.242/0.292. CR3022 bound to the RBD at an epitope centered on S glycoprotein residues 377-386 with a total buried surface area of 871 Å. This region is highly conserved between SARS-CoV and SARS-CoV-2. Comparison of the CR3022 epitope site with previously described antibody-complex structures for SARS-CoV, and MERS-CoV indicates that CR3022 describes a novel recognition site. Further sequence analysis of the epitope indicates that this epitope is conserved in β-coronavirus clade 2b, with also some similarity in clade 2d. To confirm that this site was also shared with 240CD, an RBD knockout mutant was produced by introducing a glycan sequon at position 384, and by biolayer interferometry show that both CR3022 and 240CD binding to the RBD can be eliminated by the introduction of a glycan at this site.

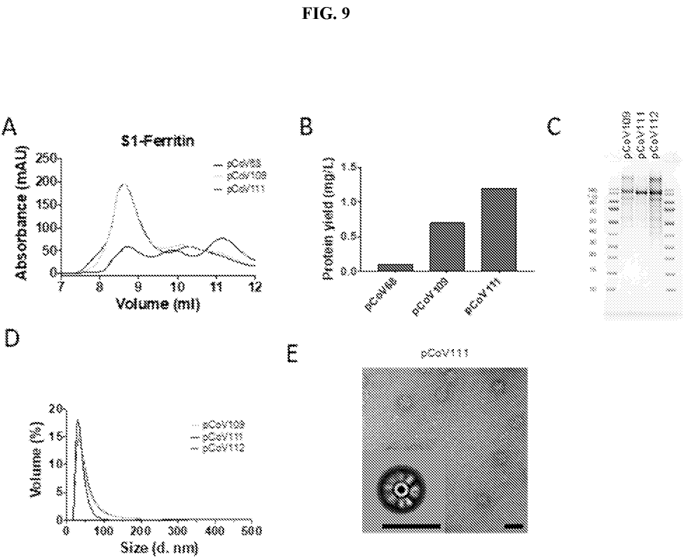

Identification of a cryptic site of vulnerability recognized by CR3022: The epitope conservation within the clade explained the antigenic cross-reactivity with both human SARS-CoV and bat SARS related CoV. To date, there has been extensive structural characterization of the SARS-CoV, and MERS-CoV spike molecule and domains, which provided a framework for understanding the novel SARS-CoV-2 spike molecule. In the context of the coronavirus trimeric S glycoproteins, the RBD displays two prototypical conformations either in an “up” or “down” position, with implications for receptor binding and cell entry. To further analyze these conformations, the CR3022 binding was modeled to the trimeric structures of SARS-CoV-2, SARS-CoV and MERS-CoV. The CR3022 epitope was occluded by adjacent spike protomers when the RBD is in the “down” conformation, but becomes more accessible when the spike is in a more open conformation here multiple RBD molecules are in the “up” conformation. These conformations are shown in FIG. 9. There was still a clash of the antibody Fc1 region with the NTD from the same protomer, or an RBD from an adjacent protomer when modeled using the static structure.

To understand whether CR3022 could bind to SARS-CoV S glycoproteins, binding to stabilized S-2P or non-stabilized versions of S was measured. Robust binding to the non-stabilized S glycoprotein was observed, while binding to SARS S-2P Trimer was low. The SARS S-2P trimer was then treated with trypsin and/or incubation with the ACE2 receptor to assess whether minimal proteolytic action or receptor binding could increase the availability of the “cryptic” CR3022 epitope. Incubation of the stabilized S-2P trimer with human ACE2 did not dramatically affect CR3022 binding, while in contrast, the trypsin treatment of the S-2P protein resulted in increased binding akin to the unstabilized S glycoprotein binding, and the level of binding was titratable, with increasing amounts of S-2P resulting in higher CR3022 binding. Given the prior neutralization and protection studies utilizing CR3022, and its ability to complement potent neutralizing antibodies, it is likely that the CR3022 epitope represents a “cryptic” epitope that becomes exposed during the processes of viral cell entry.

In summary, this data represents the most detailed structural information for the SARS-CoV-2 RBD to date and the first structure of the SARS-CoV-2 in complex with a human antibody. The presence of “cryptic” but protective epitopes for influenza (Bangaru et al., 2019), and Ebola viruses (West et al., 2018), have been previously described. The identification of a novel “cryptic” epitope for β-coronaviruses including SARS-CoV, and SARS-CoV-2 highlight a novel viral vulnerability that can be harnessed in combination with ACE2 receptor site targeting monoclonal antibodies for vaccine development.

### Example 2 - Immunogenicity of SARS-CoV-2 SpFN_1B-06-PL in Mice

Severe Acute Respiratory Syndrome associated Coronavirus 2 (SARS-CoV-2) is a zoonotic coronavirus that inflicts severe respiratory disease in humans and is the cause of the COVID-19 pandemic. Similar to the first SARS-CoV, this novel coronavirus’s surface Spike (S) glycoprotein mediates cell entry via the human angiotensin-converting enzyme 2 (ACE2) receptor, and, thus, the Spike is the principal target for the development of vaccines and immunotherapeutics. Antibodies that can bind to the Spike glycoprotein and prevent interaction with the ACE2 receptor can facilitate protection from infection. A Spike-Ferritin Protein Nanoparticle with ALFQ adjuvant (SpFN_1B-06-PL + ALFQ) vaccine has been developed to elicit protective antibody responses against SARS-CoV-2. Ferritin is a naturally occurring protein that self-assembles into a 24-member spherical particle, made up of multiple three-fold, four-fold and two-fold axes. Using the 3-fold axes, 8 trimeric SARS-CoV-2 Spike glycoproteins are presented on the surface of the self-assembling protein nanoparticle surface. The ALFQ adjuvant, a liposomal formulation containing MPLA and the QS-21 saponin, was developed by the Laboratory of Adjuvant and Antigen Research, Military HIV Research Program at WRAIR. The objective of this report was to evaluate the immunogenicity of SpFN_1B-06-PL in mice when administered intramuscularly. In this example the results from four studies were provided. Study 1 utilized a 10 µg dose of SpFN_1B-06-PL for each immunization in two mouse models (C57BL/6 and Balb/c), with ALFQ or aluminum hydroxide as an adjuvant. Study 2 utilized a reduced dose of 0.08 µg SpFN_1B-06-PL for each immunization in two mouse models with ALFQ as the adjuvant. The Spike Ferritin nanoparticle SpFN_1B-06-PL elicited antibodies that bound to SARS-CoV-2 Spike and Receptor-Binding domain, provided ACE2 blocking activity, and neutralized SARS-CoV-2 viruses in both pseudovirus and live-virus assays. The binding and neutralization responses were greater when using the ALFQ adjuvant compared to the aluminum hydroxide adjuvant. Both doses of SpFN_1B-06-PL (10 µg and 0.08 µg) gave high SARS-CoV-2 Spike and RBD binding titers and SARS-CoV-2 neutralization responses. Study 3 and Study 4 utilized a 10 µg SpFN_1B-06-PL dose with the adjuvant ALFQ for each immunization and were carried out to enable analysis of serum cytokine and CD4 and CD8 T cell responses. SpFN-1B-06-PL + ALFQ immunization elicited serum cytokine responses showed both TH1 and TH2 responses and IgG subclass usage when ALFQ was the adjuvant. In contrast, immunization with Aluminum hydroxide as the adjuvant induced a skewed antibody subclass usage in Balb/c mice. In summary, both the humoral and cellular immune response observed with the SARS-COV-2 vaccine SpFN_1B-06-PL + ALFQ elicited a robust and appropriate immune response.

### List of Abbreviations

3D-PHAD: Monophosphoryl 3-Deacyl lipid A (synthetic)
ACE2: angiotensin-converting enzyme 2
CoV: coronavirus
CTD: C-terminal domain
dPBS: Dulbecco’s phosphate buffered saline
ELISA: Enzyme-linked immunosorbent assay
GNA: Galanthus nivalis lectin
HRP: Horseradish Peroxidase
IFN-γ: Interferon gamma
ID: Inhibitory Dilution
IgG: Immunoglobulin G
IL-2: Interleukin 2
IL-4: Interleukin 4
IM: Intramuscular(ly)
MPLA: monophosphoryl lipid A
MSD: Meso scale discovery assay
NHP: Nonhuman primate
NTD: N-terminal domain
nm: nanometer
PBS: Phosphate buffered saline
PI: Percent inhibition
QS-21: One of the active fractions isolated from soap bark tree, Quillaja saponaria, purified using reverse phase high pressure liquid chromatography (RP-HPLC). QS denotes it source as Q. saponaria and no 21 is fraction 21 on reverse phase-High-performance liquid chromatography.
RBD: Receptor-binding domain
RG: research grade
RT: room temperature
S: Spike glycoprotein
s: seconds
S-2P: Spike glycoprotein stabilized in the prefusion form by modifications (proline modifications (K986P, V987P), and removal of the Furin cleavage site (RRAS to GSAS))
SARS-CoV-2: Severe Acute Respiratory Syndrome associated coronavirus 2
SD: standard deviation
SpFN: Spike Ferritin Nanoparticle
TEM: Transmission electron microscopy
TH: T helper
TMB: 3,3’,5,5’ tetramethylbenzidine
TNF-α: Tumor Necrosis Factor alpha
VAERD: Vaccine associated enhanced respiratory disease
WRAIR: The Walter Reed Army Institute of Research

### Introduction

The zoonotic transmission of SARS-CoV-2 to humans quickly developed into a global pandemic, infecting over 115 million people to date, resulting in an urgent need for a safe, stable, effective and durable vaccine. The SARS-CoV-2 spike (S) protein is the primary target for vaccine development, as it mediates virus entry, is immunogenic and encodes multiple sites of vulnerability. S is a class I fusion glycoprotein consisting of a S1 attachment subunit and S2 fusion subunit that remain non-covalently associated in a metastable, heterotrimeric spike on the virion surface. In the S1 subunit, there is a N-terminal domain (NTD) and C-terminal domain (CTD) that includes the receptor-binding domain (RBD), which can interact specifically with human angiotensin converting enzyme 2 (ACE2). The S protein has multiple antigenic epitopes that are targeted by neutralizing antibodies, including multiple distinct sites on the RBD and the S1 domain, including the NTD. Convalescent serum antibodies capable of potently inhibiting infection in vitro can reduce disease severity or mortality in primates and humans. SARS-CoV-2 vaccines may therefore be protective if capable of eliciting high titer, durable, S-specific neutralizing antibodies.

Multiple technology platforms are currently advancing SARS-CoV-2 vaccine development, including nucleic acid vaccines, whole virus vaccines, recombinant protein subunit vaccines and nanoparticle vaccines. Of these vaccine platform types, nanoparticle technologies have previously been shown to improve antigen structure and stability, as well as vaccine targeted delivery, immunogenicity, and safety. Bacterial ferritin-based nanoparticles self-assemble into a spherical protein shell consisting of 24 identical subunits and are ideal for display of trimeric antigens recombinantly expressed at the 3-fold axis of the ferritin subunit interface. Trimer-functionalized ferritin vaccines have been effective at eliciting neutralizing antibodies against vaccine targets including influenza haemagglutinin and HIV envelope.

In order, to elicit robust immune responses, vaccines typically contain an adjuvant component that enhances the level or type of immune response. The US Army has many decades of experience investigating liposome-based adjuvants and has recently developed an Army-Liposome-Formulation (ALF) containing high amounts of cholesterol together with the QS21 saponin (ALFQ). ALFQ has been used in numerous animal studies and in combination with a variety of immunogens has shown effectiveness in eliciting robust immune responses. In contrast to some adjuvants, ALFQ tends to elicit a balanced Th1/Th2 immune response avoiding a skewed immune response that has been implicated in vaccine associated enhanced respiratory disease (VAERD). VAERD has been associated with T helper 2 cell (TH2)-biased immune responses in some animal models with a set of experimental SARS-CoV candidate vaccines and also with whole-inactivated virus vaccines against respiratory syncytial virus and measles virus.

Here assessment of SpFN_1B-06-PL ferritin-based nanoparticles is reported in the mouse model. C57BL/6 and Balb/c mice were immunized using two injection amounts of SpFN_1B-06-PL to assess dose-sparing immune responses. In addition, immunogens were adjuvanted with both ALFQ and Alhydrogel to assess immune responses. Binding, ACE2-blocking, neutralization, antibody isotype usage, T cell type and frequency and serum cytokine profiles were assessed in these studies.

### Objectives

Immunogenicity: To assess the immunogenicity of SpFN_1B-06-PL in the presence of adjuvants ALFQ and Alhydrogel in two mouse models.
Dose response: Compare immune responses elicited by a 10 ug dose to a 0.08 ug dose of SpFN_1B-06-PL.
Antibody isotype usage: To assess the SARS-CoV-2 Spike reactive antibody isotype usage following immunization with SpFN_1B-06-PL with adjuvant ALFQ or Alhydrogel in two mouse models.
T cell and cytokine responses: To assess serum cytokine levels and the frequency of IFN-gamma, IL-2, TNF-alpha and IL-4 positive T cells in mice vaccinated with SpFN_1B-06-PL adjuvanted with ALFQ or Alhydrogel.

### Materials and Methods

### Materials

All reagents were equilibrated to room temperature before use. Antigens used in mouse immunizations were diluted by adding filter-sterilized dPBS.

SpFN IB-06-PL: Research-grade SpFN_1B-06-PL was produced by transient expression in Expi293F cells (Thermo Fisher Scientific) using the same expression construct sequence as that used to create the SpFN_1B-06-PL cGMP manufacture of clinical drug product. Culture supernatant was harvested four days post-transfection and purified by Galanthus nivalis lectin (GNA)-affinity chromatography and size-exclusion chromatography. Purified research grade SpFN_1B-06-PL was formulated in PBS with 5% glycerol at 1 mg/ml.

ALFQ: ALFQ (1.5X) (Lot# 07132020-ALFQ) liposomes contain 600 µg/mL 3D-PHAD and 300 ug/mL QS-21. ALFQ was gently mixed by slow speed vortex prior to use. Antigen was added to the ALFQ, vortexed at a slow speed for 1 minute, followed by mixing on a roller for 15 minutes. The vial was stored at 4° C. for 1 hour prior to immunization.

Alhydrogel: Alhydrogel stock contains 10 mg/ml aluminum (GMP grade; Brenntag). Alhydrogel stock solution was diluted to 900 µg/mL (1.5X) and appropriate volume and concentration of antigen was added. Antigen-adjuvant mixture was vortexed at low speed for 5 min and stored at 4° C. for at least 2 hours prior to immunization. SpFN_1B-06-PL was adsorbed to aluminum hydroxide (Alhydrogel, Brenntag) at 30 µg aluminum per 50 ul dose.

### Methods

Transmission Electron Microscopy (TEM): Purified research grade SpFN_1B-06-PL protein was assessed visually by TEM deposited at 0.02-0.08 mg/ml on carbon-coated copper grids and stained with uranyl formate. Grids were imaged using a FEI T20 microscope operating at 200 kV.

Animal experiments: All research in this study involving animals was conducted in compliance with the Animal Welfare Act, and other federal statutes and regulations relating to animals and experiments involving animals and adhered to the principles stated in the Guide for the Care and Use of Laboratory Animals, NRC Publication, 1996 edition. The research protocol was approved by the Institutional Animal Care and Use Committee of the Walter Reed Army Institute of Research. Balb/c and C57BL/6 mice were obtained from Jackson Laboratories (Bar Harbor, ME). Mice were housed in the animal facility of WRAIR and cared for in accordance with local, state, federal, and institutional policies in an NIH American Association for Accreditation of Laboratory Animal Care-accredited facility.

Animal Groups and Immunization/Assay Schedule:In Study 1, C57BL/6 or Balb/c mice (n=10/group) were immunized intramuscularly with 10 µg of SpFN_1B-06-PL adjuvanted with either ALFQ or Alhydrogel in alternating caudal thigh muscles three times, at 3-week intervals; blood was collected 2 weeks before the first immunization, the day of the first immunization, and 2 weeks following each immunization, and at week 10. In study 2, mice were immunized with 0.08 µg of SpFN_1B-06-PL adjuvanted with ALFQ with immunization schedule, site of injections, and timing of bleeds as for study 1. In study 3, C57BL/6 were immunized twice with 10 µg of SpFN_1B-06-PL adjuvanted with ALFQ and blood was collected at week 2 and week 6. In study 4, C57BL/6 mice were immunized intramuscularly with 10 µg of SpFN_1B-06-PL adjuvanted with either ALFQ or Alhydrogel, and 5 mice/group were euthanized at Day 3, 5, 7 and 10. Mice were randomly assigned to experimental groups and were not pre-screened or selected based on size or other gross physical characteristics. Serum was stored at 4° C. or -80° C. until analysis. Antibody responses were analyzed by Octet Biolayer Interferometry, ELISA, pseudovirus neutralization assay, and live-virus neutralization assay. Cellular immune responses were assessed by serum cytokine analysis, antibody isotype response, and T cell cytokine responses.

TABLE 6

Experimental Design
Study 1

| Group | Animal numbers | Treatment | Volume Injected (µl) | Vaccine Target Dose (µ g) | Adjuvant | No. and mouse strain | Immunization Schedule |
|---|---|---|---|---|---|---|---|
| 1 | 921-930 | SpFN_1B-06-PL | 50 | 10 | ALFQ | 10 C57BL/6 | Weeks 0, 3, 6 |
| 2 | 9811-9815; C826-C830 | SpFN_1B-06-PL | 50 | 10 | ALFQ | 10 Balb/c | Weeks 0, 3, 6 |
| 3 | E751-E760 | SpFN_1B-06-PL | 50 | 10 | Alhydrogel | 10 C57BL/6 | Weeks 0, 3, 6 |
| 4 | E851-E860 | SpFN_1B-06-PL | 50 | 10 | Alhydrogel | 10 Balb/c | Weeks 0, 3, 6 |
| Study 2 | | | | | | | |
| Group | | Treatment | Volume Injected (µl) | Vaccine Target Dose (µ g) | Adjuvant | No. and mouse strain | Immunization Schedule |
| 1 | E791-E800 | SpFN_1B-06-PL | 50 | 0.08 | ALFQ | 10 C57BL/6 | Weeks 0, 3, 6 |
| 2 | E881-E890 | SpFN_1B-06-PL | 50 | 0.08 | ALFQ | 10 Balb/c | Weeks 0, 3, 6 |
| Study 3 | | | | | | | |
| Group | | Treatment | Volume Injected (µl) | Vaccine Target Dose (µ g) | Adjuvant | No. and mouse strain | Immunization Schedule |
| 1 | C721-C730 | SpFN_1B-06-PL | 50 | 10 | ALFQ | 9 C57BL/6 | Weeks 0, 3 |
| Study 4 | | | | | | | |
| Group | | Treatment | Volume Injected (µl) | Vaccine Target Dose (µ g) | Adjuvant | No. and mouse strain | Immunization Schedule |
| 1 | 511-530 | SpFN_1B-06-PL | 50 | 10 | Alhydrogel | 20 C57BL/6 | 0 |
| 2 | 531-550 | SpFN_1B-06-PL | 50 | 10 | ALFQ | 20 C57BL/6 | 0 |
| 3 | 501-505 | No treatment | 0 | 0 | N/A | 5 C57BL/6 | 0 |

Octet Biolayer Interferometry: Biosensors were hydrated in PBS prior to use. All assay steps were performed at 30° C. with agitation set to 1,000 rpm using an Octet RED96 instrument (ForteBio). Baseline equilibration of the anti-His-tag biosensors (HIS1K biosensors with a conjugated Penta-His antibody (ForteBio)) was carried out using assay buffer (PBS) for 15 s, prior to SARS-CoV2-RBD (30 µg/ml diluted in PBS) loading for 120 s. After briefly dipping in assay buffer (15 s in PBS), the biosensors were dipped in the mouse sera samples (100-fold dilution) for 180 s. The binding response (nm) at 180 s was recorded for each sample.

ACE2 inhibition assay: The biosensors were equilibrated in assay buffer for 30 s before being dipped in SARS-CoV-2 RBD-His (30 µg/ml diluted in PBS). The SARS-CoV-2 RBD-His were immobilized on HIS1K biosensors (FortéBio) for 180 s. After briefly dipping in assay buffer (30 s, PBS), binding of week 10 mouse serum was allowed to proceed for 180 s followed by a brief equilibration for 30 s. Binding of ACE2 protein (30 ug/ml) in solution was assessed for 120 s. Percent inhibition (PI) of RBD binding to ACE2 by mouse serum was determined by an equation: PI = 100 - [(ACE2 binding in the presence of competitor mouse serum)/(ACE2 binding in the absence of competitor mouse serum)] × 100.

Enzyme Linked Immunosorbent Assay (ELISA): 96-well Immulon “U” Bottom plates were coated with 1 µg/mL of RBD or spike protein (S-2P) antigen in PBS, pH 7.4. Plates were incubated at 4° C. overnight and blocked with blocking buffer (Dulbecco’s PBS containing 0.5% milk and 0.1% Tween 20, pH 7.4, at room temperature (RT) for 2 h. Individual serum samples were serially diluted 2-fold in blocking buffer and added to triplicate wells and the plates were incubated at RT for 1 h. Horseradish peroxidase (HRP)-conjugated sheep anti-mouse IgG, gamma chain specific (The Binding Site) was added and incubated at RT for an hour, followed by the addition of 2,2′-Azinobis [3-ethylbenzothiazoline-6-sulfonic acid]-diammonium salt (ABTS) HRP substrate (KPL) for 1 h at RT. The reaction was stopped by the addition of 1% SDS per well and the absorbance was measured at 450 nm. using an ELISA reader Spectramax (Molecular Devices). Positive (anti-RBD mouse mAb; BEIresources) and negative controls were included on each plate. The results are expressed as end point titers, defined as the reciprocal dilution that gives an absorbance value that equals twice the background value (wells that did not contain RBD or S-2P protein).

The mouse isotype ELISA were performed using a similar approach as above, but with the following differences. Only spike protein (S-2P) was used to coat the wells. The plates were blocked with PBS containing 0.2% bovine serum albumin (BSA), pH 7.4 for 30 minutes. The mouse serum samples were serially diluted in duplicates either 3- or 4-fold in PBS containing 0.2% BSA and 0.05% Tween 20, pH7.4. The secondary antibodies were HRP-conjugated AffiniPure Goat Anti-Mouse antibodies from Jackson ImmunoResearch specific for either Fcγ subclass 1, Fcγ subclass 2a, or Fcγ subclass 2c. The secondary antibodies were incubated for 30 minutes. TMB (3,3’,5,5′-Tetramethylbenzidine) substrate (Thermo) was added and the plates were incubated at RT for 5-10 minutes to allow color development. Stop solution (Thermo) was added and the absorbance was measured (450 nm) on a VersaMax microplate reader (Molecular Devices). The titration curves were interpolated to determine the dilution factor where A450=1.0, and the resulting values were used to calculate the IgG1/IgG2a ratio (for Balb/c mice) or IgG1/IgG2c ratio (for C57BL/6 mice).

Serum Cytokine Levels Measured by MSD: Cytokine levels were measured using V-Plex Plus Multi-Spot Assay plates, from Meso Scale Discovery (MSD, Rockville, MD). The mouse Pro-inflammatory panel containing IFN-γ, IL-4, IL-2, and TNF-α was used. Type 1 cytokines in the panel are IFN- γ, IL-2, and TNF-α, and Type 2 cytokine is IL-4. The kit included diluent, wash buffer, detection antibody solution and read buffer, as well as calibrators and controls for each analyte, from the manufacturer. Plates were washed three times with MSD wash buffer before the addition of MSD reference standard and calibrator controls used for quantifying antibody concentrations. Serum samples were diluted at 1:2 in MSD Diluent buffer, then added to wells in duplicate. Plates were incubated for 2 hours at RT with shaking at 350 rpm, then washed three times. MSD Detection Antibody Solution was added to each well, plates were incubated for 2 hours at RT with shaking at 350 rpm then washed three times. MSD 2x Read Buffer T was added to each well. Plates were read by MESO SECTOR S 120 Reader. Analyte concentration was calculated using DISCOVERY WORKBENCH® MSD Software and reported as picograms/mL.

SARS-CoV-2 pseudovirus neutralization assay: SARS-CoV-2 pseudovirions (PSV) were produced by co-transfection of HEK293T/17 cells with a SARS-CoV-2 S plasmid (pcDNA3.4) and an HIV-1 NL4-3 luciferase reporter plasmid. The S expression plasmid sequence was derived from the Wuhan seafood market pneumonia virus isolate Wuhan-Hu-1, complete genome (GenBank accession MN908947), and was codon optimized and modified to remove an 18 amino acid endoplasmic reticulum retention signal in the cytoplasmic tail to improve S incorporation into the pseudovirions and thereby enhance infectivity. Virions pseudotyped with the vesticular stomatitis virus (VSV) G protein were used as a non-specific control. Infectivity and neutralization titers were determined using ACE2-expressing HEK293 target cells (Integral Molecular) in a semi-automated assay format using robotic liquid handling (Biomek NXp Beckman Coulter). Test sera were diluted 1:40 in growth medium and serially diluted, then 25 µL/well was added to a white 96-well plate. An equal volume of diluted SARS-CoV-2 PSV was added to each well and plates were incubated for 1 hour at 37° C. Target cells were added to each well (40,000 cells/ well) and plates were incubated for an additional 48 hours. RLUs were measured with the EnVision Multimode Plate Reader (Perkin Elmer) using the Bright-Glo Luciferase Assay System (Promega Corporation). Neutralization dose-response curves were fitted by nonlinear regression using the LabKey Server, and the final titers are reported as the reciprocal of the dilution of serum necessary to achieve 50% neutralization (ID50, 50% inhibitory dose) and 80% neutralization (ID80, 80% inhibitory dose).

SARS-CoV-2 live-virus neutralization assay: SARS-CoV-2 strain 2019-nCoV/USA_WA1/2020 was obtained from the Centers for Disease Control and Prevention (gift of N. Thornburg). Virus was passaged once in Vero CCL81 cells (ATCC) and titrated by focus-forming assay on Vero E6 cells. Mouse sera were serially diluted and incubated with 100 focus-forming units of SARS-CoV-2 for 1 h at 37° C. Serum-virus mixtures were then added to Vero E6 cells in 96-well plates and incubated for 1 h at 37° C. Cells were overlayed with 1% (w/v) methylcellulose in MEM. After 30 h, cells were fixed with 4% PFA in PBS for 20 minutes at room temperature then washed and stained overnight at 4° C. with 1 µg/ml of antibody CR3022 in PBS supplemented with 0.1% saponin and 0.1% bovine serum albumin. Cells were subsequently stained with HRP-conjugated goat anti-human IgG for 2 h at room temperature. SARS-CoV-2-infected cell foci were visualized with TrueBlue peroxidase substrate (KPL) and quantified using ImmunoSpot microanalyzer (Cellular Technologies). Neutralization curves were generated using Prism software (GraphPad Prism 8.0).

Intracellular staining (ICS) and flow cytometry: Mice (n =5/time point) were euthanized on days 3, 5, 7, and 10 following immunization and spleens were collected. Single cell suspensions from individual immunized mice as well as from 5 unimmunized naïve mice (controls) were also prepared. Cells from each mouse were frozen at approximately 30 million cells/vial and placed in liquid nitrogen until use. Cryopreserved splenocytes were quickly thawed and added to 10 mL of complete RPMI 1640 media supplemented with 5% Fetal bovine serum and 1% Pen-strep followed by viability assessment by trypan blue exclusion method. Approximately, 1×106 cells were cultured in the presence of peptide pools directed towards SARS CoV-2 spike protein (JPT) (1ug/ml) in the presence of protein transport inhibitor (BD Golgi Plug™ containing Brefeldin A, 1 µg/ml, BD Biosciences) for 6 hours at 37° C., 5% CO2. For the positive control, cells were stimulated with phorbol 12-myristate 13-acetate (PMA; Sigma; 50 ng/ml final concentration) and ionomycin (I; Sigma; 1 µg/ml final concentration) while media served as a negative control. After the incubation period, cells were stained with LIVE/DEAD Fixable Aqua Dead Cell Stain Kit (Invitrogen), followed by surface staining with antibodies specific for the following cell surface markers (BUV737 anti-CD3 BUV395 anti-CD4, BV650 anti-CD69, BV711 anti-CD8, APC-H7 anti-CD45R/B220, PE-eFluor610 anti-CXCR5, PECY-7 anti-PD-1, BV785 anti- CCR7, BV605 anti- CD154) obtained from either BD Biosciences, Thermofisher Scientific or Biolegend. Following surface staining, cells were washed twice with FACS buffer. After washing, cells were fixed/permeabilized for 40 min at 4° C. in the dark using the eBioscience™ Intracellular Fixation & Permeabilization Buffer Set (Thermofisher Scientific) as per the manufacturer’s instructions. Cells were then incubated with a panel of intracellular antibodies specific for the following cytokines (V450 anti-IFN-γ, FITC anti-TNF-A, PerCP-Cy5 anti-IL-4, and PE anti IL-2) for 30 min at 4° C., washed twice, and resuspended in FACS buffer followed by acquisition on a BD FACS ARIA II (BD Biosciences, San Diego, CA) and analyzed with FlowJo software (Tree Star, San Carlos, CA). Appropriate single-color compensation controls and fluorescence minus one (FMO) controls were prepared simultaneously and were included in each analysis. Data are shown as bar graphs (Mean + SD). Significance between the two groups was determined by Mann-Whitney test.

Data Analysis: Data analyses used GraphPad (San Diego, CA) Prism software and statistical tests as described for individual experiments.

### Results and Discussion

### SpFN_1B-06-PL In Vitro Characterization

Structure by TEM: The assembly of SpFN_1B-06-PL as a central ferritin nanoparticle with 8 protruding SARS-CoV-2 Spike trimers was confirmed by negative-stain TEM. FIGS. 6 and 11 show reference-free 2D averages and 3D reconstruction of the research-grade SpFN_1B-06-PL immunogen with the expected spherical core and protruding SARS-CoV-2 spikes.

### Study #1 Immunogenicity of SpFN_1B-06-PL With Adjuvant ALFQ or Adjuvant Alhydrogel

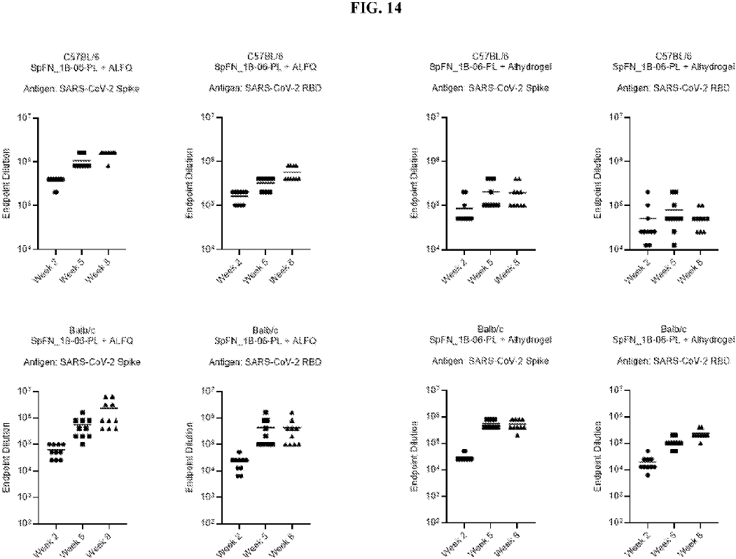

Antibody responses to SARS-CoV-2: C57BL/6 or Balb/c mice were immunized with research grade (RG) SpFN_1B-06-PL intramuscularly with 10 µg of SpFN_1B-06-PL in alternating caudal thigh muscles 3 times, at 3-week intervals (week 0, 3, and 6) using either ALFQ or Alhydrogel as an adjuvant. All mice had robust serum binding responses to SARS-CoV-2 Spike, and RBD at each two-week timepoints following immunization, assessed by Octet Biolayer Interferometry and ELISA as shown in FIG. 14. Mouse sera from week 10 showed robust ACE2 blocking activity in an in vitro high-threshold SARS-CoV-2 RBD-ACE2 blocking assay shown in FIG. 15 with the ALFQ adjuvant groups showing higher levels of ACE2 inhibition.

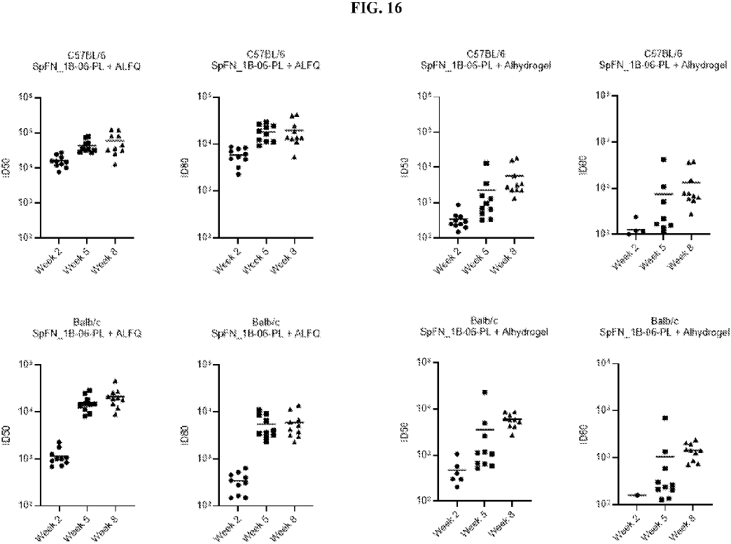

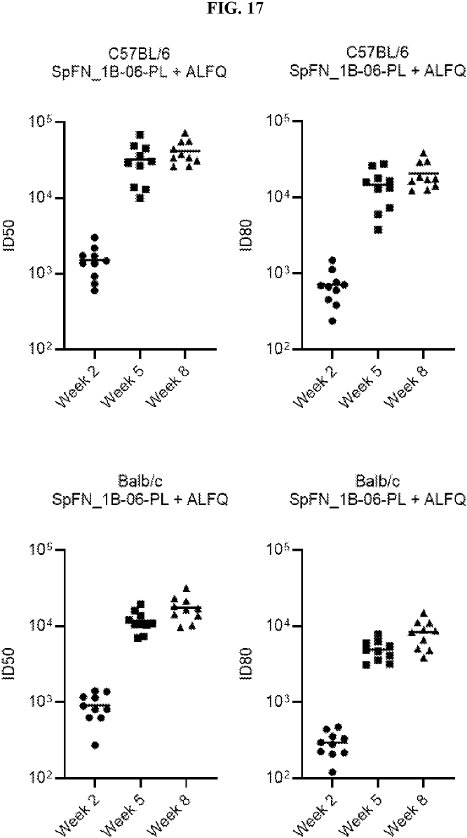

Vaccination Neutralization Titers: Sera from immunized mice two weeks after each immunization were tested for neutralization against SARS-CoV-2 in a pseudovirus neutralization assay (FIG. 16). All vaccinated animal sera exhibited neutralizing activity. Both C57BL/6 and Balb/c mice strains immunized with SpFN_1B-06-PL + ALFQ showed neutralization titers ID₅₀ > 1,000 after a single immunization that increased to ID₅₀ > 10,000 after a second immunization and were maintained or slightly increased after a third immunization. In contrast, SpFN_1B-06-PL + Alhydrogel gave approximately 10-fold lower neutralization titers with neutralization titers ID₅₀ ~ 300 after a single immunization that increased to ID₅₀ > 1,000 after a second immunization and were maintained or slightly increased after a third immunization. Sera from mice immunized with SpFN_1B-06-PL with ALFQ were assessed for neutralization of SARS-CoV-2 in a live-virus neutralization assay. All immunized mice showed robust neutralization after a single immunization, averaging ~ 1,000 which was boosted by ~ 10-fold following a second immunization (FIG. 17). The neutralization titers showed a slight increase following a third immunization.

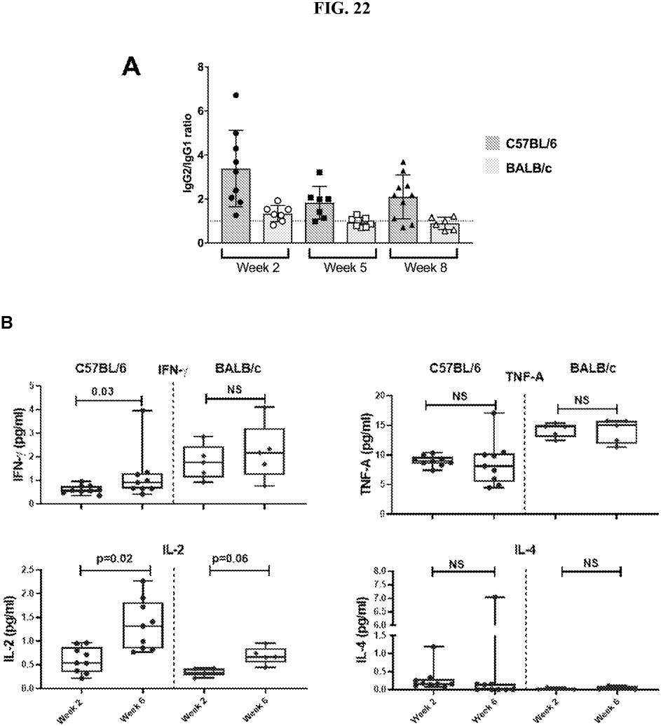

Serum Spike specific antibody isotype usage: Mouse sera was assayed for SARS-CoV-2 Spike-specific antibody response and the ratio of the isotypes, IgG2a or IgG2c (C57BL/6 and Balb/c have a different IgG2 subclass usage), and IgG1 - surrogates of TH1 and TH2 responses respectively (FIG. 22). A low ratio value for IgG2/IgG1 would indicate a TH2 bias, while a high ratio value would indicate a TH1 bias. In both mouse models when ALFQ was used as adjuvant, antibody isotype usage was very balanced with a slight ~2-fold TH1 bias in C57BL/6 mice.

### Study #2 Immunogenicity of a Low Dose of SpFN IB-06-PL With Adjuvant ALFQ

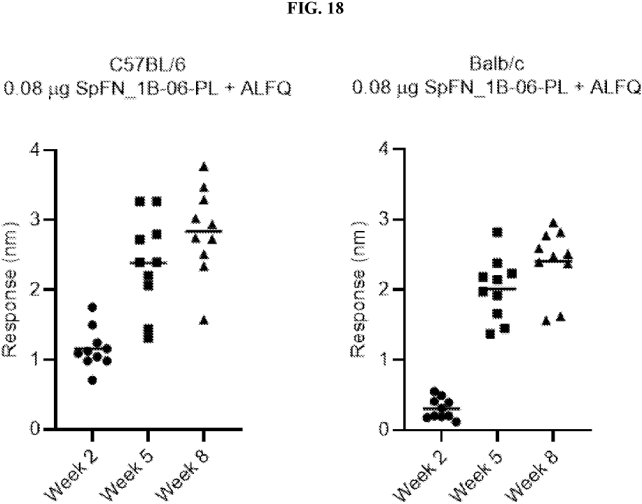

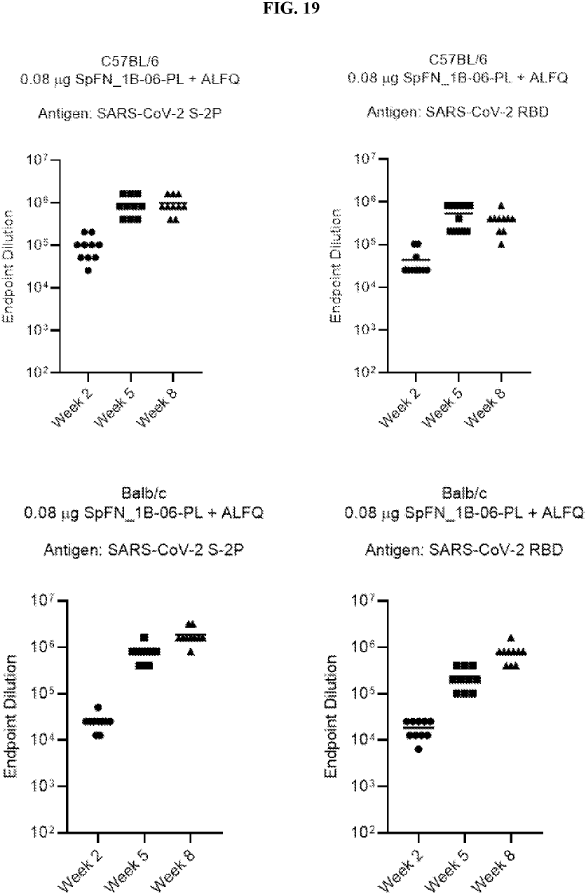

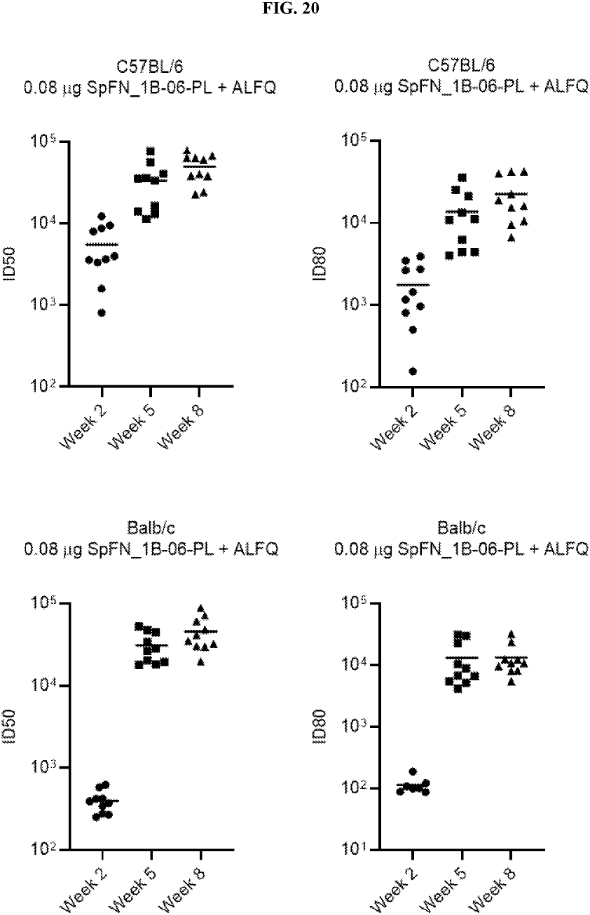

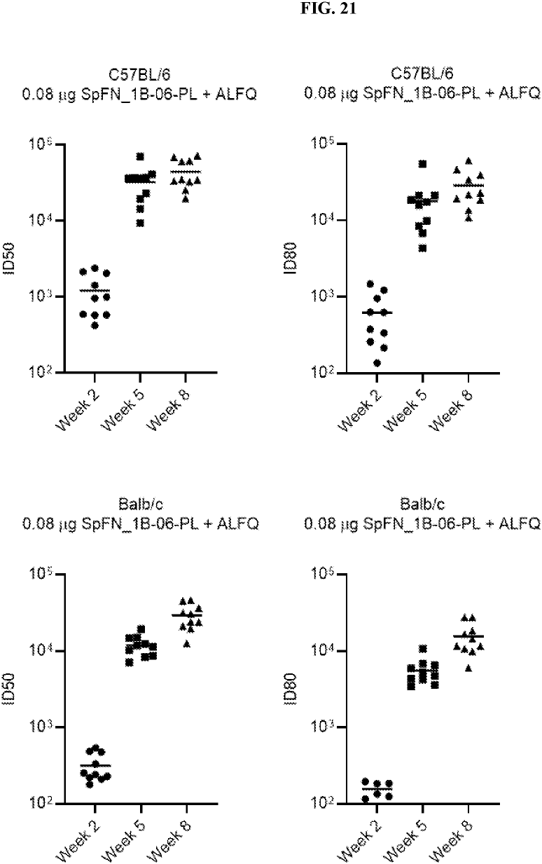

Antibody responses to SARS-CoV-2: In order to assess a lower dose of SpFN_1B-06-PL, C57BL6 or Balb/c mice were immunized with research grade (RG) SpFN_1B-06-PL intramuscularly with 0.08 µg of SpFN_1B-06-PL using ALFQ as an adjuvant. All mice had robust serum binding responses to SARS-CoV-2 Spike, and RBD at two-week timepoints following two immunizations, as shown in FIG. 18 and FIG. 19. Mouse sera from both adjuvant groups demonstrated robust neutralization activity in a pseudovirus neutralization assay against the homologous SARS-CoV-2 as shown in FIG. 20. Mouse sera from the SpFN_1B-06-PL + ALFQ group also showed robust live-virus neutralization, as shown in FIG. 21.

### Study #3 Serum Cytokine Response of SpFN_1B-06-PL With Adjuvant ALFQ

C57BL/6 mice were immunized with research grade (RG) SpFN_1B-06-PL intramuscularly with 10 µg of SpFN_1B-06-PL in alternating caudal thigh muscles twice, at 3 week intervals (week 0, and 3) using ALFQ as an adjuvant. Serum cytokine profiles at week 2 and week 6 were measured in these C57BL/6 mice immunized with SpFN_1B-06-PL + ALFQ and compared to the serum cytokine responses in Balb/c mice immunized in Study 1. A predominant TH1 cytokine response was observed in both mice types with IFN-gamma, IL-2, and TNF-alpha levels measured at week 2 and week 6 showing high levels, while serum IL-4 levels were observed at low levels as shown in FIG. 22.

### Study #4 T Cell Cytokine Response of SpFN_1B-06-PL With Adjuvant ALFQ and Alhydrogel

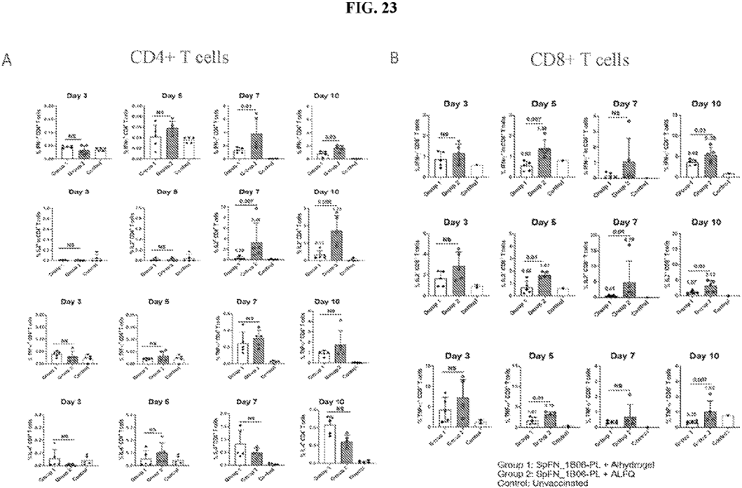

C57BL/6 mice were immunized with a single dose of 10 µg of research grade SpFN_1B-06-PL intramuscularly with of SpFN_1B-06-PL using either ALFQ or Alhydrogel as an adjuvant. Mice were euthanized and spleens were collected from 5 mice in each group on Days 3, 5, 7 and 10. Splenocytes were stimulated with SARS CoV-2 spike protein peptide pools, followed by incubation with cell surface marker antibodies and subsequent flow cytometry. Frequency of CD4 and CD8 T cells with cytokine secretion are shown in FIG. 23. Mouse cells from both adjuvant groups showed robust T cell responses with significant levels of TH1 type responses. Direct measurements of cytokine patterns in vaccine-induced T cells by intracellular cytokine staining (ICS) as shown by the Ifn-γ, IL-2, and TNF-α secreting cells exhibited a Th1-dominant response. The ALFQ adjuvant group showed higher frequency of CD4 and CD8 T cells with TH1 cytokine profiles.

### Conclusions

Research grade SpFN_1B-06-PL administered with either ALFQ or Alhydrogel adjuvants was shown to elicit antibodies that bound homologous SARS-CoV-2 S and RBD, inhibited ACE2 binding, and neutralized SARS-CoV-2 viruses in a pseudovirus assay and live virus assay. Immune responses were consistently higher when using ALFQ as an adjuvant. Use of a 0.08 µg SpFN_1B-06-PL dose with ALFQ adjuvant elicited high levels of binding and neutralizing antibodies at similar levels elicited by the higher 10 µg dose. These data indicate that SpFN_1B-06-PL with ALFQ is immunogenic in two mouse models. Analysis of antibody isotype usage in Spike-specific responses show a balanced TH1/TH2 type response with both IgG1 and IgG2 antibody subtypes in usage when the adjuvant ALFQ is used. In addition, serum cytokine profiles also indicated high levels of TH1 cytokine responses and analysis of spleen cells taken from C57BL/6 mice immunized with SpFN_1B-06-PL + ALFQ show increased frequency of Ifn-γ, IL-2, and TNF-α positive CD4 and CD8 T cells following vaccination indicative of a TH1 type immune response. The Ig subclass and T cell cytokine data together demonstrate that immunization with SpFN_1B-06-PL with ALFQ elicits a balanced TH1/TH2 response in contrast to the TH2-biased responses that have been linked to VAERD.

### Example 3 - SpFN and RBD-Ferritin Elicited Serum Provides Protective Immunity in K18-ACE2 Transgenic Mice

Animal models of SARS-CoV-2 infection are useful for characterizing vaccines and therapeutic intervention modalities and to enable understanding of mechanisms of diseases. With few exceptions, the disease pathology and severity in rodent and primate animal models does not approach the levels seen in humans. In order to develop a useful rodent model, Perlman and colleagues (McCray et al., Lethal infection of K18-hACE2 mice infected with severe acute respiratory syndrome coronavirus. J Virol. 2007 Jan; 81(2):813-21 and Zheng et al., COVID-19 treatments and pathogenesis including anosmia in K18-hACE2 mice. Nature. 2021 Jan; 589(7843):603-607) developed a transgenic mouse model which incorporated the human angiotensin-converting enzyme 2 (ACE2) in airway and other epithelia cells. The expression of ACE2 is hACE2 driven by the cytokeratin 18 (KRT18) promoter. ACE2 is the receptor for SARS-CoV-2 and SARS-CoV enabling human infection and in the K18-ACE2 transgenic mouse model, serves to enable reproducible infections following intranasal inoculation with a human strain of the virus. Depending on the SARS-CoV-2 viral dose utilized, the animals can exhibit disease leading to death.

In this study polyclonal IgG was purified from C57BL/6 mice that had been vaccinated with either SpFN_1B-06-PL (Week 6 - mice C826-C830) or RBD-Ferritin_pCoV131 (Week 17 -mice 581-590), and passively transferred three amounts of IgG from either immune serum to a set of K18-ACE2 transgenic mice, as well as naive IgG or PBS. Mice were infected with SARS-CoV-2 one day later and then monitored twice daily for clinical symptoms, weight loss and morbidity and or mortality.

Methods: Sera was purified from two groups of mice, SpFN_1B-06-PL-immunized or RBD-Ferritin_pCoV131-immunized. Sera from each group was pooled and measured for neutralization activity. The sera from each group was purified using ProteinG resin to isolate the polyclonal IgG. Sera was assessed for complete depletion and loss of RBD-binding activity, and the purified IgG was assessed for RBD-binding by Octet Biolayer Interferometry.

On study day -1 mice were injected intraperitoneally with the indicated amount of purified IgG from the pre-Immune Serum. On study day 0, all mice were infected with 4.1×104 PFU of SARS-CoV-2 USA-WA1/2020 via intranasal instillation. All mice were monitored for clinical symptoms and body weight twice daily, every 12 hours, from study day 0 to study day 14. Mice were euthanized if they displayed any signs of pain or distress as indicated by the failure to move after stimulated or inappetence, or if mice have greater than 20% weight loss compared to their study day 0 body weight.

### Results/Conclusions

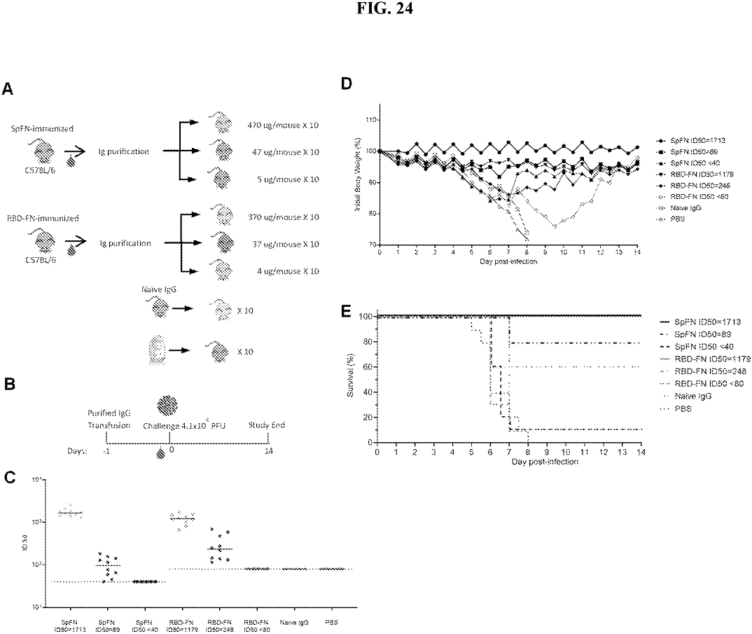

To assess vaccine-elicited antibodies ability to prevent SARS-CoV-2 related mortality and morbidity in the K18-ACE2 mouse model, reducing amounts of purified IgG were transfused from either SpFN_1B-06-PL-immunized or RBD-Ferritin-immunized mice (FIG. 24 and Table 7). Animals were challenged one day following antibody transfusion, and the serum neutralizing antibody titer assessed. The challenged mice were assessed for change in body weight and mortality over 14 days following challenge. Since the C57BL/6 vaccinated mice showed high neutralization titers, and in an effort to understand the levels of antibody that provide protection, and the levels that would allow SARS-CoV-2 related mortality, levels of antibody with neutralization ID50 > 1,000, and decreasing to <40 were provided (Table 7). These amounts are significantly lower than levels observed following SpFN_1B-06-PL or RBD-Ferritin_pCoV131 vaccination. K18-ACE2 mice that received the highest antibody amounts from either SpFN-1B-06-PL-vaccinated animals or RBD-Ferritin_pCoV131-vaccinated animals did not show body weight loss, and all survived. Mice that received approximately one tenth that level of antibody, also showed significant levels of survival, 80% for the SpFN_1B-06-PL group, and 60% for the RBD-Ferritin_pCoV131 group. Animals that received the lowest amount of antibody, did not show any increased antibody-enhanced rates of morbidity or mortality, and in both group3, and 6, a single animal survived. All animals in both control groups succumbed to disease by day 8 post-challenge. In conclusion, passive transfer of antibody alone from either SpFN_1B-06-PL- or RBD-Ferritin_pCoV131-vaccinated animals is suitable to provide protection from SARS-CoV-2 morbidity, and mortality in the K18-ACE2 mouse model.

TABLE 7

Experimental design, pseudovirus neutralization titer and survival percentage

| Group | Immunogen | Animal sera type | Pooled sera neut (ID50) prior to purification | Amount of purified IgG transferred (ug/mouse) | GMT of sera at time of challenge | % survival |
|---|---|---|---|---|---|---|
| 1 | pCoV1B-06-PL | SpFN_1B-06PL-immunized | 33106 | 470 | 1713 | 100 |
| 2 | pCoV1B-06-PL | SpFN_1B-06PL--immunized | 33106 | 47 | 89 | 80 |
| 3 | pCoV1B-06-PL | SpFN_1B-06PL--immunized | 33106 | 5 | <40 | 10 |
| 4 | pCoV131 | RBD-Ferr_pCoV131 immunized | 23126 | 370 | 1179 | 100 |
| 5 | pCoV131 | RBD-Ferr_pCoV131 immunized | 23126 | 37 | 248 | 60 |
| 6 | pCoV131 | RBD-Ferr_pCoV131 immunized | 23126 | 4 | <80 | 10 |
| 7 | N/A | Naive IgG | N/A | N/A | <80 | 0 |
| 8 | N/A | PBS | N/A | N/A | <80 | 0 |

Each of the 8 study groups contained 5 male and 5 female mice.

### Example 4 - Immunization of Mice With SARS-CoV-2 Immunogens Provides a Broadly Neutralizing Immune Response

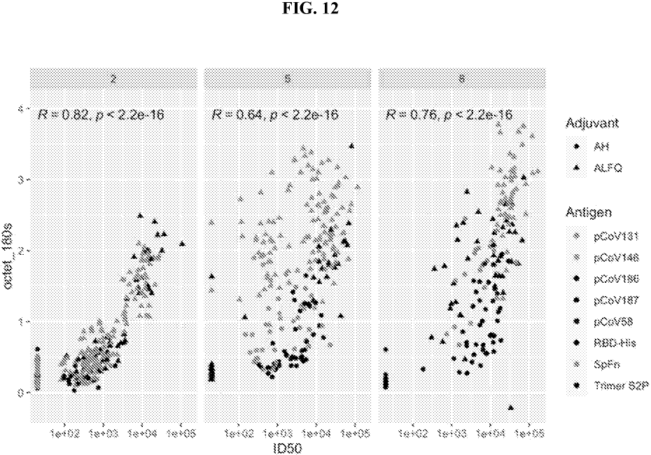

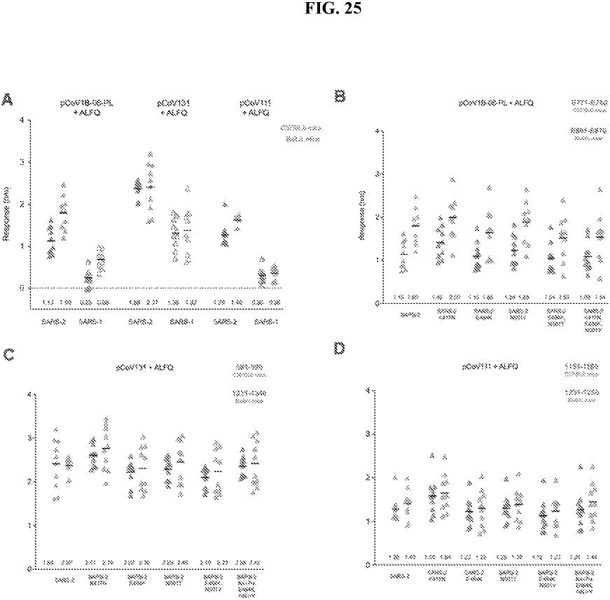

As shown in FIG. 25, mouse sera from animals immunized with SARS-CoV-2 nanoparticles including but not limited to SpFN_1B-06-PL, RBD-Ferritin_pCoV131, and S1-Ferritin_pCoV111 showed binding response as measured by Octet Biolayer Interferometry to the RBD of the homologous SARS-CoV-2 RBD, but also measurable binding to the distantly related SARS-CoV-1 virus. The levels of binding are greater than 0.5 nm which based on the correlation of Octet binding response to RBD molecules and pseudovirus neutralization as shown in FIG. 12 indicated that this level of binding would indicate significant neutralization activity in the mouse sera. In addition, binding was measure for the mouse sera to a set of RBD variant mutations that match to mutations observed in circulating strains of SARS-CoV-2 including mutations at residue 417, 484, and 501. In all tested cases, no dramatic change was seen in the binding responses between SARS-CoV-2 RBD or versions that had mutations.

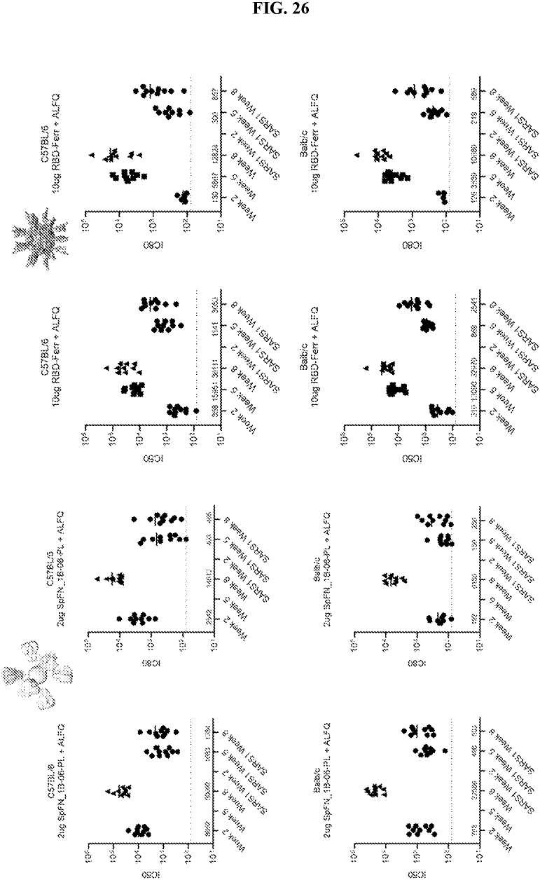

As shown in FIG. 26, the mouse sera was measured for pseudovirus neutralization activity against SARS-CoV-2 and SARS-CoV-1 and saw high levels of neutralization ID50 titers of >1,000 for the animals immunized with SpFN_1B-06-PL and >3,000 for the RBD-Ferritin_pCoV131 immunized mice.

### Example 5 - Non-Human Primate Immunogenicity and Efficacy

The Spike protein is a surface protein of Severe Acute Respiratory Syndrome associated Coronavirus 2 (SARS-CoV and attaches to human angiotensin converting enzyme (ACE)-2 cell surface receptors facilitating human infection. Antibodies that can bind to the Spike glycoprotein and prevent interaction with the ACE2 receptor can facilitate protection from infection. The present inventors developed a Spike Ferritin Protein Nanoparticle with ALFQ adjuvant (SpFN_1B-06-PL + ALFQ) vaccine to elicit protective antibody responses. Ferritin is a naturally occurring protein that self-assembles into a 24-member spherical particle, made up of multiple three-fold, four-fold and two-fold axes. Using the 3-fold axes, 8 trimeric SARS-CoV-2 Spike glycoproteins are presented on the surface of the self-assembling protein nanoparticle surface. The ALFQ adjuvant, a liposomal formulation containing the QS-21 saponin, was developed by the Laboratory of Adjuvant and Antigen Research (LAAR) at Walter Reed Army Institute of Research (WRAIR). It is a liposomal formulation containing MPLA and QS-21 saponin. In this study in Chinese-origin rhesus macaques, the Spike Ferritin nanoparticle pCoV-1B-06-PL elicited antibodies that bind to SARS-CoV-2 Spike and Receptor-Binding domain (RBD), neutralize homologous SARS-CoV-2 in a pseudovirus assay, and inhibit Spike and RBD binding to the human ACE-2 receptor. In addition, following respiratory tract SARS-CoV-2 challenge, vaccinated animals were protected from infection as evidenced by lack of viral replication in the upper and lower airways.

### List of Abbreviations

SpFN: Spike Ferritin Nanoparticle
RBD: Receptor-binding domain
MPLA: monophosphoryl lipid A
NHP: Nonhuman primate
ACE-2: angiotensin-converting enzyme 2
NP: nasopharyngeal
BAL: bronchoalveolar lavage
TND: target not detected

Introduction: In order to extend observations made in murine models evaluating the immunogenicity of SpFN adjuvanted with ALFQ, here the immunogenicity and efficacy of ALFQ-adjuvanted SpFN was investigated in rhesus macaques. Immune responses elicited in nonhuman primate (NHP) species are expected to resemble those in humans due to close genetic similarity. Moreover, NHP species offer an important model for evaluating the effect of SARS-CoV-2 vaccines on viral replication in both upper and lower airways. Important questions addressed here include the dose of immunogen required to elicit protective immune responses, and whether a single immunization is sufficient to mount robust responses and protection.

### Objectives

Measure humoral immune responses in rhesus macaques vaccinated with SpFN_1B-06-PL adjuvanted with ALFQ, including SARS-CoV-2-specific binding and neutralizing antibodies.
Compare immune responses following two versus one SpFN_1B-06-PL immunization
Compare immune responses elicited by 50 µg versus 5 µg SpFN_1B-06-PL
Assess protective efficacy against intranasal/intratracheal SARS-CoV-2 challenge in SpFN_1B-06-PL vaccinated macaques

### Materials and Methods

### Materials

SpFN_1B-06-PL: Endotoxin-free, research grade material was used for vaccinations. Research-grade SpFN_1B06-PL was produced by transient expression in Expi293F cells (ThermoFisher Scientific) using the same expression construct sequence as that used to create the SpFN_1B06-PL cGMP manufacture of clinical drug product. Culture supernatant was harvested four days post-transfection and purified by GNA-lectin affinity chromatography and size-exclusion chromatography. Purified research grade SpFN_1B06-PL was formulated in PBS at 1 mg/ml.

ALFQ: ALFQ liposomes (human dose) contained 200 µg/mL 3D-PHAD (MPLA:PL=1:88; Avanti Polar Lipids) and 100 ug/mL QS-21 (Desert King International), 11.45 mM phospholipids (DMPC:DMPG=9:1), 55% cholesterol, 200 µg/mL 3D-PHAD. ALFQ was gently mixed by slow speed vortex prior to use.

All reagents were equilibrated to room temperature before use. Antigen was diluted to 0.1 mg/mL in dPBS (Lot#723188, Quality Biological) and mixed 1:1 (50 µg dose) or 1:10 (5 µg dose) with 2X ALFQ liposomes on a tilted roller at slow speed at room temperature for 10 min, followed by incubation at 4° C. for 50 min. Immunizations were performed within 3 hours of vaccine formulation.

SARS-CoV-2 challenge stock: SARS-CoV-2 virus (strain 2019-nCoV/USA-WA1/2020, Lot# 70038893, 1.99 × 10⁶ TCID₅₀/mL) used for rhesus challenge was obtained from NIAID. Virus was stored at -80° C. prior to use, thawed by hand and placed immediately on wet ice. Stock was diluted to 5×10⁵ TCID₅₀/mL in PBS and vortexed gently for 5 seconds prior to inoculation of macaques.

### Methods

TEST ANIMAL HOUSING AND CARE: Thirty-two male and female specific-pathogen-free, research naive Chinese-origin rhesus macaques were acquired. In vivo procedures were carried out in accordance to institutional, local, state, and national guidelines and laws governing research in animals including the Animal Welfare Act. Animal protocols and procedures were reviewed and approved by the Animal Care and Use Committee of both the US Army Medical Research and Material Command (USAMRMC, protocol 11355007.03) Animal Care and Use Review Office as well as the Institutional Animal Care and Use Committee of Bioqual, Inc. (protocol number 20-092), where non-human primates were housed for the duration of the study. Bioqual, Inc. and the USAMRMC are both accredited by the Association for Assessment and Accreditation of Laboratory Animal Care and are in full compliance with the Animal Welfare Act and Public Health Service Policy on Humane Care and Use of Laboratory Animals.

ANIMAL STUDY DESIGN AND PROCEDURES: Thirty-two rhesus macaques (n=8/group) were immunized intramuscularly with either 50 or 5 µg of SpFN_1B-06-PL in alternating anterior proximal quadricep muscles. SpFN was administered in a 1.0 mL dose formulated in ALFQ. Study groups, balanced for animal sex and weight, were as follows:
PBS
SpFN_1B-06-PL (50ug) + ALFQ adjuvant, prime+boost
SpFN_1B-06-PL (5ug) + ALFQ adjuvant, prime+boost
SpFN_1B-06-PL (50ug) + ALFQ adjuvant, 1 immunization (study week 4)

Immunizations were administered twice 4 weeks apart (groups 2-3) or once (group 4). Blood was collected every 2 weeks following each immunization for 8 weeks. Serum was stored at -80° C. until analysis. Antibody responses were analyzed by Octet Biolayer Interferometry, MSD, pseudovirus neutralization, and wild-type live virus neutralization assays. Animals were challenged at study week 8 via combined intratracheal (IT, 1.0 mL) and intranasal (IN, 0.5 mL per nostril) inoculation of a 10⁶ TCID50 dose of SARS-CoV-2 strain 2019-nCoV/USA-WA1/2020. The IN/IT challenge route was selected due to its widespread usage and establishment as the current standard in the field for NHP challenge studies. The 10⁶ TCID50 dose was intended to provide a rigorous challenge model with robust viral replication in all control animals. Animals were followed for 7 (N=16) or 14 days (N=16) following challenge. Respiratory tract specimens, nasopharyngeal (NP) swabs and bronchoalveolar lavage (BAL), were collected to assess viral replication in the upper and lower airways, respectively, at days 1, 2, 4, 7, 10, and 14 post-challenge.

### Experimental Procedures

Octet Biolayer Interferometry: All biosensors were hydrated in PBS prior to use. All assay steps were performed at 30° C. with agitation set at 1,000 rpm in a Octet 96red instrument (ForteBio). Baseline equilibration of the anti-His-tag biosensors (HIS1K biosensors with a conjugated Penta-His antibody (ForteBio)) was carried out using assay buffer (PBS) for 15 s, prior to SARS-CoV2-RBD (30ug/ml diluted in PBS) loading for 120 s. After briefly dipping in assay buffer (15 s in PBS), the biosensors were dipped in the mouse sera samples (100-fold dilution) for 180 s. The binding response (nm) at 180 s was recorded for each sample.

Binding antibody measurements by MSD: SARS-CoV-2-specific binding IgG antibody responses were measured using MULTI-SPOT® 96-well plates, (Meso Scare Discovery [MSD}, Rockville, MD). Multiplex wells were coated with three SARS-CoV-2 antigens, Spike, RBD and Nucleocapsid (S, RBD and N) at a concentration of 200-400 ng/ml and BSA which served as a negative control. 4 plex MULTISPOT plates were blocked with MSD Blocker A buffer for 1 hour at room temperature (RT) while shaking at 700 rpm. Plates were washed with MSD wash buffer before the addition of MSD reference standard and calibrator controls used for quantifying antibody concentrations. Serum samples were diluted at 1:1,000 - 1:100,000 in MSD Diluent buffer, then added to each of four wells. Plates were incubated for 2 hours at RT with shaking at 700 rpm, then washed. MSD SULFO-TAG™ anti-IgG antibody was added to each well, plates were incubated for 1 hour at RT with shaking at 700 rpm, washed, then MSD GOLDTM Read buffer B was added to each well. Plates were read by MESO SECTOR S 120 Reader. IgG concentration was calculated using DISCOVERY WORKBENCH® MSD Software and reported as arbitrary units (AU)/mL.

ACE-2 binding inhibition antibody measurements by MSD: SARS-CoV-2 Spike-specific binding antibody responses able to inhibit Spike or RBD binding to the ACE-2 receptor competition were measured using MULTI-SPOT® 96-well plates (MSD}, Rockville, MD). Antigen-coated plates were blocked and washed as described above. Assay calibrator and samples were diluted at 1:25-1:1,000 in MSD Diluent buffer, then added to the wells. Plates were incubated for 1 hour at RT with shaking at 700 rpm. ACE2 protein conjugated with MSD SULFO-TAG™ was added, plates were incubated for 1 hour at RT with shaking at 700 rpm. Plates were washed and read as described above. Percent inhibition was calculated relative to the assay calibrator (maximum 100% inhibition). AU/mL concentration of the inhibitory antibody was calculated with DISCOVERY WORKBENCH® MSD Software.

SARS-CoV-2 pseudovirus neutralization assay: SARS-CoV-2 pseudovirions (PSV) were produced by co-transfection of HEK293T/17 cells with a SARS-CoV-2 S plasmid (pcDNA3.4) and an HIV-1 NL4-3 luciferase reporter plasmid. The S expression plasmid sequence was derived from the (Wuhan strain) genome (GenBank #), and was codon optimized and modified to remove an 18 amino acid endoplasmic reticulum retention signal in the cytoplasmic tail to improve S incorporation into the pseudovirions and thereby enhance infectivity. Virions pseudotyped with the vesicular stomatitis virus (VSV) G protein were used as a non-specific control. Infectivity and neutralization titers were determined using ACE2-expressing HEK293 target cells (Integral Molecular) in a semi-automated assay format using robotic liquid handling (Biomek NXp Beckman Coulter). Test sera were diluted 1:40 in growth medium and serially diluted, then 25 µL/well was added to a white 96-well plate. An equal volume of diluted SARS-CoV-2 PSV was added to each well and plates were incubated for 1 hour at 37° C. Target cells were added to each well (40,000 cells/ well) and plates were incubated for an additional 48 hours. RLUs were measured with the EnVision Multimode Plate Reader (Perkin Elmer) using the Bright-Glo Luciferase Assay System (Promega Corporation). Neutralization dose-response curves were fitted by nonlinear regression using the LabKey Server, and the final titers are reported as the reciprocal of the dilution of serum necessary to achieve 50% neutralization (ID50, 50% inhibitory dose) and 80% neutralization (ID80, 80% inhibitory dose).

SARS-CoV-2 live-virus neutralization assay: SARS-CoV-2 strain 2019-nCoV/USA WA1/2020 was obtained from the Centers for Disease Control and Prevention (gift of N. Thornburg). Virus was passaged once in Vero CCL81 cells (ATCC) and titrated by focus-forming assay on Vero E6 cells. Rhesus sera were serially diluted and incubated with 100 focus-forming units of SARS-CoV-2 for 1 hr at 37° C. Serum-virus mixtures were then added to Vero E6 cells in 96-well plates and incubated for 1 hr at 37° C. Cells were overlaid with 1% (w/v) methylcellulose in MEM. After 30 hrs, cells were fixed with 4% PFA in PBS for 20 minutes at room temperature then washed and stained overnight at 4° C. with 1 µg/ml of antibody CR3022 in PBS supplemented with 0.1% saponin and 0.1% bovine serum albumin. Cells were subsequently stained with HRP-conjugated goat anti-human IgG for 2 hrs at room temperature. SARS-CoV-2-infected cell foci were visualized with TrueBlue peroxidase substrate (KPL) and quantified using ImmunoSpot microanalyzer (Cellular Technologies). Neutralization curves were generated using Prism software (GraphPad Prism 8.0).

Antigen-specific T cell intracellular cytokine staining (ICS): Cryopreserved PBMC were thawed, rested for 6 h in R10 with 50 U/ml Benzonase Nuclease (Sigma-Aldrich), and stimulated with peptide pools for 12 h. Stimulations consisted of either SARS-CoV-2 Spike or Nucleoprotein peptide pools (1 µg/ml, JPT, PM-WCPV-S And PM-WCPV-NCAP respectively) in the presence of Brefeldin A (0.65 µl/ml, GolgiPlugTM, BD Cytofix/Cytoperm Kit, Cat. 555028), co-stimulatory antibodies anti-CD28 (BD Biosciences Cat. 555725 1 ug/ml) and anti-CD49d (BD Biosciences Cat. 555501; 1 ug/ml) and CD107a (H4A3, BD Biosciences Cat. 561348, Lot 9143920). Following stimulation, cells were stained serially with LIVE/DEAD Fixable Blue Dead Cell Stain (ThermoFisher #L23105) and a cocktail of fluorescent-labeled antibodies (BD Biosciences unless otherwise indicated) to cell surface markers CD4-PE-Cy5.5 (S3.5, ThermoFisher #MHCD0418, Lot 2118390), CD8-BV570 (RPA-T8, BioLegend #301038, Lot B281322), CD45RABUV395 (5H9, #552888, Lot 154382 and 259854), CD28 BUV737 (CD28.2, #612815, Lot 0113886), CCR7-BV650 (GO43H7, # 353234, LotB297645) and HLA-DR BV480 (G46-6, # 566113, Lot 0055314). Intracellular cytokine staining was performed following fixation and permeabilization (BD Cytofix/Cytoperm, Becton Dickenson) with CD3-Cy7APC (SP34-2, #557757, Lot 6140803), CD154-Cy7PE (24-31, BioLegend # 310842, Lot B264810), IFNy-AF700 (B27, # 506516, LotB187646), TNFα-FITC (MAb11, # 554512, Lot 15360), IL-2-BV650 (MQ1-17H12, BioLegend #500334, Lot B214940), IL-4 BB700 (MP4-25D2, Lot 0133487), MIP-1b (D21-1351, # 550078, Lot 9298609), CD69-ECD (TP1.55.3, Beckman Coulter # 6607110, Lot 7620070), IL-21-AF647 (3A3-N2.1, # 560493, Lot 9199272), IL-13-BV421 (JES10-5A2, # 563580, Lot 9322765) and IL-17a-BV605 (BL168, Biolegend #512326, B289357). Sample staining was measured on a FACSymphony™ A5 SORP (Becton Dickenson) and data analyzed using FlowJo v.9.9 software (Tree Star, Inc.). CD4 and CD8 T cell subsets were pre-gated on memory markers prior to assessing cytokine expression as follows: single-positive or double-negative for CD45RA and CD28. Boolean combinations of cells expressing one or more cytokines were used to calculate the sum total of antigen-specific memory CD4 or CD8 T cells. Statistical analysis and display of multicomponent distributions were performed with SPICE v6.0 (NIAID, NIH).

SARS-CoV-2 Sub-genomic messenger (sgm) and viral load RNA quantitative assays: RT-qPCR assays were developed targeting the Envelope (E) gene region of SARS-CoV-2 for sgmRNA and viral load RNA quantification. The sgmRNA assay uses the subgenomic (sg) Leader sequence as the forward primer (SARS-CoV-2 sg Leader) in combination with SARS-CoV-2 TAL E1 reverse (R) and SARS-CoV-2 TAL E1 Probe for amplification of the E gene messenger RNA. Quantitative amplification for viral load is performed using the SARS-CoV-2 TAL E1 forward (F) primer with SARS-CoV-2 TAL E1 R and SARS-CoV-2 TAL E1 Probe. All primers and probes are listed in Table 8.

TABLE 8

Primers and Probes for SARS-CoV-2 sgmRNA and Viral Load Assays

| Primer/Probe Name | Sequence 5′- 3′ | Nucleotide Length |
|---|---|---|
| SARS-CoV-2 TAL E1 F | TCGTGGTATTCTTGCTAG | 18 |
| SARS-CoV-2 TAL E1 R | GAAGGTTTTACAAGACTCAC | 20 |
| SARS-CoV-2 TALE1 Probe | FAM -ACACTAGCCATCCTTACTGCG-BHQ1 | 21 |
| SARS-CoV-2 sg Leader | CGATCTCTTGTAGATCTGTTCTC | 23 |
| MS2 F | CTCTGAGAGCGGCTCTATTGG | 21 |
| MS2 R | GTTCCCTACAACGAGCCTAAATTC | 24 |
| MS2 Probe | JOE-TCAGACACGCGGTCCGCTATAACGAT- BHQ2 | 26 |
| T7-Leader Forward | TAATACGACTCACTATAGGGGAATTGTGCGTGGATGAG GCGATCTCTTGTAGATCTGTTCTC | 62 |

F=forward; R=reverse

An RNA transcript for the SARS-CoV-2 envelope gene was used as a calibration standard. T7-Leader and SARS-CoV-2 TAL E1 R primers amplified a 237 base pair sgm E RNA. sgm E RNA transcripts were generated from the T7 - Leader E gene PCR product using the MEGAscript™ T7 Transcription Kit (AM1333: Thermo Fisher Scientific, Inc. Carlsbad, CA). Avogadro’s number was used to convert the sgm E RNA standard concentration from µg/ml to copies/ml.

RNA was extracted from 200 µl of Nasopharyngeal (NP) swab media or Bronchoalveolar Lavage (BAL) specimens using the EZ1 DSP Virus kit (62724: QIAQEN) on the EZ1 Advanced XL instrument (9001874: QIAGEN). Samples were lysed in 200 µl of ATL buffer (19076: QIAGEN), then transferred to the Qiagen EZ1 for extraction. Bacteriophage MS2 (ATCC, Manassas, VA) was added to the RNA carrier and used as an Extraction Control to monitor the efficiency of RNA extraction and amplification. Purified RNA was eluted in 90 µl. A SARS-CoV-2 negative control (NEG) and two contrived SARS-CoV-2 positive controls at 1E6 HIGH and 1E3 LOW concentrations were extracted in each run and used to assess performance of both assays.

The RT-qPCR amplification reactions were performed in separate wells on a 96-well Fast plate for the 3 targets: sgmRNA, RNA viral load, and MS2 RNA. Extraction Controls (NEG, HIGH and LOW) and no template control (NTC) for each primer/probe set were included on each plate. RT-qPCR reactions contained 0.72 uM each Primer and 0.2 uM probe and 1x TaqPath™ 1-Step RT-qPCR (A15299: Life Technologies, Thermo Fisher Scientific, Inc.); amplification was performed on the 7500 Fast Dx thermocycler (4406985: Applied Biosystems, Thermo Fisher Scientific, Inc.). Ten-fold serial dilutions of the sgm E RNA standard in 20 ng/µl t-RNA (stabilizer) was performed to generate calibrators at 1E6, 1E5, 1E4, 1E3, 1E2 and 1E1 RNA copies/10 µl; RNA calibration standards were amplified in duplicate to generate the standard curve. Ten µl of sample RNA and calibration standards were amplified using the following cycling conditions: 2 min at 25° C., 15 min at 50° C., 2 min at 95° C. and 45 cycles of 3 sec at 94° C. and 30 sec at 55° C. with fluorescent read at 55° C. RNA copy values were extrapolated from the standard curve and multiplied by 45 to obtain RNA copies/ml.

Validity of the RT-qPCR result was based upon the following criteria: 1) slope of standard curve, 2) Y intercept, 3) value of high copy SARS-CoV-2 control, 4) value of low copy SARS-CoV-2 control, 5) cycle threshold (C_{T}) value for the MS2 phage extraction control 6) no SARS-CoV-2 amplification in NTC and negative extraction controls, and 7) MS2 target must be detected in all extracted RNA samples.

### Results

### Immunogenicity of SpFN or RBD-Ferritin Adjuvanted With ALFQ in Rhesus Macaques

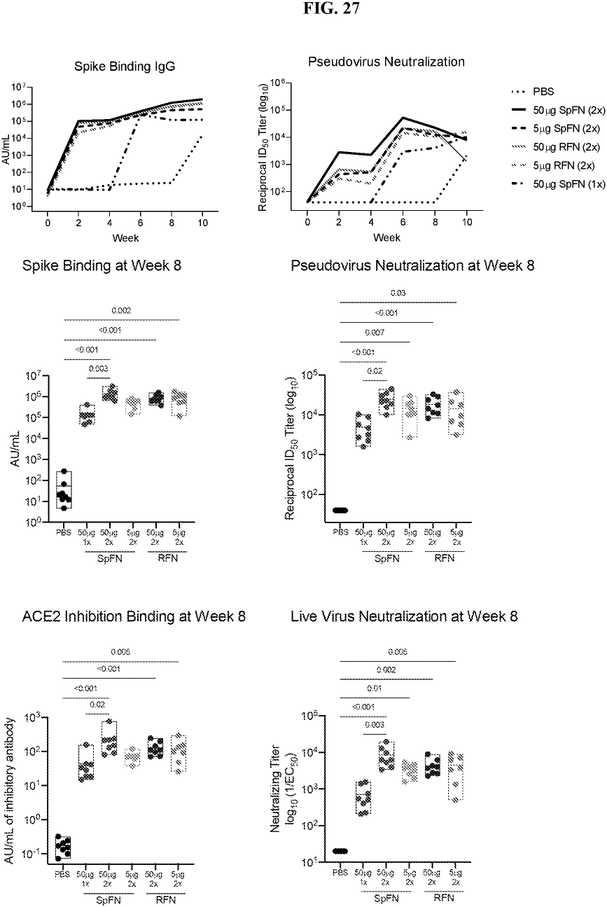

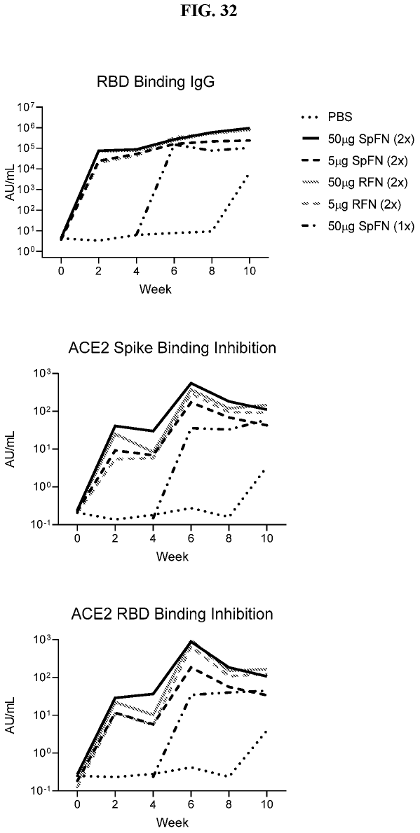

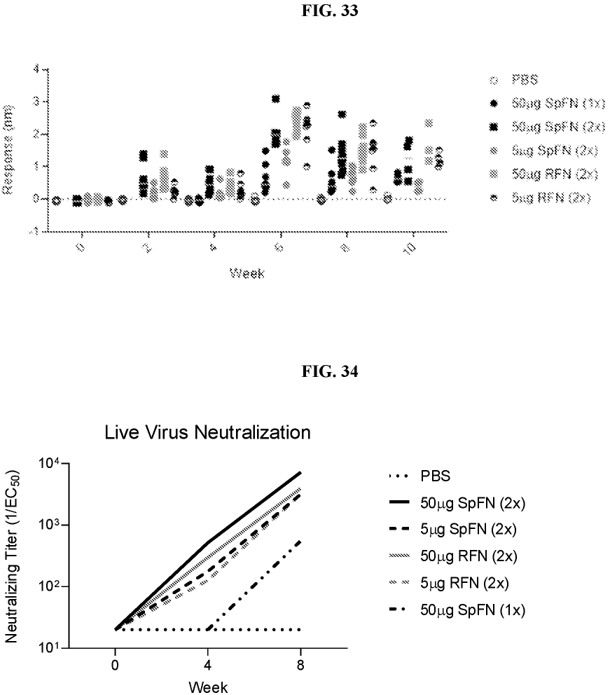

Binding antibody responses to SARS-CoV-2 and SARS-CoV-1: Rhesus macaques were immunized with research grade SpFN_1B-06-PL or RBD-Ferritin intramuscularly at doses of 50 or 5 µg of SpFN_1B-06-PL in alternating anterior proximal quadricep muscles twice (weeks 0 and 4) or once for SpFN_1B-06-PL (50 µg dose at study week 4). Immunogens were formulated with ALFQ adjuvant (human dose). All macaques mounted serum binding responses to SARS-CoV-2 Spike at all time points following immunization as measured by Octet and by MSD (FIG. 27 and FIGS. 31-32). Greater magnitude responses were elicited by the 50 µg dose than by 5 µg and titers were generally sustained from 2 to 4 weeks post-immunization at both doses. Responses increased following boosting by ~10-fold, regardless of dose. Lower levels of antibody responses to the SARS-CoV-1 RBD molecule were observed, but a similar pattern held, with the higher dose immunizations resulting in higher immune responses.

Binding antibody responses to SARS-CoV-2 that inhibit ACE-2 receptor engagement: To assess the ability of SARS-CoV-2-specific humoral responses to block binding between the viral Spike protein and the ACE-2 host cellular receptor, serum was evaluated for activity in an ACE-2 inhibition assay using the MSD platform. SpFN_1B-06-PL and RBD-Ferritin immunization elicited antibody responses that blocked interaction of both the Spike and RBD subunit with the ACE-2 receptor (FIG. 27 and FIGS. 31-32). Inhibitory responses to the priming immunization were robust following immunization with either 50 µg or 5 µg doses (FIG. 27). Boosting increased responses by >10-fold at both doses and responses were well-maintained between 2 and 4 weeks following each immunization.

Pseudovirus neutralizing antibody responses: To evaluate antibody responses able to neutralize SARS-CoV-2 Spike, a pseudovirus neutralization assay was performed with sera collected at weeks 0, 2, 4, 6 and 8. All vaccinated animal sera exhibited neutralizing activity (FIG. 27). For the SpFN 50 µg dose group, geometric mean IC50 titers ranged from 300-20,000 (median 3315) and IC80 titers ranged from 100-2,700 (median 600). Neutralization titers in the SpFN_1B-06-PL 5 µg dose group were ~10-fold lower. Similar responses were seen for the RBD-Ferritin immunized animals. Responses were maintained several weeks following vaccination. Homologous boosting increased neutralizing responses by ~20- and ~70-fold for the high- and low-dose animals, respectively, achieving IC80 titers of ~10,000 and 5,000.

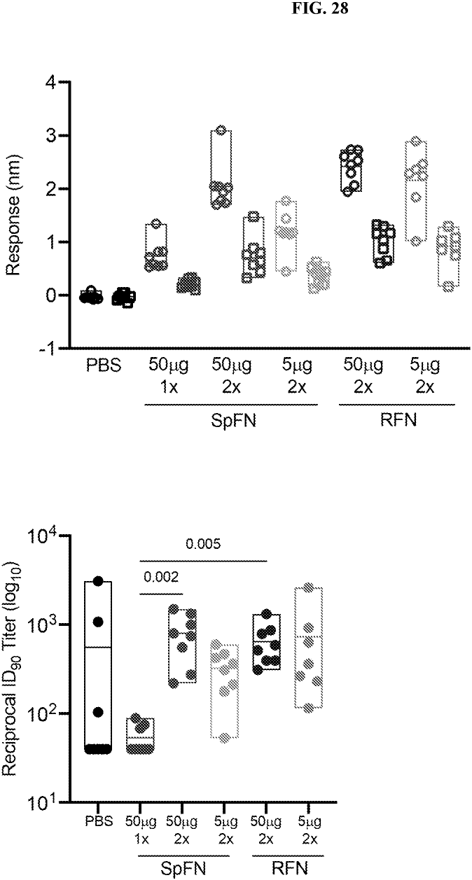

Live-virus neutralizing antibody responses: Neutralizing activity was also assessed using a live-virus assay with wild-type, intact SARS-CoV-2 in sera collected at weeks 0, 4, and 8. Vaccination with 50 µg of SpFN_1B-06-PL resulted in serum neutralizing activity following a single immunization, with reciprocal EC₅₀ GMTs of 581 at week 4 (FIG. 27). Following the boosting immunization, GMTs were 8,455 and 3,395 in animals vaccinated with 50 or 5 µg, respectively. Similar responses were seen for the RBD-Ferritin immunized animals. Neutralization of a wild-type, intact SARS-CoV-1 was also assessed with sera collected at weeks 6. ID90 titers for the majority of animals immunized twice had GMT titers of ~1,000 (FIG. 28). FIG. 34 shows the live-virus neutralization assay for SARS-CoV-2 assessed responses in serum 4 weeks following each immunization.

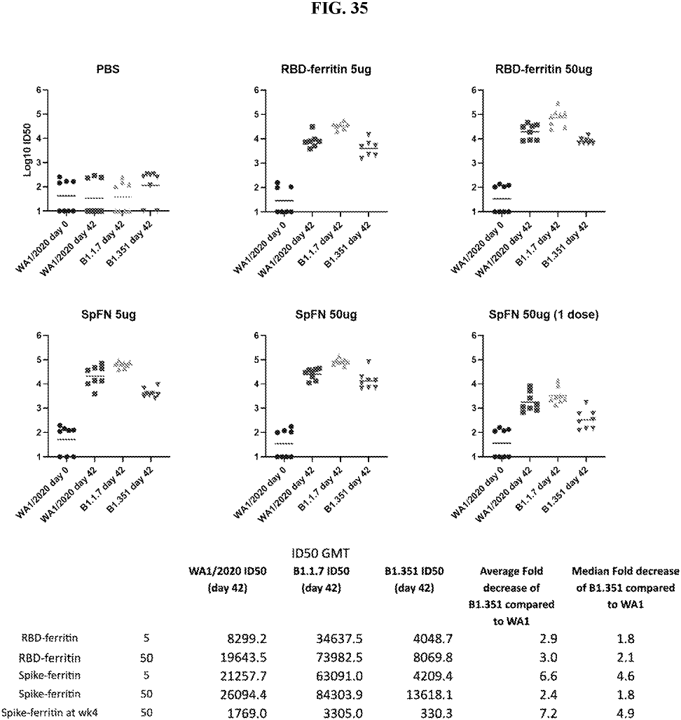

Live-virus neutralizing antibody responses against SARS-CoV-2 strains B1.1.7 and B1.351: Neutralizing activity was also assessed using a live-virus assay with wild-type, intact SARS-CoV-2 variants with sera collected at weeks 0, and 6 (FIG. 35).

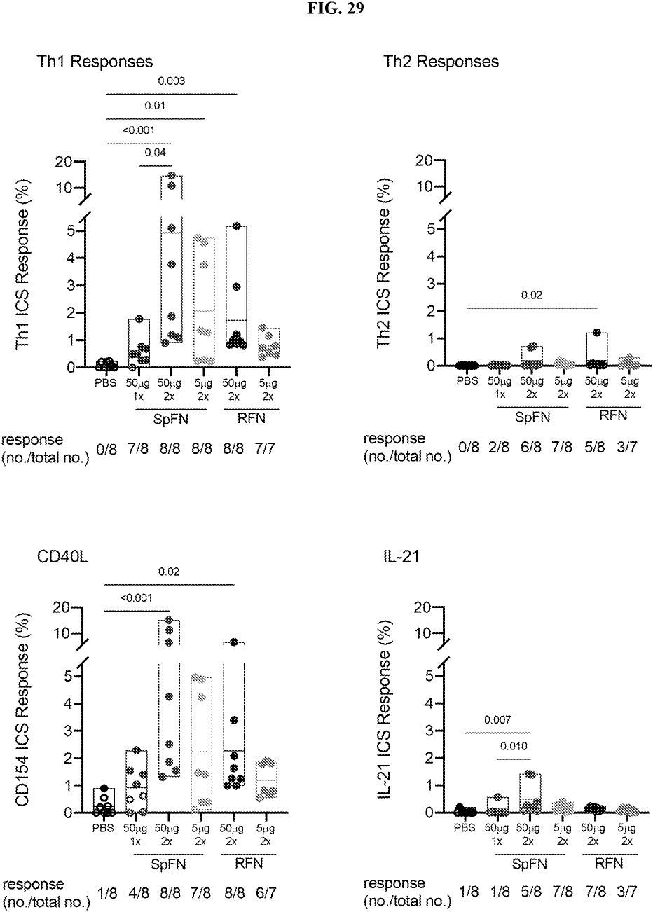

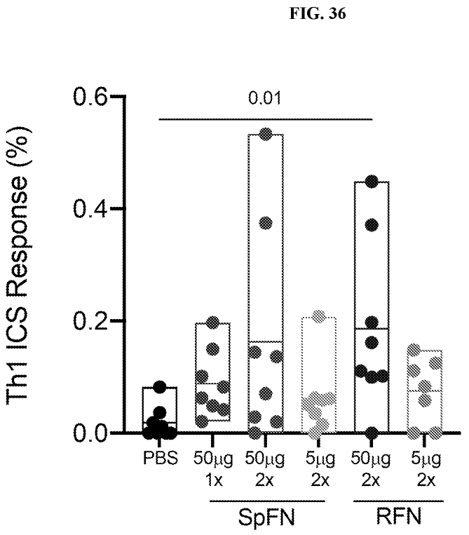

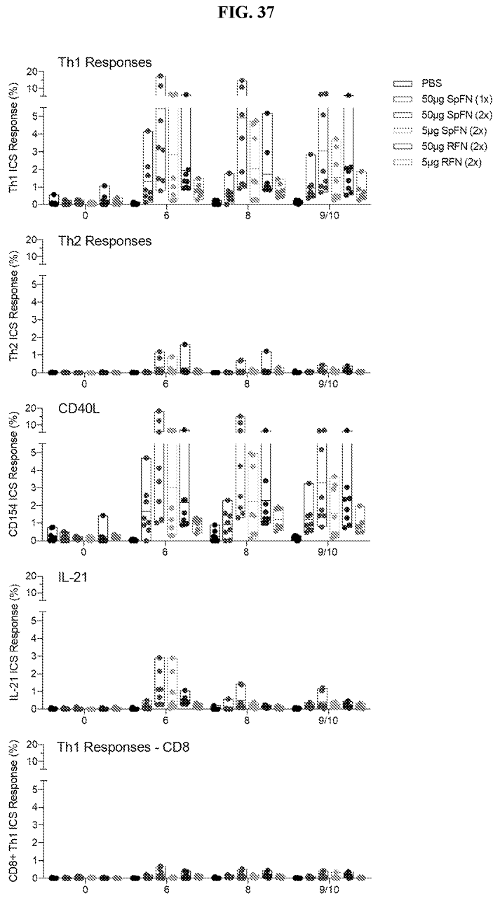

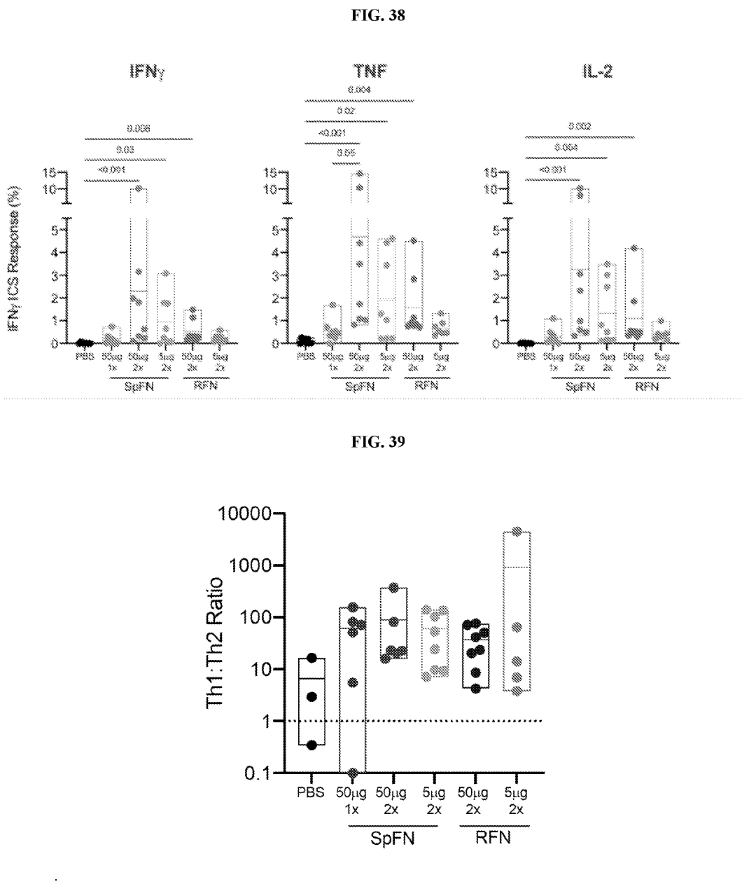

Antigen-specific T cell responses: SARS-CoV-2 Spike-specific T cells were assessed by in vitro stimulation of PBMC collected at weeks 0 and 6 with Spike peptide pools followed by intracellular cytokine staining (ICS). Prime-boost vaccination with 50 µg of SpFN_1B-06-PL or RBD-Ferritin_ppCoV131 generated Spike-specific CD4 T cells exhibiting a type 1 T helper (Th1) profile based on expression of TNFα, INFγ, and IL-2 in all animals (FIGS. 29, 36-38), ranging from ~1-18% of memory CD4 T cells. Single immunization with the 50 µg dose or prime-boost vaccination with the 5 µg dose elicited responses in most animals. Limited type 2 T helper (Th2) responses were observed by ICS for IL-4 and IL-13 and averaged ~10-fold lower in magnitude than Th1 responses. Analysis of the ratio of Th1 to Th2 cell responding cells indicated that both SpFN and RFN-vaccination elicited a predominant Th1 type response (FIG. 39).

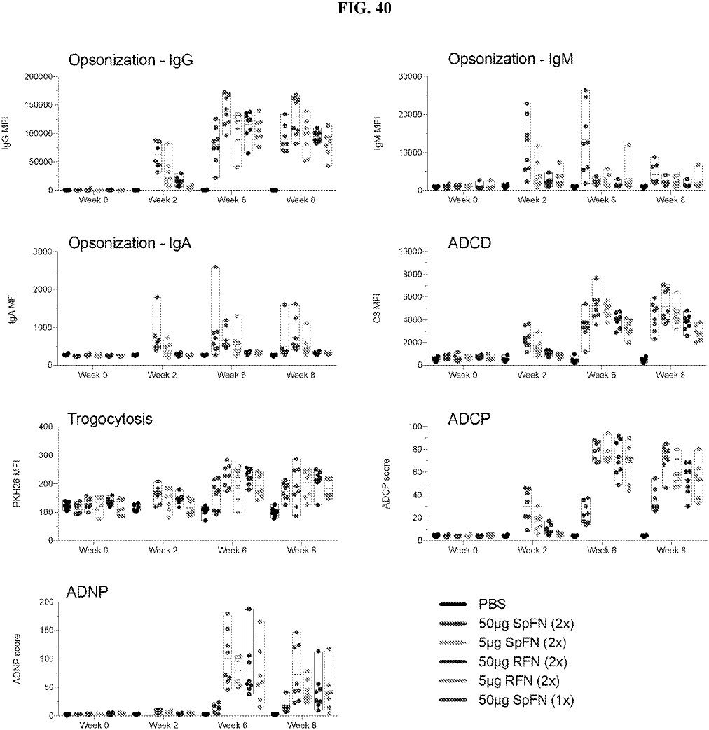

Effector Binding antibody responses to SARS-CoV-2: To assess the ability of SARS-CoV-2-specific humoral responses to facilitate cell effector functions such as Opsonization, ADCD, ADCP, ADNP, and trogocytosis, were measured at week 0 - 8. Robust effector functions were clearly observable following the initial immunization, and the subsequent second immunization boosted these immune responses (FIG. 40).

### Efficacy of SpFN Adjuvanted With ALFQ in Rhesus Macaques Following SARS-CoV-2 Challenge

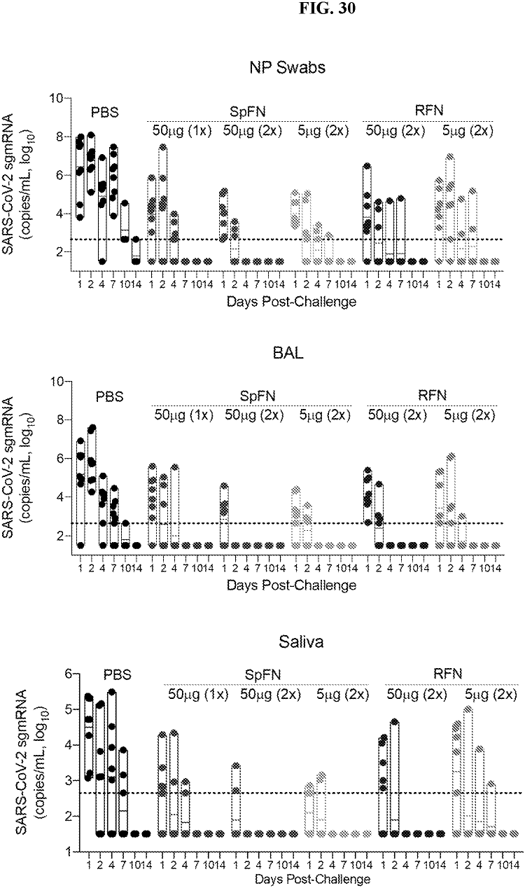

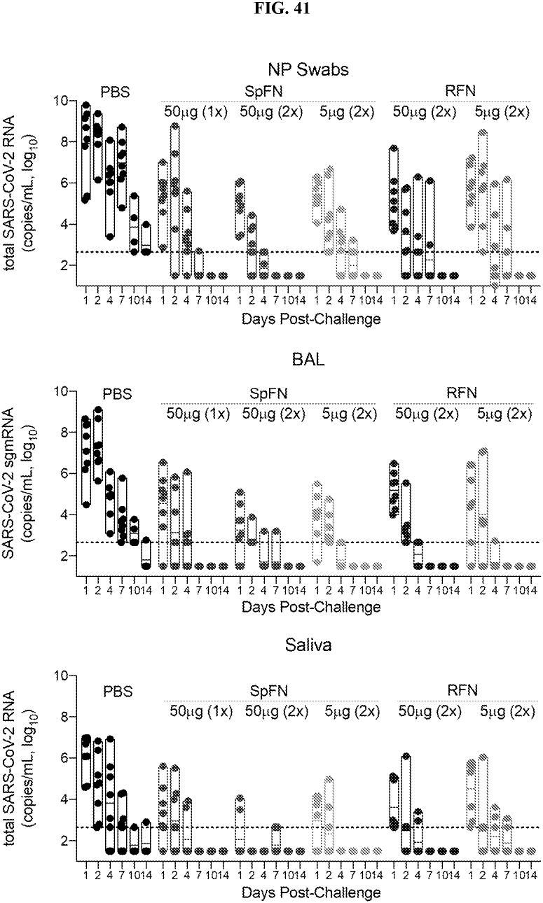

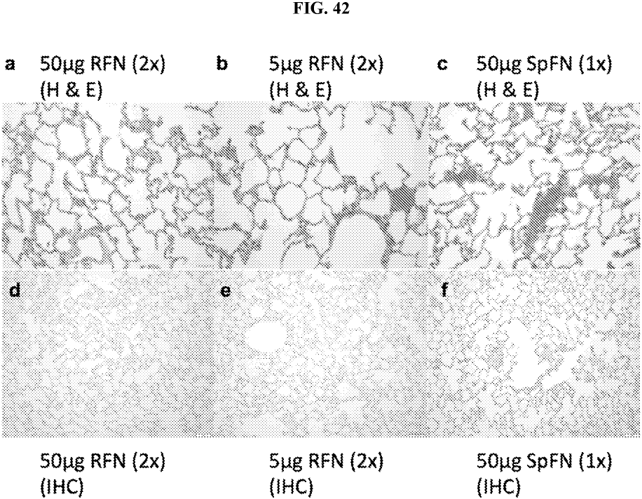

SARS-CoV-2 replication in respiratory tract: To evaluate vaccine efficacy against infection, macaques were challenged with high-dose 10⁶ TCID50 SARS-CoV-2 via the IN/IT routes four weeks after the boost (study week 8). Viral infection was assessed by RT-qPCR for viral subgenomic mRNA (sgmRNA) and total RNA in both NP swabs and BAL collected days 1, 2, 4, and 7 post-challenge. Half of the animals were also monitored at days 10 and 14 post-challenge. Total RNA includes genomic nucleic acid abundant in virions introduced by the challenge inoculum, while sgmRNA is considered a more specific indicator of active replication. All control animals showed evidence of robust infection with high levels of sgmRNA and total RNA in NP swabs, BAL and saliva from days 1-7 (FIGS. 30 and 41). In contrast, animals vaccinated with two doses of 50 µg SpFN or RFN showed little to no evidence of viral replication in both NP swabs and BAL. sgmRNA was not detected in BAL for 8 of 8 animals by day 2 and in NP swabs of 5 of 8 animals by day 2 and all animals by day 4. Viral replication was also minimal in the prime-boost 5 µg dose and single 50 µg dose groups, with very low or undetectable sgmRNA by day 4 in most animals.

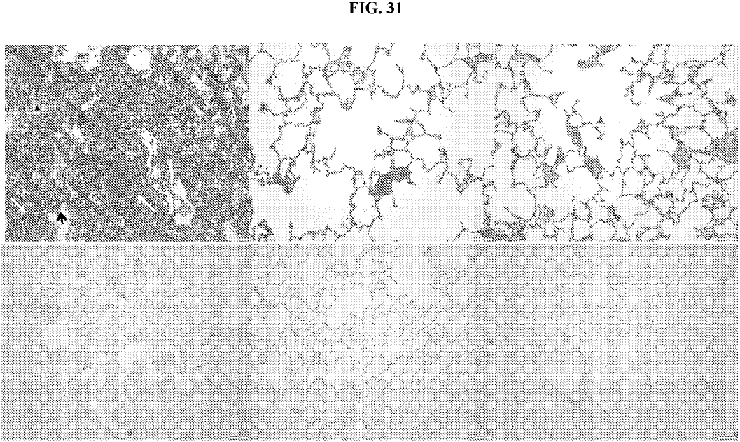

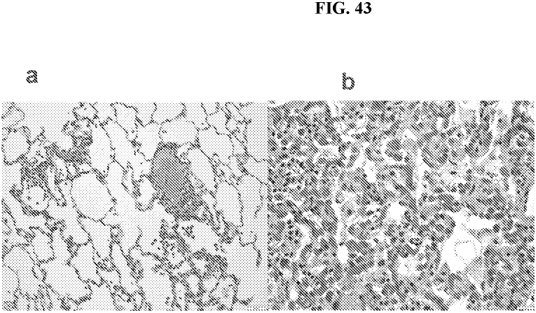

Lung pathology: Unvaccinated control animals developed histopathologic evidence of multifocal, moderate interstitial pneumonia at 7 days after challenge (FIGS. 31, 42, and 43). The pneumonia was characterized by type II pneumocyte hyperplasia, alveolar septal thickening, edema and necrotic debris, pulmonary macrophage infiltration and vasculitis of smaller caliber blood vessels. None of the vaccinated animals had evidence of interstitial pneumonia. Immunohistochemistry revealed viral antigen in alveolar pneumocytes and pulmonary macrophages in at least one lung section of every control animal (FIGS. 27, 42, and 43). No viral antigen was detected in any vaccinated animals (FIGS. 31, 42, and 43).

### Conclusions

Research grade SpFN_1B-06-PL or RBD-Ferritin formulated with ALFQ adjuvant given at 5 or 50 µg doses twice, or in a single dose of 50 µg, elicited in all animals sera that bound to the SARS-CoV-2 Spike protein and RBD subunit. These responses were maintained for at least four weeks following both the prime as well as the boost. In addition, the sera neutralized SARS-CoV-2 pseudovirions and live virus from multiple variants, SARS-CoV-1 pseudovirus and live virus, and also inhibited binding of SARS-CoV-2 Spike and RBD to the host cell ACE-2 receptor. Spike-specific CD4 Th1 T cell responses were present in PBMC, while Th2 responses were limited. Following high dose SARS-CoV-2 respiratory tract challenge, viral replication was not detectable in the lower airways (BAL) by day 2 in 17 of 24 SpFN_1B-06-PL vaccinated animals, while controls exhibited consistent and robust replication. In the upper airways, no viral replication was observed 15 of 24 vaccinated animals by day 4, including all 8 animals vaccinated twice with 50 µg SpFN₋1B-06-PL. No enhanced disease outcomes were observed in vaccinated rhesus macaques compared to control animals. These data demonstrate that ALFQ adjuvanted SpFN-1B-06-PL and RBD-Ferritin is immunogenic and efficacious in a macaque model.

### Example 6 - Immunization of Mice With a Mixture of SARS-CoV-2 Immunogen and SARS-CoV-1 Immunogens Provides a Broadly Neutralizing Immune Response

In order to assess whether SARS-CoV-2 immunogens could be combined with SARS-CoV-1 immunogens and whether the design procedure could be translated to other β-coronaviruses including SARS-like coronaviruses, a SARS-CoV-1 immunogen was designed based on the SARS-COV-2 SpFN_1B-06-PL format.

This SpFN_SARS-CoV-1 immunogen (SEQ ID NO: 255) was produced and purified in a similar manner as that described for the SARS-CoV-2 Spike Ferritin immunogens. A set of BALB/c and C57BL/6 mice was then immunized using two dose amounts (10 µg total or 2 µg total), which was a 50:50 mixture of the two immunogens. The resulting immune response was then analyzed in these animals for antibody responses.

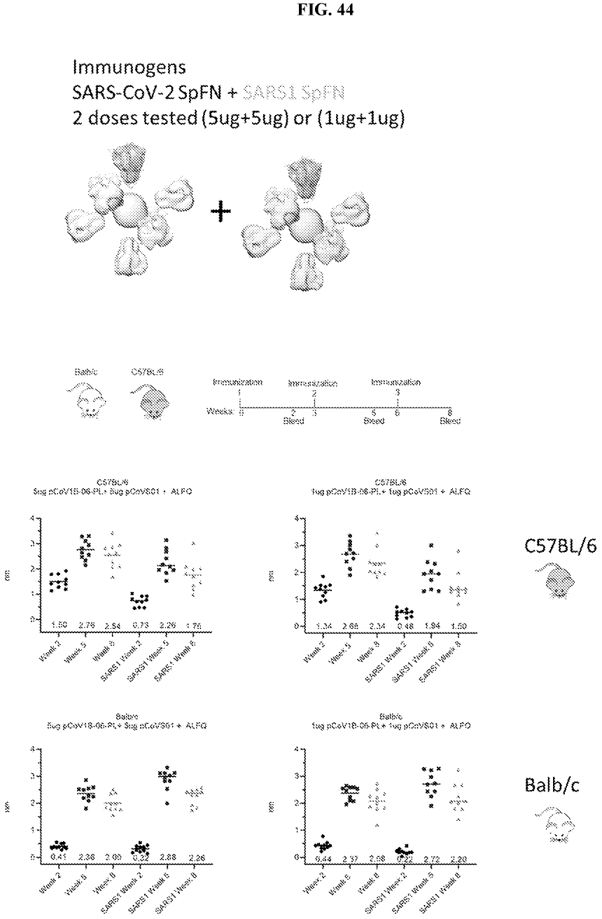

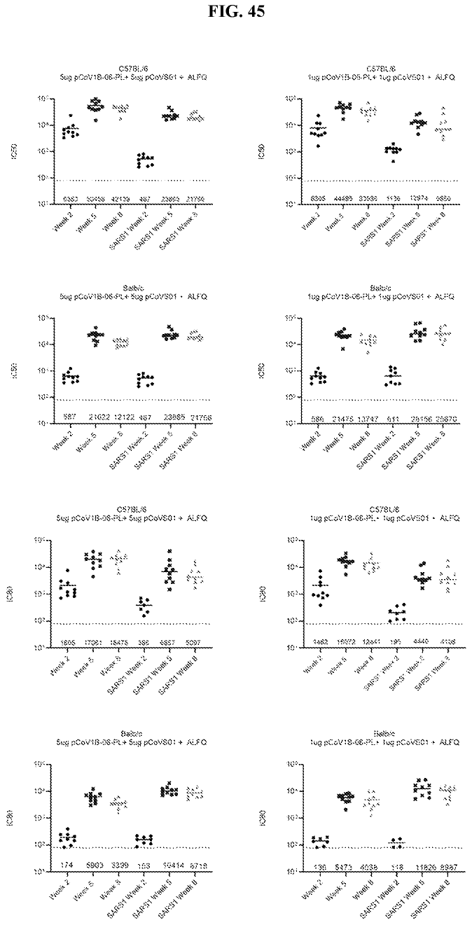

As shown in FIG. 44 and FIG. 45 and Table 9, mouse sera from animals immunized with the combination SARS-CoV-1 and SARS-CoV-2 SpFN immunogens produced high binding and high pseudovirus neutralizing titers against both SARS-CoV-1 and SARS-CoV-2 indicating that there was no immune competition between the two immunogens and that robust broad immune responses could be elicited in vivo with pseudovirus neutralization ID50 titers ranging from 10,000 to more than 20,000.

TABLE 9

SARS-CoV-1 pseudovirus neutralization GMT titers

| Animal Identifiers | Immunogen | Vaccine Dose (µg) | Mouse strain | Adjuvant | Immunization Schedule | Week 2 ID50 ID80 | | Week 5 ID50 ID80 | | Week 8 ID50 ID80 | |
|---|---|---|---|---|---|---|---|---|---|---|---|
| J1041-J1050 | SpFN_1B-06-PL and SpFN_SARS1 | 5 + 5 | BALB/c | ALFQ | Weeks 0, 3, 6 | 466 | 152 | 23865 | 10413 | 21766 | 8718 |
| J1051-J1060 | SpFN_1B-06-PL and SpFN_SARS1 | 1 + 1 | BALB/c | ALFQ | Weeks 0, 3, 6 | 611 | 118 | 28155 | 11826 | 25670 | 8989 |
| 301-310 | SpFN_1-06-PL and SpFN_SARS1 | 5 + 5 | C57BL/6 | ALFQ | Weeks 0, 3, 6 | 1208 | 365 | 17560 | 6658 | 10847 | 5097 |
| 311-320 | SpFN_1B-06-PL and SpFN_SARS1 | 1 + 1 | C57BL/6 | ALFQ | Weeks 0, 3, 6 | 1137 | 195 | 12974 | 4439 | 9651 | 4108 |

### Example 7 - Production of Spike-Ferritin Nanoparticles for HKU-1 and 229E Coronaviruses

In order to assess whether the design procedure for Spike-Ferritin constructs could be translated to other β-coronaviruses including HKU-1 and 229E coronaviruses, stabilized Spike-Ferritin immunogens were designed for HKU-1 (SEQ ID NOs: 268-275) and 229E (SEQ ID NOs: 264 and 265) based on the SARS-COV-2 SpFN_1B-06-PL format.

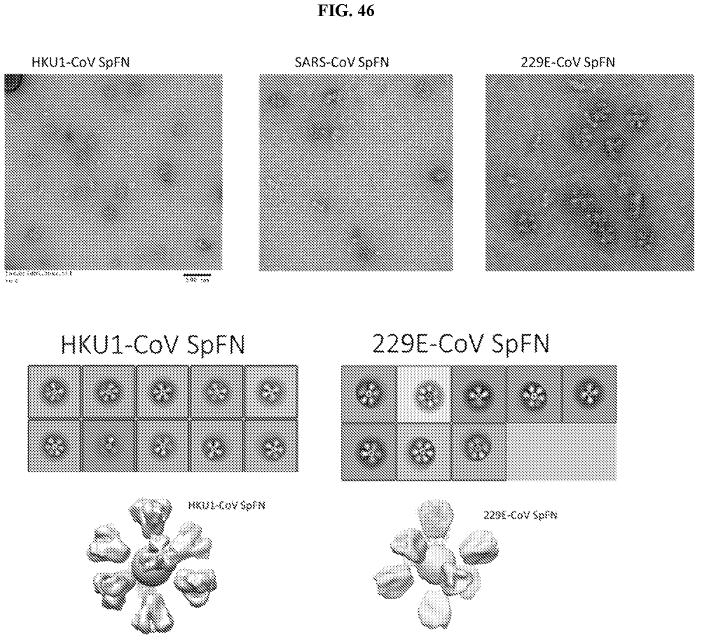

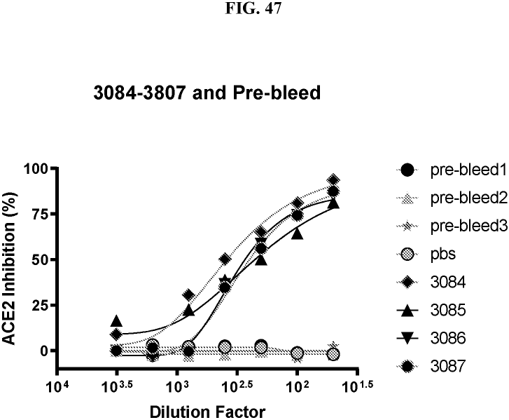

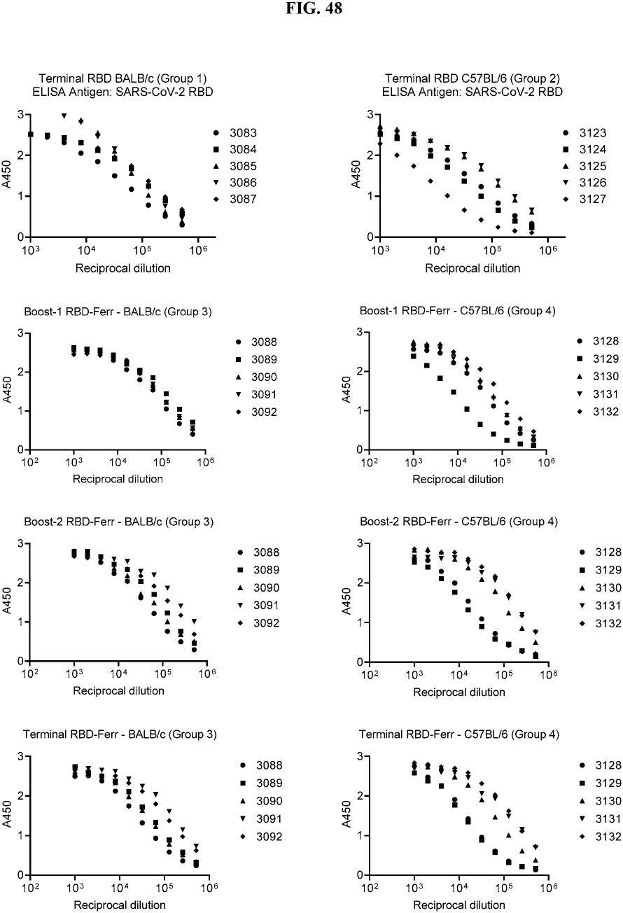

As shown in FIG. 46, stable Spike ferritin nanoparticles could be produced in mammalian cells, purified, and visualized by negative-stain EM. In both of these examples, the Spike-Ferritin nanoparticle shows the distinctive central ferritin region, with the protruding Spike. Three-dimensional reconstruction of the negative-stain images showed the closed pre-fusion Spikes on the surface of the Ferritin nanoparticle.

### Example 8 -Immunization of Mice With SARS-CoV-2 RBD DNA and Protein Immunogens Elicits Potent Neutralizing Antibody Responses

In order to assess whether using DNA encoding a SARS-CoV-2 RBD construct as a prime followed by a protein boost could elicit immune responses, a set of mice were immunized and the immune response was characterized as follows.

Methods: 96-well ELISA plates were coated with 1 µg/mL of RBD-His or a control His-tagged protein antigen in PBS, pH 7.4. Plates were incubated at 4° C. overnight and blocked with blocking buffer (Dulbecco’s PBS containing 0.5% milk and 0.1% Tween 20, pH 7.4, at room temperature (RT) for 2 h. Individual serum samples were serially diluted 2-fold in blocking buffer and added to triplicate wells and the plates were incubated at RT for 1 hour (h). Peroxidase-AffiniPure Goat Anti-Mouse IgG, Fcγ Fragment Specific was added and incubated at RT for an hour, followed by the addition of 2,2′-Azinobis [3-ethylbenzothiazoline-6-sulfonic acid]-diammonium salt (ABTS) HRP substrate (KPL) for 1 h at RT. The reaction was stopped by the addition of 1% SDS per well and the absorbance was measured at 450 nm. Results are shown in FIG. 48.

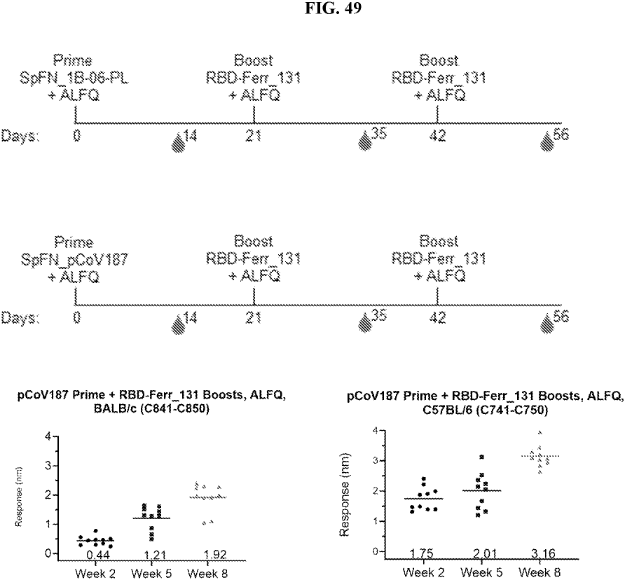

ACE2 inhibition assay: The biosensors were equilibrated in assay buffer for 30 s before being dipped in SARS-CoV-2 RBD-His (30 µg/ml diluted in PBS). The SARS-CoV-2 RBD-His were immobilized on HIS1K biosensors (FortéBio) for 180 s. After briefly dipping in assay buffer (30 s, PBS), binding of week 10 mouse serum was allowed to proceed for 180 s followed by a brief equilibration for 30 s. Binding of recombinant ACE2 protein (30 µg/ml) in solution was assessed for 120 s. Percent inhibition (PI) of RBD binding to ACE2 by mouse serum was determined by an equation: PI = 100 - [(ACE2 binding in the presence of competitor mouse serum) /(ACE2 binding in the absence of competitor mouse serum)] x 100. Results are show in FIG. 47.

Antigen Preparation: DNA encoding the SARS-Cov-2 RBD (residues 331-527) was synthesized (Genscript) with a C-terminal His6 purification tag and cloned into a CMVR plasmid, and protein was expressed by transient transfection in 293F cells for six days. The SARS-CoV-2 RBD (residues 331-527), with a C-terminal His-tag, was expressed in 293F cells. The RBD-Ferr construct was named pCoV03 (N-terminal His8 with HRV-3C cleavage site, GSGGGG linker between the RBD (residues 331-527 and Ferritin molecule). Proteins were purified from media supernatant by NiNTA affinity, and size-exclusion chromatography.

Animal Groups and Immunization/Assay Schedule: Four groups of female mice (C57BL/6 or BALB/c) aged 8 weeks old (n=5/group) were immunized using a DNA plasmid encoding a CMVR vector with the SARS-COV-2 RBD as the insert. Immunizations were carried out using a gene-gun, using 3 immunization sites with 1 ug of DNA per site, i.e. 3ug total DNA per immunization. These animals subsequently received two additional immunizations using GS-adjuvant (GenScript) with either RBD or RBD-Ferritin protein immunogens (Table 10). Intraperitoneal (IP) route was used for all mice immunizations. Sera samples were collected either 7 days or 14 days after each immunization for ELISA and other analyses.

TABLE 10

Immunization Regimen and Schedule

| Group | Animal Identifier | Strain | Immunization 1 | Immunization 2 | Immunization 3 | Immunization Schedule |
|---|---|---|---|---|---|---|
| 1 | 3083-3087 | BALB/c | SARS-CoV-2 RBD (3 µg CMVR DNA plasmid) | SARS-CoV-2 RBD (10 µg protein) | SARS-CoV-2 RBD (10 µg protein) | Weeks 0, 4, 7 |
| 2 | 3123-3127 | C57BL/6 | | | | |
| 3 | 3088-3092 | BALB/c | SARS-CoV-2 RBD (3 µg CMVR DNA plasmid) | SARS-CoV-2 RBD-Ferr (10 µg protein) | SARS-CoV-2 RBD-Ferr (10 µg protein) | Weeks 0, 4, 7 |
| 4 | 3128-3132 | C57BL/6 | | | | |

TABLE 11

Mice were sacrificed at 18 weeks and samples from groups 2,3 and 4 were analyzed for both SARS-CoV and SARS-CoV-2 pseudovirus neutralization titers

| Animal Identifiers | Immunogen | Mouse strain | SARS-CoV-1 | | SARS-CoV-2 | |
|---|---|---|---|---|---|---|
| | | | ID50 | ID80 | ID50 | ID80 |
| 3123-3127 | RBD (DNA prime) RBD (protein boost) | C57BL/6 | 183 | <40 | 2638 | 876 |
| 3088-3092 | RBD (DNA prime) RBD-Ferritin (protein boost) | BALB/c | 157 | <40 | 697 | 382 |
| 3128-3132 | RBD (DNA prime) RBD-Ferritin (protein boost) | C57BL/6 | 743 | 146 | 3503 | 1116 |

The use of a DNA prime followed by RBD or RBD-Ferritin protein boosts in the mouse model clearly showed robust induction of antibodies targeting the SARS-CoV-2 RBD, that are capable of blocking ACE2 binding and also provide robust and long lived neutralization activity against SARS-CoV-2 and SARS-CoV-1 up to 18 weeks after the first immunization, and more than 10 weeks after the final immunization (Table 11).

### Example 9 Dose Ranging Study Using Developmental Grade SpFN_1B-06-PL Material

Developmental grade material was produced in the WRAIR Pilot Bioproduction Facility according to cGMP procedures, and purified by anion exchange, filtered and stored at 4oC. This material was used to immunize BALB/c and C57BL/6 mice as shown in Table 12.

TABLE 12

Immunization Regimen and Schedule

| Group | Animal Identifier | Treatment | Volume Injected (µL) | Vaccine Dose (µg) | Adjuvant | Animals | Immunization Schedule |
|---|---|---|---|---|---|---|---|
| 1 | 1101-1110 | SpFN_1B-06-PL | 50 | 10 | ALFQ | 10 BALB/c | Weeks 0, 3, 6 |
| 2 | 1111-1120 | SpFN_1B-06-PL | 50 | 2 | ALFQ | 10 BALB/c | Weeks 0, 3, 6 |
| 3 | 1121-1130 | SpFN_1B-06-PL | 50 | 0.08 | ALFQ | 10 BALB/c | Weeks 0, 4, 8 |
| 4 | 1131-1140 | SpFN_1B-06-PL | 50 | 10 | ALFQ | 10 C57BL/6 | Weeks 0, 4, 8 |
| 5 | 1141-1150 | SpFN_1B-06-PL | 50 | 2 | ALFQ | 10 C57BL/6 | Weeks 0, 3, 6 |
| 6 | 1151-1160 | SpFN_1B-06-PL | 50 | 0.08 | ALFQ | 10 C57BL/6 | Weeks 0, 3, 6 |

Mice were bled to provide serum samples at regular intervals and samples were analyzed by ELISA for reactivity against SARS-CoV-2 S-2P and RBD as shown in Table 13.

TABLE 13

ELISA serum response against SARS-CoV-2 S-2P and RBD

| Group | Vaccine Dose (µg) | Week 2 | | Week 5 | | Week 8 | | Week 10 | | Week 12 | |
|---|---|---|---|---|---|---|---|---|---|---|---|
| | | S-2P RBD | | S-2P RBD | | S-2P RBD | | S-2P RBD | | S-2P | RBD |
| 1 | 10 | 30720 | 10880 | 368640 | 240640 | 327680 | 215040 | 471040 | 409600 | 204800 | 158720 |
| 2 | 2 | 17920 | 7680 | 389120 | 209920 | 256000 | 215040 | 634880 | 153600 | 199680 | 148480 |
| 3 | 0.08 | 7840 | 4160 | 271360 | 179200 | 324267 | 296960 | 450560 | 286720 | 675840 | 153600 |
| 4 | 10 | 174080 | 112640 | 942080 | 307200 | 573440 | 215040 | 573440 | 225280 | 471040 | 148480 |
| 5 | 2 | 143360 | 69120 | 491520 | 286720 | 389120 | 204800 | 696320 | 235520 | 368640 | 209920 |
| 6 | 0.08 | 40960 | 32000 | 225280 | 135680 | 450560 | 348160 | 655360 | 296960 | 455111 | 648533 |

Clear immune responses are seen against both the SARS-CoV-2 Spike and RBD after a single immunization and can be boosted by subsequent immunizations. The ELISA binding titers persist over the duration of the study with high levels of reactive antibodies observed 6 weeks after the last immunization.

### Example 10 Dose Ranging Study Using Research Grade SpFN_1B-06-PL Material

In order to understand the dose response of SpFN_1B-06-PL, a dose decrease study was carried out in two strains of mice, BALB/c and C57BL/6, with doses decreasing from 10 µg in 5-fold dilutions to a final tested concentration of 0.0032 µg (Table 14). Each dose was adjuvanted with ALFQ as previously described, and animals were immunized three times. Samples were taken at regular intervals to measure the immune response by ELISA against SARS-CoV-2 S-2P and RBD proteins.

TABLE 14

Immunization Regimen and Schedule

| Group | Animal Identifier | Treatment | Volume Injected (µL) | Vaccine Dose (µg) | Adjuvant | Animals | Immunization Schedule |
|---|---|---|---|---|---|---|---|
| 1 | 1761-1770 | SpFN_1B-06-PL | 50 | 10 | ALFQ | 10 C57BL/6 | Weeks 0, 3, 6 |
| 2 | 1771-1780 | SpFN_1B-06-PL | 50 | 2 | ALFQ | 10 C57BL/6 | Weeks 0, 3, 6 |
| 3 | 1781-1790 | SpFN_1B-06-PL | 50 | 0.4 | ALFQ | 10 C57BL/6 | Weeks 0, 4, 8 |
| 4 | 1791-1800 | SpFN_1B-06-PL | 50 | 0.08 | ALFQ | 10 C57BL/6 | Weeks 0, 4, 8 |
| 5 | 101-110 | SpFN_1B-06-PL | 50 | 0.016 | ALFQ | 10 C57BL/6 | Weeks 0, 3, 6 |
| 6 | 111-120 | SpFN_1B-06-PL | 50 | 0.0032 | ALFQ | 10 C57BL/6 | Weeks 0, 3, 6 |
| 7 | 1701-1710 | SpFN_1B-06-PL | 50 | 10 | ALFQ | 10 BALB/c | Weeks 0, 3, 6 |
| 8 | 1711-1720 | SpFN_1B-06-PL | 50 | 2 | ALFQ | 10 BALB/c | Weeks 0, 3, 6 |
| 9 | 1721-1730 | SpFN_1B-06-PL | 50 | 0.4 | ALFQ | 10 BALB/c | Weeks 0, 3, 6 |
| 10 | 1731-1740 | SpFN_1B-06-PL | 50 | 0.08 | ALFQ | 10 BALB/c | Weeks 0, 3, 6 |
| 11 | 1741-1750 | SpFN_1B-06-PL | 50 | 0.016 | ALFQ | 10 BALB/c | Weeks 0, 3, 6 |
| 12 | 1751-1760 | SpFN_1B-06-PL | 50 | 0.0032 | ALFQ | 10 BALB/c | Weeks 0, 3, 6 |

Even at the lowest dose (0.0032 µg), which is a 3125-fold dilution from the typical 10 ug dose, clear binding antibodies were observed to both the SARS-CoV-2 S-2P and RBD, that were ~ 3-4 fold lower in titer value compared to the 10 ug dose at the week 12 time point for the BALB/c mice (Table 15). In the three-immunization schedule, the lower doses responded to a greater magnitude with the third immunization, which partially explains the comparable final immune responses despite large differences in the immunogen amount.

TABLE 15

ELISA serum response against SARS-CoV-2 S-2P and RBD

| Group | Vaccine Dose(□g) | Week 2 | | Week 5 | | Week 8 | | Week 10 | | Week 12 | |
|---|---|---|---|---|---|---|---|---|---|---|---|
| | | S-2P | RBD | S-2P | RBD | S-2P | RBD | S-2P | RBD | S-2P | RBD |
| 1 | 10 | 204800 | 112640 | 327680 | 256000 | 471040 | 276480 | 552960 | 235520 | 471040 | 163840 |
| 2 | 2 | 163840 | 102400 | 348160 | 266240 | 655360 | 389120 | 532480 | 307200 | 450560 | 307200 |
| 3 | 0.4 | 92160 | 87040 | 261120 | 179200 | 552960 | 317440 | 655360 | 368640 | 471040 | 337920 |
| 4 | 0.08 | 48640 | 43520 | 261120 | 199680 | 614400 | 552960 | 839680 | 1413120 | 614400 | 471040 |
| 5 | 0.016 | 9980 | 5260 | 50880 | 32480 | 320000 | 206080 | 601600 | 313600 | 256000 | 190720 |
| 6 | 0.0032 | 260 | 220 | 2200 | 1580 | 7720 | 10520 | 23400 | 12040 | 11360 | 17480 |
| 7 | 10 | 87040 | 37120 | 512000 | 225280 | 593920 | 307200 | 737280 | 235520 | 235520 | 168960 |
| 8 | 2 | 51200 | 24320 | 696320 | 307200 | 860160 | 675840 | 1228800 | 327680 | 389120 | 337920 |
| 9 | 0.4 | 24320 | 4320 | 1310720 | 430080 | 1085440 | 209920 | 655360 | 215040 | 245760 | 133120 |
| 10 | 0.08 | 16000 | 4040 | 1024000 | 501760 | 901120 | 266240 | 573440 | 296960 | 327680 | 107520 |
| 11 | 0.016 | 5440 | 2800 | 343040 | 171520 | 389120 | 143360 | 440320 | 399360 | 204800 | 84480 |
| 12 | 0.0032 | 2400 | 2080 | 76200 | 54880 | 235520 | 61440 | 187733 | 170666 | 84480 | 56320 |

### Example 11 Analysis of Mouse Sera for Pseudovirus Neutralization Against SARS-CoV-1

In order to understand whether mouse sera immunized with SARS-CoV-2 immunogens could elicit antibody responses with broad reactivity against other related but distant SARS-like viruses, the serum from mice immunized with multiple Spike-nanoparticle immunogens was assessed for their ability to neutralize SARS-CoV-1 pseudoviruses.

Shown in Table 16 below are the ID50 and ID80 GMT titers for mice immunized with multiple types of immunogen and with either ALFQ or Alhydrogel as the adjuvant. High SARS-CoV-1 neutralizing antibody titers are routinely observed after three immunizations with the SARS-CoV-2 immunogens with the ALFQ adjuvant.

TABLE 16

SARS-CoV-1 pseudovirus neutralization GMT - mouse samples analyzed

| Animal Identifiers | Immunogen | Vaccine Dose(µg) | Mouse strain | Adjuvant | Immunization Schedule | Week 2 | | Week 5 | | Week 8 | |
|---|---|---|---|---|---|---|---|---|---|---|---|
| | | | | | | ID50 | ID80 | ID50 | ID80 | ID50 | ID80 |
| E771-E780 | SpFN_lB-06-PL | 2 | C57BL/6 | ALFQ | Weeks 0, 3, 6 | 106 | <40 | 1083 | 482 | 1384 | 465 |
| E861-E870 | SpFN_lB-06-PL | 2 | BALB/c | ALFQ | Weeks 0, 3, 6 | 59 | <40 | 460 | 160 | 803 | 256 |
| J1001-J1010 | SpFN_1B-06-PL + RBD-Ferr_131 Boost | 10 | BALB/c | ALFQ | Weeks 0, 3, 6 | 67 | <40 | 153 | 90 | 316 | 153 |
| J1061-J1070 | SpFN_1B-06-PL + RBD-Ferr_131 Boost | 10 | C57BL/6 | ALFQ | Weeks 0, 3, 6 | 64 | <40 | 181 | 84 | 371 | 118 |
| Z891-Z900 | pCoV187 (1B-08-PL with D614G) | 10 | BALB/c | ALFQ | Weeks 0, 3, 6 | NT | NT | 108 | <40 | 200 | 88 |
| Z761-Z770 | pCoV187 (1B-08-PL with D614G) | 10 | C57BL/6 | ALFQ | Weeks 0, 3, 6 | NT | NT | 276 | 107 | 517 | 149 |
| 581-590 | RBD-Ferr_pCoV131 | 10 | C57BL/6 | ALFQ | Weeks 0, 3, 6 | 303 | <80 | 1141 | 301 | 3053 | 857 |
| 1331-1340 | RBD-Ferr_pCoV131 | 10 | BALB/c | ALFQ | Weeks 0, 3, 6 | 596 | 102 | 889 | 219 | 2541 | 589 |
| 1341-1350 | RBD-Ferr_pCoV131 | 10 | BALB/c | Alhydrogel | Weeks 0, 3, 6 | 120 | >40 | 182 | 103 | 240 | 100 |
| 1391-1400 | pCoV146 | 10 | BALB/c | ALFQ | Weeks 0, 3, 6 | NT | NT | 201 | 57 | 246 | 87 |
| C831-C840 | pCoV146 | 10 | BALB/c | Alhydrogel | Weeks 0, 3, 6 | NT | NT | 153 | >40 | 177 | 47 |
| 901-910 | pCoV146 | 10 | C57BL/6 | ALFQ | Weeks 0, 3, 6 | NT | NT | 220 | 65 | 320 | 90 |
| C731-C740 | pCoV146 | 10 | C57BL/6 | Alhydrogel | Weeks 0, 3, 6 | NT | NT | NT | NT | 67 | <40 |

NT: not tested

### Example 12 Priming With Spike-Ferritin and Boosting With RBD-Ferritin_pCoV131

In order to assess whether an increased immune response could be generated by “Immune-focusing” responses to the RBD, a heterologous prime-boost study was carried out. Mice were primed with either SpFN_1B-06-PL or SpFN_pCoV187, followed by two subsequent immunizations with RBD-Ferritin_pCoV131 (FIG. 49). Sera were assessed for immune responses.

Shown in Table 17 below are the ID50 and ID80 GMT titers for mice immunized with pCoV187 followed by boost with pCoV131.

TABLE 17

SARS-CoV-2 Pseudovirus neutralization

| Animal Identifier | Treatment | Vaccine Dose (µg) | Mouse strain | Adjuvant | Immunization Schedule | Week 2 | | Week 5 | |
|---|---|---|---|---|---|---|---|---|---|
| | | | | | | ID50 | ID80 | ID50 | ID80 |
| C841-C850 | pCoV187 + RBD-Ferr_131 Boost | 10 | BALB/c | ALFQ | Weeks 0, 3, 6 | 849 | 231 | 5567 | 1569 |
| C741-C750 | pCoV187 + RBD-Ferr_131 Boost | 10 | C57BL/6 | ALFQ | Weeks 0, 3, 6 | 11489 | 4002 | 23076 | 8064 |

TABLE 18

Amino Acid Sequences for Exemplary Nanoparticle-Forming Proteins

| Table 18 Notes | ID | Construct | # of Residues | Amino Acid Sequence | SEQ ID NO |
|---|---|---|---|---|---|
| | pCoV01 | RBD-Ferritin | 378 | NITNLCPFGEVFNATRFASVYAWNRKRISNCVADYSVLYNSASFSTFKCY GVSPTKLNDLCFTNVYADSFVIRGDEVRQIAPGQTGKIADYNYKLPDDFT GCVIAWNSNNLDSKVGGNYNYLYRLFRKSNLKPFERDISTEIYQAGSTPC NGVEGFNCYFPLQSYGFQPTNGVGYQPYRVVVLSFELLHAPATVCGPGSG GGGESQVRQQFSKDIEKLLNEQVNKEMQSSNLYMSMSSWCYTHSLDGAGL FLFDHAAEEYEHAKKLIIFLNENNVPVQLTSISAPEHKFEGLTQIFQKAY EHEQHISESINNIVDHAIKSKDHATFNFLQWYVAEQHEEEVLFKDILDKI ELIGNENHGLYLADQYVKGIAKSRKSGS | 23 |
| | pCoV02 | S1-Ferritin | 844 | VNLTTRTQLPPAYTNSFTRGVYYPDKVFRSSVLHSTQDLFLPFFSNVTWF HAIHVSGTNGTKRFDNPVLPFNDGVYFASTEKSNIIRGWIFGTTLDSKTQ SLLIVNNATNVVIKVCEFQFCNDPFLGVYYHKNNKSWMESEFRVYSSANN CTFEYVSQPFLMDLEGKQGNFKNLREFVFKNIDGYFKIYSKHTPINLVRD LPQGFSALEPLVDLPIGINITRFQTLLALHRSYLTPGDSSSGWTAGAAAY YVGYLQPRTFLLKYNENGTITDAVDCALDPLSETKCTLKSFTVEKGIYQT SNFRVQPTESIVRFPNITNLCPFGEVFNATRFASVYAWNRKRISNCVADY SVLYNSASFSTFKCYGVSPTKLNDLCFTNVYADSFVIRGDEVRQIAPGQT GKIADYNYKLPDDFTGCVIAWNSNNLDSKVGGNYNYLYRLFRKSNLKPFE RDISTEIYQAGSTPCNGVEGFNCYFPLQSYGFQPTNGVGYQPYRVVVLSF ELLHAPATVCGPKKSTNLVKNKCVNFNFNGLTGTGVLTESNKKFLPFQQF GRDIADTTDAVRDPQTLEILDITPCSFGGVSVITPGTNTSNQVAVLYQDV NCTEVPVAIHADQLTPTWRVYSTGSNVFQTRAGCLIGAEHVNNSYECDIP IGAGICASYQTQTGSGGGGESQVRQQFSKDIEKLLNEQVNKEMQSSNLYM SMSSWCYTHSLDGAGLFLFDHAAEEYEHAKKLIIFLNENNVPVQLTSISA PEHKFEGLTQIFQKAYEHEQHISESINNIVDHAIKSKDHATFNFLQWYVA EQHEEEVLFKDILDKIELIGNENHGLYLADQYVKGIAKSRKSGS | 24 |
| | pCoV03 | His8-3c-RBD-Ferritin | 396 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYLYRLFRKSNLK PFERDISTEIYQAGSTPCNGVEGFNCYFPLQSYGFQPTNGVGYQPYRVVV LSFELLHAPATVCGPGSGGGGESQVRQQFSKDIEKLLNEQVNKEMQSSNL YMSMSSWCYTHSLDGAGLFLFDHAAEEYEHAKKLIIFLNENNVPVQLTSI SAPEHKFEGLTQIFQKAYEHEQHISESINNIVDHAIKSKDHATFNFLQWY VAEQHEEEVLFKDILDKIELIGNENHGLYLADQYVKGIAKSRKSGS | 25 |
| | pCoV04 | His8-3c-S1-Ferritin | 862 | HHHHHHHHGPLEVLFQGPVNLTTRTQLPPAYTNSFTRGVYYPDKVFRSSV LHSTQDLFLPFFSNVTWFHAIHVSGTNGTKRFDNPVLPFNDGVYFASTEK SNIIRGWIFGTTLDSKTQSLLIVNNATNVVIKVCEFQFCNDPFLGVYYHK NNKSWMESEFRVYSSANNCTFEYVSQPFLMDLEGKQGNFKNLREFVFKNI DGYFKIYSKHTPINLVRDLPQGFSALEPLVDLPIGINITRFQTLLALHRS YLTPGDSSSGWTAGAAAYYVGYLQPRTFLLKYNENGTITDAVDCALDPLS ETKCTLKSFTVEKGIYQTSNFRVQPTESIVRFPNITNLCPFGEVFNATRF ASVYAWNRKRISNCVADYSVLYNSASFSTFKCYGVSPTKLNDLCFTNVYA DSFVIRGDEVRQIAPGQTGKIADYNYKLPDDFTGCVIAWNSNNLDSKVGG NYNYLYRLFRKSNLKPFERDISTEIYQAGSTPCNGVEGFNCYFPLQSYGF QPTNGVGYQPYRVVVLSFELLHAPATVCGPKKSTNLVKNKCVNFNFNGLT GTGVLTESNKKFLPFQQFGRDIADTTDAVRDPQTLEILDITPCSFGGVSV ITPGTNTSNQVAVLYQDVNCTEVPVAIHADQLTPTWRVYSTGSNVFQTRA GCLIGAEHVNNSYECDIPIGAGICASYQTQTGSGGGGESQVRQQFSKDIE KLLNEQVNKEMQSSNLYMSMSSWCYTHSLDGAGLFLFDHAAEEYEHAKKL IIFLNENNVPVQLTSISAPEHKFEGLTQIFQKAYEHEQHISESINNIVDH AIKSKDHATFNFLQWYVAEQHEEEVLFKDILDKIELIGNENHGLYLADQY VKGIAKSRKSGS | 26 |
| | pCoV12 | RBDDNIT-Ferritin | 375 | NLCPFGEVFNATRFASVYAWNRKRISNCVADYSVLYNSASFSTFKCYGVS PTKLNDLCFTNVYADSFVIRGDEVRQIAPGQTGKIADYNYKLPDDFTGCV IAWNSNNLDSKVGGNYNYLYRLFRKSNLKPFERDISTEIYQAGSTPCNGV EGFNCYFPLQSYGFQPTNGVGYQPYRVVVLSFELLHAPATVCGPGSGGGG ESQVRQQFSKDIEKLLNEQVNKEMQSSNLYMSMSSWCYTHSLDGAGLFLF DHAAEEYEHAKKLIIFLNENNVPVQLTSISAPEHKFEGLTQIFQKAYEHE QHISESINNIVDHAIKSKDHATFNFLQWYVAEQHEEEVLFKDILDKIELI GNENHGLYLADQYVKGIAKSRKSGS | 27 |
| | pCoV15 | RBD-Ferritin (short linker) | | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYLYRLFRKSNLK PFERDISTEIYQAGSTPCNGVEGFNCYFPLQSYGFQPTNGVGYQPYRVVV LSFELLHAPATVCGPGSGESQVRQQFSKDIEKLLNEQVNKEMQSSNLYMS MSSWCYTHSLDGAGLFLFDHAAEEYEHAKKLIIFLNENNVPVQLTSISAP EHKFEGLTQIFQKAYEHEQHISESINNIVDHAIKSKDHATFNFLQWYVAE QHEEEVLFKDILDKIELIGNENHGLYLADQYVKGIAKSRKSGS | 28 |
| | pCoV20 | His8-3c-RBDDNIT-Ferritin | | HHHHHHHHGPLEVLFQGPNLCPFGEVFNATRFASVYAWNRKRISNCVADY SVLYNSASFSTFKCYGVSPTKLNDLCFTNVYADSFVIRGDEVRQIAPGQT GKIADYNYKLPDDFTGCVIAWNSNNLDSKVGGNYNYLYRLFRKSNLKPFE RDISTEIYQAGSTPCNGVEGFNCYFPLQSYGFQPTNGVGYQPYRVVVLSF ELLHAPATVCGPGSGGGGESQVRQQFSKDIEKLLNEQVNKEMQSSNLYMS MSSWCYTHSLDGAGLFLFDHAAEEYEHAKKLIIFLNENNVPVQLTSISAP EHKFEGLTQIFQKAYEHEQHISESINNIVDHAIKSKDHATFNFLQWYVAE QHEEEVLFKDILDKIELIGNENHGLYLADQYVKGIAKSRKSGS | 29 |
| | pCoV21 | His8-3c-RBD-6-LS | 375 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYLYRLFRKSNLK PFERDISTEIYQAGSTPCNGVEGFNCYFPLQSYGFQPTNGVGYQPYRVVV LSFELLHAPATVCGPGSGGGGMQIYEGKLTAEGLRFGIVASRFNHALVDR LVEGAIDAIVRHGGREEDITLVRVPGSWEIPVAAGELARKEDIDAVIAIG VLIRGATPHFDYIASEVSKGLADLSLELRKPITFGVITADTLEQAIERAG TKHGNKGWEAALSAIEMANLFKSLR | 30 |
| | pCoV22 | LS-15-RBD-3c-Strep-His8 | 414 | MQIYEGKLTAEGLRFGIVASRFNHALVDRLVEGAIDAIVRHGGREEDITL VRVPGSWEIPVAAGELARKEDIDAVIAIGVLIRGATPHFDYIASEVSKGL ADLSLELRKPITFGVITADTLEQAIERAGTKHGNKGWEAALSAIEMANLF KSLRGGSGGSGGSGGSGGGNITNLCPFGEVFNATRFASVYAWNRKRISNC VADYSVLYNSASFSTFKCYGVSPTKLNDLCFTNVYADSFVIRGDEVRQIA PGQTGKIADYNYKLPDDFTGCVIAWNSNNLDSKVGGNYNYLYRLFRKSNL KPFERDISTEIYQAGSTPCNGVEGFNCYFPLQSYGFQPTNGVGYQPYRVV VLSFELLHAPATVCGPLEVLFQGPSAWSHPQFEKGGGSGGGSGGSAWSHP QFEKGSHHHHHHHH | 31 |
| | pCoV23 | His8-3c-NTD-Ferritin | 487 | HHHHHHHHGPLEVLFQGPVNLTTRTQLPPAYTNSFTRGVYYPDKVFRSSV LHSTQDLFLPFFSNVTWFHAIHVSGTNGTKRFDNPVLPFNDGVYFASTEK SNIIRGWIFGTTLDSKTQSLLIVNNATNVVIKVCEFQFCNDPFLGVYYHK NNKSWMESEFRVYSSANNCTFEYVSQPFLMDLEGKQGNFKNLREFVFKNI DGYFKIYSKHTPINLVRDLPQGFSALEPLVDLPIGINITRFQTLLALHRS YLTPGDSSSGWTAGAAAYYVGYLQPRTFLLKYNENGTITDAVDCALDPLS ETKCTLGSGGGGESQVRQQFSKDIEKLLNEQVNKEMQSSNLYMSMSSWCY THSLDGAGLFLFDHAAEEYEHAKKLIIFLNENNVPVQLTSISAPEHKFEG LTQIFQKAYEHEQHISESINNIVDHAIKSKDHATFNFLQWYVAEQHEEEV LFKDILDKIELIGNENHGLYLADQYVKGIAKSRKSGS | 32 |
| | pCoV29 | His8c-3c-RBD-3-Ferritin | 393 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYLYRLFRKSNLK PFERDISTEIYQAGSTPCNGVEGFNCYFPLQSYGFQPTNGVGYQPYRVVV LSFELLHAPATVCGGSGGESQVRQQFSKDIEKLLNEQVNKEMQSSNLYMS MSSWCYTHSLDGAGLFLFDHAAEEYEHAKKLIIFLNENNVPVQLTSISAP EHKFEGLTQIFQKAYEHEQHISESINNIVDHAIKSKDHATFNFLQWYVAE QHEEEVLFKDILDKIELIGNENHGLYLADQYVKGIAKSRKSGS | 33 |
| | pCoV30 | His8-3c-RBD-3-del-Ferritin | 383 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYLYRLFRKSNLK PFERDISTEIYQAGSTPCNGVEGFNCYFPLQSYGFQPTNGVGYQPYRVVV LSFELLHAPATVCGGSGGDIIKLLNEQVNKEMQSSNLYMSMSSWCYTHSL DGAGLFLFDHAAEEYEHAKKLIIFLNENNVPVQLTSISAPEHKFEGLTQI FQKAYEHEQHISESINNIVDHAIKSKDHATFNFLQWYVAEQHEEEVLFKD ILDKIELIGNENHGLYLADQYVKGIAKSRKSGS | 34 |
| | pCoV31 | His8-3c-RBD-6-del-Ferritin | 388 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYLYRLFRKSNLK PFERDISTEIYQAGSTPCNGVEGFNCYFPLQSYGFQPTNGVGYQPYRVVV LSFELLHAPATVCGGGSGGGGSKDIIKLLNEQVNKEMQSSNLYMSMSSWC YTHSLDGAGLFLFDHAAEEYEHAKKLIIFLNENNVPVQLTSISAPEHKFE GLTQIFQKAYEHEQHISESINNIVDHAIKSKDHATFNFLQWYVAEQHEEE VLFKDILDKIELIGNENHGLYLADQYVKGIAKSRKSGS | 35 |
| | pCoV32 | His8-3c-RBDSD1-Ferritin | 472 | HHHHHHHHGPLEVLFQGPRVQPTESIVRFPNITNLCPFGEVFNATRFASV YAWNRKRISNCVADYSVLYNSASFSTFKCYGVSPTKLNDLCFTNVYADSF VIRGDEVRQIAPGQTGKIADYNYKLPDDFTGCVIAWNSNNLDSKVGGNYN YLYRLFRKSNLKPFERDISTEIYQAGSTPCNGVEGFNCYFPLQSYGFQPT NGVGYQPYRVVVLSFELLHAPATVCGPKKSTNLVKNKCVNFNFNGLTGTG VLTESNKKFLPFQQFGRDIADTTDAVRDPQTLEILDITPCSGSGGGGESQ VRQQFSKDIEKLLNEQVNKEMQSSNLYMSMSSWCYTHSLDGAGLFLFDHA AEEYEHAKKLIIFLNENNVPVQLTSISAPEHKFEGLTQIFQKAYEHEQHI SESINNIVDHAIKSKDHATFNFLQWYVAEQHEEEVLFKDILDKIELIGNE NHGLYLADQYVKGIAKSRKSGS | 36 |
| | pCoV33 | Ferritin | 175 | ESQVRQQFSKDIEKLLNEQVNKEMQSSNLYMSMSSWCYTHSLDGAGLFLF DHAAEEYEHAKKLIIFLNENNVPVQLTSISAPEHKFEGLTQIFQKAYEHE QHISESINNIVDHAIKSKDHATFNFLQWYVAEQHEEEVLFKDILDKIELI GNENHGLYLADQYVKGIAKSRKSGS | 37 |
| | pCoV34 | His8-3c-NTD-6-LS | 466 | HHHHHHHHGPLEVLFQGPVNLTTRTQLPPAYTNSFTRGVYYPDKVFRSSV LHSTQDLFLPFFSNVTWFHAIHVSGTNGTKRFDNPVLPFNDGVYFASTEK SNIIRGWIFGTTLDSKTQSLLIVNNATNVVIKVCEFQFCNDPFLGVYYHK NNKSWMESEFRVYSSANNCTFEYVSQPFLMDLEGKQGNFKNLREFVFKNI DGYFKIYSKHTPINLVRDLPQGFSALEPLVDLPIGINITRFQTLLALHRS YLTPGDSSSGWTAGAAAYYVGYLQPRTFLLKYNENGTITDAVDCALDPLS ETKCTLGSGGGGMQIYEGKLTAEGLRFGIVASRFNHALVDRLVEGAIDAI VRHGGREEDITLVRVPGSWEIPVAAGELARKEDIDAVIAIGVLIRGATPH FDYIASEVSKGLADLSLELRKPITFGVITADTLEQAIERAGTKHGNKGWE AALSAIEMANLFKSLR | 38 |
| | pCoV35 | LS-15-NTD-3c-Strep-His8 | 475 | MQIYEGKLTAEGLRFGIVASRFNHALVDRLVEGAIDAIVRHGGREEDITL VRVPGSWEIPVAAGELARKEDIDAVIAIGVLIRGATPHFDYIASEVSKGL ADLSLELRKPITFGVITADTLEQAIERAGTKHGNKGWEAALSAIEMANLF KSLRGGSGGSGGSGGSGGGVNLTTRTQLPPAYTNSFTRGVYYPDKVFRSS VLHSTQDLFLPFFSNVTWFHAIHVSGTNGTKRFDNPVLPFNDGVYFASTE KSNIIRGWIFGTTLDSKTQSLLIVNNATNVVIKVCEFQFCNDPFLGVYYH KNNKSWMESEFRVYSSANNCTFEYVSQPFLMDLEGKQGNFKNLREFVFKN IDGYFKIYSKHTPINLVRDLPQGFSALEPLVDLPIGINITRFQTLLALHR SYLTPGDSSSGWTAGAAAYYVGYLQPRTFLLKYNENGTITDAVDCALDPL SETKCTLGSLEVLFQGPHHHHHHHH | 39 |
| | pCoV39 | RBD-3-Ferritin | 375 | NITNLCPFGEVFNATRFASVYAWNRKRISNCVADYSVLYNSASFSTFKCY GVSPTKLNDLCFTNVYADSFVIRGDEVRQIAPGQTGKIADYNYKLPDDFT GCVIAWNSNNLDSKVGGNYNYLYRLFRKSNLKPFERDISTEIYQAGSTPC NGVEGFNCYFPLQSYGFQPTNGVGYQPYRVVVLSFELLHAPATVCGGSGG ESQVRQQFSKDIEKLLNEQVNKEMQSSNLYMSMSSWCYTHSLDGAGLFLF DHAAEEYEHAKKLIIFLNENNVPVQLTSISAPEHKFEGLTQIFQKAYEHE QHISESINNIVDHAIKSKDHATFNFLQWYVAEQHEEEVLFKDILDKIELI GNENHGLYLADQYVKGIAKSRKSGS | 40 |
| | pCoV40 | RBD-3-delFerritin | 365 | NITNLCPFGEVFNATRFASVYAWNRKRISNCVADYSVLYNSASFSTFKCY GVSPTKLNDLCFTNVYADSFVIRGDEVRQIAPGQTGKIADYNYKLPDDFT GCVIAWNSNNLDSKVGGNYNYLYRLFRKSNLKPFERDISTEIYQAGSTPC NGVEGFNCYFPLQSYGFQPTNGVGYQPYRVVVLSFELLHAPATVCGGSGG DIIKLLNEQVNKEMQSSNLYMSMSSWCYTHSLDGAGLFLFDHAAEEYEHA KKLIIFLNENNVPVQLTSISAPEHKFEGLTQIFQKAYEHEQHISESINNI VDHAIKSKDHATFNFLQWYVAEQHEEEVLFKDILDKIELIGNENHGLYLA DQYVKGIAKSRKSGS | 41 |
| | pCoV46 | His8-3c-RBDSD1-6-del-Ferritin | 464 | HHHHHHHHGPLEVLFQGPRVQPTESIVRFPNITNLCPFGEVFNATRFASV YAWNRKRISNCVADYSVLYNSASFSTFKCYGVSPTKLNDLCFTNVYADSF VIRGDEVRQIAPGQTGKIADYNYKLPDDFTGCVIAWNSNNLDSKVGGNYN YLYRLFRKSNLKPFERDISTEIYQAGSTPCNGVEGFNCYFPLQSYGFQPT NGVGYQPYRVVVLSFELLHAPATVCGPKKSTNLVKNKCVNFNFNGLTGTG VLTESNKKFLPFQQFGRDIADTTDAVRDPQTLEILDITPCSGSGGGGSKD IIKLLNEQVNKEMQSSNLYMSMSSWCYTHSLDGAGLFLFDHAAEEYEHAK KLIIFLNENNVPVQLTSISAPEHKFEGLTQIFQKAYEHEQHISESINNIV DHAIKSKDHATFNFLQWYVAEQHEEEVLFKDILDKIELIGNENHGLYLAD QYVKGIAKSRKSGS | 42 |
| | pCov1A-01 | His8-3C-RBD-PPII-Ferritin | 409 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYLYRLFRKSNLK PFERDISTEIYQAGSTPCNGVEGFNCYFPLQSYGFQPTNGVGYQPYRVVV LSFELLHAPATVCGGSGGPPPPPPPPPPGSGGGGESQVRQQFSKDIEKLL NEQVNKEMQSSNLYMSMSSWCYTHSLDGAGLFLFDHAAEEYEHAKKLIIF LNENNVPVQLTSISAPEHKFEGLTQIFQKAYEHEQHISESINNIVDHAIK SKDHATFNFLQWYVAEQHEEEVLFKDILDKIELIGNENHGLYLADQYVKG IAKSRKSGS | 43 |
| | pCoV1A-02 | His8-3C-RBD-alpha1-Ferritin | 410 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYLYRLFRKSNLK PFERDISTEIYQAGSTPCNGVEGFNCYFPLQSYGFQPTNGVGYQPYRVVV LSFELLHAPATVCGGSGGEKDSHKEEKDSHKGSGGESQVRQQFSKDIEKL LNEQVNKEMQSSNLYMSMSSWCYTHSLDGAGLFLFDHAAEEYEHAKKLII FLNENNVPVQLTSISAPEHKFEGLTQIFQKAYEHEQHISESINNIVDHAI KSKDHATFNFLQWYVAEQHEEEVLFKDILDKIELIGNENHGLYLADQYVK GIAKSRKSGS | 44 |
| | pCoV1A-03 | His8-3C-RBD-alpha2-Ferritin | 412 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYLYRLFRKSNLK PFERDISTEIYQAGSTPCNGVEGFNCYFPLQSYGFQPTNGVGYQPYRVVV LSFELLHAPATVCGGSGGEDNAQHTSADNEATKGSGGESQVRQQFSKDIE KLLNEQVNKEMQSSNLYMSMSSWCYTHSLDGAGLFLFDHAAEEYEHAKKL IIFLNENNVPVQLTSISAPEHKFEGLTQIFQKAYEHEQHISESINNIVDH AIKSKDHATFNFLQWYVAEQHEEEVLFKDILDKIELIGNENHGLYLADQY VKGIAKSRKSGS | 45 |
| | pCoV1A-04 | His8-3C-RBD-GCN4-del-Ferritin | 423 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYLYRLFRKSNLK PFERDISTEIYQAGSTPCNGVEGFNCYFPLQSYGFQPTNGVGYQPYRVVV LSFELLHAPATVCGGSGSGGEMKQIEDKIEEILSKIYHIENEIARIKKLI GRGSGGSGDIIKLLNEQVNKEMQSSNLYMSMSSWCYTHSLDGAGLFLFDH AAEEYEHAKKLIIFLNENNVPVQLTSISAPEHKFEGLTQIFQKAYEHEQH ISESINNIVDHAIKSKDHATFNFLQWYVAEQHEEEVLFKDILDKIELIGN ENHGLYLADQYVKGIAKSRKSGS | 46 |
| | pCoV1A-05 | His8-3C-RBD-1141_1158opl -del-Ferritin | 406 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYLYRLFRKSNLK PFERDISTEIYQAGSTPCNGVEGFNCYFPLQSYGFQPTNGVGYQPYRVVV LSFELLHAPATVCGGSGSGGELQSELDSIKEELDKGSGGSGDIIKLLNEQ VNKEMQSSNLYMSMSSWCYTHSLDGAGLFLFDHAAEEYEHAKKLIIFLNE NNVPVQLTSISAPEHKFEGLTQIFQKAYEHEQHISESINNIVDHAIKSKD HATFNFLQWYVAEQHEEEVLFKDILDKIELIGNENHGLYLADQYVKGIAK SRKSGS | 47 |
| | pCoV1A-06 | His8-3C-RBD-1141_1158opl x2-del-Ferritin | 424 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYLYRLFRKSNLK PFERDISTEIYQAGSTPCNGVEGFNCYFPLQSYGFQPTNGVGYQPYRVVV LSFELLHAPATVCGGSGSGGELQSELDSIKEELDKIHKNLDSIKEELDKI HKNGSGGSGDIIKLLNEQVNKEMQSSNLYMSMSSWCYTHSLDGAGLFLFD HAAEEYEHAKKLIIFLNENNVPVQLTSISAPEHKFEGLTQIFQKAYEHEQ HISESINNIVDHAIKSKDHATFNFLQWYVAEQHEEEVLFKDILDKIELIG NENHGLYLADQYVKGIAKSRKSGS | 48 |
| | pCoV49 | His8-3c-RBD-Ferritin-F456N/K458T | 396 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYLYRLNRTSNLK PFERDISTEIYQAGSTPCNGVEGFNCYFPLQSYGFQPTNGVGYQPYRVVV LSFELLHAPATVCGPGSGGGGESQVRQQFSKDIEKLLNEQVNKEMQSSNL YMSMSSWCYTHSLDGAGLFLFDHAAEEYEHAKKLIIFLNENNVPVQLTSI SAPEHKFEGLTQIFQKAYEHEQHISESINNIVDHAIKSKDHATFNFLQWY VAEQHEEEVLFKDILDKIELIGNENHGLYLADQYVKGIAKSRKSGS | 49 |
| | pCoV50 | His8-3c-RBD-Ferritin-L455R/Y449K /F490R | 396 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNKNYLYRRFRKSNLK PFERDISTEIYQAGSTPCNGVEGFNCYRPLQSYGFQPTNGVGYQPYRVVV LSFELLHAPATVCGPGSGGGGESQVRQQFSKDIEKLLNEQVNKEMQSSNL YMSMSSWCYTHSLDGAGLFLFDHAAEEYEHAKKLIIFLNENNVPVQLTSI SAPEHKFEGLTQIFQKAYEHEQHISESINNIVDHAIKSKDHATFNFLQWY V AEQHEEEVLFKDILDKIELIGNENHGLYLADQYVKGIAKSRKSGS | 50 |
| | pCoV51 | His8-3c-RBD-Ferritin-L455R | 396 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYLYRRFRKSNLK PFERDISTEIYQAGSTPCNGVEGFNCYFPLQSYGFQPTNGVGYQPYRVVV LSFELLHAPATVCGPGSGGGGESQVRQQFSKDIEKLLNEQVNKEMQSSNL YMSMSSWCYTHSLDGAGLFLFDHAAEEYEHAKKLIIFLNENNVPVQLTSI SAPEHKFEGLTQIFQKAYEHEQHISESINNIVDHAIKSKDHATFNFLQWY V AEQHEEEVLFKDILDKIELIGNENHGLYLADQYVKGIAKSRKSGS | 51 |
| | pCoV52 | His8-3c-RBD-Ferritin-I468R | 396 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYLYRLFRKSNLK PFERDRSTEIYQAGSTPCNGVEGFNCYFPLQSYGFQPTNGVGYQPYRVVV LSFELLHAPATVCGPGSGGGGESQVRQQFSKDIEKLLNEQVNKEMQSSNL YMSMSSWCYTHSLDGAGLFLFDHAAEEYEHAKKLIIFLNENNVPVQLTSI SAPEHKFEGLTQIFQKAYEHEQHISESINNIVDHAIKSKDHATFNFLQWY V AEQHEEEVLFKDILDKIELIGNENHGLYLADQYVKGIAKSRKSGS | 52 |
| | pCoV53 | His8-3c-RBD-Ferritin-Y453R | 396 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYLRRLFRKSNLK PFERDISTEIYQAGSTPCNGVEGFNCYFPLQSYGFQPTNGVGYQPYRVVV LSFELLHAPATVCGPGSGGGGESQVRQQFSKDIEKLLNEQVNKEMQSSNL YMSMSSWCYTHSLDGAGLFLFDHAAEEYEHAKKLIIFLNENNVPVQLTSI SAPEHKFEGLTQIFQKAYEHEQHISESINNIVDHAIKSKDHATFNFLQWY V AEQHEEEVLFKDILDKIELIGNENHGLYLADQYVKGIAKSRKSGS | 53 |
| | pCoV54 | His8-3c-RBD-Ferritin-L452R | 396 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYRYRLFRKSNLK PFERDISTEIYQAGSTPCNGVEGFNCYFPLQSYGFQPTNGVGYQPYRVVV LSFELLHAPATVCGPGSGGGGESQVRQQFSKDIEKLLNEQVNKEMQSSNL YMSMSSWCYTHSLDGAGLFLFDHAAEEYEHAKKLIIFLNENNVPVQLTSI SAPEHKFEGLTQIFQKAYEHEQHISESINNIVDHAIKSKDHATFNFLQWY V AEQHEEEVLFKDILDKIELIGNENHGLYLADQYVKGIAKSRKSGS | 54 |
| | pCoV55 | His8-3c-RBD-Ferritin-L492R | 396 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYLYRLFRKSNLK PFERDISTEIYQAGSTPCNGVEGFNCYFPRQSYGFQPTNGVGYQPYRVVV LSFELLHAPATVCGPGSGGGGESQVRQQFSKDIEKLLNEQVNKEMQSSNL YMSMSSWCYTHSLDGAGLFLFDHAAEEYEHAKKLIIFLNENNVPVQLTSI SAPEHKFEGLTQIFQKAYEHEQHISESINNIVDHAIKSKDHATFNFLQWY VAEQHEEEVLFKDILDKIELIGNENHGLYLADQYVKGIAKSRKSGS | 55 |
| | pCoV56 | His8-3c-RBD-Ferritin-F490R | 396 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYLYRLFRKSNLK PFERDISTEIYQAGSTPCNGVEGFNCYRPLQSYGFQPTNGVGYQPYRVVV LSFELLHAPATVCGPGSGGGGESQVRQQFSKDIEKLLNEQVNKEMQSSNL YMSMSSWCYTHSLDGAGLFLFDHAAEEYEHAKKLIIFLNENNVPVQLTSI SAPEHKFEGLTQIFQKAYEHEQHISESINNIVDHAIKSKDHATFNFLQWY V AEQHEEEVLFKDILDKIELIGNENHGLYLADQYVKGIAKSRKSGS | 56 |
| | pCoV57 | His8-3c-RBD-Ferritin-F490A | 396 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYLYRLFRKSNLK PFERDISTEIYQAGSTPCNGVEGFNCYAPLQSYGFQPTNGVGYQPYRVVV LSFELLHAPATVCGPGSGGGGESQVRQQFSKDIEKLLNEQVNKEMQSSNL YMSMSSWCYTHSLDGAGLFLFDHAAEEYEHAKKLIIFLNENNVPVQLTSI SAPEHKFEGLTQIFQKAYEHEQHISESINNIVDHAIKSKDHATFNFLQWY V AEQHEEEVLFKDILDKIELIGNENHGLYLADQYVKGIAKSRKSGS | 57 |
| | pCoV58 | His8-3c-RBD-Ferritin-518LLH to 518 NKS | 396 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYLYRLFRKSNLK PFERDISTEIYQAGSTPCNGVEGFNCYFPLQSYGFQPTNGVGYQPYRVVV LSFENKSAPATVCGPGSGGGGESQVRQQFSKDIEKLLNEQVNKEMQSSNL YMSMSSWCYTHSLDGAGLFLFDHAAEEYEHAKKLIIFLNENNVPVQLTSI SAPEHKFEGLTQIFQKAYEHEQHISESINNIVDHAIKSKDHATFNFLQWY VAEQHEEEVLFKDILDKIELIGNENHGLYLADQYVKGIAKSRKSGS | 58 |
| | pCoV59 | His8-3c-RBD-Ferritin-L518R | 396 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYLYRLFRKSNLK PFERDISTEIYQAGSTPCNGVEGFNCYFPLQSYGFQPTNGVGYQPYRVVV LSFELRHAPATVCGPGSGGGGESQVRQQFSKDIEKLLNEQVNKEMQSSNL YMSMSSWCYTHSLDGAGLFLFDHAAEEYEHAKKLIIFLNENNVPVQLTSI SAPEHKFEGLTQIFQKAYEHEQHISESINNIVDHAIKSKDHATFNFLQWY VAEQHEEEVLFKDILDKIELIGNENHGLYLADQYVKGIAKSRKSGS | 59 |
| | pCoV60 | His8-3c-RBD-Ferritin-V367T/L335N | 396 | HHHHHHHHGPLEVLFQGPNITNNCPFGEVFNATRFASVYAWNRKRISNCV ADYSTLYNSASFSTFKCYGVSPTKLNDLCFTNVYADSFVIRGDEVRQIAP GQTGKIADYNYKLPDDFTGCVIAWNSNNLDSKVGGNYNYLYRLFRKSNLK PFERDISTEIYQAGSTPCNGVEGFNCYFPLQSYGFQPTNGVGYQPYRVVV LSFELLHAPATVCGPGSGGGGESQVRQQFSKDIEKLLNEQVNKEMQSSNL YMSMSSWCYTHSLDGAGLFLFDHAAEEYEHAKKLIIFLNENNVPVQLTSI SAPEHKFEGLTQIFQKAYEHEQHISESINNIVDHAIKSKDHATFNFLQWY VAEQHEEEVLFKDILDKIELIGNENHGLYLADQYVKGIAKSRKSGS | 60 |
| | pCoV61 | His8-3c-RBD-Ferritin-T385N/L387T | 396 | HHHHHHHHGPLEVLFQGPNITNLCPFGEVFNATRFASVYAWNRKRISNCV ADYSVLYNSASFSTFKCYGVSPNKTNDLCFTNVYADSFVIRGDEVRQIAP GQTGKIADYNYKLPDDFTGCVIAWNSNNLDSKVGGNYNYLYRLFRKSNLK PFERDISTEIYQAGSTPCNGVEGFNCYFPLQSYGFQPTNGVGYQPYRVVV LSFELLHAPATVCGPGSGGGGESQVRQQFSKDIEKLLNEQVNKEMQSSNL YMSMSSWCYTHSLDGAGLFLFDHAAEEYEHAKKLIIFLNENNVPVQLTSI SAPEHKFEGLTQIFQKAYEHEQHISESINNIVDHAIKSKDHATFNFLQWY VAEQHEEEVLFKDILDKIELIGNENHGLYLADQYVKGIAKSRKSGS | 61 |
| | pCoV62 | His8-3c-RBD-Ferritin-V382R | 396 | HHHHHHHHGPLEVLFQGPNITNLCPFGEVFNATRFASVYAWNRKRISNCV ADYSVLYNSASFSTFKCYGRSPTKLNDLCFTNVYADSFVIRGDEVRQIAP GQTGKIADYNYKLPDDFTGCVIAWNSNNLDSKVGGNYNYLYRLFRKSNLK PFERDISTEIYQAGSTPCNGVEGFNCYFPLQSYGFQPTNGVGYQPYRVVV LSFELLHAPATVCGPGSGGGGESQVRQQFSKDIEKLLNEQVNKEMQSSNL YMSMSSWCYTHSLDGAGLFLFDHAAEEYEHAKKLIIFLNENNVPVQLTSI SAPEHKFEGLTQIFQKAYEHEQHISESINNIVDHAIKSKDHATFNFLQWY VAEQHEEEVLFKDILDKIELIGNENHGLYLADQYVKGIAKSRKSGS | 62 |
| | pCoV63 | His8-3c-RBD-Ferritin-F377R | 396 | HHHHHHHHGPLEVLFQGPNITNLCPFGEVFNATRFASVYAWNRKRISNCV ADYSVLYNSASFSTRKCYGVSPTKLNDLCFTNVYADSFVIRGDEVRQIAP GQTGKIADYNYKLPDDFTGCVIAWNSNNLDSKVGGNYNYLYRLFRKSNLK PFERDISTEIYQAGSTPCNGVEGFNCYFPLQSYGFQPTNGVGYQPYRVVV LSFELLHAPATVCGPGSGGGGESQVRQQFSKDIEKLLNEQVNKEMQSSNL YMSMSSWCYTHSLDGAGLFLFDHAAEEYEHAKKLIIFLNENNVPVQLTSI SAPEHKFEGLTQIFQKAYEHEQHISESINNIVDHAIKSKDHATFNFLQWY VAEQHEEEVLFKDILDKIELIGNENHGLYLADQYVKGIAKSRKSGS | 63 |
| | pCoV64 | NTD-Ferritin | | VNLTTRTQLPPAYTNSFTRGVYYPDKVFRSSVLHSTQDLFLPFFSNVTWF HAIHVSGTNGTKRFDNPVLPFNDGVYFASTEKSNIIRGWIFGTTLDSKTQ SLLIVNNATNVVIKVCEFQFCNDPFLGVYYHKNNKSWMESEFRVYSSANN CTFEYVSQPFLMDLEGKQGNFKNLREFVFKNIDGYFKIYSKHTPINLVRD LPQGFSALEPLVDLPIGINITRFQTLLALHRSYLTPGDSSSGWTAGAAAY YVGYLQPRTFLLKYNENGTITDAVDCALDPLSETKCTLGSGGGGESQVRQ QFSKDIEKLLNEQVNKEMQSSNLYMSMSSWCYTHSLDGAGLFLFDHAAEE YEHAKKLIIFLNENNVPVQLTSISAPEHKFEGLTQIFQKAYEHEQHISES INNIVDHAIKSKDHATFNFLQWYVAEQHEEEVLFKDILDKIELIGNENHG LYLADQYVKGIAKSRKSGS | 64 |
| | pCoV65 | His8-3c NTD-SSQC-Ferrritin | 491 | HHHHHHHHGPLEVLFQGPSSQCVNLTTRTQLPPAYTNSFTRGVYYPDKVF RSSVLHSTQDLFLPFFSNVTWFHAIHVSGTNGTKRFDNPVLPFNDGVYFA STEKSNIIRGWIFGTTLDSKTQSLLIVNNATNVVIKVCEFQFCNDPFLGV YYHKNNKSWMESEFRVYSSANNCTFEYVSQPFLMDLEGKQGNFKNLREFV FKNIDGYFKIYSKHTPINLVRDLPQGFSALEPLVDLPIGINITRFQTLLA LHRSYLTPGDSSSGWTAGAAAYYVGYLQPRTFLLKYNENGTITDAVDCAL DPLSETKCTLGSGGGGESQVRQQFSKDIEKLLNEQVNKEMQSSNLYMSMS SWCYTHSLDGAGLFLFDHAAEEYEHAKKLIIFLNENNVPVQLTSISAPEH KFEGLTQIFQKAYEHEQHISESINNIVDHAIKSKDHATFNFLQWYVAEQH EEEVLFKDILDKIELIGNENHGLYLADQYVKGIAKSRKSGS | 65 |
| | pCoV66 | NTD-SSQC-Ferrritin | 473 | SSQCVNLTTRTQLPPAYTNSFTRGVYYPDKVFRSSVLHSTQDLFLPFFSN VTWFHAIHVSGTNGTKRFDNPVLPFNDGVYFASTEKSNIIRGWIFGTTLD SKTQSLLIVNNATNVVIKVCEFQFCNDPFLGVYYHKNNKSWMESEFRVYS SANNCTFEYVSQPFLMDLEGKQGNFKNLREFVFKNIDGYFKIYSKHTPIN LVRDLPQGFSALEPLVDLPIGINITRFQTLLALHRSYLTPGDSSSGWTAG AAAYYVGYLQPRTFLLKYNENGTITDAVDCALDPLSETKCTLGSGGGGES QVRQQFSKDIEKLLNEQVNKEMQSSNLYMSMSSWCYTHSLDGAGLFLFDH AAEEYEHAKKLIIFLNENNVPVQLTSISAPEHKFEGLTQIFQKAYEHEQH ISESINNIVDHAIKSKDHATFNFLQWYVAEQHEEEVLFKDILDKIELIGN ENHGLYLADQYVKGIAKSRKSGS | 66 |
| | pCoV67 | His8-3c-S1-SSQC-Ferritin | 866 | HHHHHHHHGPLEVLFQGPSSQCVNLTTRTQLPPAYTNSFTRGVYYPDKVF RSSVLHSTQDLFLPFFSNVTWFHAIHVSGTNGTKRFDNPVLPFNDGVYFA STEKSNIIRGWIFGTTLDSKTQSLLIVNNATNVVIKVCEFQFCNDPFLGV YYHKNNKSWMESEFRVYSSANNCTFEYVSQPFLMDLEGKQGNFKNLREFV FKNIDGYFKIYSKHTPINLVRDLPQGFSALEPLVDLPIGINITRFQTLLA LHRSYLTPGDSSSGWTAGAAAYYVGYLQPRTFLLKYNENGTITDAVDCAL DPLSETKCTLKSFTVEKGIYQTSNFRVQPTESIVRFPNITNLCPFGEVFN ATRFASVYAWNRKRISNCVADYSVLYNSASFSTFKCYGVSPTKLNDLCFT NVYADSFVIRGDEVRQIAPGQTGKIADYNYKLPDDFTGCVIAWNSNNLDS KVGGNYNYLYRLFRKSNLKPFERDISTEIYQAGSTPCNGVEGFNCYFPLQ SYGFQPTNGVGYQPYRVVVLSFELLHAPATVCGPKKSTNLVKNKCVNFNF NGLTGTGVLTESNKKFLPFQQFGRDIADTTDAVRDPQTLEILDITPCSFG GVSVITPGTNTSNQVAVLYQDVNCTEVPVAIHADQLTPTWRVYSTGSNVF QTRAGCLIGAEHVNNSYECDIPIGAGICASYQTQTGSGGGGESQVRQQFS KDIEKLLNEQVNKEMQSSNLYMSMSSWCYTHSLDGAGLFLFDHAAEEYEH AKKLIIFLNENNVPVQLTSISAPEHKFEGLTQIFQKAYEHEQHISESINN IVDHAIKSKDHATFNFLQWYVAEQHEEEVLFKDILDKIELIGNENHGLYL ADQYVKGIAKSRKSGS | 67 |
| | pCoV68 | S1-SSQC-Ferritin | 848 | SSQCVNLTTRTQLPPAYTNSFTRGVYYPDKVFRSSVLHSTQDLFLPFFSN VTWFHAIHVSGTNGTKRFDNPVLPFNDGVYFASTEKSNIIRGWIFGTTLD SKTQSLLIVNNATNVVIKVCEFQFCNDPFLGVYYHKNNKSWMESEFRVYS SANNCTFEYVSQPFLMDLEGKQGNFKNLREFVFKNIDGYFKIYSKHTPIN LVRDLPQGFSALEPLVDLPIGINITRFQTLLALHRSYLTPGDSSSGWTAG AAAYYVGYLQPRTFLLKYNENGTITDAVDCALDPLSETKCTLKSFTVEKG IYQTSNFRVQPTESIVRFPNITNLCPFGEVFNATRFASVYAWNRKRISNC VADYSVLYNSASFSTFKCYGVSPTKLNDLCFTNVYADSFVIRGDEVRQIA PGQTGKIADYNYKLPDDFTGCVIAWNSNNLDSKVGGNYNYLYRLFRKSNL KPFERDISTEIYQAGSTPCNGVEGFNCYFPLQSYGFQPTNGVGYQPYRVV VLSFELLHAPATVCGPKKSTNLVKNKCVNFNFNGLTGTGVLTESNKKFLP FQQFGRDIADTTDAVRDPQTLEILDITPCSFGGVSVITPGTNTSNQVAVL YQDVNCTEVPVAIHADQLTPTWRVYSTGSNVFQTRAGCLIGAEHVNNSYE CDIPIGAGICASYQTQTGSGGGGESQVRQQFSKDIEKLLNEQVNKEMQSS NLYMSMSSWCYTHSLDGAGLFLFDHAAEEYEHAKKLIIFLNENNVPVQLT SISAPEHKFEGLTQIFQKAYEHEQHISESINNIVDHAIKSKDHATFNFLQ WYVAEQHEEEVLFKDILDKIELIGNENHGLYLADQYVKGIAKSRKSGS | 68 |
| | pCoV69 | PrefLead-S2P-Foldon-3c-Strep-His8 | 1,275 | SSQCVNLTTRTQLPPAYTNSFTRGVYYPDKVFRSSVLHSTQDLFLPFFSN VTWFHAIHVSGTNGTKRFDNPVLPFNDGVYFASTEKSNIIRGWIFGTTLD SKTQSLLIVNNATNVVIKVCEFQFCNDPFLGVYYHKNNKSWMESEFRVYS SANNCTFEYVSQPFLMDLEGKQGNFKNLREFVFKNIDGYFKIYSKHTPIN LVRDLPQGFSALEPLVDLPIGINITRFQTLLALHRSYLTPGDSSSGWTAG AAAYYVGYLQPRTFLLKYNENGTITDAVDCALDPLSETKCTLKSFTVEKG IYQTSNFRVQPTESIVRFPNITNLCPFGEVFNATRFASVYAWNRKRISNC VADYSVLYNSASFSTFKCYGVSPTKLNDLCFTNVYADSFVIRGDEVRQIA PGQTGKIADYNYKLPDDFTGCVIAWNSNNLDSKVGGNYNYLYRLFRKSNL KPFERDISTEIYQAGSTPCNGVEGFNCYFPLQSYGFQPTNGVGYQPYRVV VLSFELLHAPATVCGPKKSTNLVKNKCVNFNFNGLTGTGVLTESNKKFLP FQQFGRDIADTTDAVRDPQTLEILDITPCSFGGVSVITPGTNTSNQVAVL YQDVNCTEVPVAIHADQLTPTWRVYSTGSNVFQTRAGCLIGAEHVNNSYE CDIPIGAGICASYQTQTNSPGSASSVASQSIIAYTMSLGAENSVAYSNNS IAIPTNFTISVTTEILPVSMTKTSVDCTMYICGDSTECSNLLLQYGSFCT QLNRALTGIAVEQDKNTQEVFAQVKQIYKTPPIKDFGGFNFSQILPDPSK PSKRSFIEDLLFNKVTLADAGFIKQYGDCLGDIAARDLICAQKFNGLTVL PPLLTDEMIAQYTSALLAGTITSGWTFGAGAALQIPFAMQMAYRFNGIGV TQNVLYENQKLIANQFNSAIGKIQDSLSSTASALGKLQDVVNQNAQALNT LVKQLSSNFGAISSVLNDILSRLDPPEAEVQIDRLITGRLQSLQTYVTQQ LIRAAEIRASANLAATKMSECVLGQSKRVDFCGKGYHLMSFPQSAPHGVV FLHVTYVPAQEKNFTTAPAICHDGKAHFPREGVFVSNGTHWFVTQRNFYE PQIITTDNTFVSGNCDVVIGIVNNTVYDPLQPELDSFKEELDKYFKNHTS PDVDLGDISGINASVVNIQKEIDRLNEVAKNLNESLIDLQELGKYEQGSG YIPEAPRDGQAYVRKDGEWVLLSTFLGLEVLFQGPSAWSHPQFEKGGGSG GGSGGSAWSHPQFEKGSHHHHHHHH | 69 |
| Includes Native spike leader | pCoVIB-01 | S2P.1137-del-4-Ferritin | 1,291 | VNLTTRTQLPPAYTNSFTRGVYYPDKVFRSSVLHSTQDLFLPFFSNVTWF HAIHVSGTNGTKRFDNPVLPFNDGVYFASTEKSNIIRGWIFGTTLDSKTQ SLLIVNNATNVVIKVCEFQFCNDPFLGVYYHKNNKSWMESEFRVYSSANN CTFEYVSQPFLMDLEGKQGNFKNLREFVFKNIDGYFKIYSKHTPINLVRD LPQGFSALEPLVDLPIGINITRFQTLLALHRSYLTPGDSSSGWTAGAAAY YVGYLQPRTFLLKYNENGTITDAVDCALDPLSETKCTLKSFTVEKGIYQT SNFRVQPTESIVRFPNITNLCPFGEVFNATRFASVYAWNRKRISNCVADY SVLYNSASFSTFKCYGVSPTKLNDLCFTNVYADSFVIRGDEVRQIAPGQT GKIADYNYKLPDDFTGCVIAWNSNNLDSKVGGNYNYLYRLFRKSNLKPFE RDISTEIYQAGSTPCNGVEGFNCYFPLQSYGFQPTNGVGYQPYRVVVLSF ELLHAPATVCGPKKSTNLVKNKCVNFNFNGLTGTGVLTESNKKFLPFQQF GRDIADTTDAVRDPQTLEILDITPCSFGGVSVITPGTNTSNQVAVLYQDV NCTEVPVAIHADQLTPTWRVYSTGSNVFQTRAGCLIGAEHVNNSYECDIP IGAGICASYQTQTNSPGSASSVASQSIIAYTMSLGAENSVAYSNNSIAIP TNFTISVTTEILPVSMTKTSVDCTMYICGDSTECSNLLLQYGSFCTQLNR ALTGIAVEQDKNTQEVFAQVKQIYKTPPIKDFGGFNFSQILPDPSKPSKR SFIEDLLFNKVTLADAGFIKQYGDCLGDIAARDLICAQKFNGLTVLPPLL TDEMIAQYTSALLAGTITSGWTFGAGAALQIPFAMQMAYRFNGIGVTQNV LYENQKLIANQFNSAIGKIQDSLSSTASALGKLQDVVNQNAQALNTLVKQ LSSNFGAISSVLNDILSRLDPPEAEVQIDRLITGRLQSLQTYVTQQLIRA AEIRASANLAATKMSECVLGQSKRVDFCGKGYHLMSFPQSAPHGVVFLHV TYVPAQEKNFTTAPAICHDGKAHFPREGVFVSNGTHWFVTQRNFYEPQII TTDNTFVSGNCDVVIGIVNNTVGSGGDIIKLLNEQVNKEMQSSNLYMSMS SWCYTHSLDGAGLFLFDHAAEEYEHAKKLIIFLNENNVPVQLTSISAPEH KFEGLTQIFQKAYEHEQHISESINNIVDHAIKSKDHATFNFLQWYVAEQH EEEVLFKDILDKIELIGNENHGLYLADQYVKGIAKSRKSGS | 70 |
| | pCoV1B-02 | S2P.1137-del-6-Ferritin | 1,293 | VNLTTRTQLPPAYTNSFTRGVYYPDKVFRSSVLHSTQDLFLPFFSNVTWF HAIHVSGTNGTKRFDNPVLPFNDGVYFASTEKSNIIRGWIFGTTLDSKTQ SLLIVNNATNVVIKVCEFQFCNDPFLGVYYHKNNKSWMESEFRVYSSANN CTFEYVSQPFLMDLEGKQGNFKNLREFVFKNIDGYFKIYSKHTPINLVRD LPQGFSALEPLVDLPIGINITRFQTLLALHRSYLTPGDSSSGWTAGAAAY YVGYLQPRTFLLKYNENGTITDAVDCALDPLSETKCTLKSFTVEKGIYQT SNFRVQPTESIVRFPNITNLCPFGEVFNATRFASVYAWNRKRISNCVADY SVLYNSASFSTFKCYGVSPTKLNDLCFTNVYADSFVIRGDEVRQIAPGQT GKIADYNYKLPDDFTGCVIAWNSNNLDSKVGGNYNYLYRLFRKSNLKPFE RDISTEIYQAGSTPCNGVEGFNCYFPLQSYGFQPTNGVGYQPYRVVVLSF ELLHAPATVCGPKKSTNLVKNKCVNFNFNGLTGTGVLTESNKKFLPFQQF GRDIADTTDAVRDPQTLEILDITPCSFGGVSVITPGTNTSNQVAVLYQDV NCTEVPVAIHADQLTPTWRVYSTGSNVFQTRAGCLIGAEHVNNSYECDIP IGAGICASYQTQTNSPGSASSVASQSIIAYTMSLGAENSVAYSNNSIAIP TNFTISVTTEILPVSMTKTSVDCTMYICGDSTECSNLLLQYGSFCTQLNR ALTGIAVEQDKNTQEVFAQVKQIYKTPPIKDFGGFNFSQILPDPSKPSKR SFIEDLLFNKVTLADAGFIKQYGDCLGDIAARDLICAQKFNGLTVLPPLL TDEMIAQYTSALLAGTITSGWTFGAGAALQIPFAMQMAYRFNGIGVTQNV LYENQKLIANQFNSAIGKIQDSLSSTASALGKLQDVVNQNAQALNTLVKQ LSSNFGAISSVLNDILSRLDPPEAEVQIDRLITGRLQSLQTYVTQQLIRA AEIRASANLAATKMSECVLGQSKRVDFCGKGYHLMSFPQSAPHGVVFLHV TYVPAQEKNFTTAPAICHDGKAHFPREGVFVSNGTHWFVTQRNFYEPQII TTDNTFVSGNCDVVIGIVNNTVGSGGSGDIIKLLNEQVNKEMQSSNLYMS MSSWCYTHSLDGAGLFLFDHAAEEYEHAKKLIIFLNENNVPVQLTSISAP EHKFEGLTQIFQKAYEHEQHISESINNIVDHAIKSKDHATFNFLQWYVAE QHEEEVLFKDILDKIELIGNENHGLYLADQYVKGIAKSRKSGS | 71 |
| | pCoV1B-03 | S2P. 1208-del-Ferritin | 1,364 | VNLTTRTQLPPAYTNSFTRGVYYPDKVFRSSVLHSTQDLFLPFFSNVTWF HAIHVSGTNGTKRFDNPVLPFNDGVYFASTEKSNIIRGWIFGTTLDSKTQ SLLIVNNATNVVIKVCEFQFCNDPFLGVYYHKNNKSWMESEFRVYSSANN CTFEYVSQPFLMDLEGKQGNFKNLREFVFKNIDGYFKIYSKHTPINLVRD LPQGFSALEPLVDLPIGINITRFQTLLALHRSYLTPGDSSSGWTAGAAAY YVGYLQPRTFLLKYNENGTITDAVDCALDPLSETKCTLKSFTVEKGIYQT SNFRVQPTESIVRFPNITNLCPFGEVFNATRFASVYAWNRKRISNCVADY SVLYNSASFSTFKCYGVSPTKLNDLCFTNVYADSFVIRGDEVRQIAPGQT GKIADYNYKLPDDFTGCVIAWNSNNLDSKVGGNYNYLYRLFRKSNLKPFE RDISTEIYQAGSTPCNGVEGFNCYFPLQSYGFQPTNGVGYQPYRVVVLSF ELLHAPATVCGPKKSTNLVKNKCVNFNFNGLTGTGVLTESNKKFLPFQQF GRDIADTTDAVRDPQTLEILDITPCSFGGVSVITPGTNTSNQVAVLYQDV NCTEVPVAIHADQLTPTWRVYSTGSNVFQTRAGCLIGAEHVNNSYECDIP IGAGICASYQTQTNSPGSASSVASQSIIAYTMSLGAENSVAYSNNSIAIP TNFTISVTTEILPVSMTKTSVDCTMYICGDSTECSNLLLQYGSFCTQLNR ALTGIAVEQDKNTQEVFAQVKQIYKTPPIKDFGGFNFSQILPDPSKPSKR SFIEDLLFNKVTLADAGFIKQYGDCLGDIAARDLICAQKFNGLTVLPPLL TDEMIAQYTSALLAGTITSGWTFGAGAALQIPFAMQMAYRFNGIGVTQNV LYENQKLIANQFNSAIGKIQDSLSSTASALGKLQDVVNQNAQALNTLVKQ LSSNFGAISSVLNDILSRLDPPEAEVQIDRLITGRLQSLQTYVTQQLIRA AEIRASANLAATKMSECVLGQSKRVDFCGKGYHLMSFPQSAPHGVVFLHV TYVPAQEKNFTTAPAICHDGKAHFPREGVFVSNGTHWFVTQRNFYEPQII TTDNTFVSGNCDVVIGIVNNTVYDPLQPELDSFKEELDKYFKNHTSPDVD LGDISGINASVVNIQKEIDRLNEVAKNLNESLIDLQELGKYEQGSGGSGD IIKLLNEQVNKEMQSSNLYMSMSSWCYTHSLDGAGLFLFDHAAEEYEHAK KLIIFLNENNVPVQLTSISAPEHKFEGLTQIFQKAYEHEQHISESINNIV DHAIKSKDHATFNFLQWYVAEQHEEEVLFKDILDKIELIGNENHGLYLAD QYVKGIAKSRKSGS | 72 |
| | pCoV1B-04 | S2P.1208-GCN4-Ferritin | 1,400 | VNLTTRTQLPPAYTNSFTRGVYYPDKVFRSSVLHSTQDLFLPFFSNVTWF HAIHVSGTNGTKRFDNPVLPFNDGVYFASTEKSNIIRGWIFGTTLDSKTQ SLLIVNNATNVVIKVCEFQFCNDPFLGVYYHKNNKSWMESEFRVYSSANN CTFEYVSQPFLMDLEGKQGNFKNLREFVFKNIDGYFKIYSKHTPINLVRD LPQGFSALEPLVDLPIGINITRFQTLLALHRSYLTPGDSSSGWTAGAAAY YVGYLQPRTFLLKYNENGTITDAVDCALDPLSETKCTLKSFTVEKGIYQT SNFRVQPTESIVRFPNITNLCPFGEVFNATRFASVYAWNRKRISNCVADY SVLYNSASFSTFKCYGVSPTKLNDLCFTNVYADSFVIRGDEVRQIAPGQT GKIADYNYKLPDDFTGCVIAWNSNNLDSKVGGNYNYLYRLFRKSNLKPFE RDISTEIYQAGSTPCNGVEGFNCYFPLQSYGFQPTNGVGYQPYRVVVLSF ELLHAPATVCGPKKSTNLVKNKCVNFNFNGLTGTGVLTESNKKFLPFQQF GRDIA DTTDAVRDPQTLEILDITPCSFGGVSVITPGTNTSNQVAVLYQDVNCTEV PVAIHADQLTPTWRVYSTGSNVFQTRAGCLIGAEHVNNSYECDIPIGAGI CASYQTQTNSPGSASSVASQSIIAYTMSLGAENSVAYSNNSIAIPTNFTI SVTTEILPVSMTKTSVDCTMYICGDSTECSNLLLQYGSFCTQLNRALTGI AVEQDKNTQEVFAQVKQIYKTPPIKDFGGFNFSQILPDPSKPSKRSFIED LLFNKVTLADAGFIKQYGDCLGDIAARDLICAQKFNGLTVLPPLLTDEMI AQYTSALLAGTITSGWTFGAGAALQIPFAMQMAYRFNGIGVTQNVLYENQ KLIANQFNSAIGKIQDSLSSTASALGKLQDVVNQNAQALNTLVKQLSSNF GAISSVLNDILSRLDPPEAEVQIDRLITGRLQSLQTYVTQQLIRAAEIRA SANLAATKMSECVLGQSKRVDFCGKGYHLMSFPQSAPHGVVFLHVTYVPA QEKNFTTAPAICHDGKAHFPREGVFVSNGTHWFVTQRNFYEPQIITTDNT FVSGNCDVVIGIVNNTVYDPLQPELDSFKEELDKYFKNHTSPDVDLGDIS GINASVVNIQKEIDRLNEVAKNLNESLIDLQELGKYEQGSGGEMKQIEDK IEEILSKIYHIENEIARIKKLIGRGSGGSGDIIKLLNEQVNKEMQSSNLY MSMSSWCYTHSLDGAGLFLFDHAAEEYEHAKKLIIFLNENNVPVQLTSIS APEHKFEGLTQIFQKAYEHEQHISESINNIVDHAIKSKDHATFNFLQWYV AEQHEEEVLFKDILDKIELIGNENHGLYLADQYVKGIAKSRKSGS | 73 |
| | pCoV1B-05 | S2P.1154-del-Ferritin | 1,310 | VNLTTRTQLPPAYTNSFTRGVYYPDKVFRSSVLHSTQDLFLPFFSNVTWF HAIHVSGTNGTKRFDNPVLPFNDGVYFASTEKSNIIRGWIFGTTLDSKTQ SLLIVNNATNVVIKVCEFQFCNDPFLGVYYHKNNKSWMESEFRVYSSANN CTFEYVSQPFLMDLEGKQGNFKNLREFVFKNIDGYFKIYSKHTPINLVRD LPQGFSALEPLVDLPIGINITRFQTLLALHRSYLTPGDSSSGWTAGAAAY YVGYLQPRTFLLKYNENGTITDAVDCALDPLSETKCTLKSFTVEKGIYQT SNFRVQPTESIVRFPNITNLCPFGEVFNATRFASVYAWNRKRISNCVADY SVLYNSASFSTFKCYGVSPTKLNDLCFTNVYADSFVIRGDEVRQIAPGQT GKIADYNYKLPDDFTGCVIAWNSNNLDSKVGGNYNYLYRLFRKSNLKPFE RDISTEIYQAGSTPCNGVEGFNCYFPLQSYGFQPTNGVGYQPYRVVVLSF ELLHAPATVCGPKKSTNLVKNKCVNFNFNGLTGTGVLTESNKKFLPFQQF GRDIADTTDAVRDPQTLEILDITPCSFGGVSVITPGTNTSNQVAVLYQDV NCTEVPVAIHADQLTPTWRVYSTGSNVFQTRAGCLIGAEHVNNSYECDIP IGAGICASYQTQTNSPGSASSVASQSIIAYTMSLGAENSVAYSNNSIAIP TNFTISVTTEILPVSMTKTSVDCTMYICGDSTECSNLLLQYGSFCTQLNR ALTGIAVEQDKNTQEVFAQVKQIYKTPPIKDFGGFNFSQILPDPSKPSKR SFIEDLLFNKVTLADAGFIKQYGDCLGDIAARDLICAQKFNGLTVLPPLL TDEMIAQYTSALLAGTITSGWTFGAGAALQIPFAMQMAYRFNGIGVTQNV LYENQKLIANQFNSAIGKIQDSLSSTASALGKLQDVVNQNAQALNTLVKQ LSSNFGAISSVLNDILSRLDPPEAEVQIDRLITGRLQSLQTYVTQQLIRA AEIRASANLAATKMSECVLGQSKRVDFCGKGYHLMSFPQSAPHGVVFLHV TYVPAQEKNFTTAPAICHDGKAHFPREGVFVSNGTHWFVTQRNFYEPQII TTDNTFVSGNCDVVIGIVNNTVYDPLQPELDSFKEELDKGSGGSGDIIKL LNEQVNKEMQSSNLYMSMSSWCYTHSLDGAGLFLFDHAAEEYEHAKKLII FLNENNVPVQLTSISAPEHKFEGLTQIFQKAYEHEQHISESINNIVDHAI KSKDHATFNFLQWYVAEQHEEEVLFKDILDKIELIGNENHGLYLADQYVK GIAKSRKSGS | 74 |
| | pCoV1B-06 | S2P.1158op1-del-Ferritin | 1,314 | VNLTTRTQLPPAYTNSFTRGVYYPDKVFRSSVLHSTQDLFLPFFSNVTWF HAIHVSGTNGTKRFDNPVLPFNDGVYFASTEKSNIIRGWIFGTTLDSKTQ SLLIVNNATNVVIKVCEFQFCNDPFLGVYYHKNNKSWMESEFRVYSSANN CTFEYVSQPFLMDLEGKQGNFKNLREFVFKNIDGYFKIYSKHTPINLVRD LPQGFSALEPLVDLPIGINITRFQTLLALHRSYLTPGDSSSGWTAGAAAY YVGYLQPRTFLLKYNENGTITDAVDCALDPLSETKCTLKSFTVEKGIYQT SNFRVQPTESIVRFPNITNLCPFGEVFNATRFASVYAWNRKRISNCVADY SVLYNSASFSTFKCYGVSPTKLNDLCFTNVYADSFVIRGDEVRQIAPGQT GKIADYNYKLPDDFTGCVIAWNSNNLDSKVGGNYNYLYRLFRKSNLKPFE RDISTEIYQAGSTPCNGVEGFNCYFPLQSYGFQPTNGVGYQPYRVVVLSF ELLHAPATVCGPKKSTNLVKNKCVNFNFNGLTGTGVLTESNKKFLPFQQF GRDIADTTDAVRDPQTLEILDITPCSFGGVSVITPGTNTSNQVAVLYQDV NCTEVPVAIHADQLTPTWRVYSTGSNVFQTRAGCLIGAEHVNNSYECDIP IGAGICASYQTQTNSPGSASSVASQSIIAYTMSLGAENSVAYSNNSIAIP TNFTISVTTEILPVSMTKTSVDCTMYICGDSTECSNLLLQYGSFCTQLNR ALTGIAVEQDKNTQEVFAQVKQIYKTPPIKDFGGFNFSQILPDPSKPSKR SFIEDLLFNKVTLADAGFIKQYGDCLGDIAARDLICAQKFNGLTVLPPLL TDEMIAQYTSALLAGTITSGWTFGAGAALQIPFAMQMAYRFNGIGVTQNV LYENQKLIANQFNSAIGKIQDSLSSTASALGKLQDVVNQNAQALNTLVKQ LSSNFGAISSVLNDILSRLDPPEAEVQIDRLITGRLQSLQTYVTQQLIRA AEIRASANLAATKMSECVLGQSKRVDFCGKGYHLMSFPQSAPHGVVFLHV TYVPAQEKNFTTAPAICHDGKAHFPREGVFVSNGTHWFVTQRNFYEPQII TTDNTFVSGNCDVVIGIVNNTVYDPLQSELDSIKEELDKIHKNGSGGSGD IIKLLNEQVNKEMQSSNLYMSMSSWCYTHSLDGAGLFLFDHAAEEYEHAK KLIIFLNENNVPVQLTSISAPEHKFEGLTQIFQKAYEHEQHISESINNIV DHAIKSKDHATFNFLQWYVAEQHEEEVLFKDILDKIELIGNENHGLYLAD QYVKGIAKSRKSGS | 75 |
| | pCoV1B-07 | S2P.1158op2-del-Ferritin | 1,314 | VNLTTRTQLPPAYTNSFTRGVYYPDKVFRSSVLHSTQDLFLPFFSNVTWF HAIHVSGTNGTKRFDNPVLPFNDGVYFASTEKSNIIRGWIFGTTLDSKTQ SLLIVNNATNVVIKVCEFQFCNDPFLGVYYHKNNKSWMESEFRVYSSANN CTFEYVSQPFLMDLEGKQGNFKNLREFVFKNIDGYFKIYSKHTPINLVRD LPQGFSALEPLVDLPIGINITRFQTLLALHRSYLTPGDSSSGWTAGAAAY YVGYLQPRTFLLKYNENGTITDAVDCALDPLSETKCTLKSFTVEKGIYQT SNFRVQPTESIVRFPNITNLCPFGEVFNATRFASVYAWNRKRISNCVADY SVLYNSASFSTFKCYGVSPTKLNDLCFTNVYADSFVIRGDEVRQIAPGQT GKIADYNYKLPDDFTGCVIAWNSNNLDSKVGGNYNYLYRLFRKSNLKPFE RDISTEIYQAGSTPCNGVEGFNCYFPLQSYGFQPTNGVGYQPYRVVVLSF ELLHAPATVCGPKKSTNLVKNKCVNFNFNGLTGTGVLTESNKKFLPFQQF GRDIADTTDAVRDPQTLEILDITPCSFGGVSVITPGTNTSNQVAVLYQDV NCTEVPVAIHADQLTPTWRVYSTGSNVFQTRAGCLIGAEHVNNSYECDIP IGAGICASYQTQTNSPGSASSVASQSIIAYTMSLGAENSVAYSNNSIAIP TNFTISVTTEILPVSMTKTSVDCTMYICGDSTECSNLLLQYGSFCTQLNR ALTGIAVEQDKNTQEVFAQVKQIYKTPPIKDFGGFNFSQILPDPSKPSKR SFIEDLLFNKVTLADAGFIKQYGDCLGDIAARDLICAQKFNGLTVLPPLL TDEMIAQYTSALLAGTITSGWTFGAGAALQIPFAMQMAYRFNGIGVTQNV LYENQKLIANQFNSAIGKIQDSLSSTASALGKLQDVVNQNAQALNTLVKQ LSSNFGAISSVLNDILSRLDPPEAEVQIDRLITGRLQSLQTYVTQQLIRA AEIRASANLAATKMSECVLGQSKRVDFCGKGYHLMSFPQSAPHGVVFLHV TYVPAQEKNFTTAPAICHDGKAHFPREGVFVSNGTHWFVTQRNFYEPQII TTDNTFVSGNCDVVIGIVNNTVYDPLQSEIDSIKEEIDKIHKNGSGGSGD IIKLLNEQVNKEMQSSNLYMSMSSWCYTHSLDGAGLFLFDHAAEEYEHAK KLIIFLNENNVPVQLTSISAPEHKFEGLTQIFQKAYEHEQHISESINNIV DHAIKSKDHATFNFLQWYVAEQHEEEVLFKDILDKIELIGNENHGLYLAD QYVKGIAKSRKSGS | 76 |
| | pCoV1B-08 | S2P.1158oplx 2-del-Ferritin | 1,328 | VNLTTRTQLPPAYTNSFTRGVYYPDKVFRSSVLHSTQDLFLPFFSNVTWF HAIHVSGTNGTKRFDNPVLPFNDGVYFASTEKSNIIRGWIFGTTLDSKTQ S LLIVNNATNVVIKVCEFQFCNDPFLGVYYHKNNKSWMESEFRVYSSANN CTFEYVSQPFLMDLEGKQGNFKNLREFVFKNIDGYFKIYSKHTPINLVRD L PQGFSALEPLVDLPIGINITRFQTLLALHRSYLTPGDSSSGWTAGAAAYY V GYLQPRTFLLKYNENGTITDAVDCALDPLSETKCTLKSFTVEKGIYQTSN F RVQPTESIVRFPNITNLCPFGEVFNATRFASVYAWNRKRISNCVADYSVL Y NSASFSTFKCYGVSPTKLNDLCFTNVYADSFVIRGDEVRQIAPGQTGKIA D YNYKLPDDFTGCVIAWNSNNLDSKVGGNYNYLYRLFRKSNLKPFERDIST EIYQAGSTPCNGVEGFNCYFPLQSYGFQPTNGVGYQPYRVVVLSFELLHA PATVCGPKKSTNLVKNKCVNFNFNGLTGTGVLTESNKKFLPFQQFGRDIA DTTDAVRDPQTLEILDITPCSFGGVSVITPGTNTSNQVAVLYQDVNCTEV P VAIHADQLTPTWRVYSTGSNVFQTRAGCLIGAEHVNNSYECDIPIGAGIC A SYQTQTNSPGSASSVASQSIIAYTMSLGAENSVAYSNNSIAIPTNFTISV TTE ILPVSMTKTSVDCTMYICGDSTECSNLLLQYGSFCTQLNRALTGIAVEQD K NTQEVFAQVKQIYKTPPIKDFGGFNFSQILPDPSKPSKRSFIEDLLFNKV TL ADAGFIKQYGDCLGDIAARDLICAQKFNGLTVLPPLLTDEMIAQYTSALL AGTITSGWTFGAGAALQIPFAMQMAYRFNGIGVTQNVLYENQKLIANQF NSAIGKIQDSLSSTASALGKLQDVVNQNAQALNTLVKQLSSNFGAISSVL N DILSRLDPPEAEVQIDRLITGRLQSLQTYVTQQLIRAAEIRASANLAATK MS ECVLGQSKRVDFCGKGYHLMSFPQSAPHGVVFLHVTYVPAQEKNFTTAP AICHDGKAHFPREGVFVSNGTHWFVTQRNFYEPQIITTDNTFVSGNCDVV I GIVNNTVYDPLQSELDSIKEELDKIHKNLDSIKEELDKIHKNGSGGSGDI IK LLNEQVNKEMQSSNLYMSMSSWCYTHSLDGAGLFLFDHAAEEYEHAKK LIIFLNENNVPVQLTSISAPEHKFEGLTQIFQKAYEHEQHISESINNIVD HAIK SKDHATFNFLQWYVAEQHEEEVLFKDILDKIELIGNENHGLYLADQYVKG IAKSRKSGS | 77 |
| | pCoV1B-09 | S2P.1158op2x 2-del-Ferritin | 1,328 | VNLTTRTQLPPAYTNSFTRGVYYPDKVFRSSVLHSTQDLFLPFFSNVTWF HAIHVSGTNGTKRFDNPVLPFNDGVYFASTEKSNIIRGWIFGTTLDSKTQ S LLIVNNATNVVIKVCEFQFCNDPFLGVYYHKNNKSWMESEFRVYSSANN CTFEYVSQPFLMDLEGKQGNFKNLREFVFKNIDGYFKIYSKHTPINLVRD L PQGFSALEPLVDLPIGINITRFQTLLALHRSYLTPGDSSSGWTAGAAAYY V GYLQPRTFLLKYNENGTITDAVDCALDPLSETKCTLKSFTVEKGIYQTSN F RVQPTESIVRFPNITNLCPFGEVFNATRFASVYAWNRKRISNCVADYSVL Y NSASFSTFKCYGVSPTKLNDLCFTNVYADSFVIRGDEVRQIAPGQTGKIA D YNYKLPDDFTGCVIAWNSNNLDSKVGGNYNYLYRLFRKSNLKPFERDIST EIYQAGSTPCNGVEGFNCYFPLQSYGFQPTNGVGYQPYRVVVLSFELLHA PATVCGPKKSTNLVKNKCVNFNFNGLTGTGVLTESNKKFLPFQQFGRDIA DTTDAVRDPQTLEILDITPCSFGGVSVITPGTNTSNQVAVLYQDVNCTEV P VAIHADQLTPTWRVYSTGSNVFQTRAGCLIGAEHVNNSYECDIPIGAGIC A SYQTQTNSPGSASSVASQSIIAYTMSLGAENSVAYSNNSIAIPTNFTISV TTE ILPVSMTKTSVDCTMYICGDSTECSNLLLQYGSFCTQLNRALTGIAVEQD K NTQEVFAQVKQIYKTPPIKDFGGFNFSQILPDPSKPSKRSFIEDLLFNKV TL ADAGFIKQYGDCLGDIAARDLICAQKFNGLTVLPPLLTDEMIAQYTSALL AGTITSGWTFGAGAALQIPFAMQMAYRFNGIGVTQNVLYENQKLIANQF NSAIGKIQDSLSSTASALGKLQDVVNQNAQALNTLVKQLSSNFGAISSVL N DILSRLDPPEAEVQIDRLITGRLQSLQTYVTQQLIRAAEIRASANLAATK MS ECVLGQSKRVDFCGKGYHLMSFPQSAPHGVVFLHVTYVPAQEKNFTTAP AICHDGKAHFPREGVFVSNGTHWFVTQRNFYEPQIITTDNTFVSGNCDVV I GIVNNTVYDPLQSEIDSIKEEIDKIHKNIDSIKEEIDKIHKNGSGGSGDI IKLL NEQVNKEMQSSNLYMSMSSWCYTHSLDGAGLFLFDHAAEEYEHAKKLII FLNENNVPVQLTSISAPEHKFEGLTQIFQKAYEHEQHISESINNIVDHAI KS KDHATFNFLQWYVAEQHEEEVLFKDILDKIELIGNENHGLYLADQYVKGI AKSRKSGS | 78 |
| | pCoV1B-10 | S2P.1158op1-fGCN4-del-Ferritin | 1,345 | VNLTTRTQLPPAYTNSFTRGVYYPDKVFRSSVLHSTQDLFLPFFSNVTWF HAIHVSGTNGTKRFDNPVLPFNDGVYFASTEKSNIIRGWIFGTTLDSKTQ S LLIVNNATNVVIKVCEFQFCNDPFLGVYYHKNNKSWMESEFRVYSSANN CTFEYVSQPFLMDLEGKQGNFKNLREFVFKNIDGYFKIYSKHTPINLVRD L PQGFSALEPLVDLPIGINITRFQTLLALHRSYLTPGDSSSGWTAGAAAYY V GYLQPRTFLLKYNENGTITDAVDCALDPLSETKCTLKSFTVEKGIYQTSN F RVQPTESIVRFPNITNLCPFGEVFNATRFASVYAWNRKRISNCVADYSVL Y NSASFSTFKCYGVSPTKLNDLCFTNVYADSFVIRGDEVRQIAPGQTGKIA D YNYKLPDDFTGCVIAWNSNNLDSKVGGNYNYLYRLFRKSNLKPFERDIST EIYQAGSTPCNGVEGFNCYFPLQSYGFQPTNGVGYQPYRVVVLSFELLHA PATVCGPKKSTNLVKNKCVNFNFNGLTGTGVLTESNKKFLPFQQFGRDIA DTTDAVRDPQTLEILDITPCSFGGVSVITPGTNTSNQVAVLYQDVNCTEV P VAIHADQLTPTWRVYSTGSNVFQTRAGCLIGAEHVNNSYECDIPIGAGIC A SYQTQTNSPGSASSVASQSIIAYTMSLGAENSVAYSNNSIAIPTNFTISV TTE ILPVSMTKTSVDCTMYICGDSTECSNLLLQYGSFCTQLNRALTGIAVEQD K NTQEVFAQVKQIYKTPPIKDFGGFNFSQILPDPSKPSKRSFIEDLLFNKV TL ADAGFIKQYGDCLGDIAARDLICAQKFNGLTVLPPLLTDEMIAQYTSALL AGTITSGWTFGAGAALQIPFAMQMAYRFNGIGVTQNVLYENQKLIANQF NSAIGKIQDSLSSTASALGKLQDVVNQNAQALNTLVKQLSSNFGAISSVL N DILSRLDPPEAEVQIDRLITGRLQSLQTYVTQQLIRAAEIRASANLAATK MS ECVLGQSKRVDFCGKGYHLMSFPQSAPHGVVFLHVTYVPAQEKNFTTAP AICHDGKAHFPREGVFVSNGTHWFVTQRNFYEPQIITTDNTFVSGNCDVV I GIVNNTVYDPLQSELDSIKEELDKIHKNMKQIEDKIEEILSKIYHIENEI ARI KKLIGRGSGGSGDIIKLLNEQVNKEMQSSNLYMSMSSWCYTHSLDGAGL FLFDHAAEEYEHAKKLIIFLNENNVPVQLTSISAPEHKFEGLTQIFQKAY EH EQHISESINNIVDHAIKSKDHATFNFLQWYVAEQHEEEVLFKDILDKIEL IG NENHGLYLADQYVKGIAKSRKSGS | 79 |
| | pCoV1B-01-PL | PrefLead-S2P.1137-del-4-Ferritin | 1,295 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSFIEDLL FN KVTLADAGFIKQYGDCLGDIAARDLICAQKFNGLTVLPPLLTDEMIAQYT SALLAGTITSGWTFGAGAALQIPFAMQMAYRFNGIGVTQNVLYENQKLIA NQFNSAIGKIQDSLSSTASALGKLQDVVNQNAQALNTLVKQLSSNFGAIS S VLNDILSRLDPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVGSGGDIIKLLNEQVNKEMQSSNLYMSMSSWCYTHSLDG AGLFLFDHAAEEYEHAKKLIIFLNENNVPVQLTSISAPEHKFEGLTQIFQ KA YEHEQHISESINNIVDHAIKSKDHATFNFLQWYVAEQHEEEVLFKDILDK I ELIGNENHGLYLADQYVKGIAKSRKSGS | 80 |
| | pCoV1B-02-PL | PrefLead-S2P.1137-del-6-Ferritin | 1,297 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSFIEDLL FN KVTLADAGFIKQYGDCLGDIAARDLICAQKFNGLTVLPPLLTDEMIAQYT SALLAGTITSGWTFGAGAALQIPFAMQMAYRFNGIGVTQNVLYENQKLIA NQFNSAIGKIQDSLSSTASALGKLQDVVNQNAQALNTLVKQLSSNFGAIS S VLNDILSRLDPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVGSGGSGDIIKLLNEQVNKEMQSSNLYMSMSSWCYTHSLD GAGLFLFDHAAEEYEHAKKLIIFLNENNVPVQLTSISAPEHKFEGLTQIF QK AYEHEQHISESINNIVDHAIKSKDHATFNFLQWYVAEQHEEEVLFKDILD K IELIGNENHGLYLADQYVKGIAKSRKSGS | 81 |
| | pCoV1B-03-PL | PrefLead-S2P.1208-del-Ferritin | 1,368 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSFIEDLL FN KVTLADAGFIKQYGDCLGDIAARDLICAQKFNGLTVLPPLLTDEMIAQYT SALLAGTITSGWTFGAGAALQIPFAMQMAYRFNGIGVTQNVLYENQKLIA NQFNSAIGKIQDSLSSTASALGKLQDVVNQNAQALNTLVKQLSSNFGAIS S VLNDILSRLDPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQPELDSFKEELDKYFKNHTSPDVDLGDISGINASV V NIQKEIDRLNEVAKNLNESLIDLQELGKYEQGSGGSGDIIKLLNEQVNKE M QSSNLYMSMSSWCYTHSLDGAGLFLFDHAAEEYEHAKKLIIFLNENNVPV QLTSISAPEHKFEGLTQIFQKAYEHEQHISESINNIVDHAIKSKDHATFN FL QWYVAEQHEEEVLFKDILDKIELIGNENHGLYLADQYVKGIAKSRKSGS | 82 |
| | pCoV1B-04-PL | PrefLead-S2P.1208-GCN4-Ferritin | 1,404 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSFIEDLL FN KVTLADAGFIKQYGDCLGDIAARDLICAQKFNGLTVLPPLLTDEMIAQYT SALLAGTITSGWTFGAGAALQIPFAMQMAYRFNGIGVTQNVLYENQKLIA NQFNSAIGKIQDSLSSTASALGKLQDVVNQNAQALNTLVKQLSSNFGAIS S VLNDILSRLDPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQPELDSFKEELDKYFKNHTSPDVDLGDISGINASV V NIQKEIDRLNEVAKNLNESLIDLQELGKYEQGSGGEMKQIEDKIEEILSK IY HIENEIARIKKLIGRGSGGSGDIIKLLNEQVNKEMQSSNLYMSMSSWCYT H SLDGAGLFLFDHAAEEYEHAKKLIIFLNENNVPVQLTSISAPEHKFEGLT QI FQKAYEHEQHISESINNIVDHAIKSKDHATFNFLQWYVAEQHEEEVLFKD I LDKIELIGNENHGLYLADQYVKGIAKSRKSGS | 83 |
| | pCoV1B-05-PL | PrefLead-S2P.1154-del-Ferritin | 1,314 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSFIEDLL FN KVTLADAGFIKQYGDCLGDIAARDLICAQKFNGLTVLPPLLTDEMIAQYT SALLAGTITSGWTFGAGAALQIPFAMQMAYRFNGIGVTQNVLYENQKLIA NQFNSAIGKIQDSLSSTASALGKLQDVVNQNAQALNTLVKQLSSNFGAIS S VLNDILSRLDPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQPELDSFKEELDKGSGGSGDIIKLLNEQVNKEMQS S NLYMSMSSWCYTHSLDGAGLFLFDHAAEEYEHAKKLIIFLNENNVPVQL TSISAPEHKFEGLTQIFQKAYEHEQHISESINNIVDHAIKSKDHATFNFL QW YVAEQHEEEVLFKDILDKIELIGNENHGLYLADQYVKGIAKSRKSGS | 84 |
| | pCoV1B-06-PL | PrefLead-S2P.1158op1-del-Ferritin | 1,318 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSFIEDLL FN KVTLADAGFIKQYGDCLGDIAARDLICAQKFNGLTVLPPLLTDEMIAQYT SALLAGTITSGWTFGAGAALQIPFAMQMAYRFNGIGVTQNVLYENQKLIA NQFNSAIGKIQDSLSSTASALGKLQDVVNQNAQALNTLVKQLSSNFGAIS S VLNDILSRLDPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQSELDSIKEELDKIHKNGSGGSGDIIKLLNEQVNK E MQSSNLYMSMSSWCYTHSLDGAGLFLFDHAAEEYEHAKKLIIFLNENNV PVQLTSISAPEHKFEGLTQIFQKAYEHEQHISESINNIVDHAIKSKDHAT FNF LQWYVAEQHEEEVLFKDILDKIELIGNENHGLYLADQYVKGIAKSRKSGS | 85 |
| | pCoV1B-07-PL | PrefLead-S2P.1158op2-del-Ferritin | 1,318 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSFIEDLL FN KVTLADAGFIKQYGDCLGDIAARDLICAQKFNGLTVLPPLLTDEMIAQYT SALLAGTITSGWTFGAGAALQIPFAMQMAYRFNGIGVTQNVLYENQKLIA NQFNSAIGKIQDSLSSTASALGKLQDVVNQNAQALNTLVKQLSSNFGAIS S VLNDILSRLDPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQSEIDSIKEEIDKIHKNGSGGSGDIIKLLNEQVNK EM QSSNLYMSMSSWCYTHSLDGAGLFLFDHAAEEYEHAKKLIIFLNENNVPV QLTSISAPEHKFEGLTQIFQKAYEHEQHISESINNIVDHAIKSKDHATFN FL QWYVAEQHEEEVLFKDILDKIELIGNENHGLYLADQYVKGIAKSRKSGS | 86 |
| | pCoV1B-08-PL | PrefLead-S2P.1158op1x 2-del-Ferritin | 1,332 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSFIEDLL FN KVTLADAGFIKQYGDCLGDIAARDLICAQKFNGLTVLPPLLTDEMIAQYT SALLAGTITSGWTFGAGAALQIPFAMQMAYRFNGIGVTQNVLYENQKLIA NQFNSAIGKIQDSLSSTASALGKLQDVVNQNAQALNTLVKQLSSNFGAIS S VLNDILSRLDPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQSELDSIKEELDKIHKNLDSIKEELDKIHKNGSGG SG DIIKLLNEQVNKEMQSSNLYMSMSSWCYTHSLDGAGLFLFDHAAEEYEH AKKLIIFLNENNVPVQLTSISAPEHKFEGLTQIFQKAYEHEQHISESINN IVD HAIKSKDHATFNFLQWYVAEQHEEEVLFKDILDKIELIGNENHGLYLADQ YVKGIAKSRKSGS | 87 |
| | pCoV1B-09-PL | PrefLead-S2P.1158op2x 2-del-Ferritin | 1,332 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSFIEDLL FN KVTLADAGFIKQYGDCLGDIAARDLICAQKFNGLTVLPPLLTDEMIAQYT SALLAGTITSGWTFGAGAALQIPFAMQMAYRFNGIGVTQNVLYENQKLIA NQFNSAIGKIQDSLSSTASALGKLQDVVNQNAQALNTLVKQLSSNFGAIS S VLNDILSRLDPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQSEIDSIKEEIDKIHKNIDSIKEEIDKIHKNGSGG SGDI IKLLNEQVNKEMQSSNLYMSMSSWCYTHSLDGAGLFLFDHAAEEYEHAK KLIIFLNENNVPVQLTSISAPEHKFEGLTQIFQKAYEHEQHISESINNIV DHAI KSKDHATFNFLQWYVAEQHEEEVLFKDILDKIELIGNENHGLYLADQYVK GIAKSRKSGS | 88 |
| | pCoV1B-10-PL | PrefLead-S2P.1158op1-fGCN4-del-Ferritin | 1,349 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSFIEDLL FN KVTLADAGFIKQYGDCLGDIAARDLICAQKFNGLTVLPPLLTDEMIAQYT SALLAGTITSGWTFGAGAALQIPFAMQMAYRFNGIGVTQNVLYENQKLIA NQFNSAIGKIQDSLSSTASALGKLQDVVNQNAQALNTLVKQLSSNFGAIS S VLNDILSRLDPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQSELDSIKEELDKIHKNMKQIEDKIEEILSKIYHI ENE IARIKKLIGRGSGGSGDIIKLLNEQVNKEMQSSNLYMSMSSWCYTHSLDG AGLFLFDHAAEEYEHAKKLIIFLNENNVPVQLTSISAPEHKFEGLTQIFQ KA YEHEQHISESINNIVDHAIKSKDHATFNFLQWYVAEQHEEEVLFKDILDK I ELIGNENHGLYLADQYVKGIAKSRKSGS | 89 |
| | pCoV89 | His8-3c NTD-SSQC-Ferrritin-301CTLKSFT 307 | 495 | HHHHHHHHGPLEVLFQGPSSQCVNLTTRTQLPPAYTNSFTRGVYYPDKVF RSSVLHSTQDLFLPFFSNVTWFHAIHVSGTNGTKRFDNPVLPFNDGVYFA S TEKSNIIRGWIFGTTLDSKTQSLLIVNNATNVVIKVCEFQFCNDPFLGVY Y HKNNKSWMESEFRVYSSANNCTFEYVSQPFLMDLEGKQGNFKNLREFVF KNIDGYFKIYSKHTPINLVRDLPQGFSALEPLVDLPIGINITRFQTLLAL HRS YLTPGDSSSGWTAGAAAYYVGYLQPRTFLLKYNENGTITDAVDCALDPL SETKCTLKSFTGSGGGGESQVRQQFSKDIEKLLNEQVNKEMQSSNLYMS MSSWCYTHSLDGAGLFLFDHAAEEYEHAKKLIIFLNENNVPVQLTSISAP E HKFEGLTQIFQKAYEHEQHISESINNIVDHAIKSKDHATFNFLQWYVAEQ H EEEVLFKDILDKIELIGNENHGLYLADQYVKGIAKSRKSGS | 90 |
| | pCoV90 | His8-3c NTD-SSQC-Ferrritin-L296K-L303D | 491 | HHHHHHHHGPLEVLFQGPSSQCVNLTTRTQLPPAYTNSFTRGVYYPDKVF RSSVLHSTQDLFLPFFSNVTWFHAIHVSGTNGTKRFDNPVLPFNDGVYFA S TEKSNIIRGWIFGTTLDSKTQSLLIVNNATNVVIKVCEFQFCNDPFLGVY Y HKNNKSWMESEFRVYSSANNCTFEYVSQPFLMDLEGKQGNFKNLREFVF KNIDGYFKIYSKHTPINLVRDLPQGFSALEPLVDLPIGINITRFQTLLAL HRS YLTPGDSSSGWTAGAAAYYVGYLQPRTFLLKYNENGTITDAVDCALDPK SETKCTDGSGGGGESQVRQQFSKDIEKLLNEQVNKEMQSSNLYMSMSSW CYTHSLDGAGLFLFDHAAEEYEHAKKLIIFLNENNVPVQLTSISAPEHKF E GLTQIFQKAYEHEQHISESINNIVDHAIKSKDHATFNFLQWYVAEQHEEE V LFKDILDKIELIGNENHGLYLADQYVKGIAKSRKSGS | 91 |
| | pCoV91 | His8-3c NTD-SSQC-Ferrritin-301CTLKSFT 307-L296K-L303D | 495 | HHHHHHHHGPLEVLFQGPSSQCVNLTTRTQLPPAYTNSFTRGVYYPDKVF RSSVLHSTQDLFLPFFSNVTWFHAIHVSGTNGTKRFDNPVLPFNDGVYFA S TEKSNIIRGWIFGTTLDSKTQSLLIVNNATNVVIKVCEFQFCNDPFLGVY Y HKNNKSWMESEFRVYSSANNCTFEYVSQPFLMDLEGKQGNFKNLREFVF KNIDGYFKIYSKHTPINLVRDLPQGFSALEPLVDLPIGINITRFQTLLAL HRS YLTPGDSSSGWTAGAAAYYVGYLQPRTFLLKYNENGTITDAVDCALDPK SETKCTDKSFTGSGGGGESQVRQQFSKDIEKLLNEQVNKEMQSSNLYMS MSSWCYTHSLDGAGLFLFDHAAEEYEHAKKLIIFLNENNVPVQLTSISAP E HKFEGLTQIFQKAYEHEQHISESINNIVDHAIKSKDHATFNFLQWYVAEQ H EEEVLFKDILDKIELIGNENHGLYLADQYVKGIAKSRKSGS | 92 |
| | pCoV92 | His8-3c NTD-SSQC-Ferrritin-291 CALDP to CGNDT | 491 | HHHHHHHHGPLEVLFQGPSSQCVNLTTRTQLPPAYTNSFTRGVYYPDKVF RSSVLHSTQDLFLPFFSNVTWFHAIHVSGTNGTKRFDNPVLPFNDGVYFA S TEKSNIIRGWIFGTTLDSKTQSLLIVNNATNVVIKVCEFQFCNDPFLGVY Y HKNNKSWMESEFRVYSSANNCTFEYVSQPFLMDLEGKQGNFKNLREFVF KNIDGYFKIYSKHTPINLVRDLPQGFSALEPLVDLPIGINITRFQTLLAL HRS YLTPGDSSSGWTAGAAAYYVGYLQPRTFLLKYNENGTITDAVDCGNDTL SETKCTLGSGGGGESQVRQQFSKDIEKLLNEQVNKEMQSSNLYMSMSSW CYTHSLDGAGLFLFDHAAEEYEHAKKLIIFLNENNVPVQLTSISAPEHKF E GLTQIFQKAYEHEQHISESINNIVDHAIKSKDHATFNFLQWYVAEQHEEE V LFKDILDKIELIGNENHGLYLADQYVKGIAKSRKSGS | 93 |
| | pCoV93 | His8-3c NTD-SSQC-Ferrritin-F59T | 491 | HHHHHHHHGPLEVLFQGPSSQCVNLTTRTQLPPAYTNSFTRGVYYPDKVF RSSVLHSTQDLFLPFTSNVTWFHAIHVSGTNGTKRFDNPVLPFNDGVYFA STEKSNIIRGWIFGTTLDSKTQSLLIVNNATNVVIKVCEFQFCNDPFLGV YY HKNNKSWMESEFRVYSSANNCTFEYVSQPFLMDLEGKQGNFKNLREFVF KNIDGYFKIYSKHTPINLVRDLPQGFSALEPLVDLPIGINITRFQTLLAL HRS YLTPGDSSSGWTAGAAAYYVGYLQPRTFLLKYNENGTITDAVDCALDPL SETKCTLGSGGGGESQVRQQFSKDIEKLLNEQVNKEMQSSNLYMSMSSW CYTHSLDGAGLFLFDHAAEEYEHAKKLIIFLNENNVPVQLTSISAPEHKF E GLTQIFQKAYEHEQHISESINNIVDHAIKSKDHATFNFLQWYVAEQHEEE V LFKDILDKIELIGNENHGLYLADQYVKGIAKSRKSGS | 94 |
| | pCoV94 | His8-3c NTD-SSQC-Ferrritin-Y28T | 491 | HHHHHHHHGPLEVLFQGPSSQCVNLTTRTQLPPATTNSFTRGVYYPDKVF RSSVLHSTQDLFLPFFSNVTWFHAIHVSGTNGTKRFDNPVLPFNDGVYFA S TEKSNIIRGWIFGTTLDSKTQSLLIVNNATNVVIKVCEFQFCNDPFLGVY Y HKNNKSWMESEFRVYSSANNCTFEYVSQPFLMDLEGKQGNFKNLREFVF KNIDGYFKIYSKHTPINLVRDLPQGFSALEPLVDLPIGINITRFQTLLAL HRS YLTPGDSSSGWTAGAAAYYVGYLQPRTFLLKYNENGTITDAVDCALDPL SETKCTLGSGGGGESQVRQQFSKDIEKLLNEQVNKEMQSSNLYMSMSSW CYTHSLDGAGLFLFDHAAEEYEHAKKLIIFLNENNVPVQLTSISAPEHKF E GLTQIFQKAYEHEQHISESINNIVDHAIKSKDHATFNFLQWYVAEQHEEE V LFKDILDKIELIGNENHGLYLADQYVKGIAKSRKSGS | 95 |
| | pCoV95 | His8-3c NTD-SSQC-Ferrritin-Y200A | 491 | HHHHHHHHGPLEVLFQGPSSQCVNLTTRTQLPPAYTNSFTRGVYYPDKVF RSSVLHSTQDLFLPFFSNVTWFHAIHVSGTNGTKRFDNPVLPFNDGVYFA S TEKSNIIRGWIFGTTLDSKTQSLLIVNNATNVVIKVCEFQFCNDPFLGVY Y HKNNKSWMESEFRVYSSANNCTFEYVSQPFLMDLEGKQGNFKNLREFVF KNIDGAFKIYSKHTPINLVRDLPQGFSALEPLVDLPIGINITRFQTLLAL HRS YLTPGDSSSGWTAGAAAYYVGYLQPRTFLLKYNENGTITDAVDCALDPL SETKCTLGSGGGGESQVRQQFSKDIEKLLNEQVNKEMQSSNLYMSMSSW CYTHSLDGAGLFLFDHAAEEYEHAKKLIIFLNENNVPVQLTSISAPEHKF E GLTQIFQKAYEHEQHISESINNIVDHAIKSKDHATFNFLQWYVAEQHEEE V LFKDILDKIELIGNENHGLYLADQYVKGIAKSRKSGS | 96 |
| | pCoV96 | His8-3c NTD-SSQC-Ferrritin-Y200N-K202T | 491 | HHHHHHHHGPLEVLFQGPSSQCVNLTTRTQLPPAYTNSFTRGVYYPDKVF RSSVLHSTQDLFLPFFSNVTWFHAIHVSGTNGTKRFDNPVLPFNDGVYFA S TEKSNIIRGWIFGTTLDSKTQSLLIVNNATNVVIKVCEFQFCNDPFLGVY Y HKNNKSWMESEFRVYSSANNCTFEYVSQPFLMDLEGKQGNFKNLREFVF KNIDGNFTIYSKHTPINLVRDLPQGFSALEPLVDLPIGINITRFQTLLAL HRS YLTPGDSSSGWTAGAAAYYVGYLQPRTFLLKYNENGTITDAVDCALDPL SETKCTLGSGGGGESQVRQQFSKDIEKLLNEQVNKEMQSSNLYMSMSSW CYTHSLDGAGLFLFDHAAEEYEHAKKLIIFLNENNVPVQLTSISAPEHKF E GLTQIFQKAYEHEQHISESINNIVDHAIKSKDHATFNFLQWYVAEQHEEE V LFKDILDKIELIGNENHGLYLADQYVKGIAKSRKSGS | 97 |
| | pCoV97 | His8-3c NTD-SSQC-Ferrritin-Y38T | 491 | HHHHHHHHGPLEVLFQGPSSQCVNLTTRTQLPPAYTNSFTRGVYTPDKVF RSSVLHSTQDLFLPFFSNVTWFHAIHVSGTNGTKRFDNPVLPFNDGVYFA S TEKSNIIRGWIFGTTLDSKTQSLLIVNNATNVVIKVCEFQFCNDPFLGVY Y HKNNKSWMESEFRVYSSANNCTFEYVSQPFLMDLEGKQGNFKNLREFVF KNIDGYFKIYSKHTPINLVRDLPQGFSALEPLVDLPIGINITRFQTLLAL HRS YLTPGDSSSGWTAGAAAYYVGYLQPRTFLLKYNENGTITDAVDCALDPL SETKCTLGSGGGGESQVRQQFSKDIEKLLNEQVNKEMQSSNLYMSMSSW CYTHSLDGAGLFLFDHAAEEYEHAKKLIIFLNENNVPVQLTSISAPEHKF E GLTQIFQKAYEHEQHISESINNIVDHAIKSKDHATFNFLQWYVAEQHEEE V LFKDILDKIELIGNENHGLYLADQYVKGIAKSRKSGS | 98 |
| | pCoV98 | His8-3c NTD-SSQC-Ferrritin-V42T-F43A-V47T | 491 | HHHHHHHHGPLEVLFQGPSSQCVNLTTRTQLPPAYTNSFTRGVYYPDKT ARSSTLHSTQDLFLPFFSNVTWFHAIHVSGTNGTKRFDNPVLPFNDGVYF ASTEKSNIIRGWIFGTTLDSKTQSLLIVNNATNVVIKVCEFQFCNDPFLG VY YHKNNKSWMESEFRVYSSANNCTFEYVSQPFLMDLEGKQGNFKNLREFV FKNIDGYFKIYSKHTPINLVRDLPQGFSALEPLVDLPIGINITRFQTLLA LHR SYLTPGDSSSGWTAGAAAYYVGYLQPRTFLLKYNENGTITDAVDCALDP LSETKCTLGSGGGGESQVRQQFSKDIEKLLNEQVNKEMQSSNLYMSMSS WCYTHSLDGAGLFLFDHAAEEYEHAKKLIIFLNENNVPVQLTSISAPEHK F EGLTQIFQKAYEHEQHISESINNIVDHAIKSKDHATFNFLQWYVAEQHEE E VLFKDILDKIELIGNENHGLYLADQYVKGIAKSRKSGS | 99 |
| | pCoV99 | His8-3c NTD-SSQC-Ferrritin-118LIV to NIT | 491 | HHHHHHHHGPLEVLFQGPSSQCVNLTTRTQLPPAYTNSFTRGVYYPDKVF RSSVLHSTQDLFLPFFSNVTWFHAIHVSGTNGTKRFDNPVLPFNDGVYFA S TEKSNIIRGWIFGTTLDSKTQSLNITNNATNVVIKVCEFQFCNDPFLGVY Y HKNNKSWMESEFRVYSSANNCTFEYVSQPFLMDLEGKQGNFKNLREFVF KNIDGYFKIYSKHTPINLVRDLPQGFSALEPLVDLPIGINITRFQTLLAL HRS YLTPGDSSSGWTAGAAAYYVGYLQPRTFLLKYNENGTITDAVDCALDPL SETKCTLGSGGGGESQVRQQFSKDIEKLLNEQVNKEMQSSNLYMSMSSW CYTHSLDGAGLFLFDHAAEEYEHAKKLIIFLNENNVPVQLTSISAPEHKF E GLTQIFQKAYEHEQHISESINNIVDHAIKSKDHATFNFLQWYVAEQHEEE V LFKDILDKIELIGNENHGLYLADQYVKGIAKSRKSGS | 100 |
| | pCoV100 | His8-3c NTD-SSQC-Ferrritin-133FQF to NQT-I105F | 491 | HHHHHHHHGPLEVLFQGPSSQCVNLTTRTQLPPAYTNSFTRGVYYPDKVF RSSVLHSTQDLFLPFFSNVTWFHAIHVSGTNGTKRFDNPVLPFNDGVYFA S TEKSNIIRGWFFGTTLDSKTQSLLIVNNATNVVIKVCENQTCNDPFLGVY Y HKNNKSWMESEFRVYSSANNCTFEYVSQPFLMDLEGKQGNFKNLREFVF KNIDGYFKIYSKHTPINLVRDLPQGFSALEPLVDLPIGINITRFQTLLAL HRS YLTPGDSSSGWTAGAAAYYVGYLQPRTFLLKYNENGTITDAVDCALDPL SETKCTLGSGGGGESQVRQQFSKDIEKLLNEQVNKEMQSSNLYMSMSSW CYTHSLDGAGLFLFDHAAEEYEHAKKLIIFLNENNVPVQLTSISAPEHKF E GLTQIFQKAYEHEQHISESINNIVDHAIKSKDHATFNFLQWYVAEQHEEE V LFKDILDKIELIGNENHGLYLADQYVKGIAKSRKSGS | 101 |
| | pCoV101 | His8-3c NTD-SSQC-Ferrritin-126VVI to NVT | 491 | HHHHHHHHGPLEVLFQGPSSQCVNLTTRTQLPPAYTNSFTRGVYYPDKVF RSSVLHSTQDLFLPFFSNVTWFHAIHVSGTNGTKRFDNPVLPFNDGVYFA S TEKSNIIRGWIFGTTLDSKTQSLLIVNNATNNVTKVCEFQFCNDPFLGVY Y HKNNKSWMESEFRVYSSANNCTFEYVSQPFLMDLEGKQGNFKNLREFVF KNIDGYFKIYSKHTPINLVRDLPQGFSALEPLVDLPIGINITRFQTLLAL HRS YLTPGDSSSGWTAGAAAYYVGYLQPRTFLLKYNENGTITDAVDCALDPL SETKCTLGSGGGGESQVRQQFSKDIEKLLNEQVNKEMQSSNLYMSMSSW CYTHSLDGAGLFLFDHAAEEYEHAKKLIIFLNENNVPVQLTSISAPEHKF E GLTQIFQKAYEHEQHISESINNIVDHAIKSKDHATFNFLQWYVAEQHEEE V LFKDILDKIELIGNENHGLYLADQYVKGIAKSRKSGS | 102 |
| | pCoV102 | His8-3c NTD-SSQC-Ferrritin-226LVD to NVT | 491 | HHHHHHHHGPLEVLFQGPSSQCVNLTTRTQLPPAYTNSFTRGVYYPDKVF RSSVLHSTQDLFLPFFSNVTWFHAIHVSGTNGTKRFDNPVLPFNDGVYFA S TEKSNIIRGWIFGTTLDSKTQSLLIVNNATNVVIKVCEFQFCNDPFLGVY Y HKNNKSWMESEFRVYSSANNCTFEYVSQPFLMDLEGKQGNFKNLREFVF KNIDGYFKIYSKHTPINLVRDLPQGFSALEPNVTLPIGINITRFQTLLAL HRS YLTPGDSSSGWTAGAAAYYVGYLQPRTFLLKYNENGTITDAVDCALDPL SETKCTLGSGGGGESQVRQQFSKDIEKLLNEQVNKEMQSSNLYMSMSSW CYTHSLDGAGLFLFDHAAEEYEHAKKLIIFLNENNVPVQLTSISAPEHKF E GLTQIFQKAYEHEQHISESINNIVDHAIKSKDHATFNFLQWYVAEQHEEE V LFKDILDKIELIGNENHGLYLADQYVKGIAKSRKSGS | 103 |
| | pCoV103 | His8-3c NTD-SSQC-Ferrritin-227VDL to NDT | 491 | HHHHHHHHGPLEVLFQGPSSQCVNLTTRTQLPPAYTNSFTRGVYYPDKVF RSSVLHSTQDLFLPFFSNVTWFHAIHVSGTNGTKRFDNPVLPFNDGVYFA S TEKSNIIRGWIFGTTLDSKTQSLLIVNNATNVVIKVCEFQFCNDPFLGVY Y HKNNKSWMESEFRVYSSANNCTFEYVSQPFLMDLEGKQGNFKNLREFVF KNIDGYFKIYSKHTPINLVRDLPQGFSALEPLNDTPIGINITRFQTLLAL HRS YLTPGDSSSGWTAGAAAYYVGYLQPRTFLLKYNENGTITDAVDCALDPL SETKCTLGSGGGGESQVRQQFSKDIEKLLNEQVNKEMQSSNLYMSMSSW CYTHSLDGAGLFLFDHAAEEYEHAKKLIIFLNENNVPVQLTSISAPEHKF E GLTQIFQKAYEHEQHISESINNIVDHAIKSKDHATFNFLQWYVAEQHEEE V LFKDILDKIELIGNENHGLYLADQYVKGIAKSRKSGS | 104 |
| | pCoV107 | S1-SSQC-endH655-Ferritin | 825 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHGSGGGGESQVRQ QFSKDIEKLLNEQVNKEMQSSNLYMSMSSWCYTHSLDGAGLFLFDHAAE EYEHAKKLIIFLNENNVPVQLTSISAPEHKFEGLTQIFQKAYEHEQHISE SIN NIVDHAIKSKDHATFNFLQWYVAEQHEEEVLFKDILDKIELIGNENHGLY L ADQYVKGIAKSRKSGS | 105 |
| | pCoV108 | S1-SSQC-endH655-611LYQ to NYT-Ferritin | 825 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVNYTDVNC T EVPVAIHADQLTPTWRVYSTGSNVFQTRAGCLIGAEHGSGGGGESQVRQ QFSKDIEKLLNEQVNKEMQSSNLYMSMSSWCYTHSLDGAGLFLFDHAAE EYEHAKKLIIFLNENNVPVQLTSISAPEHKFEGLTQIFQKAYEHEQHISE SIN NIVDHAIKSKDHATFNFLQWYVAEQHEEEVLFKDILDKIELIGNENHGLY L ADQYVKGIAKSRKSGS | 106 |
| | pCoV109 | S1-SSQC-endT696-Ferritin | 866 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTNSPGSASSVASQSIIAYTGSGGGGESQVRQQFSKDIEKLL NE QVNKEMQSSNLYMSMSSWCYTHSLDGAGLFLFDHAAEEYEHAKKLIIFL NENNVPVQLTSISAPEHKFEGLTQIFQKAYEHEQHISESINNIVDHAIKS KD HATFNFLQWYVAEQHEEEVLFKDILDKIELIGNENHGLYLADQYVKGIAK SRKSGS | 107 |
| | pCoV110 | S1-SSQC-T676-G-SQSIIAYT696-Ferritin | 855 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTGSQSIIAYTGSGGGGESQVRQQFSKDIEKLLNEQVNKEMQSS N LYMSMSSWCYTHSLDGAGLFLFDHAAEEYEHAKKLIIFLNENNVPVQLTS ISAPEHKFEGLTQIFQKAYEHEQHISESINNIVDHAIKSKDHATFNFLQW YV AEQHEEEVLFKDILDKIELIGNENHGLYLADQYVKGIAKSRKSGS | 108 |
| | pCoV111 | S1-SSQC-T676-GG-SQSIIAYT696-Ferritin | 856 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTGGSQSIIAYTGSGGGGESQVRQQFSKDIEKLLNEQVNKEMQS S NLYMSMSSWCYTHSLDGAGLFLFDHAAEEYEHAKKLIIFLNENNVPVQL TSISAPEHKFEGLTQIFQKAYEHEQHISESINNIVDHAIKSKDHATFNFL QW YVAEQHEEEVLFKDILDKIELIGNENHGLYLADQYVKGIAKSRKSGS | 109 |
| | pCoV112 | S1-SSQC-T676-PG-SQSIIAYT696-Ferritin | 856 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTPGSQSIIAYTGSGGGGESQVRQQFSKDIEKLLNEQVNKEMQS S NLYMSMSSWCYTHSLDGAGLFLFDHAAEEYEHAKKLIIFLNENNVPVQL TSISAPEHKFEGLTQIFQKAYEHEQHISESINNIVDHAIKSKDHATFNFL QW YVAEQHEEEVLFKDILDKIELIGNENHGLYLADQYVKGIAKSRKSGS | 110 |
| | pCoV113 | S1-SSQC-312YQT to NYT-Ferritin | 848 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGNYTT SNFRVQPTESIVRFPNITNLCPFGEVFNATRFASVYAWNRKRISNCVADY S VLYNSASFSTFKCYGVSPTKLNDLCFTNVYADSFVIRGDEVRQIAPGQTG KIADYNYKLPDDFTGCVIAWNSNNLDSKVGGNYNYLYRLFRKSNLKPFE RDISTEIYQAGSTPCNGVEGFNCYFPLQSYGFQPTNGVGYQPYRVVVLSF E LLHAPATVCGPKKSTNLVKNKCVNFNFNGLTGTGVLTESNKKFLPFQQFG RDIADTTDAVRDPQTLEILDITPCSFGGVSVITPGTNTSNQVAVLYQDVN C TEVPVAIHADQLTPTWRVYSTGSNVFQTRAGCLIGAEHVNNSYECDIPIG A GICASYQTQTGSGGGGESQVRQQFSKDIEKLLNEQVNKEMQSSNLYMSM SSWCYTHSLDGAGLFLFDHAAEEYEHAKKLIIFLNENNVPVQLTSISAPE H KFEGLTQIFQKAYEHEQHISESINNIVDHAIKSKDHATFNFLQWYVAEQH E EEVLFKDILDKIELIGNENHGLYLADQYVKGIAKSRKSGS | 111 |
| | pCoV114 | S1-SSQC-651IGA to NGS-Ferritin | 848 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLNGSEHVNNSYECDIPIGA GICASYQTQTGSGGGGESQVRQQFSKDIEKLLNEQVNKEMQSSNLYMSM SSWCYTHSLDGAGLFLFDHAAEEYEHAKKLIIFLNENNVPVQLTSISAPE H KFEGLTQIFQKAYEHEQHISESINNIVDHAIKSKDHATFNFLQWYVAEQH E EEVLFKDILDKIELIGNENHGLYLADQYVKGIAKSRKSGS | 112 |
| | pCoV115 | S1-SSQC-S316C-V595C-Ferritin | 848 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT C NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGCSVITPGTNTSNQVAVLYQDVNC T EVPVAIHADQLTPTWRVYSTGSNVFQTRAGCLIGAEHVNNSYECDIPIGA GICASYQTQTGSGGGGESQVRQQFSKDIEKLLNEQVNKEMQSSNLYMSM SSWCYTHSLDGAGLFLFDHAAEEYEHAKKLIIFLNENNVPVQLTSISAPE H KFEGLTQIFQKAYEHEQHISESINNIVDHAIKSKDHATFNFLQWYVAEQH E EEVLFKDILDKIELIGNENHGLYLADQYVKGIAKSRKSGS | 113 |
| | pCoV116 | S1-SSQC-V320C-S591C-Ferritin | 848 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRCQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCCFGGVSVITPGTNTSNQVAVLYQDVNC T EVPVAIHADQLTPTWRVYSTGSNVFQTRAGCLIGAEHVNNSYECDIPIGA GICASYQTQTGSGGGGESQVRQQFSKDIEKLLNEQVNKEMQSSNLYMSM SSWCYTHSLDGAGLFLFDHAAEEYEHAKKLIIFLNENNVPVQLTSISAPE H KFEGLTQIFQKAYEHEQHISESINNIVDHAIKSKDHATFNFLQWYVAEQH E EEVLFKDILDKIELIGNENHGLYLADQYVKGIAKSRKSGS | 114 |
| | pCoV117 | S1-SSQC-L560Q-F562H-Ferritin | 848 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFQPHQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTGSGGGGESQVRQQFSKDIEKLLNEQVNKEMQSSNLYMSM SSWCYTHSLDGAGLFLFDHAAEEYEHAKKLIIFLNENNVPVQLTSISAPE H KFEGLTQIFQKAYEHEQHISESINNIVDHAIKSKDHATFNFLQWYVAEQH E EEVLFKDILDKIELIGNENHGLYLADQYVKGIAKSRKSGS | 115 |
| | pCoV118 | S1-SSQC-562FQQ to NQT-Ferritin | 848 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPNQTFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTGSGGGGESQVRQQFSKDIEKLLNEQVNKEMQSSNLYMSM SSWCYTHSLDGAGLFLFDHAAEEYEHAKKLIIFLNENNVPVQLTSISAPE H KFEGLTQIFQKAYEHEQHISESINNIVDHAIKSKDHATFNFLQWYVAEQH E EEVLFKDILDKIELIGNENHGLYLADQYVKGIAKSRKSGS | 116 |
| | pCoV119 | S1-SSQC-F490R-Ferritin | 848 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYRPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTGSGGGGESQVRQQFSKDIEKLLNEQVNKEMQSSNLYMSM SSWCYTHSLDGAGLFLFDHAAEEYEHAKKLIIFLNENNVPVQLTSISAPE H KFEGLTQIFQKAYEHEQHISESINNIVDHAIKSKDHATFNFLQWYVAEQH E EEVLFKDILDKIELIGNENHGLYLADQYVKGIAKSRKSGS | 117 |
| | pCoV120 | S1-SSQC-F490A-Ferritin | 848 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYAPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTGSGGGGESQVRQQFSKDIEKLLNEQVNKEMQSSNLYMSM SSWCYTHSLDGAGLFLFDHAAEEYEHAKKLIIFLNENNVPVQLTSISAPE H KFEGLTQIFQKAYEHEQHISESINNIVDHAIKSKDHATFNFLQWYVAEQH E EEVLFKDILDKIELIGNENHGLYLADQYVKGIAKSRKSGS | 118 |
| | pCoV1B-05-PL-KV | PrefLead-S2P.1154-KV-del-Ferritin (no PP) | 1,314 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSFIEDLL FN KVTLADAGFIKQYGDCLGDIAARDLICAQKFNGLTVLPPLLTDEMIAQYT SALLAGTITSGWTFGAGAALQIPFAMQMAYRFNGIGVTQNVLYENQKLIA NQFNSAIGKIQDSLSSTASALGKLQDVVNQNAQALNTLVKQLSSNFGAIS S VLNDILSRLDKVEAEVQIDRLITGRLQSLQTYVTQQLIRAAEIRASANLA A TKMSECVLGQSKRVDFCGKGYHLMSFPQSAPHGVVFLHVTYVPAQEKNF TTAPAICHDGKAHFPREGVFVSNGTHWFVTQRNFYEPQIITTDNTFVSGN C DVVIGIVNNTVYDPLQPELDSFKEELDKGSGGSGDIIKLLNEQVNKEMQS S NLYMSMSSWCYTHSLDGAGLFLFDHAAEEYEHAKKLIIFLNENNVPVQL TSISAPEHKFEGLTQIFQKAYEHEQHISESINNIVDHAIKSKDHATFNFL QW YVAEQHEEEVLFKDILDKIELIGNENHGLYLADQYVKGIAKSRKSGS | 119 |
| RBD-NTD-Ferr | pCoV122 | His8-3c-RBD-GSGGSG-NTD-SSQC-Ferrritin (from domain fusion sheet) | 694 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYLYRLFRKSNL KPFERDISTEIYQAGSTPCNGVEGFNCYFPLQSYGFQPTNGVGYQPYRVV VLSFELLHAPATVCGPGSGGSGSSQCVNLTTRTQLPPAYTNSFTRGVYYP DKVFRSSVLHSTQDLFLPFFSNVTWFHAIHVSGTNGTKRFDNPVLPFNDG VYFASTEKSNIIRGWIFGTTLDSKTQSLLIVNNATNVVIKVCEFQFCNDP FL GVYYHKNNKSWMESEFRVYSSANNCTFEYVSQPFLMDLEGKQGNFKNL REFVFKNIDGYFKIYSKHTPINLVRDLPQGFSALEPLVDLPIGINITRFQ TLL ALHRSYLTPGDSSSGWTAGAAAYYVGYLQPRTFLLKYNENGTITDAVDC ALDPLSETKCTLGSGGGGESQVRQQFSKDIEKLLNEQVNKEMQSSNLYMS MSSWCYTHSLDGAGLFLFDHAAEEYEHAKKLIIFLNENNVPVQLTSISAP E HKFEGLTQIFQKAYEHEQHISESINNIVDHAIKSKDHATFNFLQWYVAEQ H EEEVLFKDILDKIELIGNENHGLYLADQYVKGIAKSRKSGS | 120 |
| RBD- NTD-Ferr with 56 mutation | pCoV123 | His8-3c-RBD-F490R-GSGGSG-NTD-SSQC-Ferrritin | 694 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYLYRLFRKSNL KPFERDISTEIYQAGSTPCNGVEGFNCYRPLQSYGFQPTNGVGYQPYRVV VLSFELLHAPATVCGPGSGGSGSSQCVNLTTRTQLPPAYTNSFTRGVYYP DKVFRSSVLHSTQDLFLPFFSNVTWFHAIHVSGTNGTKRFDNPVLPFNDG VYFASTEKSNIIRGWIFGTTLDSKTQSLLIVNNATNVVIKVCEFQFCNDP FL GVYYHKNNKSWMESEFRVYSSANNCTFEYVSQPFLMDLEGKQGNFKNL REFVFKNIDGYFKIYSKHTPINLVRDLPQGFSALEPLVDLPIGINITRFQ TLL ALHRSYLTPGDSSSGWTAGAAAYYVGYLQPRTFLLKYNENGTITDAVDC ALDPLSETKCTLGSGGGGESQVRQQFSKDIEKLLNEQVNKEMQSSNLYMS MSSWCYTHSLDGAGLFLFDHAAEEYEHAKKLIIFLNENNVPVQLTSISAP E HKFEGLTQIFQKAYEHEQHISESINNIVDHAIKSKDHATFNFLQWYVAEQ H EEEVLFKDILDKIELIGNENHGLYLADQYVKGIAKSRKSGS | 121 |
| RBD-NTD-Ferr with 57 mutation | pCoV124 | His8-3c-RBD-F490A-GSGGSG-NTD-SSQC-Ferrritin | 694 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYLYRLFRKSNL KPFERDISTEIYQAGSTPCNGVEGFNCYAPLQSYGFQPTNGVGYQPYRVV VLSFELLHAPATVCGPGSGGSGSSQCVNLTTRTQLPPAYTNSFTRGVYYP DKVFRSSVLHSTQDLFLPFFSNVTWFHAIHVSGTNGTKRFDNPVLPFNDG VYFASTEKSNIIRGWIFGTTLDSKTQSLLIVNNATNVVIKVCEFQFCNDP FL GVYYHKNNKSWMESEFRVYSSANNCTFEYVSQPFLMDLEGKQGNFKNL REFVFKNIDGYFKIYSKHTPINLVRDLPQGFSALEPLVDLPIGINITRFQ TLL ALHRSYLTPGDSSSGWTAGAAAYYVGYLQPRTFLLKYNENGTITDAVDC ALDPLSETKCTLGSGGGGESQVRQQFSKDIEKLLNEQVNKEMQSSNLYMS MSSWCYTHSLDGAGLFLFDHAAEEYEHAKKLIIFLNENNVPVQLTSISAP E HKFEGLTQIFQKAYEHEQHISESINNIVDHAIKSKDHATFNFLQWYVAEQ H EEEVLFKDILDKIELIGNENHGLYLADQYVKGIAKSRKSGS | 122 |
| RBD-NTD-Ferr with 58 mutation | pCoV125 | His8-3c-RBD-518LLH to NKS-GSGGSG-NTD-SSQC-Ferrritin | 694 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYLYRLFRKSNL KPFERDISTEIYQAGSTPCNGVEGFNCYFPLQSYGFQPTNGVGYQPYRVV VLSFENKSAPATVCGPGSGGSGSSQCVNLTTRTQLPPAYTNSFTRGVYYP DKVFRSSVLHSTQDLFLPFFSNVTWFHAIHVSGTNGTKRFDNPVLPFNDG VYFASTEKSNIIRGWIFGTTLDSKTQSLLIVNNATNVVIKVCEFQFCNDP FL GVYYHKNNKSWMESEFRVYSSANNCTFEYVSQPFLMDLEGKQGNFKNL REFVFKNIDGYFKIYSKHTPINLVRDLPQGFSALEPLVDLPIGINITRFQ TLL ALHRSYLTPGDSSSGWTAGAAAYYVGYLQPRTFLLKYNENGTITDAVDC ALDPLSETKCTLGSGGGGESQVRQQFSKDIEKLLNEQVNKEMQSSNLYMS MSSWCYTHSLDGAGLFLFDHAAEEYEHAKKLIIFLNENNVPVQLTSISAP E HKFEGLTQIFQKAYEHEQHISESINNIVDHAIKSKDHATFNFLQWYVAEQ H EEEVLFKDILDKIELIGNENHGLYLADQYVKGIAKSRKSGS | 123 |
| RBD-NTD-Ferr with 59 mutation | pCoV126 | His8-3c-RBD-L518R-GSGGSG-NTD-SSQC-Ferrritin | 694 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYLYRLFRKSNL KPFERDISTEIYQAGSTPCNGVEGFNCYFPLQSYGFQPTNGVGYQPYRVV VLSFELRHAPATVCGPGSGGSGSSQCVNLTTRTQLPPAYTNSFTRGVYYP DKVFRSSVLHSTQDLFLPFFSNVTWFHAIHVSGTNGTKRFDNPVLPFNDG VYFASTEKSNIIRGWIFGTTLDSKTQSLLIVNNATNVVIKVCEFQFCNDP FL GVYYHKNNKSWMESEFRVYSSANNCTFEYVSQPFLMDLEGKQGNFKNL REFVFKNIDGYFKIYSKHTPINLVRDLPQGFSALEPLVDLPIGINITRFQ TLL ALHRSYLTPGDSSSGWTAGAAAYYVGYLQPRTFLLKYNENGTITDAVDC ALDPLSETKCTLGSGGGGESQVRQQFSKDIEKLLNEQVNKEMQSSNLYMS MSSWCYTHSLDGAGLFLFDHAAEEYEHAKKLIIFLNENNVPVQLTSISAP E HKFEGLTQIFQKAYEHEQHISESINNIVDHAIKSKDHATFNFLQWYVAEQ H EEEVLFKDILDKIELIGNENHGLYLADQYVKGIAKSRKSGS | 124 |
| 57 + 58 | pCoV127 | His8-3c-RBD-Ferritin-F490A-518LLH to 518 NKS | 396 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYLYRLFRKSNL KPFERDISTEIYQAGSTPCNGVEGFNCYAPLQSYGFQPTNGVGYQPYRVV VLSFENKSAPATVCGPGSGGGGESQVRQQFSKDIEKLLNEQVNKEMQSSN LYMSMSSWCYTHSLDGAGLFLFDHAAEEYEHAKKLIIFLNENNVPVQLTS ISAPEHKFEGLTQIFQKAYEHEQHISESINNIVDHAIKSKDHATFNFLQW YV AEQHEEEVLFKDILDKIELIGNENHGLYLADQYVKGIAKSRKSGS | 125 |
| 57 + 59 | pCoV128 | His8-3c-RBD-Ferritin-F490A-L518R | 396 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYLYRLFRKSNL KPFERDISTEIYQAGSTPCNGVEGFNCYAPLQSYGFQPTNGVGYQPYRVV VLSFELRHAPATVCGPGSGGGGESQVRQQFSKDIEKLLNEQVNKEMQSSN LYMSMSSWCYTHSLDGAGLFLFDHAAEEYEHAKKLIIFLNENNVPVQLTS ISAPEHKFEGLTQIFQKAYEHEQHISESINNIVDHAIKSKDHATFNFLQW YV AEQHEEEVLFKDILDKIELIGNENHGLYLADQYVKGIAKSRKSGS | 126 |
| 50 + 58 | pCoV129 | His8-3c-RBD-Ferritin-L455R/Y449K /F490R-518LLH to NKS | 396 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNKNYLYRRFRKSNL KPFERDISTEIYQAGSTPCNGVEGFNCYRPLQSYGFQPTNGVGYQPYRVV VLSFENKSAPATVCGPGSGGGGESQVRQQFSKDIEKLLNEQVNKEMQSSN LYMSMSSWCYTHSLDGAGLFLFDHAAEEYEHAKKLIIFLNENNVPVQLTS ISAPEHKFEGLTQIFQKAYEHEQHISESINNIVDHAIKSKDHATFNFLQW YV AEQHEEEVLFKDILDKIELIGNENHGLYLADQYVKGIAKSRKSGS | 127 |
| 50 + 59 | pCoV130 | His8-3c-RBD-Ferritin-L455R/Y449K /F490R-L518R | 396 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNKNYLYRRFRKSNL KPFERDISTEIYQAGSTPCNGVEGFNCYRPLQSYGFQPTNGVGYQPYRVV VLSFELRHAPATVCGPGSGGGGESQVRQQFSKDIEKLLNEQVNKEMQSSN LYMSMSSWCYTHSLDGAGLFLFDHAAEEYEHAKKLIIFLNENNVPVQLTS ISAPEHKFEGLTQIFQKAYEHEQHISESINNIVDHAIKSKDHATFNFLQW YV AEQHEEEVLFKDILDKIELIGNENHGLYLADQYVKGIAKSRKSGS | 128 |
| 53 + 58 | pCoV131 | His8-3c-RBD-Ferritin-Y453R-518LLH to NKS | 396 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYLRRLFRKSNL KPFERDISTEIYQAGSTPCNGVEGFNCYFPLQSYGFQPTNGVGYQPYRVV VLSFENKSAPATVCGPGSGGGGESQVRQQFSKDIEKLLNEQVNKEMQSSN LYMSMSSWCYTHSLDGAGLFLFDHAAEEYEHAKKLIIFLNENNVPVQLTS ISAPEHKFEGLTQIFQKAYEHEQHISESINNIVDHAIKSKDHATFNFLQW YV AEQHEEEVLFKDILDKIELIGNENHGLYLADQYVKGIAKSRKSGS | 129 |
| 53 + 59 | pCoV132 | His8-3c-RBD-Ferritin-Y453R-L518R | 396 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYLRRLFRKSNL KPFERDISTEIYQAGSTPCNGVEGFNCYFPLQSYGFQPTNGVGYQPYRVV VLSFELRHAPATVCGPGSGGGGESQVRQQFSKDIEKLLNEQVNKEMQSSN LYMSMSSWCYTHSLDGAGLFLFDHAAEEYEHAKKLIIFLNENNVPVQLTS ISAPEHKFEGLTQIFQKAYEHEQHISESINNIVDHAIKSKDHATFNFLQW YV AEQHEEEVLFKDILDKIELIGNENHGLYLADQYVKGIAKSRKSGS | 130 |
| Native spike leader | pCoV141 | NatLead-S2P-D614G-Foldon-3c-Strep-His8 | 1,271 | VNLTTRTQLPPAYTNSFTRGVYYPDKVFRSSVLHSTQDLFLPFFSNVTWF HAIHVSGTNGTKRFDNPVLPFNDGVYFASTEKSNIIRGWIFGTTLDSKTQ S LLIVNNATNVVIKVCEFQFCNDPFLGVYYHKNNKSWMESEFRVYSSANN CTFEYVSQPFLMDLEGKQGNFKNLREFVFKNIDGYFKIYSKHTPINLVRD L PQGFSALEPLVDLPIGINITRFQTLLALHRSYLTPGDSSSGWTAGAAAYY V GYLQPRTFLLKYNENGTITDAVDCALDPLSETKCTLKSFTVEKGIYQTSN F RVQPTESIVRFPNITNLCPFGEVFNATRFASVYAWNRKRISNCVADYSVL Y NSASFSTFKCYGVSPTKLNDLCFTNVYADSFVIRGDEVRQIAPGQTGKIA D YNYKLPDDFTGCVIAWNSNNLDSKVGGNYNYLYRLFRKSNLKPFERDIST EIYQAGSTPCNGVEGFNCYFPLQSYGFQPTNGVGYQPYRVVVLSFELLHA PATVCGPKKSTNLVKNKCVNFNFNGLTGTGVLTESNKKFLPFQQFGRDIA DTTDAVRDPQTLEILDITPCSFGGVSVITPGTNTSNQVAVLYQGVNCTEV P VAIHADQLTPTWRVYSTGSNVFQTRAGCLIGAEHVNNSYECDIPIGAGIC A SYQTQTNSPGSASSVASQSIIAYTMSLGAENSVAYSNNSIAIPTNFTISV TTE ILPVSMTKTSVDCTMYICGDSTECSNLLLQYGSFCTQLNRALTGIAVEQD K NTQEVFAQVKQIYKTPPIKDFGGFNFSQILPDPSKPSKRSFIEDLLFNKV TL ADAGFIKQYGDCLGDIAARDLICAQKFNGLTVLPPLLTDEMIAQYTSALL AGTITSGWTFGAGAALQIPFAMQMAYRFNGIGVTQNVLYENQKLIANQF NSAIGKIQDSLSSTASALGKLQDVVNQNAQALNTLVKQLSSNFGAISSVL N DILSRLDPPEAEVQIDRLITGRLQSLQTYVTQQLIRAAEIRASANLAATK MS ECVLGQSKRVDFCGKGYHLMSFPQSAPHGVVFLHVTYVPAQEKNFTTAP AICHDGKAHFPREGVFVSNGTHWFVTQRNFYEPQIITTDNTFVSGNCDVV I GIVNNTVYDPLQPELDSFKEELDKYFKNHTSPDVDLGDISGINASVVNIQ K EIDRLNEVAKNLNESLIDLQELGKYEQGSGYIPEAPRDGQAYVRKDGEWV LLSTFLGLEVLFQGPSAWSHPQFEKGGGSGGGSGGSAWSHPQFEKGSHH HHHHHH | 131 |
| | pCoV142 | NatLead-S-KV-Foldon-3c-Strep-His8 | 1,271 | VNLTTRTQLPPAYTNSFTRGVYYPDKVFRSSVLHSTQDLFLPFFSNVTWF HAIHVSGTNGTKRFDNPVLPFNDGVYFASTEKSNIIRGWIFGTTLDSKTQ S LLIVNNATNVVIKVCEFQFCNDPFLGVYYHKNNKSWMESEFRVYSSANN CTFEYVSQPFLMDLEGKQGNFKNLREFVFKNIDGYFKIYSKHTPINLVRD L PQGFSALEPLVDLPIGINITRFQTLLALHRSYLTPGDSSSGWTAGAAAYY V GYLQPRTFLLKYNENGTITDAVDCALDPLSETKCTLKSFTVEKGIYQTSN F RVQPTESIVRFPNITNLCPFGEVFNATRFASVYAWNRKRISNCVADYSVL Y NSASFSTFKCYGVSPTKLNDLCFTNVYADSFVIRGDEVRQIAPGQTGKIA D YNYKLPDDFTGCVIAWNSNNLDSKVGGNYNYLYRLFRKSNLKPFERDIST EIYQAGSTPCNGVEGFNCYFPLQSYGFQPTNGVGYQPYRVVVLSFELLHA PATVCGPKKSTNLVKNKCVNFNFNGLTGTGVLTESNKKFLPFQQFGRDIA DTTDAVRDPQTLEILDITPCSFGGVSVITPGTNTSNQVAVLYQDVNCTEV P VAIHADQLTPTWRVYSTGSNVFQTRAGCLIGAEHVNNSYECDIPIGAGIC A SYQTQTNSPGSASSVASQSIIAYTMSLGAENSVAYSNNSIAIPTNFTISV TTE ILPVSMTKTSVDCTMYICGDSTECSNLLLQYGSFCTQLNRALTGIAVEQD K NTQEVFAQVKQIYKTPPIKDFGGFNFSQILPDPSKPSKRSFIEDLLFNKV TL ADAGFIKQYGDCLGDIAARDLICAQKFNGLTVLPPLLTDEMIAQYTSALL AGTITSGWTFGAGAALQIPFAMQMAYRFNGIGVTQNVLYENQKLIANQF NSAIGKIQDSLSSTASALGKLQDVVNQNAQALNTLVKQLSSNFGAISSVL N DILSRLDKVEAEVQIDRLITGRLQSLQTYVTQQLIRAAEIRASANLAATK M SECVLGQSKRVDFCGKGYHLMSFPQSAPHGVVFLHVTYVPAQEKNFTTA PAICHDGKAHFPREGVFVSNGTHWFVTQRNFYEPQIITTDNTFVSGNCDV VIGIVNNTVYDPLQPELDSFKEELDKYFKNHTSPDVDLGDISGINASVVN I QKEIDRLNEVAKNLNESLIDLQELGKYEQGSGYIPEAPRDGQAYVRKDGE WVLLSTFLGLEVLFQGPSAWSHPQFEKGGGSGGGSGGSAWSHPQFEKGS HHHHHHHH | 132 |
| | pCoV143 | NatLead-S-KV-D614G-Foldon-3c-Strep-His8 | 1,271 | VNLTTRTQLPPAYTNSFTRGVYYPDKVFRSSVLHSTQDLFLPFFSNVTWF HAIHVSGTNGTKRFDNPVLPFNDGVYFASTEKSNIIRGWIFGTTLDSKTQ S LLIVNNATNVVIKVCEFQFCNDPFLGVYYHKNNKSWMESEFRVYSSANN CTFEYVSQPFLMDLEGKQGNFKNLREFVFKNIDGYFKIYSKHTPINLVRD L PQGFSALEPLVDLPIGINITRFQTLLALHRSYLTPGDSSSGWTAGAAAYY V GYLQPRTFLLKYNENGTITDAVDCALDPLSETKCTLKSFTVEKGIYQTSN F RVQPTESIVRFPNITNLCPFGEVFNATRFASVYAWNRKRISNCVADYSVL Y NSASFSTFKCYGVSPTKLNDLCFTNVYADSFVIRGDEVRQIAPGQTGKIA D YNYKLPDDFTGCVIAWNSNNLDSKVGGNYNYLYRLFRKSNLKPFERDIST EIYQAGSTPCNGVEGFNCYFPLQSYGFQPTNGVGYQPYRVVVLSFELLHA PATVCGPKKSTNLVKNKCVNFNFNGLTGTGVLTESNKKFLPFQQFGRDIA DTTDAVRDPQTLEILDITPCSFGGVSVITPGTNTSNQVAVLYQGVNCTEV P VAIHADQLTPTWRVYSTGSNVFQTRAGCLIGAEHVNNSYECDIPIGAGIC A SYQTQTNSPGSASSVASQSIIAYTMSLGAENSVAYSNNSIAIPTNFTISV TTE ILPVSMTKTSVDCTMYICGDSTECSNLLLQYGSFCTQLNRALTGIAVEQD K NTQEVFAQVKQIYKTPPIKDFGGFNFSQILPDPSKPSKRSFIEDLLFNKV TL ADAGFIKQYGDCLGDIAARDLICAQKFNGLTVLPPLLTDEMIAQYTSALL AGTITSGWTFGAGAALQIPFAMQMAYRFNGIGVTQNVLYENQKLIANQF NSAIGKIQDSLSSTASALGKLQDVVNQNAQALNTLVKQLSSNFGAISSVL N DILSRLDKVEAEVQIDRLITGRLQSLQTYVTQQLIRAAEIRASANLAATK M SECVLGQSKRVDFCGKGYHLMSFPQSAPHGVVFLHVTYVPAQEKNFTTA PAICHDGKAHFPREGVFVSNGTHWFVTQRNFYEPQIITTDNTFVSGNCDV VIGIVNNTVYDPLQPELDSFKEELDKYFKNHTSPDVDLGDISGINASVVN I QKEIDRLNEVAKNLNESLIDLQELGKYEQGSGYIPEAPRDGQAYVRKDGE WVLLSTFLGLEVLFQGPSAWSHPQFEKGGGSGGGSGGSAWSHPQFEKGS HHHHHHHH | 133 |
| | pCoV144 | NatLead-S-KV-RRAR-Foldon-3c-Strep-His8 | 1,271 | VNLTTRTQLPPAYTNSFTRGVYYPDKVFRSSVLHSTQDLFLPFFSNVTWF HAIHVSGTNGTKRFDNPVLPFNDGVYFASTEKSNIIRGWIFGTTLDSKTQ S LLIVNNATNVVIKVCEFQFCNDPFLGVYYHKNNKSWMESEFRVYSSANN CTFEYVSQPFLMDLEGKQGNFKNLREFVFKNIDGYFKIYSKHTPINLVRD L PQGFSALEPLVDLPIGINITRFQTLLALHRSYLTPGDSSSGWTAGAAAYY V GYLQPRTFLLKYNENGTITDAVDCALDPLSETKCTLKSFTVEKGIYQTSN F RVQPTESIVRFPNITNLCPFGEVFNATRFASVYAWNRKRISNCVADYSVL Y NSASFSTFKCYGVSPTKLNDLCFTNVYADSFVIRGDEVRQIAPGQTGKIA D YNYKLPDDFTGCVIAWNSNNLDSKVGGNYNYLYRLFRKSNLKPFERDIST EIYQAGSTPCNGVEGFNCYFPLQSYGFQPTNGVGYQPYRVVVLSFELLHA PATVCGPKKSTNLVKNKCVNFNFNGLTGTGVLTESNKKFLPFQQFGRDIA DTTDAVRDPQTLEILDITPCSFGGVSVITPGTNTSNQVAVLYQDVNCTEV P VAIHADQLTPTWRVYSTGSNVFQTRAGCLIGAEHVNNSYECDIPIGAGIC A SYQTQTNSPRRARSVASQSIIAYTMSLGAENSVAYSNNSIAIPTNFTISV TT EILPVSMTKTSVDCTMYICGDSTECSNLLLQYGSFCTQLNRALTGIAVEQ D KNTQEVFAQVKQIYKTPPIKDFGGFNFSQILPDPSKPSKRSFIEDLLFNK VT LADAGFIKQYGDCLGDIAARDLICAQKFNGLTVLPPLLTDEMIAQYTSAL L AGTITSGWTFGAGAALQIPFAMQMAYRFNGIGVTQNVLYENQKLIANQF NSAIGKIQDSLSSTASALGKLQDVVNQNAQALNTLVKQLSSNFGAISSVL N DILSRLDKVEAEVQIDRLITGRLQSLQTYVTQQLIRAAEIRASANLAATK M SECVLGQSKRVDFCGKGYHLMSFPQSAPHGVVFLHVTYVPAQEKNFTTA PAICHDGKAHFPREGVFVSNGTHWFVTQRNFYEPQIITTDNTFVSGNCDV VIGIVNNTVYDPLQPELDSFKEELDKYFKNHTSPDVDLGDISGINASVVN I QKEIDRLNEVAKNLNESLIDLQELGKYEQGSGYIPEAPRDGQAYVRKDGE WVLLSTFLGLEVLFQGPSAWSHPQFEKGGGSGGGSGGSAWSHPQFEKGS HHHHHHHH | 134 |
| | pCoV145 | NatLead-S-KV-RRAR-D614G-Foldon-3c-Strep-His8 | 1,271 | VNLTTRTQLPPAYTNSFTRGVYYPDKVFRSSVLHSTQDLFLPFFSNVTWF HAIHVSGTNGTKRFDNPVLPFNDGVYFASTEKSNIIRGWIFGTTLDSKTQ S LLIVNNATNVVIKVCEFQFCNDPFLGVYYHKNNKSWMESEFRVYSSANN CTFEYVSQPFLMDLEGKQGNFKNLREFVFKNIDGYFKIYSKHTPINLVRD L PQGFSALEPLVDLPIGINITRFQTLLALHRSYLTPGDSSSGWTAGAAAYY V GYLQPRTFLLKYNENGTITDAVDCALDPLSETKCTLKSFTVEKGIYQTSN F RVQPTESIVRFPNITNLCPFGEVFNATRFASVYAWNRKRISNCVADYSVL Y NSASFSTFKCYGVSPTKLNDLCFTNVYADSFVIRGDEVRQIAPGQTGKIA D YNYKLPDDFTGCVIAWNSNNLDSKVGGNYNYLYRLFRKSNLKPFERDIST EIYQAGSTPCNGVEGFNCYFPLQSYGFQPTNGVGYQPYRVVVLSFELLHA PATVCGPKKSTNLVKNKCVNFNFNGLTGTGVLTESNKKFLPFQQFGRDIA DTTDAVRDPQTLEILDITPCSFGGVSVITPGTNTSNQVAVLYQGVNCTEV P VAIHADQLTPTWRVYSTGSNVFQTRAGCLIGAEHVNNSYECDIPIGAGIC A SYQTQTNSPRRARSVASQSIIAYTMSLGAENSVAYSNNSIAIPTNFTISV TT EILPVSMTKTSVDCTMYICGDSTECSNLLLQYGSFCTQLNRALTGIAVEQ D KNTQEVFAQVKQIYKTPPIKDFGGFNFSQILPDPSKPSKRSFIEDLLFNK VT LADAGFIKQYGDCLGDIAARDLICAQKFNGLTVLPPLLTDEMIAQYTSAL L AGTITSGWTFGAGAALQIPFAMQMAYRFNGIGVTQNVLYENQKLIANQF NSAIGKIQDSLSSTASALGKLQDVVNQNAQALNTLVKQLSSNFGAISSVL N DILSRLDKVEAEVQIDRLITGRLQSLQTYVTQQLIRAAEIRASANLAATK M SECVLGQSKRVDFCGKGYHLMSFPQSAPHGVVFLHVTYVPAQEKNFTTA PAICHDGKAHFPREGVFVSNGTHWFVTQRNFYEPQIITTDNTFVSGNCDV VIGIVNNTVYDPLQPELDSFKEELDKYFKNHTSPDVDLGDISGINASVVN I QKEIDRLNEVAKNLNESLIDLQELGKYEQGSGYIPEAPRDGQAYVRKDGE WVLLSTFLGLEVLFQGPSAWSHPQFEKGGGSGGGSGGSAWSHPQFEKGS HHHHHHHH | 135 |
| RBD-NTD-Ferr with 53 + 58 mut | pCoV146 | His8-3c-RBD-Y453R-518LLH to NKS-GSGGSG-NTD-SSQC-Ferrritin | 694 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYLRRLFRKSNL KPFERDISTEIYQAGSTPCNGVEGFNCYFPLQSYGFQPTNGVGYQPYRVV VLSFENKSAPATVCGPGSGGSGSSQCVNLTTRTQLPPAYTNSFTRGVYYP DKVFRSSVLHSTQDLFLPFFSNVTWFHAIHVSGTNGTKRFDNPVLPFNDG VYFASTEKSNIIRGWIFGTTLDSKTQSLLIVNNATNVVIKVCEFQFCNDP FL GVYYHKNNKSWMESEFRVYSSANNCTFEYVSQPFLMDLEGKQGNFKNL REFVFKNIDGYFKIYSKHTPINLVRDLPQGFSALEPLVDLPIGINITRFQ TLL ALHRSYLTPGDSSSGWTAGAAAYYVGYLQPRTFLLKYNENGTITDAVDC ALDPLSETKCTLGSGGGGESQVRQQFSKDIEKLLNEQVNKEMQSSNLYMS MSSWCYTHSLDGAGLFLFDHAAEEYEHAKKLIIFLNENNVPVQLTSISAP E HKFEGLTQIFQKAYEHEQHISESINNIVDHAIKSKDHATFNFLQWYVAEQ H EEEVLFKDILDKIELIGNENHGLYLADQYVKGIAKSRKSGS | 136 |
| RBD-NTD-Ferr with 57 + 58 mut | pCoV147 | His8-3c-RBD-F490A-518LLH to NKS-GSGGSG-NTD-SSQC-Ferrritin | 694 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYLYRLFRKSNL KPFERDISTEIYQAGSTPCNGVEGFNCYAPLQSYGFQPTNGVGYQPYRVV VLSFENKSAPATVCGPGSGGSGSSQCVNLTTRTQLPPAYTNSFTRGVYYP DKVFRSSVLHSTQDLFLPFFSNVTWFHAIHVSGTNGTKRFDNPVLPFNDG VYFASTEKSNIIRGWIFGTTLDSKTQSLLIVNNATNVVIKVCEFQFCNDP FL GVYYHKNNKSWMESEFRVYSSANNCTFEYVSQPFLMDLEGKQGNFKNL REFVFKNIDGYFKIYSKHTPINLVRDLPQGFSALEPLVDLPIGINITRFQ TLL ALHRSYLTPGDSSSGWTAGAAAYYVGYLQPRTFLLKYNENGTITDAVDC ALDPLSETKCTLGSGGGGESQVRQQFSKDIEKLLNEQVNKEMQSSNLYMS MSSWCYTHSLDGAGLFLFDHAAEEYEHAKKLIIFLNENNVPVQLTSISAP E HKFEGLTQIFQKAYEHEQHISESINNIVDHAIKSKDHATFNFLQWYVAEQ H EEEVLFKDILDKIELIGNENHGLYLADQYVKGIAKSRKSGS | 137 |
| 111 with 58 mut | pCoV151 | S1-SSQC-T676-GG-SQSIIAYT696-518LLH to 518 NKS-Ferritin | 856 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEN K SAPATVCGPKKSTNLVKNKCVNFNFNGLTGTGVLTESNKKFLPFQQFGRD IADTTDAVRDPQTLEILDITPCSFGGVSVITPGTNTSNQVAVLYQDVNCT E VPVAIHADQLTPTWRVYSTGSNVFQTRAGCLIGAEHVNNSYECDIPIGAG I CASYQTGGSQSIIAYTGSGGGGESQVRQQFSKDIEKLLNEQVNKEMQSSN LYMSMSSWCYTHSLDGAGLFLFDHAAEEYEHAKKLIIFLNENNVPVQLTS ISAPEHKFEGLTQIFQKAYEHEQHISESINNIVDHAIKSKDHATFNFLQW YV AEQHEEEVLFKDILDKIELIGNENHGLYLADQYVKGIAKSRKSGS | 138 |
| 111 with 50 + 58 mut | pCoV152 | S1-SSQC-T676-GG-SQSIIAYT696 L455R/Y449K/F490R-518LLH to 518 NKS-Ferritin | 856 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNKNYLYRRFRKSNLKPFERD ISTEIYQAGSTPCNGVEGFNCYRPLQSYGFQPTNGVGYQPYRVVVLSFEN KSAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTGGSQSIIAYTGSGGGGESQVRQQFSKDIEKLLNEQVNKEMQS S NLYMSMSSWCYTHSLDGAGLFLFDHAAEEYEHAKKLIIFLNENNVPVQL TSISAPEHKFEGLTQIFQKAYEHEQHISESINNIVDHAIKSKDHATFNFL QW YVAEQHEEEVLFKDILDKIELIGNENHGLYLADQYVKGIAKSRKSGS | 139 |
| 111 with 57 + 58 mut | pCoV153 | S1-SSQC-T676-GG-SQSIIAYT696 -F490A-518LLH to 518 NKS-Ferritin | 856 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYAPLQSYGFQPTNGVGYQPYRVVVLSFEN KSAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTGGSQSIIAYTGSGGGGESQVRQQFSKDIEKLLNEQVNKEMQS S NLYMSMSSWCYTHSLDGAGLFLFDHAAEEYEHAKKLIIFLNENNVPVQL TSISAPEHKFEGLTQIFQKAYEHEQHISESINNIVDHAIKSKDHATFNFL QW YVAEQHEEEVLFKDILDKIELIGNENHGLYLADQYVKGIAKSRKSGS | 140 |
| NTD-RBD-Ferr with 57 + 58 mut | pCoV154 | His8-3c-NTD-RBD-F490A-518LLH to NKS-Ferritin | 694 | HHHHHHHHGPLEVLFQGPSSQCVNLTTRTQLPPAYTNSFTRGVYYPDKVF RSSVLHSTQDLFLPFFSNVTWFHAIHVSGTNGTKRFDNPVLPFNDGVYFA S TEKSNIIRGWIFGTTLDSKTQSLLIVNNATNVVIKVCEFQFCNDPFLGVY Y HKNNKSWMESEFRVYSSANNCTFEYVSQPFLMDLEGKQGNFKNLREFVF KNIDGYFKIYSKHTPINLVRDLPQGFSALEPLVDLPIGINITRFQTLLAL HRS YLTPGDSSSGWTAGAAAYYVGYLQPRTFLLKYNENGTITDAVDCALDPL SETKCTLGSGGSGNITNLCPFGEVFNATRFASVYAWNRKRISNCVADYSV LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYAPLQSYGFQPTNGVGYQPYRVVVLSFEN KSAPATVCGPGSGGGGESQVRQQFSKDIEKLLNEQVNKEMQSSNLYMSM SSWCYTHSLDGAGLFLFDHAAEEYEHAKKLIIFLNENNVPVQLTSISAPE H KFEGLTQIFQKAYEHEQHISESINNIVDHAIKSKDHATFNFLQWYVAEQH E EEVLFKDILDKIELIGNENHGLYLADQYVKGIAKSRKSGS | 141 |
| NTD-RBD-Ferr with 53 + 58 mut | pCoV155 | His8-3c-NTD-RBD-Y453R-518LLH to NKS-Ferritin | 694 | HHHHHHHHGPLEVLFQGPSSQCVNLTTRTQLPPAYTNSFTRGVYYPDKVF RSSVLHSTQDLFLPFFSNVTWFHAIHVSGTNGTKRFDNPVLPFNDGVYFA S TEKSNIIRGWIFGTTLDSKTQSLLIVNNATNVVIKVCEFQFCNDPFLGVY Y HKNNKSWMESEFRVYSSANNCTFEYVSQPFLMDLEGKQGNFKNLREFVF KNIDGYFKIYSKHTPINLVRDLPQGFSALEPLVDLPIGINITRFQTLLAL HRS YLTPGDSSSGWTAGAAAYYVGYLQPRTFLLKYNENGTITDAVDCALDPL SETKCTLGSGGSGNITNLCPFGEVFNATRFASVYAWNRKRISNCVADYSV LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPGSGGGGESQVRQQFSKDIEKLLNEQVNKEMQSSNLYMSMS SWCYTHSLDGAGLFLFDHAAEEYEHAKKLIIFLNENNVPVQLTSISAPEH K FEGLTQIFQKAYEHEQHISESINNIVDHAIKSKDHATFNFLQWYVAEQHE E EVLFKDILDKIELIGNENHGLYLADQYVKGIAKSRKSGS | 142 |
| 111 with 50 mut | pCoV156 | S1-SSQC-T676-GG-SQSIIAYT696 -L455R/Y449K /F490R-Ferritin | 856 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNKNYLYRRFRKSNLKPFERD ISTEIYQAGSTPCNGVEGFNCYRPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTGGSQSIIAYTGSGGGGESQVRQQFSKDIEKLLNEQVNKEMQS S NLYMSMSSWCYTHSLDGAGLFLFDHAAEEYEHAKKLIIFLNENNVPVQL TSISAPEHKFEGLTQIFQKAYEHEQHISESINNIVDHAIKSKDHATFNFL QW YVAEQHEEEVLFKDILDKIELIGNENHGLYLADQYVKGIAKSRKSGS | 143 |
| 1B-06-PL with D614G | pCoV159 | PL-S2P.1158op1-del-Ferritin-D614G (aka 1B-06-PL with D614G) | 1,318 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQGVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSFIEDLL FN KVTLADAGFIKQYGDCLGDIAARDLICAQKFNGLTVLPPLLTDEMIAQYT SALLAGTITSGWTFGAGAALQIPFAMQMAYRFNGIGVTQNVLYENQKLIA NQFNSAIGKIQDSLSSTASALGKLQDVVNQNAQALNTLVKQLSSNFGAIS S VLNDILSRLDPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQSELDSIKEELDKIHKNGSGGSGDIIKLLNEQVNK E MQSSNLYMSMSSWCYTHSLDGAGLFLFDHAAEEYEHAKKLIIFLNENNV PVQLTSISAPEHKFEGLTQIFQKAYEHEQHISESINNIVDHAIKSKDHAT FNF LQWYVAEQHEEEVLFKDILDKIELIGNENHGLYLADQYVKGIAKSRKSGS | 144 |
| Native spike leader, 1B-05 with DS1 | pCoV160 | S2P.1154_DS1-del-Ferritin (native leader) | 1,310 | VNLTTRTQLPPAYTNSFTRGVYYPDKVFRSSVLHSTQDLFLPFFSNVTWF HAIHVSGTNGTKRFDNPVLPFNDGVYFASTEKSNIIRGWIFGTTLDSKTQ S LLIVNNATNVVIKVCEFQFCNDPFLGVYYHKNNKSWMESEFRVYSSANN CTFEYVSQPFLMDLEGKQGNFKNLREFVFKNIDGYFKIYSKHTPINLVRD L PQGFSALEPLVDLPIGINITRFQTLLALHRSYLTPGDSSSGWTAGAAAYY V GYLQPRTFLLKYNENGTITDAVDCALDPLSETKCTLKSFTVEKGIYQTSN F RVQPTESIVRFPNITNLCPFGEVFNATRFASVYAWNRKRISNCVADYSVL Y NSASFSTFKCYGVCPTKLNDLCFTNVYADSFVIRGDEVRQIAPGQTGKIA D YNYKLPDDFTGCVIAWNSNNLDSKVGGNYNYLYRLFRKSNLKPFERDIST EIYQAGSTPCNGVEGFNCYFPLQSYGFQPTNGVGYQPYRVVVLSFELLHA PATVCGPKKSTNLVKNKCVNFNFNGLTGTGVLTESNKKFLPFQQFGRDIA DTTDAVRDPQTLEILDITPCSFGGVSVITPGTNTSNQVAVLYQDVNCTEV P VAIHADQLTPTWRVYSTGSNVFQTRAGCLIGAEHVNNSYECDIPIGAGIC A SYQTQTNSPGSASSVASQSIIAYTMSLGAENSVAYSNNSIAIPTNFTISV TTE ILPVSMTKTSVDCTMYICGDSTECSNLLLQYGSFCTQLNRALTGIAVEQD K NTQEVFAQVKQIYKTPPIKDFGGFNFSQILPDPSKPSKRSFIEDLLFNKV TL ADAGFIKQYGDCLGDIAARDLICAQKFNGLTVLPPLLTDEMIAQYTSALL AGTITSGWTFGAGAALQIPFAMQMAYRFNGIGVTQNVLYENQKLIANQF NSAIGKIQDSLSSTASALGKLQDVVNQNAQALNTLVKQLSSNFGAISSVL N DILSRLCPPEAEVQIDRLITGRLQSLQTYVTQQLIRAAEIRASANLAATK MS ECVLGQSKRVDFCGKGYHLMSFPQSAPHGVVFLHVTYVPAQEKNFTTAP AICHDGKAHFPREGVFVSNGTHWFVTQRNFYEPQIITTDNTFVSGNCDVV I GIVNNTVYDPLQPELDSFKEELDKGSGGSGDIIKLLNEQVNKEMQSSNLY MSMSSWCYTHSLDGAGLFLFDHAAEEYEHAKKLIIFLNENNVPVQLTSIS APEHKFEGLTQIFQKAYEHEQHISESINNIVDHAIKSKDHATFNFLQWYV A EQHEEEVLFKDILDKIELIGNENHGLYLADQYVKGIAKSRKSGS | 145 |
| 1B-06-PL with DS1 | pCoV161 | PL-S2P.1158op1_DS1-del-Ferritin | 1,318 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVCPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSFIEDLL FN KVTLADAGFIKQYGDCLGDIAARDLICAQKFNGLTVLPPLLTDEMIAQYT SALLAGTITSGWTFGAGAALQIPFAMQMAYRFNGIGVTQNVLYENQKLIA NQFNSAIGKIQDSLSSTASALGKLQDVVNQNAQALNTLVKQLSSNFGAIS S VLNDILSRLCPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQSELDSIKEELDKIHKNGSGGSGDIIKLLNEQVNK E MQSSNLYMSMSSWCYTHSLDGAGLFLFDHAAEEYEHAKKLIIFLNENNV PVQLTSISAPEHKFEGLTQIFQKAYEHEQHISESINNIVDHAIKSKDHAT FNF LQWYVAEQHEEEVLFKDILDKIELIGNENHGLYLADQYVKGIAKSRKSGS | 146 |
| 1B-06-PL SSQ removed (only C) | pCoV162 | PL-S2P.1158op1-del-Ferritin-C only (pref leader) | 1,315 | CVNLTTRTQLPPAYTNSFTRGVYYPDKVFRSSVLHSTQDLFLPFFSNVTW F HAIHVSGTNGTKRFDNPVLPFNDGVYFASTEKSNIIRGWIFGTTLDSKTQ S LLIVNNATNVVIKVCEFQFCNDPFLGVYYHKNNKSWMESEFRVYSSANN CTFEYVSQPFLMDLEGKQGNFKNLREFVFKNIDGYFKIYSKHTPINLVRD L PQGFSALEPLVDLPIGINITRFQTLLALHRSYLTPGDSSSGWTAGAAAYY V GYLQPRTFLLKYNENGTITDAVDCALDPLSETKCTLKSFTVEKGIYQTSN F RVQPTESIVRFPNITNLCPFGEVFNATRFASVYAWNRKRISNCVADYSVL Y NSASFSTFKCYGVSPTKLNDLCFTNVYADSFVIRGDEVRQIAPGQTGKIA D YNYKLPDDFTGCVIAWNSNNLDSKVGGNYNYLYRLFRKSNLKPFERDIST EIYQAGSTPCNGVEGFNCYFPLQSYGFQPTNGVGYQPYRVVVLSFELLHA PATVCGPKKSTNLVKNKCVNFNFNGLTGTGVLTESNKKFLPFQQFGRDIA DTTDAVRDPQTLEILDITPCSFGGVSVITPGTNTSNQVAVLYQDVNCTEV P VAIHADQLTPTWRVYSTGSNVFQTRAGCLIGAEHVNNSYECDIPIGAGIC A SYQTQTNSPGSASSVASQSIIAYTMSLGAENSVAYSNNSIAIPTNFTISV TTE ILPVSMTKTSVDCTMYICGDSTECSNLLLQYGSFCTQLNRALTGIAVEQD K NTQEVFAQVKQIYKTPPIKDFGGFNFSQILPDPSKPSKRSFIEDLLFNKV TL ADAGFIKQYGDCLGDIAARDLICAQKFNGLTVLPPLLTDEMIAQYTSALL AGTITSGWTFGAGAALQIPFAMQMAYRFNGIGVTQNVLYENQKLIANQF NSAIGKIQDSLSSTASALGKLQDVVNQNAQALNTLVKQLSSNFGAISSVL N DILSRLDPPEAEVQIDRLITGRLQSLQTYVTQQLIRAAEIRASANLAATK MS ECVLGQSKRVDFCGKGYHLMSFPQSAPHGVVFLHVTYVPAQEKNFTTAP AICHDGKAHFPREGVFVSNGTHWFVTQRNFYEPQIITTDNTFVSGNCDVV I GIVNNTVYDPLQSELDSIKEELDKIHKNGSGGSGDIIKLLNEQVNKEMQS S NLYMSMSSWCYTHSLDGAGLFLFDHAAEEYEHAKKLIIFLNENNVPVQL TSISAPEHKFEGLTQIFQKAYEHEQHISESINNIVDHAIKSKDHATFNFL QW YVAEQHEEEVLFKDILDKIELIGNENHGLYLADQYVKGIAKSRKSGS | 147 |
| 1B-06-PL ETGTQC (Weesler) | pCoV163 | PL-S2P.1158op1-del-Ferritin-ETGTQC only (pref leader) | 1,320 | ETGTQCVNLTTRTQLPPAYTNSFTRGVYYPDKVFRSSVLHSTQDLFLPFF S NVTWFHAIHVSGTNGTKRFDNPVLPFNDGVYFASTEKSNIIRGWIFGTTL D SKTQSLLIVNNATNVVIKVCEFQFCNDPFLGVYYHKNNKSWMESEFRVYS SANNCTFEYVSQPFLMDLEGKQGNFKNLREFVFKNIDGYFKIYSKHTPIN L VRDLPQGFSALEPLVDLPIGINITRFQTLLALHRSYLTPGDSSSGWTAGA A AYYVGYLQPRTFLLKYNENGTITDAVDCALDPLSETKCTLKSFTVEKGIY QTSNFRVQPTESIVRFPNITNLCPFGEVFNATRFASVYAWNRKRISNCVA D YSVLYNSASFSTFKCYGVSPTKLNDLCFTNVYADSFVIRGDEVRQIAPGQ T GKIADYNYKLPDDFTGCVIAWNSNNLDSKVGGNYNYLYRLFRKSNLKPF ERDISTEIYQAGSTPCNGVEGFNCYFPLQSYGFQPTNGVGYQPYRVVVLS F ELLHAPATVCGPKKSTNLVKNKCVNFNFNGLTGTGVLTESNKKFLPFQQF GRDIADTTDAVRDPQTLEILDITPCSFGGVSVITPGTNTSNQVAVLYQDV N CTEVPVAIHADQLTPTWRVYSTGSNVFQTRAGCLIGAEHVNNSYECDIPI G AGICASYQTQTNSPGSASSVASQSIIAYTMSLGAENSVAYSNNSIAIPTN FTI SVTTEILPVSMTKTSVDCTMYICGDSTECSNLLLQYGSFCTQLNRALTGI A VEQDKNTQEVFAQVKQIYKTPPIKDFGGFNFSQILPDPSKPSKRSFIEDL LF NKVTLADAGFIKQYGDCLGDIAARDLICAQKFNGLTVLPPLLTDEMIAQY TSALLAGTITSGWTFGAGAALQIPFAMQMAYRFNGIGVTQNVLYENQKLI ANQFNSAIGKIQDSLSSTASALGKLQDVVNQNAQALNTLVKQLSSNFGAI SSVLNDILSRLDPPEAEVQIDRLITGRLQSLQTYVTQQLIRAAEIRASAN LA ATKMSECVLGQSKRVDFCGKGYHLMSFPQSAPHGVVFLHVTYVPAQEK NFTTAPAICHDGKAHFPREGVFVSNGTHWFVTQRNFYEPQIITTDNTFVS G NCDVVIGIVNNTVYDPLQSELDSIKEELDKIHKNGSGGSGDIIKLLNEQV N KEMQSSNLYMSMSSWCYTHSLDGAGLFLFDHAAEEYEHAKKLIIFLNEN NVPVQLTSISAPEHKFEGLTQIFQKAYEHEQHISESINNIVDHAIKSKDH AT FNFLQWYVAEQHEEEVLFKDILDKIELIGNENHGLYLADQYVKGIAKSRK SGS | 148 |
| 1B-06-PL SDLDRC (SARS1) | pCoV164 | PL-S2P.1158op1-del-Ferritin-SDLDRC only (pref leader) | 1,320 | SDLDRCVNLTTRTQLPPAYTNSFTRGVYYPDKVFRSSVLHSTQDLFLPFF S NVTWFHAIHVSGTNGTKRFDNPVLPFNDGVYFASTEKSNIIRGWIFGTTL D SKTQSLLIVNNATNVVIKVCEFQFCNDPFLGVYYHKNNKSWMESEFRVYS SANNCTFEYVSQPFLMDLEGKQGNFKNLREFVFKNIDGYFKIYSKHTPIN L VRDLPQGFSALEPLVDLPIGINITRFQTLLALHRSYLTPGDSSSGWTAGA A AYYVGYLQPRTFLLKYNENGTITDAVDCALDPLSETKCTLKSFTVEKGIY QTSNFRVQPTESIVRFPNITNLCPFGEVFNATRFASVYAWNRKRISNCVA D YSVLYNSASFSTFKCYGVSPTKLNDLCFTNVYADSFVIRGDEVRQIAPGQ T GKIADYNYKLPDDFTGCVIAWNSNNLDSKVGGNYNYLYRLFRKSNLKPF ERDISTEIYQAGSTPCNGVEGFNCYFPLQSYGFQPTNGVGYQPYRVVVLS F ELLHAPATVCGPKKSTNLVKNKCVNFNFNGLTGTGVLTESNKKFLPFQQF GRDIADTTDAVRDPQTLEILDITPCSFGGVSVITPGTNTSNQVAVLYQDV N CTEVPVAIHADQLTPTWRVYSTGSNVFQTRAGCLIGAEHVNNSYECDIPI G AGICASYQTQTNSPGSASSVASQSIIAYTMSLGAENSVAYSNNSIAIPTN FTI SVTTEILPVSMTKTSVDCTMYICGDSTECSNLLLQYGSFCTQLNRALTGI A VEQDKNTQEVFAQVKQIYKTPPIKDFGGFNFSQILPDPSKPSKRSFIEDL LF NKVTLADAGFIKQYGDCLGDIAARDLICAQKFNGLTVLPPLLTDEMIAQY TSALLAGTITSGWTFGAGAALQIPFAMQMAYRFNGIGVTQNVLYENQKLI ANQFNSAIGKIQDSLSSTASALGKLQDVVNQNAQALNTLVKQLSSNFGAI SSVLNDILSRLDPPEAEVQIDRLITGRLQSLQTYVTQQLIRAAEIRASAN LA ATKMSECVLGQSKRVDFCGKGYHLMSFPQSAPHGVVFLHVTYVPAQEK NFTTAPAICHDGKAHFPREGVFVSNGTHWFVTQRNFYEPQIITTDNTFVS G NCDVVIGIVNNTVYDPLQSELDSIKEELDKIHKNGSGGSGDIIKLLNEQV N KEMQSSNLYMSMSSWCYTHSLDGAGLFLFDHAAEEYEHAKKLIIFLNEN NVPVQLTSISAPEHKFEGLTQIFQKAYEHEQHISESINNIVDHAIKSKDH AT FNFLQWYVAEQHEEEVLFKDILDKIELIGNENHGLYLADQYVKGIAKSRK SGS | 149 |
| 1B-06-PL reverted mutations | pCoV165 | PL-S2P.1158-del-Ferritin (revert mutations) | 1,318 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSFIEDLL FN KVTLADAGFIKQYGDCLGDIAARDLICAQKFNGLTVLPPLLTDEMIAQYT SALLAGTITSGWTFGAGAALQIPFAMQMAYRFNGIGVTQNVLYENQKLIA NQFNSAIGKIQDSLSSTASALGKLQDVVNQNAQALNTLVKQLSSNFGAIS S VLNDILSRLDPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQPELDSFKEELDKYFKNGSGGSGDIIKLLNEQVNK E MQSSNLYMSMSSWCYTHSLDGAGLFLFDHAAEEYEHAKKLIIFLNENNV PVQLTSISAPEHKFEGLTQIFQKAYEHEQHISESINNIVDHAIKSKDHAT FNF LQWYVAEQHEEEVLFKDILDKIELIGNENHGLYLADQYVKGIAKSRKSGS | 150 |
| 1B-06-PL some reverted mut | pCoV166 | PL-S2P.1158_F11 48I-del-Ferritin | 1,318 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSFIEDLL FN KVTLADAGFIKQYGDCLGDIAARDLICAQKFNGLTVLPPLLTDEMIAQYT SALLAGTITSGWTFGAGAALQIPFAMQMAYRFNGIGVTQNVLYENQKLIA NQFNSAIGKIQDSLSSTASALGKLQDVVNQNAQALNTLVKQLSSNFGAIS S VLNDILSRLDPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQPELDSIKEELDKYFKNGSGGSGDIIKLLNEQVNK E MQSSNLYMSMSSWCYTHSLDGAGLFLFDHAAEEYEHAKKLIIFLNENNV PVQLTSISAPEHKFEGLTQIFQKAYEHEQHISESINNIVDHAIKSKDHAT FNF LQWYVAEQHEEEVLFKDILDKIELIGNENHGLYLADQYVKGIAKSRKSGS | 151 |
| 1B-06-PL with 1143 reverted to P | pCoV167 | PL-S2P.1158-del-Ferritin (revert P1143S)-F1148I/Y1155I/F1156H | 1,318 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSFIEDLL FN KVTLADAGFIKQYGDCLGDIAARDLICAQKFNGLTVLPPLLTDEMIAQYT SALLAGTITSGWTFGAGAALQIPFAMQMAYRFNGIGVTQNVLYENQKLIA NQFNSAIGKIQDSLSSTASALGKLQDVVNQNAQALNTLVKQLSSNFGAIS S VLNDILSRLDPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQPELDSIKEELDKIHKNGSGGSGDIIKLLNEQVNK E MQSSNLYMSMSSWCYTHSLDGAGLFLFDHAAEEYEHAKKLIIFLNENNV PVQLTSISAPEHKFEGLTQIFQKAYEHEQHISESINNIVDHAIKSKDHAT FNF LQWYVAEQHEEEVLFKDILDKIELIGNENHGLYLADQYVKGIAKSRKSGS | 152 |
| 1B-06-PL removed last residue | pCoV168 | PL-S2P.1157-del-Ferritin | 1,317 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSFIEDLL FN KVTLADAGFIKQYGDCLGDIAARDLICAQKFNGLTVLPPLLTDEMIAQYT SALLAGTITSGWTFGAGAALQIPFAMQMAYRFNGIGVTQNVLYENQKLIA NQFNSAIGKIQDSLSSTASALGKLQDVVNQNAQALNTLVKQLSSNFGAIS S VLNDILSRLDPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQPELDSFKEELDKYFKGSGGSGDIIKLLNEQVNKE M QSSNLYMSMSSWCYTHSLDGAGLFLFDHAAEEYEHAKKLIIFLNENNVPV QLTSISAPEHKFEGLTQIFQKAYEHEQHISESINNIVDHAIKSKDHATFN FL QWYVAEQHEEEVLFKDILDKIELIGNENHGLYLADQYVKGIAKSRKSGS | 153 |
| 1B-06-PL added H at the end | pCoV169 | PL-S2P.1159-del-Ferritin | 1,319 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSFIEDLL FN KVTLADAGFIKQYGDCLGDIAARDLICAQKFNGLTVLPPLLTDEMIAQYT SALLAGTITSGWTFGAGAALQIPFAMQMAYRFNGIGVTQNVLYENQKLIA NQFNSAIGKIQDSLSSTASALGKLQDVVNQNAQALNTLVKQLSSNFGAIS S VLNDILSRLDPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQPELDSFKEELDKYFKNHGSGGSGDIIKLLNEQVN K EMQSSNLYMSMSSWCYTHSLDGAGLFLFDHAAEEYEHAKKLIIFLNENN VPVQLTSISAPEHKFEGLTQIFQKAYEHEQHISESINNIVDHAIKSKDHA TF NFLQWYVAEQHEEEVLFKDILDKIELIGNENHGLYLADQYVKGIAKSRKS GS | 154 |
| 1B-06-PL added HT at the end | pCoV170 | PL-S2P.1160-del-Ferritin | 1,320 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSFIEDLL FN KVTLADAGFIKQYGDCLGDIAARDLICAQKFNGLTVLPPLLTDEMIAQYT SALLAGTITSGWTFGAGAALQIPFAMQMAYRFNGIGVTQNVLYENQKLIA NQFNSAIGKIQDSLSSTASALGKLQDVVNQNAQALNTLVKQLSSNFGAIS S VLNDILSRLDPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQPELDSFKEELDKYFKNHTGSGGSGDIIKLLNEQV N KEMQSSNLYMSMSSWCYTHSLDGAGLFLFDHAAEEYEHAKKLIIFLNEN NVPVQLTSISAPEHKFEGLTQIFQKAYEHEQHISESINNIVDHAIKSKDH AT FNFLQWYVAEQHEEEVLFKDILDKIELIGNENHGLYLADQYVKGIAKSRK SGS | 155 |
| 1B-06-PL combo | pCoV171 | PL-S2P.1157op1-del-Ferritin (revert P1143S)-F1148I/Y1155I/F1156H | 1,317 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSFIEDLL FN KVTLADAGFIKQYGDCLGDIAARDLICAQKFNGLTVLPPLLTDEMIAQYT SALLAGTITSGWTFGAGAALQIPFAMQMAYRFNGIGVTQNVLYENQKLIA NQFNSAIGKIQDSLSSTASALGKLQDVVNQNAQALNTLVKQLSSNFGAIS S VLNDILSRLDPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQPELDSIKEELDKIHKGSGGSGDIIKLLNEQVNKE M QSSNLYMSMSSWCYTHSLDGAGLFLFDHAAEEYEHAKKLIIFLNENNVPV QLTSISAPEHKFEGLTQIFQKAYEHEQHISESINNIVDHAIKSKDHATFN FL QWYVAEQHEEEVLFKDILDKIELIGNENHGLYLADQYVKGIAKSRKSGS | 156 |
| 1B-06-PL combo | pCoV172 | PL-S2P.1159op1-del-Ferritin (revert P1143S)-F1148I/Y1155I/F1156H | 1,319 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSFIEDLL FN KVTLADAGFIKQYGDCLGDIAARDLICAQKFNGLTVLPPLLTDEMIAQYT SALLAGTITSGWTFGAGAALQIPFAMQMAYRFNGIGVTQNVLYENQKLIA NQFNSAIGKIQDSLSSTASALGKLQDVVNQNAQALNTLVKQLSSNFGAIS S VLNDILSRLDPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQPELDSIKEELDKIHKNHGSGGSGDIIKLLNEQVN K EMQSSNLYMSMSSWCYTHSLDGAGLFLFDHAAEEYEHAKKLIIFLNENN VPVQLTSISAPEHKFEGLTQIFQKAYEHEQHISESINNIVDHAIKSKDHA TF NFLQWYVAEQHEEEVLFKDILDKIELIGNENHGLYLADQYVKGIAKSRKS GS | 157 |
| 1B-06-PL combo | pCoV173 | PL-S2P.1160op1-del-Ferritin (revert P1143S)-F1148I/Y1155I/F1156H | 1,320 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSFIEDLL FN KVTLADAGFIKQYGDCLGDIAARDLICAQKFNGLTVLPPLLTDEMIAQYT SALLAGTITSGWTFGAGAALQIPFAMQMAYRFNGIGVTQNVLYENQKLIA NQFNSAIGKIQDSLSSTASALGKLQDVVNQNAQALNTLVKQLSSNFGAIS S VLNDILSRLDPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQPELDSIKEELDKIHKNHTGSGGSGDIIKLLNEQV N KEMQSSNLYMSMSSWCYTHSLDGAGLFLFDHAAEEYEHAKKLIIFLNEN NVPVQLTSISAPEHKFEGLTQIFQKAYEHEQHISESINNIVDHAIKSKDH AT FNFLQWYVAEQHEEEVLFKDILDKIELIGNENHGLYLADQYVKGIAKSRK SGS | 158 |
| 1B-05 with SARS 1 motif | pCoV174 | S2P.1154_SARS1-S2chimera-del-Ferritin | 1,310 | VNLTTRTQLPPAYTNSFTRGVYYPDKVFRSSVLHSTQDLFLPFFSNVTWF HAIHVSGTNGTKRFDNPVLPFNDGVYFASTEKSNIIRGWIFGTTLDSKTQ S LLIVNNATNVVIKVCEFQFCNDPFLGVYYHKNNKSWMESEFRVYSSANN CTFEYVSQPFLMDLEGKQGNFKNLREFVFKNIDGYFKIYSKHTPINLVRD L PQGFSALEPLVDLPIGINITRFQTLLALHRSYLTPGDSSSGWTAGAAAYY V GYLQPRTFLLKYNENGTITDAVDCALDPLSETKCTLKSFTVEKGIYQTSN F RVQPTESIVRFPNITNLCPFGEVFNATRFASVYAWNRKRISNCVADYSVL Y NSASFSTFKCYGVSPTKLNDLCFTNVYADSFVIRGDEVRQIAPGQTGKIA D YNYKLPDDFTGCVIAWNSNNLDSKVGGNYNYLYRLFRKSNLKPFERDIST EIYQAGSTPCNGVEGFNCYFPLQSYGFQPTNGVGYQPYRVVVLSFELLHA PATVCGPKKSTNLVKNKCVNFNFNGLTGTGVLTESNKKFLPFQQFGRDIA DTTDAVRDPQTLEILDITPCSFGGVSVITPGTNTSNQVAVLYQDVNCTEV P VAIHADQLTPTWRVYSTGSNVFQTRAGCLIGAEHVNNSYECDIPIGAGIC A SYQTQTNSPGSASSVASQSIIAYTMSLGAENSVAYSNNTIAIPTNFTISV TTE ILPVSMTKTSVDCTMYICGDSTECSNLLLQYGSFCTQLNRALTGIAVEQD K NTQEVFAQVKQIYKTPPIKDFGGFNFSQILPDPSKPSKRSFIEDLLFNKV TL ADAGFIKQYGDCLGDIAARDLICAQKFNGLTVLPPLLTDEMIAQYTSALL AGTITSGWTFGAGAALQIPFAMQMAYRFNGIGVTQNVLYENQKLIANQF NSAIGKIQDSLSSTASALGKLQDVVNQNAQALNTLVKQLSSNFGAISSVL N DILSRLDPPEAEVQIDRLITGRLQSLQTYVTQQLIRAAEIRASANLAATK MS ECVLGQSKRVDFCGKGYHLMSFPQSAPHGVVFLHVTYVPAQEKNFTTAP AICHDGKAHFPREGVFVSNGTHWFVTQRNFYEPQIITTDNTFVSGNCDVV I GIVNNTVYDPLQPELDSFKEELDKGSGGSGDIIKLLNEQVNKEMQSSNLY MSMSSWCYTHSLDGAGLFLFDHAAEEYEHAKKLIIFLNENNVPVQLTSIS APEHKFEGLTQIFQKAYEHEQHISESINNIVDHAIKSKDHATFNFLQWYV A EQHEEEVLFKDILDKIELIGNENHGLYLADQYVKGIAKSRKSGS | 159 |
| 1B-06 with SARS 1 motif | pCoV175 | PL- S2P.1158op1_SARS1-S2chimera-del-Ferritin | 1,318 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTNSPGSASSVASQSIIAYTMSLGAENSVAYSNNTIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSFIEDLL FN KVTLADAGFIKQYGDCLGDIAARDLICAQKFNGLTVLPPLLTDEMIAQYT SALLAGTITSGWTFGAGAALQIPFAMQMAYRFNGIGVTQNVLYENQKLIA NQFNSAIGKIQDSLSSTASALGKLQDVVNQNAQALNTLVKQLSSNFGAIS S VLNDILSRLDPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQSELDSIKEELDKIHKNGSGGSGDIIKLLNEQVNK E MQSSNLYMSMSSWCYTHSLDGAGLFLFDHAAEEYEHAKKLIIFLNENNV PVQLTSISAPEHKFEGLTQIFQKAYEHEQHISESINNIVDHAIKSKDHAT FNF LQWYVAEQHEEEVLFKDILDKIELIGNENHGLYLADQYVKGIAKSRKSGS | 160 |
| 1B-05 with DS2 | pCoV176 | S2P.1154_DS2-del-Ferritin | 1,310 | VNLTTRTQLPPAYTNSFTRGVYYPDKVFRSSVLHSTQDLFLPFFSNVTWF HAIHVSGTNGTKRFDNPVLPFNDGVYFASTEKSNIIRGWIFGTTLDSKTQ S LLIVNNATNVVIKVCEFQFCNDPFLGVYYHKNNKSWMESEFRVYSSANN CTFEYVSQPFLMDLEGKQGNFKNLREFVFKNIDGYFKIYSKHTPINLVRD L PQGFSALEPLVDLPIGINITRFQTLLALHRSYLTPGDSSSGWTAGAAAYY V GYLQPRTFLLKYNENGTITDAVDCALDPLSETKCTLKSFTVEKGIYQTSN F RVQPTESIVRFPNITNLCPFGEVFNATRFASVYAWNRKRISNCVADYSVL Y NSASFSTFKCYGVSPTKLNDLCFTNVYADSFVIRGDEVRQIAPGQTGKIA D YNYKLPDDFTGCVIAWNSNNLDSKVGGNYNYLYRLFRKSNLKPFERDIST EIYQAGSTPCNGVEGFNCYFPLQSYGFQPTNGVGYQPYRVVVLSFELLHA PATVCGPKKSTNLVKNKCVNFNFNGLTGTGVLTESNKKFLPFQQFGRDIA DTTDAVRDPQTLEILDITPCSFGGVSVITPGTNTSNQVAVLYQDVNCTEV P VAIHADQLTPTWRVYSTGSNVFQTRAGCLIGAEHVNNSYECDIPIGAGIC A SYQTQTNSPGSASSVASQSIIAYTMSLGAENSVAYSNNSIAIPTNFTISV TTE ILPVSMTKTSVDCTMYICGDSTECSNLLLQYGSFCTQLNRALTGIAVEQD K CTQEVFAQVKQIYKTPPIKDFGGFNFSQILPDPSKPSKRSFIEDLLFNKV TL ADAGFIKQYGDCLGDIAARDLICAQKFNGLTVLPPLLTDEMIAQYTSALL AGTITSGWTFGAGAALQIPFAMQMAYRFNGIGVTQNVLYENQKLIANQF NSAIGKIQDSLSSTASALGKLQDVVNQNAQALNTLVKQLSSNFGAISSVL N DILSRLDPPEAEVQIDRLITGRLQSLQTYVTQQLIRAAEIRASCNLAATK MS ECVLGQSKRVDFCGKGYHLMSFPQSAPHGVVFLHVTYVPAQEKNFTTAP AICHDGKAHFPREGVFVSNGTHWFVTQRNFYEPQIITTDNTFVSGNCDVV I GIVNNTVYDPLQPELDSFKEELDKGSGGSGDIIKLLNEQVNKEMQSSNLY MSMSSWCYTHSLDGAGLFLFDHAAEEYEHAKKLIIFLNENNVPVQLTSIS APEHKFEGLTQIFQKAYEHEQHISESINNIVDHAIKSKDHATFNFLQWYV A EQHEEEVLFKDILDKIELIGNENHGLYLADQYVKGIAKSRKSGS | 161 |
| 1B-06-PL with DS2 | pCoV177 | PL-S2P.1158_DS2-del-Ferritin | 1,318 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKCTQEVFAQVKQIYKTPPIKDFGGFNFSQILPDPSKPSKRSFIEDLL FN KVTLADAGFIKQYGDCLGDIAARDLICAQKFNGLTVLPPLLTDEMIAQYT SALLAGTITSGWTFGAGAALQIPFAMQMAYRFNGIGVTQNVLYENQKLIA NQFNSAIGKIQDSLSSTASALGKLQDVVNQNAQALNTLVKQLSSNFGAIS S VLNDILSRLDPPEAEVQIDRLITGRLQSLQTYVTQQLIRAAEIRASCNLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQSELDSIKEELDKIHKNGSGGSGDIIKLLNEQVNK E MQSSNLYMSMSSWCYTHSLDGAGLFLFDHAAEEYEHAKKLIIFLNENNV PVQLTSISAPEHKFEGLTQIFQKAYEHEQHISESINNIVDHAIKSKDHAT FNF LQWYVAEQHEEEVLFKDILDKIELIGNENHGLYLADQYVKGIAKSRKSGS | 162 |
| 1B-05 with DS3 | pCoV178 | S2P.1154_DS3-del-Ferritin | 1,310 | VNLTTRTQLPPAYTNSFTRGVYYPDKVFRSSVLHSTQDLFLPFFSNVTWF HAIHVSGTNGTKRFDNPVLPFNDGVYFASTEKSNIIRGWIFGTTLDSKTQ S LLIVNNATNVVIKVCEFQFCNDPFLGVYYHKNNKSWMESEFRVYSSANN CTFEYVSQPFLMDLEGKQGNFKNLREFVFKNIDGYFKIYSKHTPINLVRD L PQGFSALEPLVDLPIGINITRFQTLLALHRSYLTPGDSSSGWTAGAAAYY V GYLQPRTFLLKYNENGTITDAVDCALDPLSETKCTLKSFTVEKGIYQTSN F RVQPTESIVRFPNITNLCPFGEVFNATRFASVYAWNRKRISNCVADYSVL Y NSASFSTFKCYGVSPTKLNDLCFTNVYADSFVIRGDEVRQIAPGQTGKIA D YNYKLPDDFTGCVIAWNSNNLDSKVGGNYNYLYRLFRKSNLKPFERDIST EIYQAGSTPCNGVEGFNCYFPLQSYGFQPTNGVGYQPYRVVVLSFELLHA PATVCGPKKSTNLVKNKCVNFNFNGLTGTGVLTESNKKFLPFQQFGRDIA DTTDAVRDPQTLEILDITPCSFGGVSVITPGTNTSNQVAVLYQDVNCTEV P VAIHADQLTPTWRVYSTGSNVFQTRAGCLIGAEHVNNSYECDIPIGAGIC A SYQTQTNSPGSASSVASQSIIAYTMSLGAENSVAYSNNSIAIPTNFTISV TTE ILPVSMTCTSVDCTMYICGDSTECSNLLLQYGSFCTQLNRALTGICVEQD K NTQEVFAQVKQIYKTPPIKDFGGFNFSQILPDPSKPSKRSFIEDLLFNKV TL ADAGFIKQYGDCLGDIAARDLICAQKFNGLTVLPPLLTDEMIAQYTSALL AGTITSGWTFGAGAALQIPFAMQMAYRFNGIGVTQNVLYENQKLIANQF NSAIGKIQDSLSSTASALGKLQDVVNQNAQALNTLVKQLSSNFGAISSVL N DILSRLDPPEAEVQIDRLITGRLQSLQTYVTQQLIRAAEIRASANLAATK MS ECVLGQSKRVDFCGKGYHLMSFPQSAPHGVVFLHVTYVPAQEKNFTTAP AICHDGKAHFPREGVFVSNGTHWFVTQRNFYEPQIITTDNTFVSGNCDVV I GIVNNTVYDPLQPELDSFKEELDKGSGGSGDIIKLLNEQVNKEMQSSNLY MSMSSWCYTHSLDGAGLFLFDHAAEEYEHAKKLIIFLNENNVPVQLTSIS APEHKFEGLTQIFQKAYEHEQHISESINNIVDHAIKSKDHATFNFLQWYV A EQHEEEVLFKDILDKIELIGNENHGLYLADQYVKGIAKSRKSGS | 163 |
| 1B-06-PL with DS3 | pCoV179 | PL-S2P.1158_DS3-del-Ferritin | 1,318 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTCTSVDCTMYICGDSTECSNLLLQYGSFCTQLNRALTGIC V EQDKNTQEVFAQVKQIYKTPPIKDFGGFNFSQILPDPSKPSKRSFIEDLL FN KVTLADAGFIKQYGDCLGDIAARDLICAQKFNGLTVLPPLLTDEMIAQYT SALLAGTITSGWTFGAGAALQIPFAMQMAYRFNGIGVTQNVLYENQKLIA NQFNSAIGKIQDSLSSTASALGKLQDVVNQNAQALNTLVKQLSSNFGAIS S VLNDILSRLDPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQSELDSIKEELDKIHKNGSGGSGDIIKLLNEQVNK E MQSSNLYMSMSSWCYTHSLDGAGLFLFDHAAEEYEHAKKLIIFLNENNV PVQLTSISAPEHKFEGLTQIFQKAYEHEQHISESINNIVDHAIKSKDHAT FNF LQWYVAEQHEEEVLFKDILDKIELIGNENHGLYLADQYVKGIAKSRKSGS | 164 |
| 1B-05 with more UP | pCoV180 | S2P.1154_moreUP-del-Ferritin (Henderson, et al.) | 1,310 | VNLTTRTQLPPAYTNSFTRGVYYPDKVFRSSVLHSTQDLFLPFFSNVTWF HAIHVSGTNGTKRFDNPVLPFNDGVYFASTEKSNIIRGWIFGTTLDSKTQ S LLIVNNATNVVIKVCEFQFCNDPFLGVYYHKNNKSWMESEFRVYSSANN CTFEYVSQPFLMDLEGKQGNFKNLREFVFKNIDGYFKIYSKHTPINLVRD L PQGFSALEPLVDLPIGINITRFQTLLALHRSYLTPGDSSSGWTAGAAAYY V GYLQPRTFLLKYNENGTITDAVDCALDPLSETKCTLKSFTVEKGIYQTSN F RVQPTESIVRFPNITNLCPFGEVFNATRFASVYAWNRKRISNCVADYSVL Y NSASFSTFKCYGVSPTKLNDLCFTNVYADSFVIRGDEVRQIAPGQTGKIA D YNYKLPDDFTGCVIAWNSNNLDSKVGGNYNYLYRLFRKSNLKPFERDIST EIYQAGSTPCNGVEGFNCYFPLQSYGFQPTNGVGYQPYRVVVLSFELLHA PATVCGPKKSTNLVKNKCVNFNFNGLTGTGVLTESNKKFLPFQQFGRDIL DTIDAVRDPQTLEILDITPCSFGGVSVITPGTNTSNQVAVLYQDVNCTEV P VAIHADQLTPTWRVYSTGSNVFQTRAGCLIGAEHVNNSYECDIPIGAGIC A SYQTQTNSPGSASSVASQSIIAYTMSLGAENSVAYSNNSIAIPTNFTISV TTE ILPVSMTKTSVDCTMYICGDSTECSNLLLQYGSFCTQLNRALTGIAVEQD K NTQEVFAQVKQIYKTPPIKDFGGFNFSQILPDPSKPSKRSFIEDLLFNKV TL ADAGFIKQYGDCLGDIAARDLICAQKYIGLTVLPPLLTDEMIAQYTSALL A GTITSGWTFGAGAALQIPFAMQMAYRFNGIGVTQNVLYENQKLIANQFNS AIGKIQDSLSSTASALGKLQDVVNQNAQALNTLVKQLSSNFGAISSVLND I LSRLDPPEAEVQIDRLITGRLQSLQTYVTQQLIRAAEIRASANLAATKMS E CVLGQSKRVDFCGKGYHLMSFPQSAPHGVVFLHVTYVPAQEKNFTTAPA ICHDGKAHFPREGVFVSNGTHWFVTQRNFYEPQIITTDNTFVSGNCDVVI G IVNNTVYDPLQPELDSFKEELDKGSGGSGDIIKLLNEQVNKEMQSSNLYM SMSSWCYTHSLDGAGLFLFDHAAEEYEHAKKLIIFLNENNVPVQLTSISA P EHKFEGLTQIFQKAYEHEQHISESINNIVDHAIKSKDHATFNFLQWYVAE Q HEEEVLFKDILDKIELIGNENHGLYLADQYVKGIAKSRKSGS | 165 |
| 1B-06-PL with more UP | pCoV181 | PL-S2P.1158op1_moreUP-del-Ferritin (Henderson, et al.) | 1,318 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DILDTIDAVRDPQTLEILDITPCSFGGVSVITPGTNTSNQVAVLYQDVNC TE VPVAIHADQLTPTWRVYSTGSNVFQTRAGCLIGAEHVNNSYECDIPIGAG I CASYQTQTNSPGSASSVASQSIIAYTMSLGAENSVAYSNNSIAIPTNFTI SV TTEILPVSMTKTSVDCTMYICGDSTECSNLLLQYGSFCTQLNRALTGIAV E QDKNTQEVFAQVKQIYKTPPIKDFGGFNFSQILPDPSKPSKRSFIEDLLF NK VTLADAGFIKQYGDCLGDIAARDLICAQKYIGLTVLPPLLTDEMIAQYTS A LLAGTITSGWTFGAGAALQIPFAMQMAYRFNGIGVTQNVLYENQKLIAN QFNSAIGKIQDSLSSTASALGKLQDVVNQNAQALNTLVKQLSSNFGAISS V LNDILSRLDPPEAEVQIDRLITGRLQSLQTYVTQQLIRAAEIRASANLAA TK MSECVLGQSKRVDFCGKGYHLMSFPQSAPHGVVFLHVTYVPAQEKNFTT APAICHDGKAHFPREGVFVSNGTHWFVTQRNFYEPQIITTDNTFVSGNCD VVIGIVNNTVYDPLQSELDSIKEELDKIHKNGSGGSGDIIKLLNEQVNKE M QSSNLYMSMSSWCYTHSLDGAGLFLFDHAAEEYEHAKKLIIFLNENNVPV QLTSISAPEHKFEGLTQIFQKAYEHEQHISESINNIVDHAIKSKDHATFN FL QWYVAEQHEEEVLFKDILDKIELIGNENHGLYLADQYVKGIAKSRKSGS | 166 |
| 1B-05 with HexaPro | pCoV182 | S2P-HexaPro.1154-del-Ferritin | 1,310 | VNLTTRTQLPPAYTNSFTRGVYYPDKVFRSSVLHSTQDLFLPFFSNVTWF HAIHVSGTNGTKRFDNPVLPFNDGVYFASTEKSNIIRGWIFGTTLDSKTQ S LLIVNNATNVVIKVCEFQFCNDPFLGVYYHKNNKSWMESEFRVYSSANN CTFEYVSQPFLMDLEGKQGNFKNLREFVFKNIDGYFKIYSKHTPINLVRD L PQGFSALEPLVDLPIGINITRFQTLLALHRSYLTPGDSSSGWTAGAAAYY V GYLQPRTFLLKYNENGTITDAVDCALDPLSETKCTLKSFTVEKGIYQTSN F RVQPTESIVRFPNITNLCPFGEVFNATRFASVYAWNRKRISNCVADYSVL Y NSASFSTFKCYGVSPTKLNDLCFTNVYADSFVIRGDEVRQIAPGQTGKIA D YNYKLPDDFTGCVIAWNSNNLDSKVGGNYNYLYRLFRKSNLKPFERDIST EIYQAGSTPCNGVEGFNCYFPLQSYGFQPTNGVGYQPYRVVVLSFELLHA PATVCGPKKSTNLVKNKCVNFNFNGLTGTGVLTESNKKFLPFQQFGRDIA DTTDAVRDPQTLEILDITPCSFGGVSVITPGTNTSNQVAVLYQDVNCTEV P VAIHADQLTPTWRVYSTGSNVFQTRAGCLIGAEHVNNSYECDIPIGAGIC A SYQTQTNSPGSASSVASQSIIAYTMSLGAENSVAYSNNSIAIPTNFTISV TTE ILPVSMTKTSVDCTMYICGDSTECSNLLLQYGSFCTQLNRALTGIAVEQD K NTQEVFAQVKQIYKTPPIKDFGGFNFSQILPDPSKPSKRSPIEDLLFNKV TL ADAGFIKQYGDCLGDIAARDLICAQKFNGLTVLPPLLTDEMIAQYTSALL AGTITSGWTFGAGPALQIPFPMQMAYRFNGIGVTQNVLYENQKLIANQFN SAIGKIQDSLSSTPSALGKLQDVVNQNAQALNTLVKQLSSNFGAISSVLN D ILSRLDPPEAEVQIDRLITGRLQSLQTYVTQQLIRAAEIRASANLAATKM SE CVLGQSKRVDFCGKGYHLMSFPQSAPHGVVFLHVTYVPAQEKNFTTAPA ICHDGKAHFPREGVFVSNGTHWFVTQRNFYEPQIITTDNTFVSGNCDVVI G IVNNTVYDPLQPELDSFKEELDKGSGGSGDIIKLLNEQVNKEMQSSNLYM SMSSWCYTHSLDGAGLFLFDHAAEEYEHAKKLIIFLNENNVPVQLTSISA P EHKFEGLTQIFQKAYEHEQHISESINNIVDHAIKSKDHATFNFLQWYVAE Q HEEEVLFKDILDKIELIGNENHGLYLADQYVKGIAKSRKSGS | 167 |
| 1B-06-PL with HexaPro | pCoV183 | S2P-HexaPro.115 8 op1-del-Ferritin (aka 1B-06-PL with HexaPro) | 1,318 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSPIEDLL FN KVTLADAGFIKQYGDCLGDIAARDLICAQKFNGLTVLPPLLTDEMIAQYT SALLAGTITSGWTFGAGPALQIPFPMQMAYRFNGIGVTQNVLYENQKLIA NQFNSAIGKIQDSLSSTPSALGKLQDVVNQNAQALNTLVKQLSSNFGAIS S VLNDILSRLDPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQSELDSIKEELDKIHKNGSGGSGDIIKLLNEQVNK E MQSSNLYMSMSSWCYTHSLDGAGLFLFDHAAEEYEHAKKLIIFLNENNV PVQLTSISAPEHKFEGLTQIFQKAYEHEQHISESINNIVDHAIKSKDHAT FNF LQWYVAEQHEEEVLFKDILDKIELIGNENHGLYLADQYVKGIAKSRKSGS | 168 |
| 167 with HexaPro | pCoV184 | S2P-HexaPro.1158-del-Ferritin (F1148I/Y115 5I/F1156H) | 1,318 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSPIEDLL FN KVTLADAGFIKQYGDCLGDIAARDLICAQKFNGLTVLPPLLTDEMIAQYT SALLAGTITSGWTFGAGPALQIPFPMQMAYRFNGIGVTQNVLYENQKLIA NQFNSAIGKIQDSLSSTPSALGKLQDVVNQNAQALNTLVKQLSSNFGAIS S VLNDILSRLDPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQPELDSIKEELDKIHKNGSGGSGDIIKLLNEQVNK E MQSSNLYMSMSSWCYTHSLDGAGLFLFDHAAEEYEHAKKLIIFLNENNV PVQLTSISAPEHKFEGLTQIFQKAYEHEQHISESINNIVDHAIKSKDHAT FNF LQWYVAEQHEEEVLFKDILDKIELIGNENHGLYLADQYVKGIAKSRKSGS | 169 |
| 47 with HexaPro | pCoV185 | S2P-HexaPro.1240-Fd-His-TwinStrep (McLellan Lab) | 1,271 | VNLTTRTQLPPAYTNSFTRGVYYPDKVFRSSVLHSTQDLFLPFFSNVTWF HAIHVSGTNGTKRFDNPVLPFNDGVYFASTEKSNIIRGWIFGTTLDSKTQ S LLIVNNATNVVIKVCEFQFCNDPFLGVYYHKNNKSWMESEFRVYSSANN CTFEYVSQPFLMDLEGKQGNFKNLREFVFKNIDGYFKIYSKHTPINLVRD L PQGFSALEPLVDLPIGINITRFQTLLALHRSYLTPGDSSSGWTAGAAAYY V GYLQPRTFLLKYNENGTITDAVDCALDPLSETKCTLKSFTVEKGIYQTSN F RVQPTESIVRFPNITNLCPFGEVFNATRFASVYAWNRKRISNCVADYSVL Y NSASFSTFKCYGVSPTKLNDLCFTNVYADSFVIRGDEVRQIAPGQTGKIA D YNYKLPDDFTGCVIAWNSNNLDSKVGGNYNYLYRLFRKSNLKPFERDIST EIYQAGSTPCNGVEGFNCYFPLQSYGFQPTNGVGYQPYRVVVLSFELLHA PATVCGPKKSTNLVKNKCVNFNFNGLTGTGVLTESNKKFLPFQQFGRDIA DTTDAVRDPQTLEILDITPCSFGGVSVITPGTNTSNQVAVLYQDVNCTEV P VAIHADQLTPTWRVYSTGSNVFQTRAGCLIGAEHVNNSYECDIPIGAGIC A SYQTQTNSPGSASSVASQSIIAYTMSLGAENSVAYSNNSIAIPTNFTISV TTE ILPVSMTKTSVDCTMYICGDSTECSNLLLQYGSFCTQLNRALTGIAVEQD K NTQEVFAQVKQIYKTPPIKDFGGFNFSQILPDPSKPSKRSPIEDLLFNKV TL ADAGFIKQYGDCLGDIAARDLICAQKFNGLTVLPPLLTDEMIAQYTSALL AGTITSGWTFGAGPALQIPFPMQMAYRFNGIGVTQNVLYENQKLIANQFN SAIGKIQDSLSSTPSALGKLQDVVNQNAQALNTLVKQLSSNFGAISSVLN D ILSRLDPPEAEVQIDRLITGRLQSLQTYVTQQLIRAAEIRASANLAATKM SE CVLGQSKRVDFCGKGYHLMSFPQSAPHGVVFLHVTYVPAQEKNFTTAPA ICHDGKAHFPREGVFVSNGTHWFVTQRNFYEPQIITTDNTFVSGNCDVVI G IVNNTVYDPLQPELDSFKEELDKYFKNHTSPDVDLGDISGINASVVNIQK EI DRLNEVAKNLNESLIDLQELGKYEQGSGYIPEAPRDGQAYVRKDGEWVL LSTFLGLEVLFQGPSAWSHPQFEKGGGSGGGSGGSAWSHPQFEKGSHHH HHHHH | 170 |
| 1B-06-PL with HexaPro + D614G | pCoV186 | S2P-HexaPro.1158 op1-del-Ferritin-D614G (aka 1B-06-PL with HexaPro and D614G) | 1,318 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQGVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSPIEDLL FN KVTLADAGFIKQYGDCLGDIAARDLICAQKFNGLTVLPPLLTDEMIAQYT SALLAGTITSGWTFGAGPALQIPFPMQMAYRFNGIGVTQNVLYENQKLIA NQFNSAIGKIQDSLSSTPSALGKLQDVVNQNAQALNTLVKQLSSNFGAIS S VLNDILSRLDPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQSELDSIKEELDKIHKNGSGGSGDIIKLLNEQVNK E MQSSNLYMSMSSWCYTHSLDGAGLFLFDHAAEEYEHAKKLIIFLNENNV PVQLTSISAPEHKFEGLTQIFQKAYEHEQHISESINNIVDHAIKSKDHAT FNF LQWYVAEQHEEEVLFKDILDKIELIGNENHGLYLADQYVKGIAKSRKSGS | 171 |
| 1B-08-PL with D614G | pCoV187 | S2P.1158op1x 2-del-Ferritin-D614G | 1,332 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQGVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSFIEDLL FN KVTLADAGFIKQYGDCLGDIAARDLICAQKFNGLTVLPPLLTDEMIAQYT SALLAGTITSGWTFGAGAALQIPFAMQMAYRFNGIGVTQNVLYENQKLIA NQFNSAIGKIQDSLSSTASALGKLQDVVNQNAQALNTLVKQLSSNFGAIS S VLNDILSRLDPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQSELDSIKEELDKIHKNLDSIKEELDKIHKNGSGG SG DIIKLLNEQVNKEMQSSNLYMSMSSWCYTHSLDGAGLFLFDHAAEEYEH AKKLIIFLNENNVPVQLTSISAPEHKFEGLTQIFQKAYEHEQHISESINN IVD HAIKSKDHATFNFLQWYVAEQHEEEVLFKDILDKIELIGNENHGLYLADQ YVKGIAKSRKSGS | 172 |
| 1B-08-PL with HexaPro | pCoV188 | S2P-HexaPro.1158 op1x2-del-Ferritin | 1,332 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSPIEDLL FN KVTLADAGFIKQYGDCLGDIAARDLICAQKFNGLTVLPPLLTDEMIAQYT SALLAGTITSGWTFGAGPALQIPFPMQMAYRFNGIGVTQNVLYENQKLIA NQFNSAIGKIQDSLSSTPSALGKLQDVVNQNAQALNTLVKQLSSNFGAIS S VLNDILSRLDPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQSELDSIKEELDKIHKNLDSIKEELDKIHKNGSGG SG DIIKLLNEQVNKEMQSSNLYMSMSSWCYTHSLDGAGLFLFDHAAEEYEH AKKLIIFLNENNVPVQLTSISAPEHKFEGLTQIFQKAYEHEQHISESINN IVD HAIKSKDHATFNFLQWYVAEQHEEEVLFKDILDKIELIGNENHGLYLADQ YVKGIAKSRKSGS | 173 |
| 1B-08-PL with HexaPro + D614G | pCoV189 | S2P-HexaPro.1158 op1x2-del-Ferritin-D614G | 1,332 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQGVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSPIEDLL FN KVTLADAGFIKQYGDCLGDIAARDLICAQKFNGLTVLPPLLTDEMIAQYT SALLAGTITSGWTFGAGPALQIPFPMQMAYRFNGIGVTQNVLYENQKLIA NQFNSAIGKIQDSLSSTPSALGKLQDVVNQNAQALNTLVKQLSSNFGAIS S VLNDILSRLDPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQSELDSIKEELDKIHKNLDSIKEELDKIHKNGSGG SG DIIKLLNEQVNKEMQSSNLYMSMSSWCYTHSLDGAGLFLFDHAAEEYEH AKKLIIFLNENNVPVQLTSISAPEHKFEGLTQIFQKAYEHEQHISESINN IVD HAIKSKDHATFNFLQWYVAEQHEEEVLFKDILDKIELIGNENHGLYLADQ YVKGIAKSRKSGS | 174 |
| 1B-05 with D614G | pCoV190 | S2P.1154-del-Ferritin-D614G | 1,310 | VNLTTRTQLPPAYTNSFTRGVYYPDKVFRSSVLHSTQDLFLPFFSNVTWF HAIHVSGTNGTKRFDNPVLPFNDGVYFASTEKSNIIRGWIFGTTLDSKTQ S LLIVNNATNVVIKVCEFQFCNDPFLGVYYHKNNKSWMESEFRVYSSANN CTFEYVSQPFLMDLEGKQGNFKNLREFVFKNIDGYFKIYSKHTPINLVRD L PQGFSALEPLVDLPIGINITRFQTLLALHRSYLTPGDSSSGWTAGAAAYY V GYLQPRTFLLKYNENGTITDAVDCALDPLSETKCTLKSFTVEKGIYQTSN F RVQPTESIVRFPNITNLCPFGEVFNATRFASVYAWNRKRISNCVADYSVL Y NSASFSTFKCYGVSPTKLNDLCFTNVYADSFVIRGDEVRQIAPGQTGKIA D YNYKLPDDFTGCVIAWNSNNLDSKVGGNYNYLYRLFRKSNLKPFERDIST EIYQAGSTPCNGVEGFNCYFPLQSYGFQPTNGVGYQPYRVVVLSFELLHA PATVCGPKKSTNLVKNKCVNFNFNGLTGTGVLTESNKKFLPFQQFGRDIA DTTDAVRDPQTLEILDITPCSFGGVSVITPGTNTSNQVAVLYQGVNCTEV P VAIHADQLTPTWRVYSTGSNVFQTRAGCLIGAEHVNNSYECDIPIGAGIC A SYQTQTNSPGSASSVASQSIIAYTMSLGAENSVAYSNNSIAIPTNFTISV TTE ILPVSMTKTSVDCTMYICGDSTECSNLLLQYGSFCTQLNRALTGIAVEQD K NTQEVFAQVKQIYKTPPIKDFGGFNFSQILPDPSKPSKRSFIEDLLFNKV TL ADAGFIKQYGDCLGDIAARDLICAQKFNGLTVLPPLLTDEMIAQYTSALL AGTITSGWTFGAGAALQIPFAMQMAYRFNGIGVTQNVLYENQKLIANQF NSAIGKIQDSLSSTASALGKLQDVVNQNAQALNTLVKQLSSNFGAISSVL N DILSRLDPPEAEVQIDRLITGRLQSLQTYVTQQLIRAAEIRASANLAATK MS ECVLGQSKRVDFCGKGYHLMSFPQSAPHGVVFLHVTYVPAQEKNFTTAP AICHDGKAHFPREGVFVSNGTHWFVTQRNFYEPQIITTDNTFVSGNCDVV I GIVNNTVYDPLQPELDSFKEELDKGSGGSGDIIKLLNEQVNKEMQSSNLY MSMSSWCYTHSLDGAGLFLFDHAAEEYEHAKKLIIFLNENNVPVQLTSIS APEHKFEGLTQIFQKAYEHEQHISESINNIVDHAIKSKDHATFNFLQWYV A EQHEEEVLFKDILDKIELIGNENHGLYLADQYVKGIAKSRKSGS | 175 |
| 1B-06-PL with DS1 + HexaPro | pCoV191 | S2P-HexaPro.1158 op1-DS1-del-Ferritin | 1,318 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVCPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSPIEDLL FN KVTLADAGFIKQYGDCLGDIAARDLICAQKFNGLTVLPPLLTDEMIAQYT SALLAGTITSGWTFGAGPALQIPFPMQMAYRFNGIGVTQNVLYENQKLIA NQFNSAIGKIQDSLSSTPSALGKLQDVVNQNAQALNTLVKQLSSNFGAIS S VLNDILSRLCPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQSELDSIKEELDKIHKNGSGGSGDIIKLLNEQVNK E MQSSNLYMSMSSWCYTHSLDGAGLFLFDHAAEEYEHAKKLIIFLNENNV PVQLTSISAPEHKFEGLTQIFQKAYEHEQHISESINNIVDHAIKSKDHAT FNF LQWYVAEQHEEEVLFKDILDKIELIGNENHGLYLADQYVKGIAKSRKSGS | 176 |
| 1B-06-PL with DS1 + HexaPro + D614G | pCoV192 | S2P-HexaPro.1158 op1-DS1-del-Ferritin-D614G | 1,318 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVCPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQGVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSPIEDLL FN KVTLADAGFIKQYGDCLGDIAARDLICAQKFNGLTVLPPLLTDEMIAQYT SALLAGTITSGWTFGAGPALQIPFPMQMAYRFNGIGVTQNVLYENQKLIA NQFNSAIGKIQDSLSSTPSALGKLQDVVNQNAQALNTLVKQLSSNFGAIS S VLNDILSRLCPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQSELDSIKEELDKIHKNGSGGSGDIIKLLNEQVNK E MQSSNLYMSMSSWCYTHSLDGAGLFLFDHAAEEYEHAKKLIIFLNENNV PVQLTSISAPEHKFEGLTQIFQKAYEHEQHISESINNIVDHAIKSKDHAT FNF LQWYVAEQHEEEVLFKDILDKIELIGNENHGLYLADQYVKGIAKSRKSGS | 177 |
| 1B-08-PL with DS1 | pCoV193 | S2P.1158op1x 2-DS1-del-Ferritin | 1,332 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVCPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSFIEDLL FN KVTLADAGFIKQYGDCLGDIAARDLICAQKFNGLTVLPPLLTDEMIAQYT SALLAGTITSGWTFGAGAALQIPFAMQMAYRFNGIGVTQNVLYENQKLIA NQFNSAIGKIQDSLSSTASALGKLQDVVNQNAQALNTLVKQLSSNFGAIS S VLNDILSRLCPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQSELDSIKEELDKIHKNLDSIKEELDKIHKNGSGG SG DIIKLLNEQVNKEMQSSNLYMSMSSWCYTHSLDGAGLFLFDHAAEEYEH AKKLIIFLNENNVPVQLTSISAPEHKFEGLTQIFQKAYEHEQHISESINN IVD HAIKSKDHATFNFLQWYVAEQHEEEVLFKDILDKIELIGNENHGLYLADQ YVKGIAKSRKSGS | 178 |
| 1B-05 with HexaPro + D614G | pCoV194 | S2P-HexaPro.1154-del-Ferritin-D614G | 1,310 | VNLTTRTQLPPAYTNSFTRGVYYPDKVFRSSVLHSTQDLFLPFFSNVTWF HAIHVSGTNGTKRFDNPVLPFNDGVYFASTEKSNIIRGWIFGTTLDSKTQ S LLIVNNATNVVIKVCEFQFCNDPFLGVYYHKNNKSWMESEFRVYSSANN CTFEYVSQPFLMDLEGKQGNFKNLREFVFKNIDGYFKIYSKHTPINLVRD L PQGFSALEPLVDLPIGINITRFQTLLALHRSYLTPGDSSSGWTAGAAAYY V GYLQPRTFLLKYNENGTITDAVDCALDPLSETKCTLKSFTVEKGIYQTSN F RVQPTESIVRFPNITNLCPFGEVFNATRFASVYAWNRKRISNCVADYSVL Y NSASFSTFKCYGVSPTKLNDLCFTNVYADSFVIRGDEVRQIAPGQTGKIA D YNYKLPDDFTGCVIAWNSNNLDSKVGGNYNYLYRLFRKSNLKPFERDIST EIYQAGSTPCNGVEGFNCYFPLQSYGFQPTNGVGYQPYRVVVLSFELLHA PATVCGPKKSTNLVKNKCVNFNFNGLTGTGVLTESNKKFLPFQQFGRDIA DTTDAVRDPQTLEILDITPCSFGGVSVITPGTNTSNQVAVLYQGVNCTEV P VAIHADQLTPTWRVYSTGSNVFQTRAGCLIGAEHVNNSYECDIPIGAGIC A SYQTQTNSPGSASSVASQSIIAYTMSLGAENSVAYSNNSIAIPTNFTISV TTE ILPVSMTKTSVDCTMYICGDSTECSNLLLQYGSFCTQLNRALTGIAVEQD K NTQEVFAQVKQIYKTPPIKDFGGFNFSQILPDPSKPSKRSPIEDLLFNKV TL ADAGFIKQYGDCLGDIAARDLICAQKFNGLTVLPPLLTDEMIAQYTSALL AGTITSGWTFGAGPALQIPFPMQMAYRFNGIGVTQNVLYENQKLIANQFN SAIGKIQDSLSSTPSALGKLQDVVNQNAQALNTLVKQLSSNFGAISSVLN D ILSRLDPPEAEVQIDRLITGRLQSLQTYVTQQLIRAAEIRASANLAATKM SE CVLGQSKRVDFCGKGYHLMSFPQSAPHGVVFLHVTYVPAQEKNFTTAPA ICHDGKAHFPREGVFVSNGTHWFVTQRNFYEPQIITTDNTFVSGNCDVVI G IVNNTVYDPLQPELDSFKEELDKGSGGSGDIIKLLNEQVNKEMQSSNLYM SMSSWCYTHSLDGAGLFLFDHAAEEYEHAKKLIIFLNENNVPVQLTSISA P EHKFEGLTQIFQKAYEHEQHISESINNIVDHAIKSKDHATFNFLQWYVAE Q HEEEVLFKDILDKIELIGNENHGLYLADQYVKGIAKSRKSGS | 179 |
| 1B-05 with DS1 + HexaPro | pCoV195 | S2P-HexaPro.1154-DS1-del-Ferritin | 1,310 | VNLTTRTQLPPAYTNSFTRGVYYPDKVFRSSVLHSTQDLFLPFFSNVTWF HAIHVSGTNGTKRFDNPVLPFNDGVYFASTEKSNIIRGWIFGTTLDSKTQ S LLIVNNATNVVIKVCEFQFCNDPFLGVYYHKNNKSWMESEFRVYSSANN CTFEYVSQPFLMDLEGKQGNFKNLREFVFKNIDGYFKIYSKHTPINLVRD L PQGFSALEPLVDLPIGINITRFQTLLALHRSYLTPGDSSSGWTAGAAAYY V GYLQPRTFLLKYNENGTITDAVDCALDPLSETKCTLKSFTVEKGIYQTSN F RVQPTESIVRFPNITNLCPFGEVFNATRFASVYAWNRKRISNCVADYSVL Y NSASFSTFKCYGVCPTKLNDLCFTNVYADSFVIRGDEVRQIAPGQTGKIA D YNYKLPDDFTGCVIAWNSNNLDSKVGGNYNYLYRLFRKSNLKPFERDIST EIYQAGSTPCNGVEGFNCYFPLQSYGFQPTNGVGYQPYRVVVLSFELLHA PATVCGPKKSTNLVKNKCVNFNFNGLTGTGVLTESNKKFLPFQQFGRDIA DTTDAVRDPQTLEILDITPCSFGGVSVITPGTNTSNQVAVLYQDVNCTEV P VAIHADQLTPTWRVYSTGSNVFQTRAGCLIGAEHVNNSYECDIPIGAGIC A SYQTQTNSPGSASSVASQSIIAYTMSLGAENSVAYSNNSIAIPTNFTISV TTE ILPVSMTKTSVDCTMYICGDSTECSNLLLQYGSFCTQLNRALTGIAVEQD K NTQEVFAQVKQIYKTPPIKDFGGFNFSQILPDPSKPSKRSPIEDLLFNKV TL ADAGFIKQYGDCLGDIAARDLICAQKFNGLTVLPPLLTDEMIAQYTSALL AGTITSGWTFGAGPALQIPFPMQMAYRFNGIGVTQNVLYENQKLIANQFN SAIGKIQDSLSSTPSALGKLQDVVNQNAQALNTLVKQLSSNFGAISSVLN D ILSRLCPPEAEVQIDRLITGRLQSLQTYVTQQLIRAAEIRASANLAATKM SE CVLGQSKRVDFCGKGYHLMSFPQSAPHGVVFLHVTYVPAQEKNFTTAPA ICHDGKAHFPREGVFVSNGTHWFVTQRNFYEPQIITTDNTFVSGNCDVVI G IVNNTVYDPLQPELDSFKEELDKGSGGSGDIIKLLNEQVNKEMQSSNLYM SMSSWCYTHSLDGAGLFLFDHAAEEYEHAKKLIIFLNENNVPVQLTSISA P EHKFEGLTQIFQKAYEHEQHISESINNIVDHAIKSKDHATFNFLQWYVAE Q HEEEVLFKDILDKIELIGNENHGLYLADQYVKGIAKSRKSGS | 180 |
| 1B-05 with DS1 + HexaPro + D614G | pCoV196 | S2P-HexaPro.1154-DS1-del-Ferritin-D614G | 1,310 | VNLTTRTQLPPAYTNSFTRGVYYPDKVFRSSVLHSTQDLFLPFFSNVTWF HAIHVSGTNGTKRFDNPVLPFNDGVYFASTEKSNIIRGWIFGTTLDSKTQ S LLIVNNATNVVIKVCEFQFCNDPFLGVYYHKNNKSWMESEFRVYSSANN CTFEYVSQPFLMDLEGKQGNFKNLREFVFKNIDGYFKIYSKHTPINLVRD L PQGFSALEPLVDLPIGINITRFQTLLALHRSYLTPGDSSSGWTAGAAAYY V GYLQPRTFLLKYNENGTITDAVDCALDPLSETKCTLKSFTVEKGIYQTSN F RVQPTESIVRFPNITNLCPFGEVFNATRFASVYAWNRKRISNCVADYSVL Y NSASFSTFKCYGVCPTKLNDLCFTNVYADSFVIRGDEVRQIAPGQTGKIA D YNYKLPDDFTGCVIAWNSNNLDSKVGGNYNYLYRLFRKSNLKPFERDIST EIYQAGSTPCNGVEGFNCYFPLQSYGFQPTNGVGYQPYRVVVLSFELLHA PATVCGPKKSTNLVKNKCVNFNFNGLTGTGVLTESNKKFLPFQQFGRDIA DTTDAVRDPQTLEILDITPCSFGGVSVITPGTNTSNQVAVLYQGVNCTEV P VAIHADQLTPTWRVYSTGSNVFQTRAGCLIGAEHVNNSYECDIPIGAGIC A SYQTQTNSPGSASSVASQSIIAYTMSLGAENSVAYSNNSIAIPTNFTISV TTE ILPVSMTKTSVDCTMYICGDSTECSNLLLQYGSFCTQLNRALTGIAVEQD K NTQEVFAQVKQIYKTPPIKDFGGFNFSQILPDPSKPSKRSPIEDLLFNKV TL ADAGFIKQYGDCLGDIAARDLICAQKFNGLTVLPPLLTDEMIAQYTSALL AGTITSGWTFGAGPALQIPFPMQMAYRFNGIGVTQNVLYENQKLIANQFN SAIGKIQDSLSSTPSALGKLQDVVNQNAQALNTLVKQLSSNFGAISSVLN D ILSRLCPPEAEVQIDRLITGRLQSLQTYVTQQLIRAAEIRASANLAATKM SE CVLGQSKRVDFCGKGYHLMSFPQSAPHGVVFLHVTYVPAQEKNFTTAPA ICHDGKAHFPREGVFVSNGTHWFVTQRNFYEPQIITTDNTFVSGNCDVVI G IVNNTVYDPLQPELDSFKEELDKGSGGSGDIIKLLNEQVNKEMQSSNLYM SMSSWCYTHSLDGAGLFLFDHAAEEYEHAKKLIIFLNENNVPVQLTSISA P EHKFEGLTQIFQKAYEHEQHISESINNIVDHAIKSKDHATFNFLQWYVAE Q HEEEVLFKDILDKIELIGNENHGLYLADQYVKGIAKSRKSGS | 181 |
| 1B-08-PL with DS1 + HexaPro | pCoV197 | S2P-HexaPro.1158 op1x2-DS1-del-Ferritin | 1,332 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVCPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSPIEDLL FN KVTLADAGFIKQYGDCLGDIAARDLICAQKFNGLTVLPPLLTDEMIAQYT SALLAGTITSGWTFGAGPALQIPFPMQMAYRFNGIGVTQNVLYENQKLIA NQFNSAIGKIQDSLSSTPSALGKLQDVVNQNAQALNTLVKQLSSNFGAIS S VLNDILSRLCPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQSELDSIKEELDKIHKNLDSIKEELDKIHKNGSGG SG DIIKLLNEQVNKEMQSSNLYMSMSSWCYTHSLDGAGLFLFDHAAEEYEH AKKLIIFLNENNVPVQLTSISAPEHKFEGLTQIFQKAYEHEQHISESINN IVD HAIKSKDHATFNFLQWYVAEQHEEEVLFKDILDKIELIGNENHGLYLADQ YVKGIAKSRKSGS | 182 |
| 1B-08-PL with DS1 + HexaPro + D614G | pCoV198 | S2P-HexaPro.1158 op1x2-DS1-del-Ferritin-D614G | 1,332 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVCPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQGVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSPIEDLL FN KVTLADAGFIKQYGDCLGDIAARDLICAQKFNGLTVLPPLLTDEMIAQYT SALLAGTITSGWTFGAGPALQIPFPMQMAYRFNGIGVTQNVLYENQKLIA NQFNSAIGKIQDSLSSTPSALGKLQDVVNQNAQALNTLVKQLSSNFGAIS S VLNDILSRLCPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQSELDSIKEELDKIHKNLDSIKEELDKIHKNGSGG SG DIIKLLNEQVNKEMQSSNLYMSMSSWCYTHSLDGAGLFLFDHAAEEYEH AKKLIIFLNENNVPVQLTSISAPEHKFEGLTQIFQKAYEHEQHISESINN IVD HAIKSKDHATFNFLQWYVAEQHEEEVLFKDILDKIELIGNENHGLYLADQ YVKGIAKSRKSGS | 183 |
| Combine 50 and 53 | pCoV199 | His8-3c-RBD-Ferritin-Y453R/L455R /Y449K/F490R | 396 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNKNYLRRRFRKSNL KPFERDISTEIYQAGSTPCNGVEGFNCYRPLQSYGFQPTNGVGYQPYRVV VLSFELLHAPATVCGPGSGGGGESQVRQQFSKDIEKLLNEQVNKEMQSSN LYMSMSSWCYTHSLDGAGLFLFDHAAEEYEHAKKLIIFLNENNVPVQLTS ISAPEHKFEGLTQIFQKAYEHEQHISESINNIVDHAIKSKDHATFNFLQW YV AEQHEEEVLFKDILDKIELIGNENHGLYLADQYVKGIAKSRKSGS | 184 |
| 50, but with a 490A instead of a490R | pCoV200 | His8-3c-RBD-Ferritin-L455R/Y449K/F490A | 396 | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNKNYLYRRFRKSNL KPFERDISTEIYQAGSTPCNGVEGFNCYAPLQSYGFQPTNGVGYQPYRVV VLSFELLHAPATVCGPGSGGGGESQVRQQFSKDIEKLLNEQVNKEMQSSN LYMSMSSWCYTHSLDGAGLFLFDHAAEEYEHAKKLIIFLNENNVPVQLTS ISAPEHKFEGLTQIFQKAYEHEQHISESINNIVDHAIKSKDHATFNFLQW YV AEQHEEEVLFKDILDKIELIGNENHGLYLADQYVKGIAKSRKSGS | 185 |
| 1B-05 with PP reverted to KV | pCoV201 | S2P.1154-KV-del-Ferritin | 1,310 | VNLTTRTQLPPAYTNSFTRGVYYPDKVFRSSVLHSTQDLFLPFFSNVTWF HAIHVSGTNGTKRFDNPVLPFNDGVYFASTEKSNIIRGWIFGTTLDSKTQ S LLIVNNATNVVIKVCEFQFCNDPFLGVYYHKNNKSWMESEFRVYSSANN CTFEYVSQPFLMDLEGKQGNFKNLREFVFKNIDGYFKIYSKHTPINLVRD L PQGFSALEPLVDLPIGINITRFQTLLALHRSYLTPGDSSSGWTAGAAAYY V GYLQPRTFLLKYNENGTITDAVDCALDPLSETKCTLKSFTVEKGIYQTSN F RVQPTESIVRFPNITNLCPFGEVFNATRFASVYAWNRKRISNCVADYSVL Y NSASFSTFKCYGVSPTKLNDLCFTNVYADSFVIRGDEVRQIAPGQTGKIA D YNYKLPDDFTGCVIAWNSNNLDSKVGGNYNYLYRLFRKSNLKPFERDIST EIYQAGSTPCNGVEGFNCYFPLQSYGFQPTNGVGYQPYRVVVLSFELLHA PATVCGPKKSTNLVKNKCVNFNFNGLTGTGVLTESNKKFLPFQQFGRDIA DTTDAVRDPQTLEILDITPCSFGGVSVITPGTNTSNQVAVLYQDVNCTEV P VAIHADQLTPTWRVYSTGSNVFQTRAGCLIGAEHVNNSYECDIPIGAGIC A SYQTQTNSPGSASSVASQSIIAYTMSLGAENSVAYSNNSIAIPTNFTISV TTE ILPVSMTKTSVDCTMYICGDSTECSNLLLQYGSFCTQLNRALTGIAVEQD K NTQEVFAQVKQIYKTPPIKDFGGFNFSQILPDPSKPSKRSFIEDLLFNKV TL ADAGFIKQYGDCLGDIAARDLICAQKFNGLTVLPPLLTDEMIAQYTSALL AGTITSGWTFGAGAALQIPFAMQMAYRFNGIGVTQNVLYENQKLIANQF NSAIGKIQDSLSSTASALGKLQDVVNQNAQALNTLVKQLSSNFGAISSVL N DILSRLDKVEAEVQIDRLITGRLQSLQTYVTQQLIRAAEIRASANLAATK M SECVLGQSKRVDFCGKGYHLMSFPQSAPHGVVFLHVTYVPAQEKNFTTA PAICHDGKAHFPREGVFVSNGTHWFVTQRNFYEPQIITTDNTFVSGNCDV VIGIVNNTVYDPLQPELDSFKEELDKGSGGSGDIIKLLNEQVNKEMQSSN L YMSMSSWCYTHSLDGAGLFLFDHAAEEYEHAKKLIIFLNENNVPVQLTSI SAPEHKFEGLTQIFQKAYEHEQHISESINNIVDHAIKSKDHATFNFLQWY V AEQHEEEVLFKDILDKIELIGNENHGLYLADQYVKGIAKSRKSGS | 186 |
| 1B-06-PL with PP reverted to KV | pCoV202 | S2P.1158op1-KV-del-Ferritin | 1,318 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSFIEDLL FN KVTLADAGFIKQYGDCLGDIAARDLICAQKFNGLTVLPPLLTDEMIAQYT SALLAGTITSGWTFGAGAALQIPFAMQMAYRFNGIGVTQNVLYENQKLIA NQFNSAIGKIQDSLSSTASALGKLQDVVNQNAQALNTLVKQLSSNFGAIS S VLNDILSRLDKVEAEVQIDRLITGRLQSLQTYVTQQLIRAAEIRASANLA A TKMSECVLGQSKRVDFCGKGYHLMSFPQSAPHGVVFLHVTYVPAQEKNF TTAPAICHDGKAHFPREGVFVSNGTHWFVTQRNFYEPQIITTDNTFVSGN C DVVIGIVNNTVYDPLQSELDSIKEELDKIHKNGSGGSGDIIKLLNEQVNK E MQSSNLYMSMSSWCYTHSLDGAGLFLFDHAAEEYEHAKKLIIFLNENNV PVQLTSISAPEHKFEGLTQIFQKAYEHEQHISESINNIVDHAIKSKDHAT FNF LQWYVAEQHEEEVLFKDILDKIELIGNENHGLYLADQYVKGIAKSRKSGS | 187 |
| 1B-08-PL with PP reverted to KV | pCoV203 | S2P.1158op1x 2-KV-del-Ferritin | 1,332 | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSFIEDLL FN KVTLADAGFIKQYGDCLGDIAARDLICAQKFNGLTVLPPLLTDEMIAQYT SALLAGTITSGWTFGAGAALQIPFAMQMAYRFNGIGVTQNVLYENQKLIA NQFNSAIGKIQDSLSSTASALGKLQDVVNQNAQALNTLVKQLSSNFGAIS S VLNDILSRLDKVEAEVQIDRLITGRLQSLQTYVTQQLIRAAEIRASANLA A TKMSECVLGQSKRVDFCGKGYHLMSFPQSAPHGVVFLHVTYVPAQEKNF TTAPAICHDGKAHFPREGVFVSNGTHWFVTQRNFYEPQIITTDNTFVSGN C DVVIGIVNNTVYDPLQSELDSIKEELDKIHKNLDSIKEELDKIHKNGSGG SG DIIKLLNEQVNKEMQSSNLYMSMSSWCYTHSLDGAGLFLFDHAAEEYEH AKKLIIFLNENNVPVQLTSISAPEHKFEGLTQIFQKAYEHEQHISESINN IVD HAIKSKDHATFNFLQWYVAEQHEEEVLFKDILDKIELIGNENHGLYLADQ YVKGIAKSRKSGS | 188 |
| | pCoV122-notag | RBD-GSGGSG-NTD-SSQC-Ferrritin (from domain fusion sheet) | 694 | NITNLCPFGEVFNATRFASVYAWNRKRISNCVADYSVLYNSASFSTFKCY GVSPTKLNDLCFTNVYADSFVIRGDEVRQIAPGQTGKIADYNYKLPDDFT GCVIAWNSNNLDSKVGGNYNYLYRLFRKSNLKPFERDISTEIYQAGSTPC NGVEGFNCYFPLQSYGFQPTNGVGYQPYRVVVLSFELLHAPATVCGPGS GGSGSSQCVNLTTRTQLPPAYTNSFTRGVYYPDKVFRSSVLHSTQDLFLP F FSNVTWFHAIHVSGTNGTKRFDNPVLPFNDGVYFASTEKSNIIRGWIFGT T LDSKTQSLLIVNNATNVVIKVCEFQFCNDPFLGVYYHKNNKSWMESEFR VYSSANNCTFEYVSQPFLMDLEGKQGNFKNLREFVFKNIDGYFKIYSKHT PINLVRDLPQGFSALEPLVDLPIGINITRFQTLLALHRSYLTPGDSSSGW TA GAAAYYVGYLQPRTFLLKYNENGTITDAVDCALDPLSETKCTLGSGGGG ESQVRQQFSKDIEKLLNEQVNKEMQSSNLYMSMSSWCYTHSLDGAGLFL FDHAAEEYEHAKKLIIFLNENNVPVQLTSISAPEHKFEGLTQIFQKAYEH E QHISESINNIVDHAIKSKDHATFNFLQWYVAEQHEEEVLFKDILDKIELI GN ENHGLYLADQYVKGIAKSRKSGS | 189 |
| | pCoV123-notag | RBD-F490R-GSGGSG-NTD-SSQC-Ferrritin | 694 | NITNLCPFGEVFNATRFASVYAWNRKRISNCVADYSVLYNSASFSTFKCY GVSPTKLNDLCFTNVYADSFVIRGDEVRQIAPGQTGKIADYNYKLPDDFT GCVIAWNSNNLDSKVGGNYNYLYRLFRKSNLKPFERDISTEIYQAGSTPC NGVEGFNCYRPLQSYGFQPTNGVGYQPYRVVVLSFELLHAPATVCGPGS GGSGSSQCVNLTTRTQLPPAYTNSFTRGVYYPDKVFRSSVLHSTQDLFLP F FSNVTWFHAIHVSGTNGTKRFDNPVLPFNDGVYFASTEKSNIIRGWIFGT T LDSKTQSLLIVNNATNVVIKVCEFQFCNDPFLGVYYHKNNKSWMESEFR VYSSANNCTFEYVSQPFLMDLEGKQGNFKNLREFVFKNIDGYFKIYSKHT PINLVRDLPQGFSALEPLVDLPIGINITRFQTLLALHRSYLTPGDSSSGW TA GAAAYYVGYLQPRTFLLKYNENGTITDAVDCALDPLSETKCTLGSGGGG ESQVRQQFSKDIEKLLNEQVNKEMQSSNLYMSMSSWCYTHSLDGAGLFL FDHAAEEYEHAKKLIIFLNENNVPVQLTSISAPEHKFEGLTQIFQKAYEH E QHISESINNIVDHAIKSKDHATFNFLQWYVAEQHEEEVLFKDILDKIELI GN ENHGLYLADQYVKGIAKSRKSGS | 190 |
| | pCoV124-notag | RBD-F490A-GSGGSG-NTD-SSQC-Ferrritin | 694 | NITNLCPFGEVFNATRFASVYAWNRKRISNCVADYSVLYNSASFSTFKCY GVSPTKLNDLCFTNVYADSFVIRGDEVRQIAPGQTGKIADYNYKLPDDFT GCVIAWNSNNLDSKVGGNYNYLYRLFRKSNLKPFERDISTEIYQAGSTPC NGVEGFNCYAPLQSYGFQPTNGVGYQPYRVVVLSFELLHAPATVCGPGS GGSGSSQCVNLTTRTQLPPAYTNSFTRGVYYPDKVFRSSVLHSTQDLFLP F FSNVTWFHAIHVSGTNGTKRFDNPVLPFNDGVYFASTEKSNIIRGWIFGT T LDSKTQSLLIVNNATNVVIKVCEFQFCNDPFLGVYYHKNNKSWMESEFR VYSSANNCTFEYVSQPFLMDLEGKQGNFKNLREFVFKNIDGYFKIYSKHT PINLVRDLPQGFSALEPLVDLPIGINITRFQTLLALHRSYLTPGDSSSGW TA GAAAYYVGYLQPRTFLLKYNENGTITDAVDCALDPLSETKCTLGSGGGG ESQVRQQFSKDIEKLLNEQVNKEMQSSNLYMSMSSWCYTHSLDGAGLFL FDHAAEEYEHAKKLIIFLNENNVPVQLTSISAPEHKFEGLTQIFQKAYEH E QHISESINNIVDHAIKSKDHATFNFLQWYVAEQHEEEVLFKDILDKIELI GN ENHGLYLADQYVKGIAKSRKSGS | 191 |
| | pCoV125-notag | RBD-518LLH to NKS-GSGGSG-NTD-SSQC-Ferrritin | 694 | NITNLCPFGEVFNATRFASVYAWNRKRISNCVADYSVLYNSASFSTFKCY GVSPTKLNDLCFTNVYADSFVIRGDEVRQIAPGQTGKIADYNYKLPDDFT GCVIAWNSNNLDSKVGGNYNYLYRLFRKSNLKPFERDISTEIYQAGSTPC NGVEGFNCYFPLQSYGFQPTNGVGYQPYRVVVLSFENKSAPATVCGPGS GGSGSSQCVNLTTRTQLPPAYTNSFTRGVYYPDKVFRSSVLHSTQDLFLP F FSNVTWFHAIHVSGTNGTKRFDNPVLPFNDGVYFASTEKSNIIRGWIFGT T LDSKTQSLLIVNNATNVVIKVCEFQFCNDPFLGVYYHKNNKSWMESEFR VYSSANNCTFEYVSQPFLMDLEGKQGNFKNLREFVFKNIDGYFKIYSKHT PINLVRDLPQGFSALEPLVDLPIGINITRFQTLLALHRSYLTPGDSSSGW TA GAAAYYVGYLQPRTFLLKYNENGTITDAVDCALDPLSETKCTLGSGGGG ESQVRQQFSKDIEKLLNEQVNKEMQSSNLYMSMSSWCYTHSLDGAGLFL FDHAAEEYEHAKKLIIFLNENNVPVQLTSISAPEHKFEGLTQIFQKAYEH E QHISESINNIVDHAIKSKDHATFNFLQWYVAEQHEEEVLFKDILDKIELI GN ENHGLYLADQYVKGIAKSRKSGS | 192 |
| | pCoV126-notag | RBD-L518R-GSGGSG-NTD-SSQC-Ferrritin | 694 | NITNLCPFGEVFNATRFASVYAWNRKRISNCVADYSVLYNSASFSTFKCY GVSPTKLNDLCFTNVYADSFVIRGDEVRQIAPGQTGKIADYNYKLPDDFT GCVIAWNSNNLDSKVGGNYNYLYRLFRKSNLKPFERDISTEIYQAGSTPC NGVEGFNCYFPLQSYGFQPTNGVGYQPYRVVVLSFELRHAPATVCGPGS GGSGSSQCVNLTTRTQLPPAYTNSFTRGVYYPDKVFRSSVLHSTQDLFLP F FSNVTWFHAIHVSGTNGTKRFDNPVLPFNDGVYFASTEKSNIIRGWIFGT T LDSKTQSLLIVNNATNVVIKVCEFQFCNDPFLGVYYHKNNKSWMESEFR VYSSANNCTFEYVSQPFLMDLEGKQGNFKNLREFVFKNIDGYFKIYSKHT PINLVRDLPQGFSALEPLVDLPIGINITRFQTLLALHRSYLTPGDSSSGW TA GAAAYYVGYLQPRTFLLKYNENGTITDAVDCALDPLSETKCTLGSGGGG ESQVRQQFSKDIEKLLNEQVNKEMQSSNLYMSMSSWCYTHSLDGAGLFL FDHAAEEYEHAKKLIIFLNENNVPVQLTSISAPEHKFEGLTQIFQKAYEH E QHISESINNIVDHAIKSKDHATFNFLQWYVAEQHEEEVLFKDILDKIELI GN ENHGLYLADQYVKGIAKSRKSGS | 193 |
| | pCoV127-notag | RBD-Ferritin-F490A-518LLH to 518 NKS | 396 | NITNLCPFGEVFNATRFASVYAWNRKRISNCVADYSVLYNSASFSTFKCY GVSPTKLNDLCFTNVYADSFVIRGDEVRQIAPGQTGKIADYNYKLPDDFT GCVIAWNSNNLDSKVGGNYNYLYRLFRKSNLKPFERDISTEIYQAGSTPC NGVEGFNCYAPLQSYGFQPTNGVGYQPYRVVVLSFENKSAPATVCGPGS GGGGESQVRQQFSKDIEKLLNEQVNKEMQSSNLYMSMSSWCYTHSLDG AGLFLFDHAAEEYEHAKKLIIFLNENNVPVQLTSISAPEHKFEGLTQIFQ KA YEHEQHISESINNIVDHAIKSKDHATFNFLQWYVAEQHEEEVLFKDILDK I ELIGNENHGLYLADQYVKGIAKSRKSGS | 194 |
| | pCoV128-notag | RBD-Ferritin-F490A-L518R | 396 | NITNLCPFGEVFNATRFASVYAWNRKRISNCVADYSVLYNSASFSTFKCY GVSPTKLNDLCFTNVYADSFVIRGDEVRQIAPGQTGKIADYNYKLPDDFT GCVIAWNSNNLDSKVGGNYNYLYRLFRKSNLKPFERDISTEIYQAGSTPC NGVEGFNCYAPLQSYGFQPTNGVGYQPYRVVVLSFELRHAPATVCGPGS GGGGESQVRQQFSKDIEKLLNEQVNKEMQSSNLYMSMSSWCYTHSLDG AGLFLFDHAAEEYEHAKKLIIFLNENNVPVQLTSISAPEHKFEGLTQIFQ KA YEHEQHISESINNIVDHAIKSKDHATFNFLQWYVAEQHEEEVLFKDILDK I ELIGNENHGLYLADQYVKGIAKSRKSGS | 195 |
| | pCoV129-notag | RBD-Ferritin-L455R/Y449K /F490R-518LLH to NKS | 396 | NITNLCPFGEVFNATRFASVYAWNRKRISNCVADYSVLYNSASFSTFKCY GVSPTKLNDLCFTNVYADSFVIRGDEVRQIAPGQTGKIADYNYKLPDDFT GCVIAWNSNNLDSKVGGNKNYLYRRFRKSNLKPFERDISTEIYQAGSTPC NGVEGFNCYRPLQSYGFQPTNGVGYQPYRVVVLSFENKSAPATVCGPGS GGGGESQVRQQFSKDIEKLLNEQVNKEMQSSNLYMSMSSWCYTHSLDG AGLFLFDHAAEEYEHAKKLIIFLNENNVPVQLTSISAPEHKFEGLTQIFQ KA YEHEQHISESINNIVDHAIKSKDHATFNFLQWYVAEQHEEEVLFKDILDK I ELIGNENHGLYLADQYVKGIAKSRKSGS | 196 |
| | pCoV130-notag | RBD-Ferritin-L455R/Y449K /F490R-L518R | 396 | NITNLCPFGEVFNATRFASVYAWNRKRISNCVADYSVLYNSASFSTFKCY GVSPTKLNDLCFTNVYADSFVIRGDEVRQIAPGQTGKIADYNYKLPDDFT GCVIAWNSNNLDSKVGGNKNYLYRRFRKSNLKPFERDISTEIYQAGSTPC NGVEGFNCYRPLQSYGFQPTNGVGYQPYRVVVLSFELRHAPATVCGPGS GGGGESQVRQQFSKDIEKLLNEQVNKEMQSSNLYMSMSSWCYTHSLDG AGLFLFDHAAEEYEHAKKLIIFLNENNVPVQLTSISAPEHKFEGLTQIFQ KA YEHEQHISESINNIVDHAIKSKDHATFNFLQWYVAEQHEEEVLFKDILDK I ELIGNENHGLYLADQYVKGIAKSRKSGS | 197 |
| | pCoV131-notag | RBD-Ferritin-Y453R-518LLH to NKS | 396 | NITNLCPFGEVFNATRFASVYAWNRKRISNCVADYSVLYNSASFSTFKCY GVSPTKLNDLCFTNVYADSFVIRGDEVRQIAPGQTGKIADYNYKLPDDFT GCVIAWNSNNLDSKVGGNYNYLRRLFRKSNLKPFERDISTEIYQAGSTPC NGVEGFNCYFPLQSYGFQPTNGVGYQPYRVVVLSFENKSAPATVCGPGS GGGGESQVRQQFSKDIEKLLNEQVNKEMQSSNLYMSMSSWCYTHSLDG AGLFLFDHAAEEYEHAKKLIIFLNENNVPVQLTSISAPEHKFEGLTQIFQ KA YEHEQHISESINNIVDHAIKSKDHATFNFLQWYVAEQHEEEVLFKDILDK I ELIGNENHGLYLADQYVKGIAKSRKSGS | 198 |
| | pCoV132-notag | RBD-Ferritin-Y453R-L518R | 396 | NITNLCPFGEVFNATRFASVYAWNRKRISNCVADYSVLYNSASFSTFKCY GVSPTKLNDLCFTNVYADSFVIRGDEVRQIAPGQTGKIADYNYKLPDDFT GCVIAWNSNNLDSKVGGNYNYLRRLFRKSNLKPFERDISTEIYQAGSTPC NGVEGFNCYFPLQSYGFQPTNGVGYQPYRVVVLSFELRHAPATVCGPGS GGGGESQVRQQFSKDIEKLLNEQVNKEMQSSNLYMSMSSWCYTHSLDG AGLFLFDHAAEEYEHAKKLIIFLNENNVPVQLTSISAPEHKFEGLTQIFQ KA YEHEQHISESINNIVDHAIKSKDHATFNFLQWYVAEQHEEEVLFKDILDK I ELIGNENHGLYLADQYVKGIAKSRKSGS | 199 |
| | pCoV50-notag | RBD-Ferritin-L455R/Y449K /F490R | 396 | NITNLCPFGEVFNATRFASVYAWNRKRISNCVADYSVLYNSASFSTFKCY GVSPTKLNDLCFTNVYADSFVIRGDEVRQIAPGQTGKIADYNYKLPDDFT GCVIAWNSNNLDSKVGGNKNYLYRRFRKSNLKPFERDISTEIYQAGSTPC NGVEGFNCYRPLQSYGFQPTNGVGYQPYRVVVLSFELLHAPATVCGPGS GGGGESQVRQQFSKDIEKLLNEQVNKEMQSSNLYMSMSSWCYTHSLDG AGLFLFDHAAEEYEHAKKLIIFLNENNVPVQLTSISAPEHKFEGLTQIFQ KA YEHEQHISESINNIVDHAIKSKDHATFNFLQWYVAEQHEEEVLFKDILDK I ELIGNENHGLYLADQYVKGIAKSRKSGS | 200 |
| | pCoV51-notag | RBD-Ferritin-L455R | 396 | NITNLCPFGEVFNATRFASVYAWNRKRISNCVADYSVLYNSASFSTFKCY GVSPTKLNDLCFTNVYADSFVIRGDEVRQIAPGQTGKIADYNYKLPDDFT GCVIAWNSNNLDSKVGGNYNYLYRRFRKSNLKPFERDISTEIYQAGSTPC NGVEGFNCYFPLQSYGFQPTNGVGYQPYRVVVLSFELLHAPATVCGPGS GGGGESQVRQQFSKDIEKLLNEQVNKEMQSSNLYMSMSSWCYTHSLDG AGLFLFDHAAEEYEHAKKLIIFLNENNVPVQLTSISAPEHKFEGLTQIFQ KA YEHEQHISESINNIVDHAIKSKDHATFNFLQWYVAEQHEEEVLFKDILDK I ELIGNENHGLYLADQYVKGIAKSRKSGS | 201 |
| | pCoV52-notag | RBD-Ferritin-I468R | 396 | NITNLCPFGEVFNATRFASVYAWNRKRISNCVADYSVLYNSASFSTFKCY GVSPTKLNDLCFTNVYADSFVIRGDEVRQIAPGQTGKIADYNYKLPDDFT GCVIAWNSNNLDSKVGGNYNYLYRLFRKSNLKPFERDRSTEIYQAGSTPC NGVEGFNCYFPLQSYGFQPTNGVGYQPYRVVVLSFELLHAPATVCGPGS GGGGESQVRQQFSKDIEKLLNEQVNKEMQSSNLYMSMSSWCYTHSLDG AGLFLFDHAAEEYEHAKKLIIFLNENNVPVQLTSISAPEHKFEGLTQIFQ KA YEHEQHISESINNIVDHAIKSKDHATFNFLQWYVAEQHEEEVLFKDILDK I ELIGNENHGLYLADQYVKGIAKSRKSGS | 202 |
| | pCoV53-notag | RBD-Ferritin-Y453R | 396 | NITNLCPFGEVFNATRFASVYAWNRKRISNCVADYSVLYNSASFSTFKCY GVSPTKLNDLCFTNVYADSFVIRGDEVRQIAPGQTGKIADYNYKLPDDFT GCVIAWNSNNLDSKVGGNYNYLRRLFRKSNLKPFERDISTEIYQAGSTPC NGVEGFNCYFPLQSYGFQPTNGVGYQPYRVVVLSFELLHAPATVCGPGS GGGGESQVRQQFSKDIEKLLNEQVNKEMQSSNLYMSMSSWCYTHSLDG AGLFLFDHAAEEYEHAKKLIIFLNENNVPVQLTSISAPEHKFEGLTQIFQ KA YEHEQHISESINNIVDHAIKSKDHATFNFLQWYVAEQHEEEVLFKDILDK I ELIGNENHGLYLADQYVKGIAKSRKSGS | 203 |
| | pCoV54-notag | RBD-Ferritin-L452R | 396 | NITNLCPFGEVFNATRFASVYAWNRKRISNCVADYSVLYNSASFSTFKCY GVSPTKLNDLCFTNVYADSFVIRGDEVRQIAPGQTGKIADYNYKLPDDFT GCVIAWNSNNLDSKVGGNYNYRYRLFRKSNLKPFERDISTEIYQAGSTPC NGVEGFNCYFPLQSYGFQPTNGVGYQPYRVVVLSFELLHAPATVCGPGS GGGGESQVRQQFSKDIEKLLNEQVNKEMQSSNLYMSMSSWCYTHSLDG AGLFLFDHAAEEYEHAKKLIIFLNENNVPVQLTSISAPEHKFEGLTQIFQ KA YEHEQHISESINNIVDHAIKSKDHATFNFLQWYVAEQHEEEVLFKDILDK I ELIGNENHGLYLADQYVKGIAKSRKSGS | 204 |
| | pCoV55-notag | RBD-Ferritin-L492R | 396 | NITNLCPFGEVFNATRFASVYAWNRKRISNCVADYSVLYNSASFSTFKCY GVSPTKLNDLCFTNVYADSFVIRGDEVRQIAPGQTGKIADYNYKLPDDFT GCVIAWNSNNLDSKVGGNYNYLYRLFRKSNLKPFERDISTEIYQAGSTPC NGVEGFNCYFPRQSYGFQPTNGVGYQPYRVVVLSFELLHAPATVCGPGS GGGGESQVRQQFSKDIEKLLNEQVNKEMQSSNLYMSMSSWCYTHSLDG AGLFLFDHAAEEYEHAKKLIIFLNENNVPVQLTSISAPEHKFEGLTQIFQ KA YEHEQHISESINNIVDHAIKSKDHATFNFLQWYVAEQHEEEVLFKDILDK I ELIGNENHGLYLADQYVKGIAKSRKSGS | 205 |
| | pCoV56-notag | RBD-Ferritin-F490R | 396 | NITNLCPFGEVFNATRFASVYAWNRKRISNCVADYSVLYNSASFSTFKCY GVSPTKLNDLCFTNVYADSFVIRGDEVRQIAPGQTGKIADYNYKLPDDFT GCVIAWNSNNLDSKVGGNYNYLYRLFRKSNLKPFERDISTEIYQAGSTPC NGVEGFNCYRPLQSYGFQPTNGVGYQPYRVVVLSFELLHAPATVCGPGS GGGGESQVRQQFSKDIEKLLNEQVNKEMQSSNLYMSMSSWCYTHSLDG AGLFLFDHAAEEYEHAKKLIIFLNENNVPVQLTSISAPEHKFEGLTQIFQ KA YEHEQHISESINNIVDHAIKSKDHATFNFLQWYVAEQHEEEVLFKDILDK I ELIGNENHGLYLADQYVKGIAKSRKSGS | 206 |
| | pCoV57-notag | RBD-Ferritin-F490A | 396 | NITNLCPFGEVFNATRFASVYAWNRKRISNCVADYSVLYNSASFSTFKCY GVSPTKLNDLCFTNVYADSFVIRGDEVRQIAPGQTGKIADYNYKLPDDFT GCVIAWNSNNLDSKVGGNYNYLYRLFRKSNLKPFERDISTEIYQAGSTPC NGVEGFNCYAPLQSYGFQPTNGVGYQPYRVVVLSFELLHAPATVCGPGS GGGGESQVRQQFSKDIEKLLNEQVNKEMQSSNLYMSMSSWCYTHSLDG AGLFLFDHAAEEYEHAKKLIIFLNENNVPVQLTSISAPEHKFEGLTQIFQ KA YEHEQHISESINNIVDHAIKSKDHATFNFLQWYVAEQHEEEVLFKDILDK I ELIGNENHGLYLADQYVKGIAKSRKSGS | 207 |
| | pCoV58-notag | RBD-Ferritin-518LLH to 518 NKS | 396 | NITNLCPFGEVFNATRFASVYAWNRKRISNCVADYSVLYNSASFSTFKCY GVSPTKLNDLCFTNVYADSFVIRGDEVRQIAPGQTGKIADYNYKLPDDFT GCVIAWNSNNLDSKVGGNYNYLYRLFRKSNLKPFERDISTEIYQAGSTPC NGVEGFNCYFPLQSYGFQPTNGVGYQPYRVVVLSFENKSAPATVCGPGS GGGGESQVRQQFSKDIEKLLNEQVNKEMQSSNLYMSMSSWCYTHSLDG AGLFLFDHAAEEYEHAKKLIIFLNENNVPVQLTSISAPEHKFEGLTQIFQ KA YEHEQHISESINNIVDHAIKSKDHATFNFLQWYVAEQHEEEVLFKDILDK I ELIGNENHGLYLADQYVKGIAKSRKSGS | 208 |
| | pCoV59-notag | RBD-Ferritin-L518R | 396 | NITNLCPFGEVFNATRFASVYAWNRKRISNCVADYSVLYNSASFSTFKCY GVSPTKLNDLCFTNVYADSFVIRGDEVRQIAPGQTGKIADYNYKLPDDFT GCVIAWNSNNLDSKVGGNYNYLYRLFRKSNLKPFERDISTEIYQAGSTPC NGVEGFNCYFPLQSYGFQPTNGVGYQPYRVVVLSFELRHAPATVCGPGS GGGGESQVRQQFSKDIEKLLNEQVNKEMQSSNLYMSMSSWCYTHSLDG AGLFLFDHAAEEYEHAKKLIIFLNENNVPVQLTSISAPEHKFEGLTQIFQ KA YEHEQHISESINNIVDHAIKSKDHATFNFLQWYVAEQHEEEVLFKDILDK I ELIGNENHGLYLADQYVKGIAKSRKSGS | 209 |
| | pCoV60-notag | RBD-Ferritin-V367T/L335N | 396 | NITNNCPFGEVFNATRFASVYAWNRKRISNCVADYSTLYNSASFSTFKCY GVSPTKLNDLCFTNVYADSFVIRGDEVRQIAPGQTGKIADYNYKLPDDFT GCVIAWNSNNLDSKVGGNYNYLYRLFRKSNLKPFERDISTEIYQAGSTPC NGVEGFNCYFPLQSYGFQPTNGVGYQPYRVVVLSFELLHAPATVCGPGS GGGGESQVRQQFSKDIEKLLNEQVNKEMQSSNLYMSMSSWCYTHSLDG AGLFLFDHAAEEYEHAKKLIIFLNENNVPVQLTSISAPEHKFEGLTQIFQ KA YEHEQHISESINNIVDHAIKSKDHATFNFLQWYVAEQHEEEVLFKDILDK I ELIGNENHGLYLADQYVKGIAKSRKSGS | 210 |
| | pCoV61-notag | RBD-Ferritin-T385N/L387T | 396 | NITNLCPFGEVFNATRFASVYAWNRKRISNCVADYSVLYNSASFSTFKCY GVSPNKTNDLCFTNVYADSFVIRGDEVRQIAPGQTGKIADYNYKLPDDFT GCVIAWNSNNLDSKVGGNYNYLYRLFRKSNLKPFERDISTEIYQAGSTPC NGVEGFNCYFPLQSYGFQPTNGVGYQPYRVVVLSFELLHAPATVCGPGS GGGGESQVRQQFSKDIEKLLNEQVNKEMQSSNLYMSMSSWCYTHSLDG AGLFLFDHAAEEYEHAKKLIIFLNENNVPVQLTSISAPEHKFEGLTQIFQ KA YEHEQHISESINNIVDHAIKSKDHATFNFLQWYVAEQHEEEVLFKDILDK I ELIGNENHGLYLADQYVKGIAKSRKSGS | 211 |
| | pCoV62-notag | RBD-Ferritin-V382R | 396 | NITNLCPFGEVFNATRFASVYAWNRKRISNCVADYSVLYNSASFSTFKCY GRSPTKLNDLCFTNVYADSFVIRGDEVRQIAPGQTGKIADYNYKLPDDFT GCVIAWNSNNLDSKVGGNYNYLYRLFRKSNLKPFERDISTEIYQAGSTPC NGVEGFNCYFPLQSYGFQPTNGVGYQPYRVVVLSFELLHAPATVCGPGS GGGGESQVRQQFSKDIEKLLNEQVNKEMQSSNLYMSMSSWCYTHSLDG AGLFLFDHAAEEYEHAKKLIIFLNENNVPVQLTSISAPEHKFEGLTQIFQ KA YEHEQHISESINNIVDHAIKSKDHATFNFLQWYVAEQHEEEVLFKDILDK I ELIGNENHGLYLADQYVKGIAKSRKSGS | 212 |
| | pCoV63-notag | RBD-Ferritin-F377R | 396 | NITNLCPFGEVFNATRFASVYAWNRKRISNCVADYSVLYNSASFSTRKCY GVSPTKLNDLCFTNVYADSFVIRGDEVRQIAPGQTGKIADYNYKLPDDFT GCVIAWNSNNLDSKVGGNYNYLYRLFRKSNLKPFERDISTEIYQAGSTPC NGVEGFNCYFPLQSYGFQPTNGVGYQPYRVVVLSFELLHAPATVCGPGS GGGGESQVRQQFSKDIEKLLNEQVNKEMQSSNLYMSMSSWCYTHSLDG AGLFLFDHAAEEYEHAKKLIIFLNENNVPVQLTSISAPEHKFEGLTQIFQ KA YEHEQHISESINNIVDHAIKSKDHATFNFLQWYVAEQHEEEVLFKDILDK I ELIGNENHGLYLADQYVKGIAKSRKSGS | 213 |
| | | RBD-NTD-SD1 SARS-CoV-2 | | ITNLCPFGEVFNATRFASVYAWNRKRISNCVADYSVLYNSASFSTFKCYG VSPTKLNDLCFTNVYADSFVIRGDEVRQIAPGQTGKIADYNYKLPDDFTG CVIAWNSNNLDSKVGGNYNYLYRLFRKSNLKPFERDISTEIYQAGSTPCN GVEGFNCYFPLQSYGFQPTNGVGYQPYRVVVLSFELLHAPATVCGQPTES IVRFPNPKKSTNLVKNKCVNFNFNGLTGTGVLTESNKKFLPFQQFGRDIA DTTDAVRDPQTLEILDITPCS--AYTNSFTRGVYYPDKVFRSSVLHSTQDLFLPFFSNVTWFHDNPVLPFNDG VYFASTNIIRGWIFGTTLDSKTQSLLIVNNATNVVIKVCEFQFCNDPFFR VY SSANNCTFEYVSQPFLKNLREFVFKNIDGYFKIYSKHT PINLVRDLPQGFSALEPLVDLPIGINITRFQTLLALHGAAAYYVGYLQPR TF LLKYNENGTITDAVDCALD | 214 |
| | | RBD-NTD-SD1 MERS | | KPSGSVVEQAEGVECDFSPLLSGTPPQVYNFKRLVFTNCNYNLTKLLSLF S VNDFTCSQISPAAIASNCYSSLILDYFSYPLSMKSDLSVSSAGPISQFNY KQ SFSNPTCLILATVPHNLTTITKPLKYSYINKCSRLLSDDRTEVPQLVNAN QY SPCVSIVPSTVWEDGDYYRKQLSPLEGGGWLVASGSTVAMTEQLQMGFG ITVQYGTDTNSVCPKLEFANDTKIASQLGNCVEYSLYGVSGRGVFQNCTA VGVRQQRFVYDAYQNLVGYYSDDGNY--VDVGPDSVKSACIEVDIQQTFFDKTWPRPIDVSKADGIIYPQGRTYSNIT IT YQGLFPYQGDHGDMYVYSAGHATGTTPQKLFVANYSQDVKQFANGFVV RIGAAANSTGTVIISPSTSATIRKIYPAFMLGSSVGNFSDGKMGRFFNHT LV LLPDGCGTLLRAFYCILEPRSGNHCPAGNSYTSFATYHTPATDCSDGNYN RNASLNSFKEYFNLRNCTFMYTYNITEDEILEWFGITQTAQGVHLFSSRY V DLYGGNMFQFATLPVYDTIKYYSIIPHSIRSIQSDRKAWAAFYVYKLQPL T FLLDFSVDGYIRRAIDCGFNDLSQLHCSY | 215 |
| | | RBD-NTD-SD1 SARS-CoV | | RVVPSGDVVRFPNITNLCPFGEVFNATKFPSVYAWERKKISNCVADYSVL YNSTFFSTFKCYGVSATKLNDLCFSNVYADSFVVKGDDVRQIAPGQTGVI ADYNYKLPDDFMGCVLAWNTRNIDATSTGNYNYKYRYLRHGKLRPFER DISNVPFSPDGKPCTPPALNCYWPLNDYGFYTTTGIGYQPYRVVVLSFEL L NAPATVCGPKLSTDLIKNQCVNFNFNGLTGTGVLTPSSKRFQPFQQFGRD VSDFTDSVRDPKTSEILDISPCS--RCTTFDDVQAPNYTQHTSSMRGVYYPDEIFRSDTLYLTQDLFLPFYSNVT GFHTINHTFDNPVIPFKDGIYFAATEKSNVVRGWVFGSTMNNKSQSVIII N NSTNVVIRACNFELCDNPFFAVSKPMGTQTHTMIFDNAFNCTFEYISDAF S LDVSEKSGNFKHLREFVFKNKDGFLYVYKGYQPIDVVRDLPSGFNTLKPI FKLPLGINITNFRAILTAFSTWGTSAAAYFVGYLKPTTFMLKYDENGTIT D AVDCSQ | 216 |
| | | pCoV131-B.1.1.7/501Y.V1-N501Y | | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYLRRLFRKSNL KPFERDISTEIYQAGSTPCNGVEGFNCYFPLQSYGFQPTYGVGYQPYRVV VLSFENKSAPATVCGPGSGGGGESQVRQQFSKDIEKLLNEQVNKEMQSSN LYMSMSSWCYTHSLDGAGLFLFDHAAEEYEHAKKLIIFLNENNVPVQLTS ISAPEHKFEGLTQIFQKAYEHEQHISESINNIVDHAIKSKDHATFNFLQW YV AEQHEEEVLFKDILDKIELIGNENHGLYLADQYVKGIAKSRKSGS | 217 |
| | | pCoV131-B.1.351/501Y. V2-K417N_E484 K_N501Y | | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGNIADYNYKLPDDFTGCVIAWNSNNLDSKVGGNYNYLRRLFRKSNL KPFERDISTEIYQAGSTPCNGVKGFNCYFPLQSYGFQPTYGVGYQPYRVV VLSFENKSAPATVCGPGSGGGGESQVRQQFSKDIEKLLNEQVNKEMQSSN LYMSMSSWCYTHSLDGAGLFLFDHAAEEYEHAKKLIIFLNENNVPVQLTS ISAPEHKFEGLTQIFQKAYEHEQHISESINNIVDHAIKSKDHATFNFLQW YV AEQHEEEVLFKDILDKIELIGNENHGLYLADQYVKGIAKSRKSGS | 218 |
| | | pCoV131-P.1/501Y.V3-K417T_E484K_N501Y | | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGTIADYNYKLPDDFTGCVIAWNSNNLDSKVGGNYNYLRRLFRKSNL KPFERDISTEIYQAGSTPCNGVKGFNCYFPLQSYGFQPTYGVGYQPYRVV VLSFENKSAPATVCGPGSGGGGESQVRQQFSKDIEKLLNEQVNKEMQSSN LYMSMSSWCYTHSLDGAGLFLFDHAAEEYEHAKKLIIFLNENNVPVQLTS ISAPEHKFEGLTQIFQKAYEHEQHISESINNIVDHAIKSKDHATFNFLQW YV AEQHEEEVLFKDILDKIELIGNENHGLYLADQYVKGIAKSRKSGS | 219 |
| | | pCoV111-B.1.1.7/501Y.V1-delta69-70_delta144_N501Y_A570D_D614G | | SSQCVNLTTRTQLPPAYTNSFTRGVYYPDKVFRSSVLHSTQDLFLPFFSN V TWFHAISGTNGTKRFDNPVLPFNDGVYFASTEKSNIIRGWIFGTTLDSKT Q SLLIVNNATNVVIKVCEFQFCNDPFLGVYHKNNKSWMESEFRVYSSANN CTFEYVSQPFLMDLEGKQGNFKNLREFVFKNIDGYFKIYSKHTPINLVRD L PQGFSALEPLVDLPIGINITRFQTLLALHRSYLTPGDSSSGWTAGAAAYY V GYLQPRTFLLKYNENGTITDAVDCALDPLSETKCTLKSFTVEKGIYQTSN F RVQPTESIVRFPNITNLCPFGEVFNATRFASVYAWNRKRISNCVADYSVL Y NSASFSTFKCYGVSPTKLNDLCFTNVYADSFVIRGDEVRQIAPGQTGKIA D YNYKLPDDFTGCVIAWNSNNLDSKVGGNYNYLYRLFRKSNLKPFERDIST EIYQAGSTPCNGVEGFNCYFPLQSYGFQPTYGVGYQPYRVVVLSFELLHA PATVCGPKKSTNLVKNKCVNFNFNGLTGTGVLTESNKKFLPFQQFGRDID DTTDAVRDPQTLEILDITPCSFGGVSVITPGTNTSNQVAVLYQGVNCTEV P VAIHADQLTPTWRVYSTGSNVFQTRAGCLIGAEHVNNSYECDIPIGAGIC A SYQTGGSQSIIAYTGSGGGGESQVRQQFSKDIEKLLNEQVNKEMQSSNLY MSMSSWCYTHSLDGAGLFLFDHAAEEYEHAKKLIIFLNENNVPVQLTSIS APEHKFEGLTQIFQKAYEHEQHISESINNIVDHAIKSKDHATFNFLQWYV A EQHEEEVLFKDILDKIELIGNENHGLYLADQYVKGIAKSRKSGS | 220 |
| | | pCoV111-B.1.351/501Y.V2-L18F_D80A_ D215G _delta241-243_R246I_K 417N_E484K_ N501Y_D614G | | SSQCVNFTTRTQLPPAYTNSFTRGVYYPDKVFRSSVLHSTQDLFLPFFSN V TWFHAIHVSGTNGTKRFANPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R GLPQGFSALEPLVDLPIGINITRFQTLHISYLTPGDSSSGWTAGAAAYYV G YLQPRTFLLKYNENGTITDAVDCALDPLSETKCTLKSFTVEKGIYQTSNF R VQPTESIVRFPNITNLCPFGEVFNATRFASVYAWNRKRISNCVADYSVLY N SASFSTFKCYGVSPTKLNDLCFTNVYADSFVIRGDEVRQIAPGQTGNIAD Y NYKLPDDFTGCVIAWNSNNLDSKVGGNYNYLYRLFRKSNLKPFERDISTE IYQAGSTPCNGVKGFNCYFPLQSYGFQPTYGVGYQPYRVVVLSFELLHAP ATVCGPKKSTNLVKNKCVNFNFNGLTGTGVLTESNKKFLPFQQFGRDIAD TTDAVRDPQTLEILDITPCSFGGVSVITPGTNTSNQVAVLYQGVNCTEVP V AIHADQLTPTWRVYSTGSNVFQTRAGCLIGAEHVNNSYECDIPIGAGICA S YQTGGSQSIIAYTGSGGGGESQVRQQFSKDIEKLLNEQVNKEMQSSNLYM SMSSWCYTHSLDGAGLFLFDHAAEEYEHAKKLIIFLNENNVPVQLTSISA P EHKFEGLTQIFQKAYEHEQHISESINNIVDHAIKSKDHATFNFLQWYVAE Q HEEEVLFKDILDKIELIGNENHGLYLADQYVKGIAKSRKSGS | 221 |
| | | pCoV111-B.1.351/501Y. V2-L18F_D80_D215G _delta241-243_R246I_K417N_E484K_N501Y_D614G | | SSQCVNFTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R GLPQGFSALEPLVDLPIGINITRFQTLHISYLTPGDSSSGWTAGAAAYYV G YLQPRTFLLKYNENGTITDAVDCALDPLSETKCTLKSFTVEKGIYQTSNF R VQPTESIVRFPNITNLCPFGEVFNATRFASVYAWNRKRISNCVADYSVLY N SASFSTFKCYGVSPTKLNDLCFTNVYADSFVIRGDEVRQIAPGQTGNIAD Y NYKLPDDFTGCVIAWNSNNLDSKVGGNYNYLYRLFRKSNLKPFERDISTE IYQAGSTPCNGVKGFNCYFPLQSYGFQPTYGVGYQPYRVVVLSFELLHAP ATVCGPKKSTNLVKNKCVNFNFNGLTGTGVLTESNKKFLPFQQFGRDIAD TTDAVRDPQTLEILDITPCSFGGVSVITPGTNTSNQVAVLYQGVNCTEVP V AIHADQLTPTWRVYSTGSNVFQTRAGCLIGAEHVNNSYECDIPIGAGICA S YQTGGSQSIIAYTGSGGGGESQVRQQFSKDIEKLLNEQVNKEMQSSNLYM SMSSWCYTHSLDGAGLFLFDHAAEEYEHAKKLIIFLNENNVPVQLTSISA P EHKFEGLTQIFQKAYEHEQHISESINNIVDHAIKSKDHATFNFLQWYVAE Q HEEEVLFKDILDKIELIGNENHGLYLADQYVKGIAKSRKSGS | 222 |
| | | pCoV111-B.1.351/501Y.V2-L18F_D80A _D215_delta241243_R246I_K417N_E484K_N501Y_D614G | | SSQCVNFTTRTQLPPAYTNSFTRGVYYPDKVFRSSVLHSTQDLFLPFFSN V TWFHAIHVSGTNGTKRFANPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLHISYLTPGDSSSGWTAGAAAYYV G YLQPRTFLLKYNENGTITDAVDCALDPLSETKCTLKSFTVEKGIYQTSNF R VQPTESIVRFPNITNLCPFGEVFNATRFASVYAWNRKRISNCVADYSVLY N SASFSTFKCYGVSPTKLNDLCFTNVYADSFVIRGDEVRQIAPGQTGNIAD Y NYKLPDDFTGCVIAWNSNNLDSKVGGNYNYLYRLFRKSNLKPFERDISTE IYQAGSTPCNGVKGFNCYFPLQSYGFQPTYGVGYQPYRVVVLSFELLHAP ATVCGPKKSTNLVKNKCVNFNFNGLTGTGVLTESNKKFLPFQQFGRDIAD TTDAVRDPQTLEILDITPCSFGGVSVITPGTNTSNQVAVLYQGVNCTEVP V AIHADQLTPTWRVYSTGSNVFQTRAGCLIGAEHVNNSYECDIPIGAGICA S YQTGGSQSIIAYTGSGGGGESQVRQQFSKDIEKLLNEQVNKEMQSSNLYM SMSSWCYTHSLDGAGLFLFDHAAEEYEHAKKLIIFLNENNVPVQLTSISA P EHKFEGLTQIFQKAYEHEQHISESINNIVDHAIKSKDHATFNFLQWYVAE Q HEEEVLFKDILDKIELIGNENHGLYLADQYVKGIAKSRKSGS | 223 |
| | | pCoV111-P.1/501Y.V3-L18F_T20N_P26S_D138Y_R190S_K417T_E484K_N501Y_D614G_H655Y | | SSQCVNFTNRTQLPSAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNYPFLGVYYHKNNKSWMESEFRVYSSA NNCTFEYVSQPFLMDLEGKQGNFKNLSEFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGT I ADYNYKLPDDFTGCVIAWNSNNLDSKVGGNYNYLYRLFRKSNLKPFERD ISTEIYQAGSTPCNGVKGFNCYFPLQSYGFQPTYGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQGVNC T EVPVAIHADQLTPTWRVYSTGSNVFQTRAGCLIGAEYVNNSYECDIPIGA GICASYQTGGSQSIIAYTGSGGGGESQVRQQFSKDIEKLLNEQVNKEMQS S NLYMSMSSWCYTHSLDGAGLFLFDHAAEEYEHAKKLIIFLNENNVPVQL TSISAPEHKFEGLTQIFQKAYEHEQHISESINNIVDHAIKSKDHATFNFL QW YVAEQHEEEVLFKDILDKIELIGNENHGLYLADQYVKGIAKSRKSGS | 224 |
| | | pCoV146-B.1.1.7/501Y.V1-delta69-70_delta144_N501Y | | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGKIADYNYKLPDDFTGCVIAWNSNNLDSKVGGNYNYLRRLFRKSNL KPFERDISTEIYQAGSTPCNGVEGFNCYFPLQSYGFQPTYGVGYQPYRVV VLSFENKSAPATVCGPGSGGSGSSQCVNLTTRTQLPPAYTNSFTRGVYYP DKVFRSSVLHSTQDLFLPFFSNVTWFHAISGTNGTKRFDNPVLPFNDGVY F ASTEKSNIIRGWIFGTTLDSKTQSLLIVNNATNVVIKVCEFQFCNDPFLG VY HKNNKSWMESEFRVYSSANNCTFEYVSQPFLMDLEGKQGNFKNLREFVF KNIDGYFKIYSKHTPINL VRDLPQGFSALEPL VDLPIGINITRFQTLLALHRS YLTPGDSSSGWTAGAAAYYVGYLQPRTFLLKYNENGTITDAVDCALDPL SETKCTLGSGGGGESQVRQQFSKDIEKLLNEQVNKEMQSSNLYMSMSSW CYTHSLDGAGLFLFDHAAEEYEHAKKLIIFLNENNVPVQLTSISAPEHKF E GLTQIFQKAYEHEQHISESINNIVDHAIKSKDHATFNFLQWYVAEQHEEE V LFKDILDKIELIGNENHGLYLADQYVKGIAKSRKSGS | 225 |
| | | pCoV146-B.1.351/501Y.V2-L18F_D80A_D215G_delta241-243_R246I_K417N_E484K_N501Y | | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGNIADYNYKLPDDFTGCVIAWNSNNLDSKVGGNYNYLRRLFRKSNL KPFERDISTEIYQAGSTPCNGVKGFNCYFPLQSYGFQPTYGVGYQPYRVV VLSFENKSAPATVCGPGSGGSGSSQCVNFTTRTQLPPAYTNSFTRGVYYP DKVFRSSVLHSTQDLFLPFFSNVTWFHAIHVSGTNGTKRFANPVLPFNDG VYFASTEKSNIIRGWIFGTTLDSKTQSLLIVNNATNVVIKVCEFQFCNDP FL GVYYHKNNKSWMESEFRVYSSANNCTFEYVSQPFLMDLEGKQGNFKNL REFVFKNIDGYFKIYSKHTPINLVRGLPQGFSALEPLVDLPIGINITRFQ TLH ISYLTPGDSSSGWTAGAAAYYVGYLQPRTFLLKYNENGTITDAVDCALDP LSETKCTLGSGGGGESQVRQQFSKDIEKLLNEQVNKEMQSSNLYMSMSS WCYTHSLDGAGLFLFDHAAEEYEHAKKLIIFLNENNVPVQLTSISAPEHK F EGLTQIFQKAYEHEQHISESINNIVDHAIKSKDHATFNFLQWYVAEQHEE E VLFKDILDKIELIGNENHGLYLADQYVKGIAKSRKSGS | 226 |
| | | pCoV146-B.1.351/501Y.V2-L18F_D80_D215G_delta241-243_R246I_K417N_E484K_N501Y | | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGNIADYNYKLPDDFTGCVIAWNSNNLDSKVGGNYNYLRRLFRKSNL KPFERDISTEIYQAGSTPCNGVKGFNCYFPLQSYGFQPTYGVGYQPYRVV VLSFENKSAPATVCGPGSGGSGSSQCVNFTTRTQLPPAYTNSFTRGVYYP DKVFRSSVLHSTQDLFLPFFSNVTWFHAIHVSGTNGTKRFDNPVLPFNDG VYFASTEKSNIIRGWIFGTTLDSKTQSLLIVNNATNVVIKVCEFQFCNDP FL GVYYHKNNKSWMESEFRVYSSANNCTFEYVSQPFLMDLEGKQGNFKNL REFVFKNIDGYFKIYSKHTPINLVRGLPQGFSALEPLVDLPIGINITRFQ TLH ISYLTPGDSSSGWTAGAAAYYVGYLQPRTFLLKYNENGTITDAVDCALDP LSETKCTLGSGGGGESQVRQQFSKDIEKLLNEQVNKEMQSSNLYMSMSS WCYTHSLDGAGLFLFDHAAEEYEHAKKLIIFLNENNVPVQLTSISAPEHK F EGLTQIFQKAYEHEQHISESINNIVDHAIKSKDHATFNFLQWYVAEQHEE E VLFKDILDKIELIGNENHGLYLADQYVKGIAKSRKSGS | 227 |
| | | pCoV146-B.1.351/501Y.V2-L18F_D80A_D215_delta241243_R246I_K417N_E484K_N501Y | | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGNIADYNYKLPDDFTGCVIAWNSNNLDSKVGGNYNYLRRLFRKSNL KPFERDISTEIYQAGSTPCNGVKGFNCYFPLQSYGFQPTYGVGYQPYRVV VLSFENKSAPATVCGPGSGGSGSSQCVNFTTRTQLPPAYTNSFTRGVYYP DKVFRSSVLHSTQDLFLPFFSNVTWFHAIHVSGTNGTKRFANPVLPFNDG VYFASTEKSNIIRGWIFGTTLDSKTQSLLIVNNATNVVIKVCEFQFCNDP FL GVYYHKNNKSWMESEFRVYSSANNCTFEYVSQPFLMDLEGKQGNFKNL REFVFKNIDGYFKIYSKHTPINLVRDLPQGFSALEPLVDLPIGINITRFQ TLH ISYLTPGDSSSGWTAGAAAYYVGYLQPRTFLLKYNENGTITDAVDCALDP LSETKCTLGSGGGGESQVRQQFSKDIEKLLNEQVNKEMQSSNLYMSMSS WCYTHSLDGAGLFLFDHAAEEYEHAKKLIIFLNENNVPVQLTSISAPEHK F EGLTQIFQKAYEHEQHISESINNIVDHAIKSKDHATFNFLQWYVAEQHEE E VLFKDILDKIELIGNENHGLYLADQYVKGIAKSRKSGS | 228 |
| | | pCoV146-P.1/501Y.V3-L18F_T20N_P26S_D138Y_R190S_K417T_E484K_N501Y | | HHHHHHHHGPLEVLFQGPNITNLCPFGEVFNATRFASVYAWNRKRISNCV ADYSVLYNSASFSTFKCYGVSPTKLNDLCFTNVYADSFVIRGDEVRQIAP GQTGTIADYNYKLPDDFTGCVIAWNSNNLDSKVGGNYNYLRRLFRKSNL KPFERDISTEIYQAGSTPCNGVKGFNCYFPLQSYGFQPTYGVGYQPYRVV VLSFENKSAPATVCGPGSGGSGSSQCVNFTNRTQLPSAYTNSFTRGVYYP DKVFRSSVLHSTQDLFLPFFSNVTWFHAIHVSGTNGTKRFDNPVLPFNDG VYFASTEKSNIIRGWIFGTTLDSKTQSLLIVNNATNVVIKVCEFQFCNYP FL GVYYHKNNKSWMESEFRVYSSANNCTFEYVSQPFLMDLEGKQGNFKNL SEFVFKNIDGYFKIYSKHTPINL VRDLPQGFSALEPL VDLPIGINITRFQTLL ALHRSYLTPGDSSSGWTAGAAAYYVGYLQPRTFLLKYNENGTITDAVDC ALDPLSETKCTLGSGGGGESQVRQQFSKDIEKLLNEQVNKEMQSSNLYMS MSSWCYTHSLDGAGLFLFDHAAEEYEHAKKLIIFLNENNVPVQLTSISAP E HKFEGLTQIFQKAYEHEQHISESINNIVDHAIKSKDHATFNFLQWYVAEQ H EEEVLFKDILDKIELIGNENHGLYLADQYVKGIAKSRKSGS | 229 |
| | | pCoV1B-06-PL-B.1.1.7/501Y.V1-delta69-70_delta144-N501Y-A570D-D614G-P681H-T716I-S982A-D1118H | | SSQCVNLTTRTQLPPAYTNSFTRGVYYPDKVFRSSVLHSTQDLFLPFFSN V TWFHAISGTNGTKRFDNPVLPFNDGVYFASTEKSNIIRGWIFGTTLDSKT Q SLLIVNNATNVVIKVCEFQFCNDPFLGVYHKNNKSWMESEFRVYSSANN CTFEYVSQPFLMDLEGKQGNFKNLREFVFKNIDGYFKIYSKHTPINLVRD L PQGFSALEPLVDLPIGINITRFQTLLALHRSYLTPGDSSSGWTAGAAAYY V GYLQPRTFLLKYNENGTITDAVDCALDPLSETKCTLKSFTVEKGIYQTSN F RVQPTESIVRFPNITNLCPFGEVFNATRFASVYAWNRKRISNCVADYSVL Y NSASFSTFKCYGVSPTKLNDLCFTNVYADSFVIRGDEVRQIAPGQTGKIA D YNYKLPDDFTGCVIAWNSNNLDSKVGGNYNYLYRLFRKSNLKPFERDIST EIYQAGSTPCNGVEGFNCYFPLQSYGFQPTYGVGYQPYRVVVLSFELLHA PATVCGPKKSTNLVKNKCVNFNFNGLTGTGVLTESNKKFLPFQQFGRDID DTTDAVRDPQTLEILDITPCSFGGVSVITPGTNTSNQVAVLYQGVNCTEV P VAIHADQLTPTWRVYSTGSNVFQTRAGCLIGAEHVNNSYECDIPIGAGIC A SYQTQTNSHGSASSVASQSIIAYTMSLGAENSVAYSNNSIAIPINFTISV TTE ILPVSMTKTSVDCTMYICGDSTECSNLLLQYGSFCTQLNRALTGIAVEQD K NTQEVFAQVKQIYKTPPIKDFGGFNFSQILPDPSKPSKRSFIEDLLFNKV TL ADAGFIKQYGDCLGDIAARDLICAQKFNGLTVLPPLLTDEMIAQYTSALL AGTITSGWTFGAGAALQIPFAMQMAYRFNGIGVTQNVLYENQKLIANQF NSAIGKIQDSLSSTASALGKLQDVVNQNAQALNTLVKQLSSNFGAISSVL N DILARLDPPEAEVQIDRLITGRLQSLQTYVTQQLIRAAEIRASANLAATK M SECVLGQSKRVDFCGKGYHLMSFPQSAPHGVVFLHVTYVPAQEKNFTTA PAICHDGKAHFPREGVFVSNGTHWFVTQRNFYEPQIITTHNTFVSGNCDV VIGIVNNTVYDPLQSELDSIKEELDKIHKNGSGGSGDIIKLLNEQVNKEM Q SSNLYMSMSSWCYTHSLDGAGLFLFDHAAEEYEHAKKLIIFLNENNVPVQ LTSISAPEHKFEGLTQIFQKAYEHEQHISESINNIVDHAIKSKDHATFNF LQ WYVAEQHEEEVLFKDILDKIELIGNENHGLYLADQYVKGIAKSRKSGS | 230 |
| | | pCoV1B-06-PL-B.1.351/501Y.V2-L18F_D80A_D215G_delta241-243_R246I_K417N_E484K_N501Y_D614G_A701V | | SSQCVNFTTRTQLPPAYTNSFTRGVYYPDKVFRSSVLHSTQDLFLPFFSN V TWFHAIHVSGTNGTKRFANPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R GLPQGFSALEPLVDLPIGINITRFQTLHISYLTPGDSSSGWTAGAAAYYV G YLQPRTFLLKYNENGTITDAVDCALDPLSETKCTLKSFTVEKGIYQTSNF R VQPTESIVRFPNITNLCPFGEVFNATRFASVYAWNRKRISNCVADYSVLY N SASFSTFKCYGVSPTKLNDLCFTNVYADSFVIRGDEVRQIAPGQTGNIAD Y NYKLPDDFTGCVIAWNSNNLDSKVGGNYNYLYRLFRKSNLKPFERDISTE IYQAGSTPCNGVKGFNCYFPLQSYGFQPTYGVGYQPYRVVVLSFELLHAP ATVCGPKKSTNLVKNKCVNFNFNGLTGTGVLTESNKKFLPFQQFGRDIAD TTDAVRDPQTLEILDITPCSFGGVSVITPGTNTSNQVAVLYQGVNCTEVP V AIHADQLTPTWRVYSTGSNVFQTRAGCLIGAEHVNNSYECDIPIGAGICA S YQTQTNSPGSASSVASQSIIAYTMSLGVENSVAYSNNSIAIPTNFTISVT TEI LPVSMTKTSVDCTMYICGDSTECSNLLLQYGSFCTQLNRALTGIAVEQDK NTQEVFAQVKQIYKTPPIKDFGGFNFSQILPDPSKPSKRSFIEDLLFNKV TL ADAGFIKQYGDCLGDIAARDLICAQKFNGLTVLPPLLTDEMIAQYTSALL AGTITSGWTFGAGAALQIPFAMQMAYRFNGIGVTQNVLYENQKLIANQF NSAIGKIQDSLSSTASALGKLQDVVNQNAQALNTLVKQLSSNFGAISSVL N DILSRLDPPEAEVQIDRLITGRLQSLQTYVTQQLIRAAEIRASANLAATK MS ECVLGQSKRVDFCGKGYHLMSFPQSAPHGVVFLHVTYVPAQEKNFTTAP AICHDGKAHFPREGVFVSNGTHWFVTQRNFYEPQIITTDNTFVSGNCDVV I GIVNNTVYDPLQSELDSIKEELDKIHKNGSGGSGDIIKLLNEQVNKEMQS S NLYMSMSSWCYTHSLDGAGLFLFDHAAEEYEHAKKLIIFLNENNVPVQL TSISAPEHKFEGLTQIFQKAYEHEQHISESINNIVDHAIKSKDHATFNFL QW YVAEQHEEEVLFKDILDKIELIGNENHGLYLADQYVKGIAKSRKSGS | 231 |
| | | pCoV1B-06-PL-B.1.351/501Y.V2-L18F_D80_D215G_delta241-243_R246I_K417N_E484K_N501Y_D614G_A701V | | SSQCVNFTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R GLPQGFSALEPLVDLPIGINITRFQTLHISYLTPGDSSSGWTAGAAAYYV G YLQPRTFLLKYNENGTITDAVDCALDPLSETKCTLKSFTVEKGIYQTSNF R VQPTESIVRFPNITNLCPFGEVFNATRFASVYAWNRKRISNCVADYSVLY N SASFSTFKCYGVSPTKLNDLCFTNVYADSFVIRGDEVRQIAPGQTGNIAD Y NYKLPDDFTGCVIAWNSNNLDSKVGGNYNYLYRLFRKSNLKPFERDISTE IYQAGSTPCNGVKGFNCYFPLQSYGFQPTYGVGYQPYRVVVLSFELLHAP ATVCGPKKSTNLVKNKCVNFNFNGLTGTGVLTESNKKFLPFQQFGRDIAD TTDAVRDPQTLEILDITPCSFGGVSVITPGTNTSNQVAVLYQGVNCTEVP V AIHADQLTPTWRVYSTGSNVFQTRAGCLIGAEHVNNSYECDIPIGAGICA S YQTQTNSPGSASSVASQSIIAYTMSLGVENSVAYSNNSIAIPTNFTISVT TEI LPVSMTKTSVDCTMYICGDSTECSNLLLQYGSFCTQLNRALTGIAVEQDK NTQEVFAQVKQIYKTPPIKDFGGFNFSQILPDPSKPSKRSFIEDLLFNKV TL ADAGFIKQYGDCLGDIAARDLICAQKFNGLTVLPPLLTDEMIAQYTSALL AGTITSGWTFGAGAALQIPFAMQMAYRFNGIGVTQNVLYENQKLIANQF NSAIGKIQDSLSSTASALGKLQDVVNQNAQALNTLVKQLSSNFGAISSVL N DILSRLDPPEAEVQIDRLITGRLQSLQTYVTQQLIRAAEIRASANLAATK MS ECVLGQSKRVDFCGKGYHLMSFPQSAPHGVVFLHVTYVPAQEKNFTTAP AICHDGKAHFPREGVFVSNGTHWFVTQRNFYEPQIITTDNTFVSGNCDVV I GIVNNTVYDPLQSELDSIKEELDKIHKNGSGGSGDIIKLLNEQVNKEMQS S NLYMSMSSWCYTHSLDGAGLFLFDHAAEEYEHAKKLIIFLNENNVPVQL TSISAPEHKFEGLTQIFQKAYEHEQHISESINNIVDHAIKSKDHATFNFL QW YVAEQHEEEVLFKDILDKIELIGNENHGLYLADQYVKGIAKSRKSGS | 232 |
| | | pCoV1B-06-PL-B.1.351/501Y.V2-L18F_D80A_D215_delta241243_R246I_K417N_E484K_N501Y_D614G_A701V | | SSQCVNFTTRTQLPPAYTNSFTRGVYYPDKVFRSSVLHSTQDLFLPFFSN V TWFHAIHVSGTNGTKRFANPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLHISYLTPGDSSSGWTAGAAAYYV G YLQPRTFLLKYNENGTITDAVDCALDPLSETKCTLKSFTVEKGIYQTSNF R VQPTESIVRFPNITNLCPFGEVFNATRFASVYAWNRKRISNCVADYSVLY N SASFSTFKCYGVSPTKLNDLCFTNVYADSFVIRGDEVRQIAPGQTGNIAD Y NYKLPDDFTGCVIAWNSNNLDSKVGGNYNYLYRLFRKSNLKPFERDISTE IYQAGSTPCNGVKGFNCYFPLQSYGFQPTYGVGYQPYRVVVLSFELLHAP ATVCGPKKSTNLVKNKCVNFNFNGLTGTGVLTESNKKFLPFQQFGRDIAD TTDAVRDPQTLEILDITPCSFGGVSVITPGTNTSNQVAVLYQGVNCTEVP V AIHADQLTPTWRVYSTGSNVFQTRAGCLIGAEHVNNSYECDIPIGAGICA S YQTQTNSPGSASSVASQSIIAYTMSLGVENSVAYSNNSIAIPTNFTISVT TEI LPVSMTKTSVDCTMYICGDSTECSNLLLQYGSFCTQLNRALTGIAVEQDK NTQEVFAQVKQIYKTPPIKDFGGFNFSQILPDPSKPSKRSFIEDLLFNKV TL ADAGFIKQYGDCLGDIAARDLICAQKFNGLTVLPPLLTDEMIAQYTSALL AGTITSGWTFGAGAALQIPFAMQMAYRFNGIGVTQNVLYENQKLIANQF NSAIGKIQDSLSSTASALGKLQDVVNQNAQALNTLVKQLSSNFGAISSVL N DILSRLDPPEAEVQIDRLITGRLQSLQTYVTQQLIRAAEIRASANLAATK MS ECVLGQSKRVDFCGKGYHLMSFPQSAPHGVVFLHVTYVPAQEKNFTTAP AICHDGKAHFPREGVFVSNGTHWFVTQRNFYEPQIITTDNTFVSGNCDVV I GIVNNTVYDPLQSELDSIKEELDKIHKNGSGGSGDIIKLLNEQVNKEMQS S NLYMSMSSWCYTHSLDGAGLFLFDHAAEEYEHAKKLIIFLNENNVPVQL TSISAPEHKFEGLTQIFQKAYEHEQHISESINNIVDHAIKSKDHATFNFL QW YVAEQHEEEVLFKDILDKIELIGNENHGLYLADQYVKGIAKSRKSGS | 233 |
| | | pCoV1B-06-PL-B.1.351/501Y.V2-L18F_D80A_D215G_delta241-243_R246_K417N_E484K_N501Y_D614G_A701V | | SSQCVNFTTRTQLPPAYTNSFTRGVYYPDKVFRSSVLHSTQDLFLPFFSN V TWFHAIHVSGTNGTKRFANPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R GLPQGFSALEPLVDLPIGINITRFQTLHRSYLTPGDSSSGWTAGAAAYYV G YLQPRTFLLKYNENGTITDAVDCALDPLSETKCTLKSFTVEKGIYQTSNF R VQPTESIVRFPNITNLCPFGEVFNATRFASVYAWNRKRISNCVADYSVLY N SASFSTFKCYGVSPTKLNDLCFTNVYADSFVIRGDEVRQIAPGQTGNIAD Y NYKLPDDFTGCVIAWNSNNLDSKVGGNYNYLYRLFRKSNLKPFERDISTE IYQAGSTPCNGVKGFNCYFPLQSYGFQPTYGVGYQPYRVVVLSFELLHAP ATVCGPKKSTNLVKNKCVNFNFNGLTGTGVLTESNKKFLPFQQFGRDIAD TTDAVRDPQTLEILDITPCSFGGVSVITPGTNTSNQVAVLYQGVNCTEVP V AIHADQLTPTWRVYSTGSNVFQTRAGCLIGAEHVNNSYECDIPIGAGICA S YQTQTNSPGSASSVASQSIIAYTMSLGVENSVAYSNNSIAIPTNFTISVT TEI LPVSMTKTSVDCTMYICGDSTECSNLLLQYGSFCTQLNRALTGIAVEQDK NTQEVFAQVKQIYKTPPIKDFGGFNFSQILPDPSKPSKRSFIEDLLFNKV TL ADAGFIKQYGDCLGDIAARDLICAQKFNGLTVLPPLLTDEMIAQYTSALL AGTITSGWTFGAGAALQIPFAMQMAYRFNGIGVTQNVLYENQKLIANQF NSAIGKIQDSLSSTASALGKLQDVVNQNAQALNTLVKQLSSNFGAISSVL N DILSRLDPPEAEVQIDRLITGRLQSLQTYVTQQLIRAAEIRASANLAATK MS ECVLGQSKRVDFCGKGYHLMSFPQSAPHGVVFLHVTYVPAQEKNFTTAP AICHDGKAHFPREGVFVSNGTHWFVTQRNFYEPQIITTDNTFVSGNCDVV I GIVNNTVYDPLQSELDSIKEELDKIHKNGSGGSGDIIKLLNEQVNKEMQS S NLYMSMSSWCYTHSLDGAGLFLFDHAAEEYEHAKKLIIFLNENNVPVQL TSISAPEHKFEGLTQIFQKAYEHEQHISESINNIVDHAIKSKDHATFNFL QW YVAEQHEEEVLFKDILDKIELIGNENHGLYLADQYVKGIAKSRKSGS | 234 |
| | | pCoV1B-06-PL-B.1.351/501Y.V2-L18F_D80_D215G_delta241-243_R246_K417N_E484K_N501Y_D614G_A701V | | SSQCVNFTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R GLPQGFSALEPLVDLPIGINITRFQTLHRSYLTPGDSSSGWTAGAAAYYV G YLQPRTFLLKYNENGTITDAVDCALDPLSETKCTLKSFTVEKGIYQTSNF R VQPTESIVRFPNITNLCPFGEVFNATRFASVYAWNRKRISNCVADYSVLY N SASFSTFKCYGVSPTKLNDLCFTNVYADSFVIRGDEVRQIAPGQTGNIAD Y NYKLPDDFTGCVIAWNSNNLDSKVGGNYNYLYRLFRKSNLKPFERDISTE IYQAGSTPCNGVKGFNCYFPLQSYGFQPTYGVGYQPYRVVVLSFELLHAP ATVCGPKKSTNLVKNKCVNFNFNGLTGTGVLTESNKKFLPFQQFGRDIAD TTDAVRDPQTLEILDITPCSFGGVSVITPGTNTSNQVAVLYQGVNCTEVP V AIHADQLTPTWRVYSTGSNVFQTRAGCLIGAEHVNNSYECDIPIGAGICA S YQTQTNSPGSASSVASQSIIAYTMSLGVENSVAYSNNSIAIPTNFTISVT TEI LPVSMTKTSVDCTMYICGDSTECSNLLLQYGSFCTQLNRALTGIAVEQDK NTQEVFAQVKQIYKTPPIKDFGGFNFSQILPDPSKPSKRSFIEDLLFNKV TL ADAGFIKQYGDCLGDIAARDLICAQKFNGLTVLPPLLTDEMIAQYTSALL AGTITSGWTFGAGAALQIPFAMQMAYRFNGIGVTQNVLYENQKLIANQF NSAIGKIQDSLSSTASALGKLQDVVNQNAQALNTLVKQLSSNFGAISSVL N DILSRLDPPEAEVQIDRLITGRLQSLQTYVTQQLIRAAEIRASANLAATK MS ECVLGQSKRVDFCGKGYHLMSFPQSAPHGVVFLHVTYVPAQEKNFTTAP AICHDGKAHFPREGVFVSNGTHWFVTQRNFYEPQIITTDNTFVSGNCDVV I GIVNNTVYDPLQSELDSIKEELDKIHKNGSGGSGDIIKLLNEQVNKEMQS S NLYMSMSSWCYTHSLDGAGLFLFDHAAEEYEHAKKLIIFLNENNVPVQL TSISAPEHKFEGLTQIFQKAYEHEQHISESINNIVDHAIKSKDHATFNFL QW YVAEQHEEEVLFKDILDKIELIGNENHGLYLADQYVKGIAKSRKSGS | 235 |
| | | pCoV1B-06-PL-B.1.351/501Y.V2-L18F_D80A_D215_delta241243_R246_K417N_E484K_N501Y_D614G_A701V | | SSQCVNFTTRTQLPPAYTNSFTRGVYYPDKVFRSSVLHSTQDLFLPFFSN V TWFHAIHVSGTNGTKRFANPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLHRSYLTPGDSSSGWTAGAAAYYV G YLQPRTFLLKYNENGTITDAVDCALDPLSETKCTLKSFTVEKGIYQTSNF R VQPTESIVRFPNITNLCPFGEVFNATRFASVYAWNRKRISNCVADYSVLY N SASFSTFKCYGVSPTKLNDLCFTNVYADSFVIRGDEVRQIAPGQTGNIAD Y NYKLPDDFTGCVIAWNSNNLDSKVGGNYNYLYRLFRKSNLKPFERDISTE IYQAGSTPCNGVKGFNCYFPLQSYGFQPTYGVGYQPYRVVVLSFELLHAP ATVCGPKKSTNLVKNKCVNFNFNGLTGTGVLTESNKKFLPFQQFGRDIAD TTDAVRDPQTLEILDITPCSFGGVSVITPGTNTSNQVAVLYQGVNCTEVP V AIHADQLTPTWRVYSTGSNVFQTRAGCLIGAEHVNNSYECDIPIGAGICA S YQTQTNSPGSASSVASQSIIAYTMSLGVENSVAYSNNSIAIPTNFTISVT TEI LPVSMTKTSVDCTMYICGDSTECSNLLLQYGSFCTQLNRALTGIAVEQDK NTQEVFAQVKQIYKTPPIKDFGGFNFSQILPDPSKPSKRSFIEDLLFNKV TL ADAGFIKQYGDCLGDIAARDLICAQKFNGLTVLPPLLTDEMIAQYTSALL AGTITSGWTFGAGAALQIPFAMQMAYRFNGIGVTQNVLYENQKLIANQF NSAIGKIQDSLSSTASALGKLQDVVNQNAQALNTLVKQLSSNFGAISSVL N DILSRLDPPEAEVQIDRLITGRLQSLQTYVTQQLIRAAEIRASANLAATK MS ECVLGQSKRVDFCGKGYHLMSFPQSAPHGVVFLHVTYVPAQEKNFTTAP AICHDGKAHFPREGVFVSNGTHWFVTQRNFYEPQIITTDNTFVSGNCDVV I GIVNNTVYDPLQSELDSIKEELDKIHKNGSGGSGDIIKLLNEQVNKEMQS S NLYMSMSSWCYTHSLDGAGLFLFDHAAEEYEHAKKLIIFLNENNVPVQL TSISAPEHKFEGLTQIFQKAYEHEQHISESINNIVDHAIKSKDHATFNFL QW YVAEQHEEEVLFKDILDKIELIGNENHGLYLADQYVKGIAKSRKSGS | 236 |
| | | pCoV1B-06-P.1/501Y.V3-L18F_T20N_P26S_D138Y_R190S_K417T_E484K_N501Y_D614G_H655Y_T1027I | | SSQCVNFTNRTQLPSAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNYPFLGVYYHKNNKSWMESEFRVYSSA NNCTFEYVSQPFLMDLEGKQGNFKNLSEFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGT I ADYNYKLPDDFTGCVIAWNSNNLDSKVGGNYNYLYRLFRKSNLKPFERD ISTEIYQAGSTPCNGVKGFNCYFPLQSYGFQPTYGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQGVNC T EVPVAIHADQLTPTWRVYSTGSNVFQTRAGCLIGAEYVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSFIEDLL FN KVTLADAGFIKQYGDCLGDIAARDLICAQKFNGLTVLPPLLTDEMIAQYT SALLAGTITSGWTFGAGAALQIPFAMQMAYRFNGIGVTQNVLYENQKLIA NQFNSAIGKIQDSLSSTASALGKLQDVVNQNAQALNTLVKQLSSNFGAIS S VLNDILSRLDPPEAEVQIDRLITGRLQSLQTYVTQQLIRAAEIRASANLA AI KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQSELDSIKEELDKIHKNGSGGSGDIIKLLNEQVNK E MQSSNLYMSMSSWCYTHSLDGAGLFLFDHAAEEYEHAKKLIIFLNENNV PVQLTSISAPEHKFEGLTQIFQKAYEHEQHISESINNIVDHAIKSKDHAT FNF LQWYVAEQHEEEVLFKDILDKIELIGNENHGLYLADQYVKGIAKSRKSGS | 237 |
| | | pCoV187-B.1.1.7/501Y.V1-delta69-70_delta144-N501Y-A570D-D614G-P681H-T716I-S982A-D1118H | | SSQCVNLTTRTQLPPAYTNSFTRGVYYPDKVFRSSVLHSTQDLFLPFFSN V TWFHAISGTNGTKRFDNPVLPFNDGVYFASTEKSNIIRGWIFGTTLDSKT Q SLLIVNNATNVVIKVCEFQFCNDPFLGVYHKNNKSWMESEFRVYSSANN CTFEYVSQPFLMDLEGKQGNFKNLREFVFKNIDGYFKIYSKHTPINLVRD L PQGFSALEPLVDLPIGINITRFQTLLALHRSYLTPGDSSSGWTAGAAAYY V GYLQPRTFLLKYNENGTITDAVDCALDPLSETKCTLKSFTVEKGIYQTSN F RVQPTESIVRFPNITNLCPFGEVFNATRFASVYAWNRKRISNCVADYSVL Y NSASFSTFKCYGVSPTKLNDLCFTNVYADSFVIRGDEVRQIAPGQTGKIA D YNYKLPDDFTGCVIAWNSNNLDSKVGGNYNYLYRLFRKSNLKPFERDIST EIYQAGSTPCNGVEGFNCYFPLQSYGFQPTYGVGYQPYRVVVLSFELLHA PATVCGPKKSTNLVKNKCVNFNFNGLTGTGVLTESNKKFLPFQQFGRDID DTTDAVRDPQTLEILDITPCSFGGVSVITPGTNTSNQVAVLYQGVNCTEV P VAIHADQLTPTWRVYSTGSNVFQTRAGCLIGAEHVNNSYECDIPIGAGIC A SYQTQTNSHGSASSVASQSIIAYTMSLGAENSVAYSNNSIAIPINFTISV TTE ILPVSMTKTSVDCTMYICGDSTECSNLLLQYGSFCTQLNRALTGIAVEQD K NTQEVFAQVKQIYKTPPIKDFGGFNFSQILPDPSKPSKRSFIEDLLFNKV TL ADAGFIKQYGDCLGDIAARDLICAQKFNGLTVLPPLLTDEMIAQYTSALL AGTITSGWTFGAGAALQIPFAMQMAYRFNGIGVTQNVLYENQKLIANQF NSAIGKIQDSLSSTASALGKLQDVVNQNAQALNTLVKQLSSNFGAISSVL N DILARLDPPEAEVQIDRLITGRLQSLQTYVTQQLIRAAEIRASANLAATK M SECVLGQSKRVDFCGKGYHLMSFPQSAPHGVVFLHVTYVPAQEKNFTTA PAICHDGKAHFPREGVFVSNGTHWFVTQRNFYEPQIITTHNTFVSGNCDV VIGIVNNTVYDPLQSELDSIKEELDKIHKNLDSIKEELDKIHKNGSGGSG DII KLLNEQVNKEMQSSNLYMSMSSWCYTHSLDGAGLFLFDHAAEEYEHAK KLIIFLNENNVPVQLTSISAPEHKFEGLTQIFQKAYEHEQHISESINNIV DHAI KSKDHATFNFLQWYVAEQHEEEVLFKDILDKIELIGNENHGLYLADQYVK GIAKSRKSGS | 238 |
| | | pCoV187-B.1.351/501Y.V2-L18F_D80A_D215G_delta241-243_R246I_K417N_E484K_N501Y_D614G_A701V | | SSQCVNFTTRTQLPPAYTNSFTRGVYYPDKVFRSSVLHSTQDLFLPFFSN V TWFHAIHVSGTNGTKRFANPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R GLPQGFSALEPLVDLPIGINITRFQTLHISYLTPGDSSSGWTAGAAAYYV G YLQPRTFLLKYNENGTITDAVDCALDPLSETKCTLKSFTVEKGIYQTSNF R VQPTESIVRFPNITNLCPFGEVFNATRFASVYAWNRKRISNCVADYSVLY N SASFSTFKCYGVSPTKLNDLCFTNVYADSFVIRGDEVRQIAPGQTGNIAD Y NYKLPDDFTGCVIAWNSNNLDSKVGGNYNYLYRLFRKSNLKPFERDISTE IYQAGSTPCNGVKGFNCYFPLQSYGFQPTYGVGYQPYRVVVLSFELLHAP ATVCGPKKSTNLVKNKCVNFNFNGLTGTGVLTESNKKFLPFQQFGRDIAD TTDAVRDPQTLEILDITPCSFGGVSVITPGTNTSNQVAVLYQGVNCTEVP V AIHADQLTPTWRVYSTGSNVFQTRAGCLIGAEHVNNSYECDIPIGAGICA S YQTQTNSPGSASSVASQSIIAYTMSLGVENSVAYSNNSIAIPTNFTISVT TEI LPVSMTKTSVDCTMYICGDSTECSNLLLQYGSFCTQLNRALTGIAVEQDK NTQEVFAQVKQIYKTPPIKDFGGFNFSQILPDPSKPSKRSFIEDLLFNKV TL ADAGFIKQYGDCLGDIAARDLICAQKFNGLTVLPPLLTDEMIAQYTSALL AGTITSGWTFGAGAALQIPFAMQMAYRFNGIGVTQNVLYENQKLIANQF NSAIGKIQDSLSSTASALGKLQDVVNQNAQALNTLVKQLSSNFGAISSVL N DILSRLDPPEAEVQIDRLITGRLQSLQTYVTQQLIRAAEIRASANLAATK MS ECVLGQSKRVDFCGKGYHLMSFPQSAPHGVVFLHVTYVPAQEKNFTTAP AICHDGKAHFPREGVFVSNGTHWFVTQRNFYEPQIITTDNTFVSGNCDVV I GIVNNTVYDPLQSELDSIKEELDKIHKNLDSIKEELDKIHKNGSGGSGDI IK LLNEQVNKEMQSSNLYMSMSSWCYTHSLDGAGLFLFDHAAEEYEHAKK LIIFLNENNVPVQLTSISAPEHKFEGLTQIFQKAYEHEQHISESINNIVD HAIK SKDHATFNFLQWYVAEQHEEEVLFKDILDKIELIGNENHGLYLADQYVKG IAKSRKSGS | 239 |
| | | pCoV187-B.1.351/501Y. V2-L18F_D80_D2 15G_delta241- 243_R246I_K417N_E484K_N501Y_D614G_A701V | | SSQCVNFTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R GLPQGFSALEPLVDLPIGINITRFQTLHISYLTPGDSSSGWTAGAAAYYV G YLQPRTFLLKYNENGTITDAVDCALDPLSETKCTLKSFTVEKGIYQTSNF R VQPTESIVRFPNITNLCPFGEVFNATRFASVYAWNRKRISNCVADYSVLY N SASFSTFKCYGVSPTKLNDLCFTNVYADSFVIRGDEVRQIAPGQTGNIAD Y NYKLPDDFTGCVIAWNSNNLDSKVGGNYNYLYRLFRKSNLKPFERDISTE IYQAGSTPCNGVKGFNCYFPLQSYGFQPTYGVGYQPYRVVVLSFELLHAP ATVCGPKKSTNLVKNKCVNFNFNGLTGTGVLTESNKKFLPFQQFGRDIAD TTDAVRDPQTLEILDITPCSFGGVSVITPGTNTSNQVAVLYQGVNCTEVP V AIHADQLTPTWRVYSTGSNVFQTRAGCLIGAEHVNNSYECDIPIGAGICA S YQTQTNSPGSASSVASQSIIAYTMSLGVENSVAYSNNSIAIPTNFTISVT TEI LPVSMTKTSVDCTMYICGDSTECSNLLLQYGSFCTQLNRALTGIAVEQDK NTQEVFAQVKQIYKTPPIKDFGGFNFSQILPDPSKPSKRSFIEDLLFNKV TL ADAGFIKQYGDCLGDIAARDLICAQKFNGLTVLPPLLTDEMIAQYTSALL AGTITSGWTFGAGAALQIPFAMQMAYRFNGIGVTQNVLYENQKLIANQF NSAIGKIQDSLSSTASALGKLQDVVNQNAQALNTLVKQLSSNFGAISSVL N DILSRLDPPEAEVQIDRLITGRLQSLQTYVTQQLIRAAEIRASANLAATK MS ECVLGQSKRVDFCGKGYHLMSFPQSAPHGVVFLHVTYVPAQEKNFTTAP AICHDGKAHFPREGVFVSNGTHWFVTQRNFYEPQIITTDNTFVSGNCDVV I GIVNNTVYDPLQSELDSIKEELDKIHKNLDSIKEELDKIHKNGSGGSGDI IK LLNEQVNKEMQSSNLYMSMSSWCYTHSLDGAGLFLFDHAAEEYEHAKK LIIFLNENNVPVQLTSISAPEHKFEGLTQIFQKAYEHEQHISESINNIVD HAIK SKDHATFNFLQWYVAEQHEEEVLFKDILDKIELIGNENHGLYLADQYVKG IAKSRKSGS | 240 |
| | | pCoV187-B.1.351/501Y.V2-L18F_D80A_ D215_delta241 243_R246I_K 417N_E484K_ N501Y_D614 G_A701V | | SSQCVNFTTRTQLPPAYTNSFTRGVYYPDKVFRSSVLHSTQDLFLPFFSN V TWFHAIHVSGTNGTKRFANPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLHISYLTPGDSSSGWTAGAAAYYV G YLQPRTFLLKYNENGTITDAVDCALDPLSETKCTLKSFTVEKGIYQTSNF R VQPTESIVRFPNITNLCPFGEVFNATRFASVYAWNRKRISNCVADYSVLY N SASFSTFKCYGVSPTKLNDLCFTNVYADSFVIRGDEVRQIAPGQTGNIAD Y NYKLPDDFTGCVIAWNSNNLDSKVGGNYNYLYRLFRKSNLKPFERDISTE IYQAGSTPCNGVKGFNCYFPLQSYGFQPTYGVGYQPYRVVVLSFELLHAP ATVCGPKKSTNLVKNKCVNFNFNGLTGTGVLTESNKKFLPFQQFGRDIAD TTDAVRDPQTLEILDITPCSFGGVSVITPGTNTSNQVAVLYQGVNCTEVP V AIHADQLTPTWRVYSTGSNVFQTRAGCLIGAEHVNNSYECDIPIGAGICA S YQTQTNSPGSASSVASQSIIAYTMSLGVENSVAYSNNSIAIPTNFTISVT TEI LPVSMTKTSVDCTMYICGDSTECSNLLLQYGSFCTQLNRALTGIAVEQDK NTQEVFAQVKQIYKTPPIKDFGGFNFSQILPDPSKPSKRSFIEDLLFNKV TL ADAGFIKQYGDCLGDIAARDLICAQKFNGLTVLPPLLTDEMIAQYTSALL AGTITSGWTFGAGAALQIPFAMQMAYRFNGIGVTQNVLYENQKLIANQF NSAIGKIQDSLSSTASALGKLQDVVNQNAQALNTLVKQLSSNFGAISSVL N DILSRLDPPEAEVQIDRLITGRLQSLQTYVTQQLIRAAEIRASANLAATK MS ECVLGQSKRVDFCGKGYHLMSFPQSAPHGVVFLHVTYVPAQEKNFTTAP AICHDGKAHFPREGVFVSNGTHWFVTQRNFYEPQIITTDNTFVSGNCDVV I GIVNNTVYDPLQSELDSIKEELDKIHKNLDSIKEELDKIHKNGSGGSGDI IK LLNEQVNKEMQSSNLYMSMSSWCYTHSLDGAGLFLFDHAAEEYEHAKK LIIFLNENNVPVQLTSISAPEHKFEGLTQIFQKAYEHEQHISESINNIVD HAIK SKDHATFNFLQWYVAEQHEEEVLFKDILDKIELIGNENHGLYLADQYVKG IAKSRKSGS | 241 |
| | | pCoV187-B.1.351/501Y.V2-L18F_D80A_D215G_delta241-243_R246_K417N_E484K_N501Y_D614G_A701V | | SSQCVNFTTRTQLPPAYTNSFTRGVYYPDKVFRSSVLHSTQDLFLPFFSN V TWFHAIHVSGTNGTKRFANPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R GLPQGFSALEPLVDLPIGINITRFQTLHRSYLTPGDSSSGWTAGAAAYYV G YLQPRTFLLKYNENGTITDAVDCALDPLSETKCTLKSFTVEKGIYQTSNF R VQPTESIVRFPNITNLCPFGEVFNATRFASVYAWNRKRISNCVADYSVLY N SASFSTFKCYGVSPTKLNDLCFTNVYADSFVIRGDEVRQIAPGQTGNIAD Y NYKLPDDFTGCVIAWNSNNLDSKVGGNYNYLYRLFRKSNLKPFERDISTE IYQAGSTPCNGVKGFNCYFPLQSYGFQPTYGVGYQPYRVVVLSFELLHAP ATVCGPKKSTNLVKNKCVNFNFNGLTGTGVLTESNKKFLPFQQFGRDIAD TTDAVRDPQTLEILDITPCSFGGVSVITPGTNTSNQVAVLYQGVNCTEVP V AIHADQLTPTWRVYSTGSNVFQTRAGCLIGAEHVNNSYECDIPIGAGICA S YQTQTNSPGSASSVASQSIIAYTMSLGVENSVAYSNNSIAIPTNFTISVT TEI LPVSMTKTSVDCTMYICGDSTECSNLLLQYGSFCTQLNRALTGIAVEQDK NTQEVFAQVKQIYKTPPIKDFGGFNFSQILPDPSKPSKRSFIEDLLFNKV TL ADAGFIKQYGDCLGDIAARDLICAQKFNGLTVLPPLLTDEMIAQYTSALL AGTITSGWTFGAGAALQIPFAMQMAYRFNGIGVTQNVLYENQKLIANQF NSAIGKIQDSLSSTASALGKLQDVVNQNAQALNTLVKQLSSNFGAISSVL N DILSRLDPPEAEVQIDRLITGRLQSLQTYVTQQLIRAAEIRASANLAATK MS ECVLGQSKRVDFCGKGYHLMSFPQSAPHGVVFLHVTYVPAQEKNFTTAP AICHDGKAHFPREGVFVSNGTHWFVTQRNFYEPQIITTDNTFVSGNCDVV I GIVNNTVYDPLQSELDSIKEELDKIHKNLDSIKEELDKIHKNGSGGSGDI IK LLNEQVNKEMQSSNLYMSMSSWCYTHSLDGAGLFLFDHAAEEYEHAKK LIIFLNENNVPVQLTSISAPEHKFEGLTQIFQKAYEHEQHISESINNIVD HAIK SKDHATFNFLQWYVAEQHEEEVLFKDILDKIELIGNENHGLYLADQYVKG IAKSRKSGS | 242 |
| | | pCoV187-B.1.351/501Y.V2-L18F_D80_D215G_delta241-243_R246_K417N_E484K_N501Y_D614G_A701V | | SSQCVNFTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R GLPQGFSALEPLVDLPIGINITRFQTLHRSYLTPGDSSSGWTAGAAAYYV G YLQPRTFLLKYNENGTITDAVDCALDPLSETKCTLKSFTVEKGIYQTSNF R VQPTESIVRFPNITNLCPFGEVFNATRFASVYAWNRKRISNCVADYSVLY N SASFSTFKCYGVSPTKLNDLCFTNVYADSFVIRGDEVRQIAPGQTGNIAD Y NYKLPDDFTGCVIAWNSNNLDSKVGGNYNYLYRLFRKSNLKPFERDISTE IYQAGSTPCNGVKGFNCYFPLQSYGFQPTYGVGYQPYRVVVLSFELLHAP ATVCGPKKSTNLVKNKCVNFNFNGLTGTGVLTESNKKFLPFQQFGRDIAD TTDAVRDPQTLEILDITPCSFGGVSVITPGTNTSNQVAVLYQGVNCTEVP V AIHADQLTPTWRVYSTGSNVFQTRAGCLIGAEHVNNSYECDIPIGAGICA S YQTQTNSPGSASSVASQSIIAYTMSLGVENSVAYSNNSIAIPTNFTISVT TEI LPVSMTKTSVDCTMYICGDSTECSNLLLQYGSFCTQLNRALTGIAVEQDK NTQEVFAQVKQIYKTPPIKDFGGFNFSQILPDPSKPSKRSFIEDLLFNKV TL ADAGFIKQYGDCLGDIAARDLICAQKFNGLTVLPPLLTDEMIAQYTSALL AGTITSGWTFGAGAALQIPFAMQMAYRFNGIGVTQNVLYENQKLIANQF NSAIGKIQDSLSSTASALGKLQDVVNQNAQALNTLVKQLSSNFGAISSVL N DILSRLDPPEAEVQIDRLITGRLQSLQTYVTQQLIRAAEIRASANLAATK MS ECVLGQSKRVDFCGKGYHLMSFPQSAPHGVVFLHVTYVPAQEKNFTTAP AICHDGKAHFPREGVFVSNGTHWFVTQRNFYEPQIITTDNTFVSGNCDVV I GIVNNTVYDPLQSELDSIKEELDKIHKNLDSIKEELDKIHKNGSGGSGDI IK LLNEQVNKEMQSSNLYMSMSSWCYTHSLDGAGLFLFDHAAEEYEHAKK LIIFLNENNVPVQLTSISAPEHKFEGLTQIFQKAYEHEQHISESINNIVD HAIK SKDHATFNFLQWYVAEQHEEEVLFKDILDKIELIGNENHGLYLADQYVKG IAKSRKSGS | 243 |
| | | pCoV187-B.1.351/501Y.V2-L18F_D80A_D215_delta241243_R246_K417N_E484K_N501Y_D614G_A701V | | SSQCVNFTTRTQLPPAYTNSFTRGVYYPDKVFRSSVLHSTQDLFLPFFSN V TWFHAIHVSGTNGTKRFANPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLHRSYLTPGDSSSGWTAGAAAYYV G YLQPRTFLLKYNENGTITDAVDCALDPLSETKCTLKSFTVEKGIYQTSNF R VQPTESIVRFPNITNLCPFGEVFNATRFASVYAWNRKRISNCVADYSVLY N SASFSTFKCYGVSPTKLNDLCFTNVYADSFVIRGDEVRQIAPGQTGNIAD Y NYKLPDDFTGCVIAWNSNNLDSKVGGNYNYLYRLFRKSNLKPFERDISTE IYQAGSTPCNGVKGFNCYFPLQSYGFQPTYGVGYQPYRVVVLSFELLHAP ATVCGPKKSTNLVKNKCVNFNFNGLTGTGVLTESNKKFLPFQQFGRDIAD TTDAVRDPQTLEILDITPCSFGGVSVITPGTNTSNQVAVLYQGVNCTEVP V AIHADQLTPTWRVYSTGSNVFQTRAGCLIGAEHVNNSYECDIPIGAGICA S YQTQTNSPGSASSVASQSIIAYTMSLGVENSVAYSNNSIAIPTNFTISVT TEI LPVSMTKTSVDCTMYICGDSTECSNLLLQYGSFCTQLNRALTGIAVEQDK NTQEVFAQVKQIYKTPPIKDFGGFNFSQILPDPSKPSKRSFIEDLLFNKV TL ADAGFIKQYGDCLGDIAARDLICAQKFNGLTVLPPLLTDEMIAQYTSALL AGTITSGWTFGAGAALQIPFAMQMAYRFNGIGVTQNVLYENQKLIANQF NSAIGKIQDSLSSTASALGKLQDVVNQNAQALNTLVKQLSSNFGAISSVL N DILSRLDPPEAEVQIDRLITGRLQSLQTYVTQQLIRAAEIRASANLAATK MS ECVLGQSKRVDFCGKGYHLMSFPQSAPHGVVFLHVTYVPAQEKNFTTAP AICHDGKAHFPREGVFVSNGTHWFVTQRNFYEPQIITTDNTFVSGNCDVV I GIVNNTVYDPLQSELDSIKEELDKIHKNLDSIKEELDKIHKNGSGGSGDI IK LLNEQVNKEMQSSNLYMSMSSWCYTHSLDGAGLFLFDHAAEEYEHAKK LIIFLNENNVPVQLTSISAPEHKFEGLTQIFQKAYEHEQHISESINNIVD HAIK SKDHATFNFLQWYVAEQHEEEVLFKDILDKIELIGNENHGLYLADQYVKG IAKSRKSGS | 244 |
| | | pCoV187-P.1/501Y.V3-L18F_T20N_P26S_D138Y_R190S_K417T_E484K_N501Y_D614G_H655Y_T1027I | | SSQCVNFTNRTQLPSAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNYPFLGVYYHKNNKSWMESEFRVYSSA NNCTFEYVSQPFLMDLEGKQGNFKNLSEFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGT I ADYNYKLPDDFTGCVIAWNSNNLDSKVGGNYNYLYRLFRKSNLKPFERD ISTEIYQAGSTPCNGVKGFNCYFPLQSYGFQPTYGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQGVNC T EVPVAIHADQLTPTWRVYSTGSNVFQTRAGCLIGAEYVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSFIEDLL FN KVTLADAGFIKQYGDCLGDIAARDLICAQKFNGLTVLPPLLTDEMIAQYT SALLAGTITSGWTFGAGAALQIPFAMQMAYRFNGIGVTQNVLYENQKLIA NQFNSAIGKIQDSLSSTASALGKLQDVVNQNAQALNTLVKQLSSNFGAIS S VLNDILSRLDPPEAEVQIDRLITGRLQSLQTYVTQQLIRAAEIRASANLA AI KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQSELDSIKEELDKIHKNLDSIKEELDKIHKNGSGG SG DIIKLLNEQVNKEMQSSNLYMSMSSWCYTHSLDGAGLFLFDHAAEEYEH AKKLIIFLNENNVPVQLTSISAPEHKFEGLTQIFQKAYEHEQHISESINN IVD HAIKSKDHATFNFLQWYVAEQHEEEVLFKDILDKIELIGNENHGLYLADQ YVKGIAKSRKSGS | 245 |
| | | pCoV186-B.1.1.7/501Y.V1-delta69-70_deltal44-N501Y-A570D-D614G-P681H-T716I-S982A-D1118H | | SSQCVNLTTRTQLPPAYTNSFTRGVYYPDKVFRSSVLHSTQDLFLPFFSN V TWFHAISGTNGTKRFDNPVLPFNDGVYFASTEKSNIIRGWIFGTTLDSKT Q SLLIVNNATNVVIKVCEFQFCNDPFLGVYHKNNKSWMESEFRVYSSANN CTFEYVSQPFLMDLEGKQGNFKNLREFVFKNIDGYFKIYSKHTPINLVRD L PQGFSALEPLVDLPIGINITRFQTLLALHRSYLTPGDSSSGWTAGAAAYY V GYLQPRTFLLKYNENGTITDAVDCALDPLSETKCTLKSFTVEKGIYQTSN F RVQPTESIVRFPNITNLCPFGEVFNATRFASVYAWNRKRISNCVADYSVL Y NSASFSTFKCYGVSPTKLNDLCFTNVYADSFVIRGDEVRQIAPGQTGKIA D YNYKLPDDFTGCVIAWNSNNLDSKVGGNYNYLYRLFRKSNLKPFERDIST EIYQAGSTPCNGVEGFNCYFPLQSYGFQPTYGVGYQPYRVVVLSFELLHA PATVCGPKKSTNLVKNKCVNFNFNGLTGTGVLTESNKKFLPFQQFGRDID DTTDAVRDPQTLEILDITPCSFGGVSVITPGTNTSNQVAVLYQGVNCTEV P VAIHADQLTPTWRVYSTGSNVFQTRAGCLIGAEHVNNSYECDIPIGAGIC A SYQTQTNSHGSASSVASQSIIAYTMSLGAENSVAYSNNSIAIPINFTISV TTE ILPVSMTKTSVDCTMYICGDSTECSNLLLQYGSFCTQLNRALTGIAVEQD K NTQEVFAQVKQIYKTPPIKDFGGFNFSQILPDPSKPSKRSPIEDLLFNKV TL ADAGFIKQYGDCLGDIAARDLICAQKFNGLTVLPPLLTDEMIAQYTSALL AGTITSGWTFGAGPALQIPFPMQMAYRFNGIGVTQNVLYENQKLIANQFN SAIGKIQDSLSSTPSALGKLQDVVNQNAQALNTLVKQLSSNFGAISSVLN D ILARLDPPEAEVQIDRLITGRLQSLQTYVTQQLIRAAEIRASANLAATKM SE CVLGQSKRVDFCGKGYHLMSFPQSAPHGVVFLHVTYVPAQEKNFTTAPA ICHDGKAHFPREGVFVSNGTHWFVTQRNFYEPQIITTHNTFVSGNCDVVI G IVNNTVYDPLQSELDSIKEELDKIHKNGSGGSGDIIKLLNEQVNKEMQSS N LYMSMSSWCYTHSLDGAGLFLFDHAAEEYEHAKKLIIFLNENNVPVQLTS ISAPEHKFEGLTQIFQKAYEHEQHISESINNIVDHAIKSKDHATFNFLQW YV AEQHEEEVLFKDILDKIELIGNENHGLYLADQYVKGIAKSRKSGS | 246 |
| | | pCoV186-.1.351/501Y.V2-L18F_D80A_D215G_delta241-243_R246I_K417N_E484K_N501Y_D614G_A701V | | SSQCVNFTTRTQLPPAYTNSFTRGVYYPDKVFRSSVLHSTQDLFLPFFSN V TWFHAIHVSGTNGTKRFANPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R GLPQGFSALEPLVDLPIGINITRFQTLHISYLTPGDSSSGWTAGAAAYYV G YLQPRTFLLKYNENGTITDAVDCALDPLSETKCTLKSFTVEKGIYQTSNF R VQPTESIVRFPNITNLCPFGEVFNATRFASVYAWNRKRISNCVADYSVLY N SASFSTFKCYGVSPTKLNDLCFTNVYADSFVIRGDEVRQIAPGQTGNIAD Y NYKLPDDFTGCVIAWNSNNLDSKVGGNYNYLYRLFRKSNLKPFERDISTE IYQAGSTPCNGVKGFNCYFPLQSYGFQPTYGVGYQPYRVVVLSFELLHAP ATVCGPKKSTNLVKNKCVNFNFNGLTGTGVLTESNKKFLPFQQFGRDIAD TTDAVRDPQTLEILDITPCSFGGVSVITPGTNTSNQVAVLYQGVNCTEVP V AIHADQLTPTWRVYSTGSNVFQTRAGCLIGAEHVNNSYECDIPIGAGICA S YQTQTNSPGSASSVASQSIIAYTMSLGVENSVAYSNNSIAIPTNFTISVT TEI LPVSMTKTSVDCTMYICGDSTECSNLLLQYGSFCTQLNRALTGIAVEQDK NTQEVFAQVKQIYKTPPIKDFGGFNFSQILPDPSKPSKRSPIEDLLFNKV TL ADAGFIKQYGDCLGDIAARDLICAQKFNGLTVLPPLLTDEMIAQYTSALL AGTITSGWTFGAGPALQIPFPMQMAYRFNGIGVTQNVLYENQKLIANQFN SAIGKIQDSLSSTPSALGKLQDVVNQNAQALNTLVKQLSSNFGAISSVLN D ILSRLDPPEAEVQIDRLITGRLQSLQTYVTQQLIRAAEIRASANLAATKM SE CVLGQSKRVDFCGKGYHLMSFPQSAPHGVVFLHVTYVPAQEKNFTTAPA ICHDGKAHFPREGVFVSNGTHWFVTQRNFYEPQIITTDNTFVSGNCDVVI G IVNNTVYDPLQSELDSIKEELDKIHKNGSGGSGDIIKLLNEQVNKEMQSS N LYMSMSSWCYTHSLDGAGLFLFDHAAEEYEHAKKLIIFLNENNVPVQLTS ISAPEHKFEGLTQIFQKAYEHEQHISESINNIVDHAIKSKDHATFNFLQW YV AEQHEEEVLFKDILDKIELIGNENHGLYLADQYVKGIAKSRKSGS | 247 |
| | | pCoV186-B.1.351/501Y.V2-L18F_D80_D215G_delta241-243_R246I_K417N_E484K_N501Y_D614G_A701V | | SSQCVNFTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R GLPQGFSALEPLVDLPIGINITRFQTLHISYLTPGDSSSGWTAGAAAYYV G YLQPRTFLLKYNENGTITDAVDCALDPLSETKCTLKSFTVEKGIYQTSNF R VQPTESIVRFPNITNLCPFGEVFNATRFASVYAWNRKRISNCVADYSVLY N SASFSTFKCYGVSPTKLNDLCFTNVYADSFVIRGDEVRQIAPGQTGNIAD Y NYKLPDDFTGCVIAWNSNNLDSKVGGNYNYLYRLFRKSNLKPFERDISTE IYQAGSTPCNGVKGFNCYFPLQSYGFQPTYGVGYQPYRVVVLSFELLHAP ATVCGPKKSTNLVKNKCVNFNFNGLTGTGVLTESNKKFLPFQQFGRDIAD TTDAVRDPQTLEILDITPCSFGGVSVITPGTNTSNQVAVLYQGVNCTEVP V AIHADQLTPTWRVYSTGSNVFQTRAGCLIGAEHVNNSYECDIPIGAGICA S YQTQTNSPGSASSVASQSIIAYTMSLGVENSVAYSNNSIAIPTNFTISVT TEI LPVSMTKTSVDCTMYICGDSTECSNLLLQYGSFCTQLNRALTGIAVEQDK NTQEVFAQVKQIYKTPPIKDFGGFNFSQILPDPSKPSKRSPIEDLLFNKV TL ADAGFIKQYGDCLGDIAARDLICAQKFNGLTVLPPLLTDEMIAQYTSALL AGTITSGWTFGAGPALQIPFPMQMAYRFNGIGVTQNVLYENQKLIANQFN SAIGKIQDSLSSTPSALGKLQDVVNQNAQALNTLVKQLSSNFGAISSVLN D ILSRLDPPEAEVQIDRLITGRLQSLQTYVTQQLIRAAEIRASANLAATKM SE CVLGQSKRVDFCGKGYHLMSFPQSAPHGVVFLHVTYVPAQEKNFTTAPA ICHDGKAHFPREGVFVSNGTHWFVTQRNFYEPQIITTDNTFVSGNCDVVI G IVNNTVYDPLQSELDSIKEELDKIHKNGSGGSGDIIKLLNEQVNKEMQSS N LYMSMSSWCYTHSLDGAGLFLFDHAAEEYEHAKKLIIFLNENNVPVQLTS ISAPEHKFEGLTQIFQKAYEHEQHISESINNIVDHAIKSKDHATFNFLQW YV AEQHEEEVLFKDILDKIELIGNENHGLYLADQYVKGIAKSRKSGS | 248 |
| | | pCoV186-B.1.351/501Y.V2-L18F_D80A_D215_delta241243_R246I_K417N_E484K_N501Y_D614G_A701V | | SSQCVNFTTRTQLPPAYTNSFTRGVYYPDKVFRSSVLHSTQDLFLPFFSN V TWFHAIHVSGTNGTKRFANPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLHISYLTPGDSSSGWTAGAAAYYV G YLQPRTFLLKYNENGTITDAVDCALDPLSETKCTLKSFTVEKGIYQTSNF R VQPTESIVRFPNITNLCPFGEVFNATRFASVYAWNRKRISNCVADYSVLY N SASFSTFKCYGVSPTKLNDLCFTNVYADSFVIRGDEVRQIAPGQTGNIAD Y NYKLPDDFTGCVIAWNSNNLDSKVGGNYNYLYRLFRKSNLKPFERDISTE IYQAGSTPCNGVKGFNCYFPLQSYGFQPTYGVGYQPYRVVVLSFELLHAP ATVCGPKKSTNLVKNKCVNFNFNGLTGTGVLTESNKKFLPFQQFGRDIAD TTDAVRDPQTLEILDITPCSFGGVSVITPGTNTSNQVAVLYQGVNCTEVP V AIHADQLTPTWRVYSTGSNVFQTRAGCLIGAEHVNNSYECDIPIGAGICA S YQTQTNSPGSASSVASQSIIAYTMSLGVENSVAYSNNSIAIPTNFTISVT TEI LPVSMTKTSVDCTMYICGDSTECSNLLLQYGSFCTQLNRALTGIAVEQDK NTQEVFAQVKQIYKTPPIKDFGGFNFSQILPDPSKPSKRSPIEDLLFNKV TL ADAGFIKQYGDCLGDIAARDLICAQKFNGLTVLPPLLTDEMIAQYTSALL AGTITSGWTFGAGPALQIPFPMQMAYRFNGIGVTQNVLYENQKLIANQFN SAIGKIQDSLSSTPSALGKLQDVVNQNAQALNTLVKQLSSNFGAISSVLN D ILSRLDPPEAEVQIDRLITGRLQSLQTYVTQQLIRAAEIRASANLAATKM SE CVLGQSKRVDFCGKGYHLMSFPQSAPHGVVFLHVTYVPAQEKNFTTAPA ICHDGKAHFPREGVFVSNGTHWFVTQRNFYEPQIITTDNTFVSGNCDVVI G IVNNTVYDPLQSELDSIKEELDKIHKNGSGGSGDIIKLLNEQVNKEMQSS N LYMSMSSWCYTHSLDGAGLFLFDHAAEEYEHAKKLIIFLNENNVPVQLTS ISAPEHKFEGLTQIFQKAYEHEQHISESINNIVDHAIKSKDHATFNFLQW YV AEQHEEEVLFKDILDKIELIGNENHGLYLADQYVKGIAKSRKSGS | 249 |
| | | pCoV186-B.1.351/501Y.V2-L18F_D80A_D215G_delta241-243_R246_K417N_E484K_N501Y_D614G_A701V | | SSQCVNFTTRTQLPPAYTNSFTRGVYYPDKVFRSSVLHSTQDLFLPFFSN V TWFHAIHVSGTNGTKRFANPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R GLPQGFSALEPLVDLPIGINITRFQTLHRSYLTPGDSSSGWTAGAAAYYV G YLQPRTFLLKYNENGTITDAVDCALDPLSETKCTLKSFTVEKGIYQTSNF R VQPTESIVRFPNITNLCPFGEVFNATRFASVYAWNRKRISNCVADYSVLY N SASFSTFKCYGVSPTKLNDLCFTNVYADSFVIRGDEVRQIAPGQTGNIAD Y NYKLPDDFTGCVIAWNSNNLDSKVGGNYNYLYRLFRKSNLKPFERDISTE IYQAGSTPCNGVKGFNCYFPLQSYGFQPTYGVGYQPYRVVVLSFELLHAP ATVCGPKKSTNLVKNKCVNFNFNGLTGTGVLTESNKKFLPFQQFGRDIAD TTDAVRDPQTLEILDITPCSFGGVSVITPGTNTSNQVAVLYQGVNCTEVP V AIHADQLTPTWRVYSTGSNVFQTRAGCLIGAEHVNNSYECDIPIGAGICA S YQTQTNSPGSASSVASQSIIAYTMSLGVENSVAYSNNSIAIPTNFTISVT TEI LPVSMTKTSVDCTMYICGDSTECSNLLLQYGSFCTQLNRALTGIAVEQDK NTQEVFAQVKQIYKTPPIKDFGGFNFSQILPDPSKPSKRSPIEDLLFNKV TL ADAGFIKQYGDCLGDIAARDLICAQKFNGLTVLPPLLTDEMIAQYTSALL AGTITSGWTFGAGPALQIPFPMQMAYRFNGIGVTQNVLYENQKLIANQFN SAIGKIQDSLSSTPSALGKLQDVVNQNAQALNTLVKQLSSNFGAISSVLN D ILSRLDPPEAEVQIDRLITGRLQSLQTYVTQQLIRAAEIRASANLAATKM SE CVLGQSKRVDFCGKGYHLMSFPQSAPHGVVFLHVTYVPAQEKNFTTAPA ICHDGKAHFPREGVFVSNGTHWFVTQRNFYEPQIITTDNTFVSGNCDVVI G IVNNTVYDPLQSELDSIKEELDKIHKNGSGGSGDIIKLLNEQVNKEMQSS N LYMSMSSWCYTHSLDGAGLFLFDHAAEEYEHAKKLIIFLNENNVPVQLTS ISAPEHKFEGLTQIFQKAYEHEQHISESINNIVDHAIKSKDHATFNFLQW YV AEQHEEEVLFKDILDKIELIGNENHGLYLADQYVKGIAKSRKSGS | 250 |
| | | pCoV186-B.1.351/501Y.V2-L18F_D80_D215G_delta241-243_R246_K417N_E484K_N501Y_D614G_A701V | | SSQCVNFTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R GLPQGFSALEPLVDLPIGINITRFQTLHRSYLTPGDSSSGWTAGAAAYYV G YLQPRTFLLKYNENGTITDAVDCALDPLSETKCTLKSFTVEKGIYQTSNF R VQPTESIVRFPNITNLCPFGEVFNATRFASVYAWNRKRISNCVADYSVLY N SASFSTFKCYGVSPTKLNDLCFTNVYADSFVIRGDEVRQIAPGQTGNIAD Y NYKLPDDFTGCVIAWNSNNLDSKVGGNYNYLYRLFRKSNLKPFERDISTE IYQAGSTPCNGVKGFNCYFPLQSYGFQPTYGVGYQPYRVVVLSFELLHAP ATVCGPKKSTNLVKNKCVNFNFNGLTGTGVLTESNKKFLPFQQFGRDIAD TTDAVRDPQTLEILDITPCSFGGVSVITPGTNTSNQVAVLYQGVNCTEVP V AIHADQLTPTWRVYSTGSNVFQTRAGCLIGAEHVNNSYECDIPIGAGICA S YQTQTNSPGSASSVASQSIIAYTMSLGVENSVAYSNNSIAIPTNFTISVT TEI LPVSMTKTSVDCTMYICGDSTECSNLLLQYGSFCTQLNRALTGIAVEQDK NTQEVFAQVKQIYKTPPIKDFGGFNFSQILPDPSKPSKRSPIEDLLFNKV TL ADAGFIKQYGDCLGDIAARDLICAQKFNGLTVLPPLLTDEMIAQYTSALL AGTITSGWTFGAGPALQIPFPMQMAYRFNGIGVTQNVLYENQKLIANQFN SAIGKIQDSLSSTPSALGKLQDVVNQNAQALNTLVKQLSSNFGAISSVLN D ILSRLDPPEAEVQIDRLITGRLQSLQTYVTQQLIRAAEIRASANLAATKM SE CVLGQSKRVDFCGKGYHLMSFPQSAPHGVVFLHVTYVPAQEKNFTTAPA ICHDGKAHFPREGVFVSNGTHWFVTQRNFYEPQIITTDNTFVSGNCDVVI G IVNNTVYDPLQSELDSIKEELDKIHKNGSGGSGDIIKLLNEQVNKEMQSS N LYMSMSSWCYTHSLDGAGLFLFDHAAEEYEHAKKLIIFLNENNVPVQLTS ISAPEHKFEGLTQIFQKAYEHEQHISESINNIVDHAIKSKDHATFNFLQW YV AEQHEEEVLFKDILDKIELIGNENHGLYLADQYVKGIAKSRKSGS | 251 |
| | | pCoV186-B.1.351/501Y.V2-L18F_D80A_D215_delta241-243_R246_K417N_E484K_N501Y_D614G_A701V | | SSQCVNFTTRTQLPPAYTNSFTRGVYYPDKVFRSSVLHSTQDLFLPFFSN V TWFHAIHVSGTNGTKRFANPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLHRSYLTPGDSSSGWTAGAAAYYV G YLQPRTFLLKYNENGTITDAVDCALDPLSETKCTLKSFTVEKGIYQTSNF R VQPTESIVRFPNITNLCPFGEVFNATRFASVYAWNRKRISNCVADYSVLY N SASFSTFKCYGVSPTKLNDLCFTNVYADSFVIRGDEVRQIAPGQTGNIAD Y NYKLPDDFTGCVIAWNSNNLDSKVGGNYNYLYRLFRKSNLKPFERDISTE IYQAGSTPCNGVKGFNCYFPLQSYGFQPTYGVGYQPYRVVVLSFELLHAP ATVCGPKKSTNLVKNKCVNFNFNGLTGTGVLTESNKKFLPFQQFGRDIAD TTDAVRDPQTLEILDITPCSFGGVSVITPGTNTSNQVAVLYQGVNCTEVP V AIHADQLTPTWRVYSTGSNVFQTRAGCLIGAEHVNNSYECDIPIGAGICA S YQTQTNSPGSASSVASQSIIAYTMSLGVENSVAYSNNSIAIPTNFTISVT TEI LPVSMTKTSVDCTMYICGDSTECSNLLLQYGSFCTQLNRALTGIAVEQDK NTQEVFAQVKQIYKTPPIKDFGGFNFSQILPDPSKPSKRSPIEDLLFNKV TL ADAGFIKQYGDCLGDIAARDLICAQKFNGLTVLPPLLTDEMIAQYTSALL AGTITSGWTFGAGPALQIPFPMQMAYRFNGIGVTQNVLYENQKLIANQFN SAIGKIQDSLSSTPSALGKLQDVVNQNAQALNTLVKQLSSNFGAISSVLN D ILSRLDPPEAEVQIDRLITGRLQSLQTYVTQQLIRAAEIRASANLAATKM SE CVLGQSKRVDFCGKGYHLMSFPQSAPHGVVFLHVTYVPAQEKNFTTAPA ICHDGKAHFPREGVFVSNGTHWFVTQRNFYEPQIITTDNTFVSGNCDVVI G IVNNTVYDPLQSELDSIKEELDKIHKNGSGGSGDIIKLLNEQVNKEMQSS N LYMSMSSWCYTHSLDGAGLFLFDHAAEEYEHAKKLIIFLNENNVPVQLTS ISAPEHKFEGLTQIFQKAYEHEQHISESINNIVDHAIKSKDHATFNFLQW YV AEQHEEEVLFKDILDKIELIGNENHGLYLADQYVKGIAKSRKSGS | 252 |
| | | pCoV186-P.1/501Y.V3-L18F_T20N_P26S_D138Y_R190S_K417T_E484K_N501Y_D614G_H655Y_T1027I | | SSQCVNFTNRTQLPSAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNYPFLGVYYHKNNKSWMESEFRVYSSA NNCTFEYVSQPFLMDLEGKQGNFKNLSEFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGT I ADYNYKLPDDFTGCVIAWNSNNLDSKVGGNYNYLYRLFRKSNLKPFERD ISTEIYQAGSTPCNGVKGFNCYFPLQSYGFQPTYGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQGVNC T EVPVAIHADQLTPTWRVYSTGSNVFQTRAGCLIGAEYVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSPIEDLL FN KVTLADAGFIKQYGDCLGDIAARDLICAQKFNGLTVLPPLLTDEMIAQYT SALLAGTITSGWTFGAGPALQIPFPMQMAYRFNGIGVTQNVLYENQKLIA NQFNSAIGKIQDSLSSTPSALGKLQDVVNQNAQALNTLVKQLSSNFGAIS S VLNDILSRLDPPEAEVQIDRLITGRLQSLQTYVTQQLIRAAEIRASANLA AI KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQSELDSIKEELDKIHKNGSGGSGDIIKLLNEQVNK E MQSSNLYMSMSSWCYTHSLDGAGLFLFDHAAEEYEHAKKLIIFLNENNV PVQLTSISAPEHKFEGLTQIFQKAYEHEQHISESINNIVDHAIKSKDHAT FNF LQWYVAEQHEEEVLFKDILDKIELIGNENHGLYLADQYVKGIAKSRKSGS | 253 |
| | | pCoV1B-08-N1158glycan-N1172glycan | | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQDVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSFIEDLL FN KVTLADAGFIKQYGDCLGDIAARDLICAQKFNGLTVLPPLLTDEMIAQYT SALLAGTITSGWTFGAGAALQIPFAMQMAYRFNGIGVTQNVLYENQKLIA NQFNSAIGKIQDSLSSTASALGKLQDVVNQNAQALNTLVKQLSSNFGAIS S VLNDILSRLDPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQSELDSIKEELDKIHKNLTSIKEELDKIHKNGSGG SG DIIKLLNEQVNKEMQSSNLYMSMSSWCYTHSLDGAGLFLFDHAAEEYEH AKKLIIFLNENNVPVQLTSISAPEHKFEGLTQIFQKAYEHEQHISESINN IVD HAIKSKDHATFNFLQWYVAEQHEEEVLFKDILDKIELIGNENHGLYLADQ YVKGIAKSRKSGS | 254 |
| | | pCoVS01 | | SDLDRCTTFDDVQAPNYTQHTSSMRGVYYPDEIFRSDTLYLTQDLFLPFY SNVTGFHTINHTFGNPVIPFKDGIYFAATEKSNVVRGWVFGSTMNNKSQS VIIINNSTNVVIRACNFELCDNPFFAVSKPMGTQTHTMIFDNAFNCTFEY IS DAFSLDVSEKSGNFKHLREFVFKNKDGFLYVYKGYQPIDVVRDLPSGFNT LKPIFKLPLGINITNFRAILTAFSPAQDIWGTSAAAYFVGYLKPTTFMLK YD ENGTITDAVDCSQNPLAELKCSVKSFEIDKGIYQTSNFRVVPSGDVVRFP N ITNLCPFGEVFNATKFPSVYAWERKKISNCVADYSVLYNSTFFSTFKCYG V SATKLNDLCFSNVYADSFVVKGDDVRQIAPGQTGVIADYNYKLPDDFMG CVLAWNTRNIDATSTGNYNYKYRYLRHGKLRPFERDISNVPFSPDGKPCT PPALNCYWPLNDYGFYTTTGIGYQPYRVVVLSFELLNAPATVCGPKLSTD LIKNQCVNFNFNGLTGTGVLTPSSKRFQPFQQFGRDVSDFTDSVRDPKTS E ILDISPCAFGGVSVITPGTNASSEVAVLYQDVNCTDVSTAIHADQLTPAW R IYSTGNNVFQTQAGCLIGAEHVDTSYECDIPIGAGICASYHTVSLLRSTS QK SIVAYTMSLGADSSIAYSNNTIAIPTNFSISITTEVMPVSMAKTSVDCNM YI CGDSTECANLLLQYGSFCTQLNRALSGIAAEQDRNTREVFAQVKQMYKT PTLKYFGGFNFSQILPDPLKPTKRSFIEDLLFNKVTLADAGFMKQYGECL G DINARDLICAQKFNGLTVLPPLLTDDMIAAYTAALVSGTATAGWTFGAGA ALQIPFAMQMAYRFNGIGVTQNVLYENQKQIANQFNKAISQIQESLTTTS T ALGKLQDVVNQNAQALNTLVKQLSSNFGAISSVLNDILSRLDPPEAEVQI DRLITGRLQSLQTYVTQQLIRAAEIRASANLAATKMSECVLGQSKRVDFC GKGYHLMSFPQAAPHGVVFLHVTYVPSQERNFTTAPAICHEGKAYFPREG VFVFNGTSWFITQRNFFSPQIITTDNTFVSGNCDVVIGIINNTVYDPLQS EL DSIKEELDKIHKNGSGGSGDIIKLLNEQVNKEMQSSNLYMSMSSWCYTHS LDGAGLFLFDHAAEEYEHAKKLIIFLNENNVPVQLTSISAPEHKFEGLTQ IF QKAYEHEQHISESINNIVDHAIKSKDHATFNFLQWYVAEQHEEEVLFKDI L DKIELIGNENHGLYLADQYVKGIAKSRKSGS | 255 |
| | | OC43-1b06v0-natlead | | AVIGDLKCTSDNINDKDTGPPPISTDTVDVTNGLGTYYVLDRVYLNTTLF L NGYYPTSGSTYRNMALKGSVLLSRLWFKPPFLSDFINGIFAKVKNTKVIK DRVMYSEFPAITIGSTFVNTSYSVVVQPRTINSTQDGDNKLQGLLEVSVC Q YNMCEYPQTICHPNLGNHRKELWHLDTGVVSCLYKRNFTYDVNADYLY FHFYQEGGTFYAYFTDTGVVTKFLFNVYLGMALSHYYVMPLTCNSKLTL EYWVTPLTSRQYLLAFNQDGIIFNAVDCMSDFMSEIKCKTQSIAPPTGVY E LNGYTVQPIADVYRRKPNLPNCNIEAWLNDKSVPSPLNWERKTFSNCNFN MSSLMSFIQADSFTCNNIDAAKIYGMCFSSITIDKFAIPNGRKVDLQLGN L GYLQSFNYRIDTTATSCQLYYNLPAANVSVSRFNPSTWNKRFGFIEDSVF K PRPAGVLTNHDVVYAQHCFKAPKNFCPCKLNGSCVGSGPGKNNGIGTCP AGTNYLTCDNLCTPDPITFTGTYKCPQTKSLVGIGEHCSGLAVKSDYCGG NSCTCRPQAFLGWSADSCLQGDKCNIFANFILHDVNSGLTCSTDLQKANT DIILGVCVNYDLYGILGQGIFVEVNATYYNSWQNLLYDSNGNLYGFRDYI TNRTFMIRSCYSGRVSAAFHANSSEPALLFRNIKCNYVFNNSLTRQLQPI N YFDSYLGCVVNAYNSTAISVQTCDLTVGSGYCVDYSKNGGSGGAITTGY RFTNFEPFTVNSVNDSLEPVGGLYEIQIPSEFTIGNMVEFIQTSSPKVTI DCA AFVCGDYAACKSQLVEYGSFCDNINAILTEVNELLDTTQLQVANSLMNG VTLSTKLKDGVNFNVDDINFSPVLGCLGSECSKASSRSAIEDLLFDKVKL S DVGFVEAYNNCTGGAEIRDLICVQSYKGIKVLPPLLSENQFSGYTLAATS A SLFPPWTAAAGVPFYLNVQYRINGLGVTMDVLSQNQKLIANAFNNALYA IQEGFDATNSALVKIQAVVNANAEALNNLLQQLSNRFGAISASLQEILSR L DPPEAEAQIDRLINGRLTALNAYVSQQLSDSTLVKFSAAQAMEKVNECVK SQSSRINFCGNGNHIISLVQNAPYGLYFIHFSYVPTKYVTARVSPGLCIA GD RGIAPKSGYFVNVNNTWMYTGSGYYYPEPITENNVVVMSTCAVNYTKAP YVMLNTSISELQDFKEELDQWHKNGSGGSGDIIKLLNEQVNKEMQSSNLY MSMSSWCYTHSLDGAGLFLFDHAAEEYEHAKKLIIFLNENNVPVQLTSIS APEHKFEGLTQIFQKAYEHEQHISESINNIVDHAIKSKDHATFNFLQWYV A EQHEEEVLFKDILDKIELIGNENHGLYLADQYVKGIAKSRKSGS | 256 |
| | | OC43-1b06v0-nogly-natlead | | AVIGDLKCTSDNINDKDTGPPPISTDTVDVTNGLGTYYVLDRVYLNTTLF L NGYYPTSGSTYRNMALKGSVLLSRLWFKPPFLSDFINGIFAKVKNTKVIK DRVMYSEFPAITIGSTFVNTSYSVVVQPRTINSTQDGDNKLQGLLEVSVC Q YNMCEYPQTICHPNLGNHRKELWHLDTGVVSCLYKRNFTYDVNADYLY FHFYQEGGTFYAYFTDTGVVTKFLFNVYLGMALSHYYVMPLTCNSKLTL EYWVTPLTSRQYLLAFNQDGIIFNAVDCMSDFMSEIKCKTQSIAPPTGVY E LNGYTVQPIADVYRRKPNLPNCNIEAWLNDKSVPSPLNWERKTFSNCNFN MSSLMSFIQADSFTCNNIDAAKIYGMCFSSITIDKFAIPNGRKVDLQLGN L GYLQSFNYRIDTTATSCQLYYNLPAANVSVSRFNPSTWNKRFGFIEDSVF K PRPAGVLTNHDVVYAQHCFKAPKNFCPCKLNGSCVGSGPGKNNGIGTCP AGTNYLTCDNLCTPDPITFTGTYKCPQTKSLVGIGEHCSGLAVKSDYCGG NSCTCRPQAFLGWSADSCLQGDKCNIFANFILHDVNSGLTCSTDLQKANT DIILGVCVNYDLYGILGQGIFVEVNATYYNSWQNLLYDSNGNLYGFRDYI TNRTFMIRSCYSGRVSAAFHANSSEPALLFRNIKCNYVFNNSLTRQLQPI N YFDSYLGCVVNAYNSTAISVQTCDLTVGSGYCVDYSKNGGSGGAITTGY RFTNFEPFTVNSVNDSLEPVGGLYEIQIPSEFTIGNMVEFIQTSSPKVTI DCA AFVCGDYAACKSQLVEYGSFCDNINAILTEVNELLDTTQLQVANSLMNG VTLSTKLKDGVNFNVDDINFSPVLGCLGSECSKASSRSAIEDLLFDKVKL S DVGFVEAYNNCTGGAEIRDLICVQSYKGIKVLPPLLSENQFSGYTLAATS A SLFPPWTAAAGVPFYLNVQYRINGLGVTMDVLSQNQKLIANAFNNALYA IQEGFDATNSALVKIQAVVNANAEALNNLLQQLSNRFGAISASLQEILSR L DPPEAEAQIDRLINGRLTALNAYVSQQLSDSTLVKFSAAQAMEKVNECVK SQSSRINFCGNGNHIISLVQNAPYGLYFIHFSYVPTKYVTARVSPGLCIA GD RGIAPKSGYFVNVNNTWMYTGSGYYYPEPITENNVVVMSTCAVNYTKAP YVMLNTSISELQDFKEELDQWHKQGSGGSGDIIKLLNEQVNKEMQSSNLY MSMSSWCYTHSLDGAGLFLFDHAAEEYEHAKKLIIFLNENNVPVQLTSIS APEHKFEGLTQIFQKAYEHEQHISESINNIVDHAIKSKDHATFNFLQWYV A EQHEEEVLFKDILDKIELIGNENHGLYLADQYVKGIAKSRKSGS | 257 |
| | | OC43-1b06v1-natlead | | AVIGDLKCTSDNINDKDTGPPPISTDTVDVTNGLGTYYVLDRVYLNTTLF L NGYYPTSGSTYRNMALKGSVLLSRLWFKPPFLSDFINGIFAKVKNTKVIK DRVMYSEFPAITIGSTFVNTSYSVVVQPRTINSTQDGDNKLQGLLEVSVC Q YNMCEYPQTICHPNLGNHRKELWHLDTGVVSCLYKRNFTYDVNADYLY FHFYQEGGTFYAYFTDTGVVTKFLFNVYLGMALSHYYVMPLTCNSKLTL EYWVTPLTSRQYLLAFNQDGIIFNAVDCMSDFMSEIKCKTQSIAPPTGVY E LNGYTVQPIADVYRRKPNLPNCNIEAWLNDKSVPSPLNWERKTFSNCNFN MSSLMSFIQADSFTCNNIDAAKIYGMCFSSITIDKFAIPNGRKVDLQLGN L GYLQSFNYRIDTTATSCQLYYNLPAANVSVSRFNPSTWNKRFGFIEDSVF K PRPAGVLTNHDVVYAQHCFKAPKNFCPCKLNGSCVGSGPGKNNGIGTCP AGTNYLTCDNLCTPDPITFTGTYKCPQTKSLVGIGEHCSGLAVKSDYCGG NSCTCRPQAFLGWSADSCLQGDKCNIFANFILHDVNSGLTCSTDLQKANT DIILGVCVNYDLYGILGQGIFVEVNATYYNSWQNLLYDSNGNLYGFRDYI TNRTFMIRSCYSGRVSAAFHANSSEPALLFRNIKCNYVFNNSLTRQLQPI N YFDSYLGCVVNAYNSTAISVQTCDLTVGSGYCVDYSKNGGSGGAITTGY RFTNFEPFTVNSVNDSLEPVGGLYEIQIPSEFTIGNMVEFIQTSSPKVTI DCA AFVCGDYAACKSQLVEYGSFCDNINAILTEVNELLDTTQLQVANSLMNG VTLSTKLKDGVNFNVDDINFSPVLGCLGSECSKASSRSAIEDLLFDKVKL S DVGFVEAYNNCTGGAEIRDLICVQSYKGIKVLPPLLSENQFSGYTLAATS A SLFPPWTAAAGVPFYLNVQYRINGLGVTMDVLSQNQKLIANAFNNALYA IQEGFDATNSALVKIQAVVNANAEALNNLLQQLSNRFGAISASLQEILSR L DPPEAEAQIDRLINGRLTALNAYVSQQLSDSTLVKFSAAQAMEKVNECVK SQSSRINFCGNGNHIISLVQNAPYGLYFIHFSYVPTKYVTARVSPGLCIA GD RGIAPKSGYFVNVNNTWMYTGSGYYYPEPITENNVVVMSTCAVNYTKAP YVMLNTLQSNLQDIKEELDQIHKNGSGGSGDIIKLLNEQVNKEMQSSNLY MSMSSWCYTHSLDGAGLFLFDHAAEEYEHAKKLIIFLNENNVPVQLTSIS APEHKFEGLTQIFQKAYEHEQHISESINNIVDHAIKSKDHATFNFLQWYV A EQHEEEVLFKDILDKIELIGNENHGLYLADQYVKGIAKSRKSGS | 258 |
| | | OC43-1b06v1-nogly-natlead | | AVIGDLKCTSDNINDKDTGPPPISTDTVDVTNGLGTYYVLDRVYLNTTLF L NGYYPTSGSTYRNMALKGSVLLSRLWFKPPFLSDFINGIFAKVKNTKVIK DRVMYSEFPAITIGSTFVNTSYSVVVQPRTINSTQDGDNKLQGLLEVSVC Q YNMCEYPQTICHPNLGNHRKELWHLDTGVVSCLYKRNFTYDVNADYLY FHFYQEGGTFYAYFTDTGVVTKFLFNVYLGMALSHYYVMPLTCNSKLTL EYWVTPLTSRQYLLAFNQDGIIFNAVDCMSDFMSEIKCKTQSIAPPTGVY E LNGYTVQPIADVYRRKPNLPNCNIEAWLNDKSVPSPLNWERKTFSNCNFN MSSLMSFIQADSFTCNNIDAAKIYGMCFSSITIDKFAIPNGRKVDLQLGN L GYLQSFNYRIDTTATSCQLYYNLPAANVSVSRFNPSTWNKRFGFIEDSVF K PRPAGVLTNHDVVYAQHCFKAPKNFCPCKLNGSCVGSGPGKNNGIGTCP AGTNYLTCDNLCTPDPITFTGTYKCPQTKSLVGIGEHCSGLAVKSDYCGG NSCTCRPQAFLGWSADSCLQGDKCNIFANFILHDVNSGLTCSTDLQKANT DIILGVCVNYDLYGILGQGIFVEVNATYYNSWQNLLYDSNGNLYGFRDYI TNRTFMIRSCYSGRVSAAFHANSSEPALLFRNIKCNYVFNNSLTRQLQPI N YFDSYLGCVVNAYNSTAISVQTCDLTVGSGYCVDYSKNGGSGGAITTGY RFTNFEPFTVNSVNDSLEPVGGLYEIQIPSEFTIGNMVEFIQTSSPKVTI DCA AFVCGDYAACKSQLVEYGSFCDNINAILTEVNELLDTTQLQVANSLMNG VTLSTKLKDGVNFNVDDINFSPVLGCLGSECSKASSRSAIEDLLFDKVKL S DVGFVEAYNNCTGGAEIRDLICVQSYKGIKVLPPLLSENQFSGYTLAATS A SLFPPWTAAAGVPFYLNVQYRINGLGVTMDVLSQNQKLIANAFNNALYA IQEGFDATNSALVKIQAVVNANAEALNNLLQQLSNRFGAISASLQEILSR L DPPEAEAQIDRLINGRLTALNAYVSQQLSDSTLVKFSAAQAMEKVNECVK SQSSRINFCGNGNHIISLVQNAPYGLYFIHFSYVPTKYVTARVSPGLCIA GD RGIAPKSGYFVNVNNTWMYTGSGYYYPEPITENNVVVMSTCAVNYTKAP YVMLNTLQSNLQDIKEELDQIHKQGSGGSGDIIKLLNEQVNKEMQSSNLY MSMSSWCYTHSLDGAGLFLFDHAAEEYEHAKKLIIFLNENNVPVQLTSIS APEHKFEGLTQIFQKAYEHEQHISESINNIVDHAIKSKDHATFNFLQWYV A EQHEEEVLFKDILDKIELIGNENHGLYLADQYVKGIAKSRKSGS | 259 |
| | | OC43-1b06v2-natlead | | AVIGDLKCTSDNINDKDTGPPPISTDTVDVTNGLGTYYVLDRVYLNTTLF L NGYYPTSGSTYRNMALKGSVLLSRLWFKPPFLSDFINGIFAKVKNTKVIK DRVMYSEFPAITIGSTFVNTSYSVVVQPRTINSTQDGDNKLQGLLEVSVC Q YNMCEYPQTICHPNLGNHRKELWHLDTGVVSCLYKRNFTYDVNADYLY FHFYQEGGTFYAYFTDTGVVTKFLFNVYLGMALSHYYVMPLTCNSKLTL EYWVTPLTSRQYLLAFNQDGIIFNAVDCMSDFMSEIKCKTQSIAPPTGVY E LNGYTVQPIADVYRRKPNLPNCNIEAWLNDKSVPSPLNWERKTFSNCNFN MSSLMSFIQADSFTCNNIDAAKIYGMCFSSITIDKFAIPNGRKVDLQLGN L GYLQSFNYRIDTTATSCQLYYNLPAANVSVSRFNPSTWNKRFGFIEDSVF K PRPAGVLTNHDVVYAQHCFKAPKNFCPCKLNGSCVGSGPGKNNGIGTCP AGTNYLTCDNLCTPDPITFTGTYKCPQTKSLVGIGEHCSGLAVKSDYCGG NSCTCRPQAFLGWSADSCLQGDKCNIFANFILHDVNSGLTCSTDLQKANT DIILGVCVNYDLYGILGQGIFVEVNATYYNSWQNLLYDSNGNLYGFRDYI TNRTFMIRSCYSGRVSAAFHANSSEPALLFRNIKCNYVFNNSLTRQLQPI N YFDSYLGCVVNAYNSTAISVQTCDLTVGSGYCVDYSKNGGSGGAITTGY RFTNFEPFTVNSVNDSLEPVGGLYEIQIPSEFTIGNMVEFIQTSSPKVTI DCA AFVCGDYAACKSQLVEYGSFCDNINAILTEVNELLDTTQLQVANSLMNG VTLSTKLKDGVNFNVDDINFSPVLGCLGSECSKASSRSAIEDLLFDKVKL S DVGFVEAYNNCTGGAEIRDLICVQSYKGIKVLPPLLSENQFSGYTLAATS A SLFPPWTAAAGVPFYLNVQYRINGLGVTMDVLSQNQKLIANAFNNALYA IQEGFDATNSALVKIQAVVNANAEALNNLLQQLSNRFGAISASLQEILSR L DPPEAEAQIDRLINGRLTALNAYVSQQLSDSTLVKFSAAQAMEKVNECVK SQSSRINFCGNGNHIISLVQNAPYGLYFIHFSYVPTKYVTARVSPGLCIA GD RGIAPKSGYFVNVNNTWMYTGSGYYYPEPITENNVVVMSTCAVNYTKAP YVMLNTLQSNLQDLKEELDQLHKNGSGGSGDIIKLLNEQVNKEMQSSNL YMSMSSWCYTHSLDGAGLFLFDHAAEEYEHAKKLIIFLNENNVPVQLTSI SAPEHKFEGLTQIFQKAYEHEQHISESINNIVDHAIKSKDHATFNFLQWY V AEQHEEEVLFKDILDKIELIGNENHGLYLADQYVKGIAKSRKSGS | 260 |
| | | OC43-1b06v2-nogly-natlead | | AVIGDLKCTSDNINDKDTGPPPISTDTVDVTNGLGTYYVLDRVYLNTTLF L NGYYPTSGSTYRNMALKGSVLLSRLWFKPPFLSDFINGIFAKVKNTKVIK DRVMYSEFPAITIGSTFVNTSYSVVVQPRTINSTQDGDNKLQGLLEVSVC Q YNMCEYPQTICHPNLGNHRKELWHLDTGVVSCLYKRNFTYDVNADYLY FHFYQEGGTFYAYFTDTGVVTKFLFNVYLGMALSHYYVMPLTCNSKLTL EYWVTPLTSRQYLLAFNQDGIIFNAVDCMSDFMSEIKCKTQSIAPPTGVY E LNGYTVQPIADVYRRKPNLPNCNIEAWLNDKSVPSPLNWERKTFSNCNFN MSSLMSFIQADSFTCNNIDAAKIYGMCFSSITIDKFAIPNGRKVDLQLGN L GYLQSFNYRIDTTATSCQLYYNLPAANVSVSRFNPSTWNKRFGFIEDSVF K PRPAGVLTNHDVVYAQHCFKAPKNFCPCKLNGSCVGSGPGKNNGIGTCP AGTNYLTCDNLCTPDPITFTGTYKCPQTKSLVGIGEHCSGLAVKSDYCGG NSCTCRPQAFLGWSADSCLQGDKCNIFANFILHDVNSGLTCSTDLQKANT DIILGVCVNYDLYGILGQGIFVEVNATYYNSWQNLLYDSNGNLYGFRDYI TNRTFMIRSCYSGRVSAAFHANSSEPALLFRNIKCNYVFNNSLTRQLQPI N YFDSYLGCVVNAYNSTAISVQTCDLTVGSGYCVDYSKNGGSGGAITTGY RFTNFEPFTVNSVNDSLEPVGGLYEIQIPSEFTIGNMVEFIQTSSPKVTI DCA AFVCGDYAACKSQLVEYGSFCDNINAILTEVNELLDTTQLQVANSLMNG VTLSTKLKDGVNFNVDDINFSPVLGCLGSECSKASSRSAIEDLLFDKVKL S DVGFVEAYNNCTGGAEIRDLICVQSYKGIKVLPPLLSENQFSGYTLAATS A SLFPPWTAAAGVPFYLNVQYRINGLGVTMDVLSQNQKLIANAFNNALYA IQEGFDATNSALVKIQAVVNANAEALNNLLQQLSNRFGAISASLQEILSR L DPPEAEAQIDRLINGRLTALNAYVSQQLSDSTLVKFSAAQAMEKVNECVK SQSSRINFCGNGNHIISLVQNAPYGLYFIHFSYVPTKYVTARVSPGLCIA GD RGIAPKSGYFVNVNNTWMYTGSGYYYPEPITENNVVVMSTCAVNYTKAP YVMLNTLQSNLQDLKEELDQLHKQGSGGSGDIIKLLNEQVNKEMQSSNL YMSMSSWCYTHSLDGAGLFLFDHAAEEYEHAKKLIIFLNENNVPVQLTSI SAPEHKFEGLTQIFQKAYEHEQHISESINNIVDHAIKSKDHATFNFLQWY V AEQHEEEVLFKDILDKIELIGNENHGLYLADQYVKGIAKSRKSGS | 261 |
| | | OC43-1b06v3-nogly-natlead | | AVIGDLKCTSDNINDKDTGPPPISTDTVDVTNGLGTYYVLDRVYLNTTLF L NGYYPTSGSTYRNMALKGSVLLSRLWFKPPFLSDFINGIFAKVKNTKVIK DRVMYSEFPAITIGSTFVNTSYSVVVQPRTINSTQDGDNKLQGLLEVSVC Q YNMCEYPQTICHPNLGNHRKELWHLDTGVVSCLYKRNFTYDVNADYLY FHFYQEGGTFYAYFTDTGVVTKFLFNVYLGMALSHYYVMPLTCNSKLTL EYWVTPLTSRQYLLAFNQDGIIFNAVDCMSDFMSEIKCKTQSIAPPTGVY E LNGYTVQPIADVYRRKPNLPNCNIEAWLNDKSVPSPLNWERKTFSNCNFN MSSLMSFIQADSFTCNNIDAAKIYGMCFSSITIDKFAIPNGRKVDLQLGN L GYLQSFNYRIDTTATSCQLYYNLPAANVSVSRFNPSTWNKRFGFIEDSVF K PRPAGVLTNHDVVYAQHCFKAPKNFCPCKLNGSCVGSGPGKNNGIGTCP AGTNYLTCDNLCTPDPITFTGTYKCPQTKSLVGIGEHCSGLAVKSDYCGG NSCTCRPQAFLGWSADSCLQGDKCNIFANFILHDVNSGLTCSTDLQKANT DIILGVCVNYDLYGILGQGIFVEVNATYYNSWQNLLYDSNGNLYGFRDYI TNRTFMIRSCYSGRVSAAFHANSSEPALLFRNIKCNYVFNNSLTRQLQPI N YFDSYLGCVVNAYNSTAISVQTCDLTVGSGYCVDYSKNGGSGGAITTGY RFTNFEPFTVNSVNDSLEPVGGLYEIQIPSEFTIGNMVEFIQTSSPKVTI DCA AFVCGDYAACKSQLVEYGSFCDNINAILTEVNELLDTTQLQVANSLMNG VTLSTKLKDGVNFNVDDINFSPVLGCLGSECSKASSRSAIEDLLFDKVKL S DVGFVEAYNNCTGGAEIRDLICVQSYKGIKVLPPLLSENQFSGYTLAATS A SLFPPWTAAAGVPFYLNVQYRINGLGVTMDVLSQNQKLIANAFNNALYA IQEGFDATNSALVKIQAVVNANAEALNNLLQQLSNRFGAISASLQEILSR L DPPEAEAQIDRLINGRLTALNAYVSQQLSDSTLVKFSAAQAMEKVNECVK SQSSRINFCGNGNHIISLVQNAPYGLYFIHFSYVPTKYVTARVSPGLCIA GD RGIAPKSGYFVNVNNTWMYTGSGYYYPEPITENNVVVMSTCAVNYTKAP YVMLNTSISELQDIKEELDQIHKQGSGGSGDIIKLLNEQVNKEMQSSNLY M SMSSWCYTHSLDGAGLFLFDHAAEEYEHAKKLIIFLNENNVPVQLTSISA P EHKFEGLTQIFQKAYEHEQHISESINNIVDHAIKSKDHATFNFLQWYVAE Q HEEEVLFKDILDKIELIGNENHGLYLADQYVKGIAKSRKSGS | 262 |
| | | OC43-1b06v4-nogly-natlead | | AVIGDLKCTSDNINDKDTGPPPISTDTVDVTNGLGTYYVLDRVYLNTTLF L NGYYPTSGSTYRNMALKGSVLLSRLWFKPPFLSDFINGIFAKVKNTKVIK DRVMYSEFPAITIGSTFVNTSYSVVVQPRTINSTQDGDNKLQGLLEVSVC Q YNMCEYPQTICHPNLGNHRKELWHLDTGVVSCLYKRNFTYDVNADYLY FHFYQEGGTFYAYFTDTGVVTKFLFNVYLGMALSHYYVMPLTCNSKLTL EYWVTPLTSRQYLLAFNQDGIIFNAVDCMSDFMSEIKCKTQSIAPPTGVY E LNGYTVQPIADVYRRKPNLPNCNIEAWLNDKSVPSPLNWERKTFSNCNFN MSSLMSFIQADSFTCNNIDAAKIYGMCFSSITIDKFAIPNGRKVDLQLGN L GYLQSFNYRIDTTATSCQLYYNLPAANVSVSRFNPSTWNKRFGFIEDSVF K PRPAGVLTNHDVVYAQHCFKAPKNFCPCKLNGSCVGSGPGKNNGIGTCP AGTNYLTCDNLCTPDPITFTGTYKCPQTKSLVGIGEHCSGLAVKSDYCGG NSCTCRPQAFLGWSADSCLQGDKCNIFANFILHDVNSGLTCSTDLQKANT DIILGVCVNYDLYGILGQGIFVEVNATYYNSWQNLLYDSNGNLYGFRDYI TNRTFMIRSCYSGRVSAAFHANSSEPALLFRNIKCNYVFNNSLTRQLQPI N YFDSYLGCVVNAYNSTAISVQTCDLTVGSGYCVDYSKNGGSGGAITTGY RFTNFEPFTVNSVNDSLEPVGGLYEIQIPSEFTIGNMVEFIQTSSPKVTI DCA AFVCGDYAACKSQLVEYGSFCDNINAILTEVNELLDTTQLQVANSLMNG VTLSTKLKDGVNFNVDDINFSPVLGCLGSECSKASSRSAIEDLLFDKVKL S DVGFVEAYNNCTGGAEIRDLICVQSYKGIKVLPPLLSENQFSGYTLAATS A SLFPPWTAAAGVPFYLNVQYRINGLGVTMDVLSQNQKLIANAFNNALYA IQEGFDATNSALVKIQAVVNANAEALNNLLQQLSNRFGAISASLQEILSR L DPPEAEAQIDRLINGRLTALNAYVSQQLSDSTLVKFSAAQAMEKVNECVK SQSSRINFCGNGNHIISLVQNAPYGLYFIHFSYVPTKYVTARVSPGLCIA GD RGIAPKSGYFVNVNNTWMYTGSGYYYPEPITENNVVVMSTCAVNYTKAP YVMLNTSISELQDLKEELDQLHKQGSGGSGDIIKLLNEQVNKEMQSSNLY MSMSSWCYTHSLDGAGLFLFDHAAEEYEHAKKLIIFLNENNVPVQLTSIS APEHKFEGLTQIFQKAYEHEQHISESINNIVDHAIKSKDHATFNFLQWYV A EQHEEEVLFKDILDKIELIGNENHGLYLADQYVKGIAKSRKSGS | 263 |
| | | 229E-1b06-v0-natlead | | CQTTNGLNTSYSVCNGCVGYSENVFAVESGGYIPSDFAFNNWFLLTNTSS VVDGVVRSFQPLLLNCLWSVSGLRFTTGFVYFNGTGRGDCKGFSSDVLS DVIRYNLNFEENLRRGTILFKTSYGVVVFYCTNNTLVSGDAHIPFGTVLG N FYCFVNTTIGNETTSAFVGALPKTVREFVISRTGHFYINGYRYFTLGNVE A VNFNVTTAETTDFCTVALASYADVLVNVSQTSIANIIYCNSVINRLRCDQ L SFDVPDGFYSTSPIQSVELPVSIVSLPVYHKHTFIVLYVDFKPQSGGGKC FN CYPAGVNITLANFNETKGPLCVDTSHFTTKYVAVYANVGRWSASINTGN CPFSFGKVNNFVKFGSVCFSLKDIPGGCAMPIVANWAYSKYYTIGSLYVS WSDGDGITGVPQPVEGVSSFMNVTLDKCTKYNIYDVSGVGVIRVSNDTFL NGITYTSTSGNLLGFKDVTKGTIYSITPCNPPDQLVVYQQAVVGAMLSEN FTSYGFSNVVELPKFFYASNGTYNCTDAVLTYSSFGVCADGSIIAVQPRN V SYDSVSAIVTANLSIPSNWTTSVQVEYLQITSTPIVVDCSTYVCNGNVRC V ELLKQYTSACKTIEDALRNSAMLESADVSEMLTFDKKAFTLANVSSFGDY NLSSVIPSLPRSGSRVAGRSAIEDILFSKLVTSGLGTVDADYKKCTKGLS IA DLACAQYYNGIMVLPGVADAERMAMYTGSLIGGIALGGLTSAASIPFSLA IQSRLNYVALQTDVLQENQKILAASFNKAMTNIVDAFTGVNDAITQTSQA LQTVATALNKIQDVVNQQGNSLNHLTSQLRQNFQAISSSIQAIYDRLDPP Q ADQQVDRLITGRLAALNVFVSHTLTKYTEVRASRQLAQQKVNECVKSQS KRYGFCGNGTHIFSLVNAAPEGLVFLHTVLLPTQYKDVEAWSGLCVDGR NGYVLRQPNLALYKEGNYYRITSRIMFEPRIPTIADFVQIENCNVTFVNI SR SELQTIVPEYIDLNKTLQELSYKLPNLTVKGSGGSGDIIKLLNEQVNKEM Q SSNLYMSMSSWCYTHSLDGAGLFLFDHAAEEYEHAKKLIIFLNENNVPVQ LTSISAPEHKFEGLTQIFQKAYEHEQHISESINNIVDHAIKSKDHATFNF LQ WYVAEQHEEEVLFKDILDKIELIGNENHGLYLADQYVKGIAKSRKSGS | 264 |
| | | 229E-1b06-v1-natlead | | CQTTNGLNTSYSVCNGCVGYSENVFAVESGGYIPSDFAFNNWFLLTNTSS VVDGVVRSFQPLLLNCLWSVSGLRFTTGFVYFNGTGRGDCKGFSSDVLS DVIRYNLNFEENLRRGTILFKTSYGVVVFYCTNNTLVSGDAHIPFGTVLG N FYCFVNTTIGNETTSAFVGALPKTVREFVISRTGHFYINGYRYFTLGNVE A VNFNVTTAETTDFCTVALASYADVLVNVSQTSIANIIYCNSVINRLRCDQ L SFDVPDGFYSTSPIQSVELPVSIVSLPVYHKHTFIVLYVDFKPQSGGGKC FN CYPAGVNITLANFNETKGPLCVDTSHFTTKYVAVYANVGRWSASINTGN CPFSFGKVNNFVKFGSVCFSLKDIPGGCAMPIVANWAYSKYYTIGSLYVS WSDGDGITGVPQPVEGVSSFMNVTLDKCTKYNIYDVSGVGVIRVSNDTFL NGITYTSTSGNLLGFKDVTKGTIYSITPCNPPDQLVVYQQAVVGAMLSEN FTSYGFSNVVELPKFFYASNGTYNCTDAVLTYSSFGVCADGSIIAVQPRN V SYDSVSAIVTANLSIPSNWTTSVQVEYLQITSTPIVVDCSTYVCNGNVRC V ELLKQYTSACKTIEDALRNSAMLESADVSEMLTFDKKAFTLANVSSFGDY NLSSVIPSLPRSGSRVAGRSAIEDILFSKLVTSGLGTVDADYKKCTKGLS IA DLACAQYYNGIMVLPGVADAERMAMYTGSLIGGIALGGLTSAASIPFSLA IQSRLNYVALQTDVLQENQKILAASFNKAMTNIVDAFTGVNDAITQTSQA LQTVATALNKIQDVVNQQGNSLNHLTSQLRQNFQAISSSIQAIYDRLDPP Q ADQQVDRLITGRLAALNVFVSHTLTKYTEVRASRQLAQQKVNECVKSQS KRYGFCGNGTHIFSLVNAAPEGLVFLHTVLLPTQYKDVEAWSGLCVDGR NGYVLRQPNLALYKEGNYYRITSRIMFEPRIPTIADFVQIENCNVTFVNI SR SELQTIVPEYIGDLNKTLQELSYKLPNLTVKGSGGSGDIIKLLNEQVNKE M QSSNLYMSMSSWCYTHSLDGAGLFLFDHAAEEYEHAKKLIIFLNENNVPV QLTSISAPEHKFEGLTQIFQKAYEHEQHISESINNIVDHAIKSKDHATFN FL QWYVAEQHEEEVLFKDILDKIELIGNENHGLYLADQYVKGIAKSRKSGS | 265 |
| | | NL63-1b06v0-natlead | | CNSNANLSMLQLGVPDNSSTIVTGLLPTHWFCANQSTSVYSANGFFYIDV GNHRSAFALHTGYYDANQYYIYVTNEIGLNASVTLKICKFSRNTTFDFLS NASSSFDCIVNLLFTEQLGAPLGITISGETVRLHLYNVTRTFYVPAAYKL T KLSVKCYFNYSCVFSVVNATVTVNVTTHNGRVVNYTVCDDCNGYTDNIF SVQQDGRIPNGFPFNNWFLLTNGSTLVDGVSRLYQPLRLTCLWPVPGLKS STGFVYFNATGSDVNCNGYQHNSVVDVMRYNLNFSANSLDNLKSGVIVF KTLQYDVLFYCSNSSSGVLDTTIPFGPSSQPYYCFINSTINTTTIVSTFV GILP PTVREIVVARTGQFYINGFKYFDLGFIEAVNFNVTTASATDFWTVAFATF V DVLVNVSATNIQNLLYCDSPFEKLQCEHLQFGLQDGFYSANFLDDNVLPE TYVALPIYYQHTDINFTATASFGGSCYVCKPHQVNISLNGNTSVCVRTSH F SIRYIYNRVKSGSPGDSSWHIYLKSGTCPFSFSKLNNFQKFKTICFSTVE VP GSCNFPLEATWHYTSYTIVGALYVTWSEGNSITGVPYPVSGIREFSNLVL N NCTKYNIYDYVGTGIIRSSNQSLAGGITYVSNSGNLLGFKNVSTGNIFIV TP CNQPDQVAVYQQSIIGAMTAVNESRYGLQNLLQLPNFYYVSNGGNNCTT AVMTYSNFGICADGSLIPVRPRNSSDNGISAIITANLSIPSNWTTSVQVE YL QITSTPIVVDCATYVCNGNPRCKNLLKQYTSACKTIEDALRLSAHLETND VSSMLTFDSNAFSLANVTSFGDYNLSSVLPQRNIRSSRIAGRSALEDLLF SK VVTSGLGTVDVDYKSCTKGLSIADLACAQYYNGIMVLPGVADAERMAM YTGSLIGGMVLGGLTSAAAIPFSLALQARLNYVALQTDVLQENQKILAAS FNKAINNIVASFSSVNDAITQTAEAIHTVTIALNKIQDVVNQQGSALNHL TS QLRHNFQAISNSIQAIYDRLDPPQADQQVDRLITGRLAALNAFVSQVLNK YTEVRGSRRLAQQKINECVKSQSNRYGFCGNGTHIFSIVNSAPDGLLFLH T VLLPTDYKNVKAWSGICVDGIYGYVLRQPNLVLYSDNGVFRVTSRVMFQ PRLPVLSDFVQIYNCNVTFVNISRVELHTVIPDYVDLNKTLQELAQNLEK L VKKGSGGSGDIIKLLNEQVNKEMQSSNLYMSMSSWCYTHSLDGAGLFLF DHAAEEYEHAKKLIIFLNENNVPVQLTSISAPEHKFEGLTQIFQKAYEHE Q HISESINNIVDHAIKSKDHATFNFLQWYVAEQHEEEVLFKDILDKIELIG NE NHGLYLADQYVKGIAKSRKSGS | 266 |
| | | NL63-1b06v1-natlead | | CNSNANLSMLQLGVPDNSSTIVTGLLPTHWFCANQSTSVYSANGFFYIDV GNHRSAFALHTGYYDANQYYIYVTNEIGLNASVTLKICKFSRNTTFDFLS NASSSFDCIVNLLFTEQLGAPLGITISGETVRLHLYNVTRTFYVPAAYKL T KLSVKCYFNYSCVFSVVNATVTVNVTTHNGRVVNYTVCDDCNGYTDNIF SVQQDGRIPNGFPFNNWFLLTNGSTLVDGVSRLYQPLRLTCLWPVPGLKS STGFVYFNATGSDVNCNGYQHNSVVDVMRYNLNFSANSLDNLKSGVIVF KTLQYDVLFYCSNSSSGVLDTTIPFGPSSQPYYCFINSTINTTTIVSTFV GILP PTVREIVVARTGQFYINGFKYFDLGFIEAVNFNVTTASATDFWTVAFATF V DVLVNVSATNIQNLLYCDSPFEKLQCEHLQFGLQDGFYSANFLDDNVLPE TYVALPIYYQHTDINFTATASFGGSCYVCKPHQVNISLNGNTSVCVRTSH F SIRYIYNRVKSGSPGDSSWHIYLKSGTCPFSFSKLNNFQKFKTICFSTVE VP GSCNFPLEATWHYTSYTIVGALYVTWSEGNSITGVPYPVSGIREFSNLVL N NCTKYNIYDYVGTGIIRSSNQSLAGGITYVSNSGNLLGFKNVSTGNIFIV TP CNQPDQVAVYQQSIIGAMTAVNESRYGLQNLLQLPNFYYVSNGGNNCTT AVMTYSNFGICADGSLIPVRPRNSSDNGISAIITANLSIPSNWTTSVQVE YL QITSTPIVVDCATYVCNGNPRCKNLLKQYTSACKTIEDALRLSAHLETND VSSMLTFDSNAFSLANVTSFGDYNLSSVLPQRNIRSSRIAGRSALEDLLF SK VVTSGLGTVDVDYKSCTKGLSIADLACAQYYNGIMVLPGVADAERMAM YTGSLIGGMVLGGLTSAAAIPFSLALQARLNYVALQTDVLQENQKILAAS FNKAINNIVASFSSVNDAITQTAEAIHTVTIALNKIQDVVNQQGSALNHL TS QLRHNFQAISNSIQAIYDRLDPPQADQQVDRLITGRLAALNAFVSQVLNK YTEVRGSRRLAQQKINECVKSQSNRYGFCGNGTHIFSIVNSAPDGLLFLH T VLLPTDYKNVKAWSGICVDGIYGYVLRQPNLVLYSDNGVFRVTSRVMFQ PRLPVLSDFVQIYNCNVTFVNISRVELHTVIPDYIGDLNKTLQELAQNLE K LVKKGSGGSGDIIKLLNEQVNKEMQSSNLYMSMSSWCYTHSLDGAGLFL FDHAAEEYEHAKKLIIFLNENNVPVQLTSISAPEHKFEGLTQIFQKAYEH E QHISESINNIVDHAIKSKDHATFNFLQWYVAEQHEEEVLFKDILDKIELI GN ENHGLYLADQYVKGIAKSRKSGS | 267 |
| | | HKU1-1b06v0-natlead | | VIGDFNCTNSFINDYNKTIPRISEDVVDVSLGLGTYYVLNRVYLNTTLLF T GYFPKSGANFRDLALKGSIYLSTLWYKPPFLSDFNNGIFSKVKNTKLYVN NTLYSEFSTIVIGSVFVNTSYTIVVQPHNGILEITACQYTMCEYPHTVCK SK GSIRNESWHIDSSEPLCLFKKNFTYNVSADWLYFHFYQERGVFYAYYADV GMPTTFLFSLYLGTILSHYYVMPLTCNAISSNTDNETLEYWVTPLSRRQY L LNFDEHGVITNAVDCSSSFLSEIQCKTQSFAPNTGVYDLSGFTVKPVATV Y RRIPNLPDCDIDNWLNNVSVPSPLNWERRIFSNCNFNLSTLLRLVHVDSF S CNNLDKSKIFGSCFNSITVDKFAIPNRRRDDLQLGSSGFLQSSNYKIDIS SSS CQLYYSLPLVNVTINNFNPSSWNRRYGFGSFNLSSYDVVYSDHCFSVNSD FCPCADPSVVNSCAKSKPPSAICPAGTKYRHCDLDTTLYVKNWCRCSCLP DPISTYSPNTCPQKKVVVGIGEHCPGLGINEEKCGTQLNHSSCFCSPDAF L GWSFDSCISNNRCNIFSNFIFNGINSGTTCSNDLLYSNTEISTGVCVNYD LY GITGQGIFKEVSAAYYNNWQNLLYDSNGNIIGFKDFLTNKTYTILPCYSG R VSAAFYQNSSSPALLYRNLKCSYVLNNISFISQPFYFDSYLGCVLNAVNL T SYSVSSCDLRMGSGFCIDYALPSSGGSGSGISSPYRFVTFEPFNVSFVND SV ETVGGLFEIQIPTNFTIAGHEEFIQTSSPKVTIDCSAFVCSNYAACHDLL SEY GTFCDNINSILNEVNDLLDITQLQVANALMQGVTLSSNLNTNLHSDVDNI DFKSLLGCLGSQCGSSSRSLLEDLLFNKVKLSDVGFVEAYNNCTGGSEIR D LLCVQSFNGIKVLPPILSETQISGYTTAATVAAMFPPWSAAAGVPFSLNV Q YRINGLGVTMDVLNKNQKLIANAFNKALLSIQNGFTATNSALAKIQSVVN ANAQALNSLLQQLFNKFGAISSSLQEILSRLDPPEAQVQIDRLINGRLTA LN AYVSQQLSDITLIKAGASRAIEKVNECVKSQSPRINFCGNGNHILSLVQN A PYGLLFIHFSYKPTSFKTVLVSPGLCLSGDRGIAPKQGYFIKQNDSWMFT G SSYYYPEPISDKNVVFMNSCSVNFTKAPFIYLNNSISELSDFEAELSQWH K NGSGGSGDIIKLLNEQVNKEMQSSNLYMSMSSWCYTHSLDGAGLFLFDH AAEEYEHAKKLIIFLNENNVPVQLTSISAPEHKFEGLTQIFQKAYEHEQH IS ESINNIVDHAIKSKDHATFNFLQWYVAEQHEEEVLFKDILDKIELIGNEN H GLYLADQYVKGIAKSRKSGS | 268 |
| | | HKU1-1b06v0-natlead-nogly | | VIGDFNCTNSFINDYNKTIPRISEDVVDVSLGLGTYYVLNRVYLNTTLLF T GYFPKSGANFRDLALKGSIYLSTLWYKPPFLSDFNNGIFSKVKNTKLYVN NTLYSEFSTIVIGSVFVNTSYTIVVQPHNGILEITACQYTMCEYPHTVCK SK GSIRNESWHIDSSEPLCLFKKNFTYNVSADWLYFHFYQERGVFYAYYADV GMPTTFLFSLYLGTILSHYYVMPLTCNAISSNTDNETLEYWVTPLSRRQY L LNFDEHGVITNAVDCSSSFLSEIQCKTQSFAPNTGVYDLSGFTVKPVATV Y RRIPNLPDCDIDNWLNNVSVPSPLNWERRIFSNCNFNLSTLLRLVHVDSF S CNNLDKSKIFGSCFNSITVDKFAIPNRRRDDLQLGSSGFLQSSNYKIDIS SSS CQLYYSLPLVNVTINNFNPSSWNRRYGFGSFNLSSYDVVYSDHCFSVNSD FCPCADPSVVNSCAKSKPPSAICPAGTKYRHCDLDTTLYVKNWCRCSCLP DPISTYSPNTCPQKKVVVGIGEHCPGLGINEEKCGTQLNHSSCFCSPDAF L GWSFDSCISNNRCNIFSNFIFNGINSGTTCSNDLLYSNTEISTGVCVNYD LY GITGQGIFKEVSAAYYNNWQNLLYDSNGNIIGFKDFLTNKTYTILPCYSG R VSAAFYQNSSSPALLYRNLKCSYVLNNISFISQPFYFDSYLGCVLNAVNL T SYSVSSCDLRMGSGFCIDYALPSSGGSGSGISSPYRFVTFEPFNVSFVND SV ETVGGLFEIQIPTNFTIAGHEEFIQTSSPKVTIDCSAFVCSNYAACHDLL SEY GTFCDNINSILNEVNDLLDITQLQVANALMQGVTLSSNLNTNLHSDVDNI DFKSLLGCLGSQCGSSSRSLLEDLLFNKVKLSDVGFVEAYNNCTGGSEIR D LLCVQSFNGIKVLPPILSETQISGYTTAATVAAMFPPWSAAAGVPFSLNV Q YRINGLGVTMDVLNKNQKLIANAFNKALLSIQNGFTATNSALAKIQSVVN ANAQALNSLLQQLFNKFGAISSSLQEILSRLDPPEAQVQIDRLINGRLTA LN AYVSQQLSDITLIKAGASRAIEKVNECVKSQSPRINFCGNGNHILSLVQN A PYGLLFIHFSYKPTSFKTVLVSPGLCLSGDRGIAPKQGYFIKQNDSWMFT G SSYYYPEPISDKNVVFMNSCSVNFTKAPFIYLNNSISELSDFEAELSQWH K QGSGGSGDIIKLLNEQVNKEMQSSNLYMSMSSWCYTHSLDGAGLFLFDH AAEEYEHAKKLIIFLNENNVPVQLTSISAPEHKFEGLTQIFQKAYEHEQH IS ESINNIVDHAIKSKDHATFNFLQWYVAEQHEEEVLFKDILDKIELIGNEN H GLYLADQYVKGIAKSRKSGS | 269 |
| | | HKU1-1b06v1-natlead | | VIGDFNCTNSFINDYNKTIPRISEDVVDVSLGLGTYYVLNRVYLNTTLLF T GYFPKSGANFRDLALKGSIYLSTLWYKPPFLSDFNNGIFSKVKNTKLYVN NTLYSEFSTIVIGSVFVNTSYTIVVQPHNGILEITACQYTMCEYPHTVCK SK GSIRNESWHIDSSEPLCLFKKNFTYNVSADWLYFHFYQERGVFYAYYADV GMPTTFLFSLYLGTILSHYYVMPLTCNAISSNTDNETLEYWVTPLSRRQY L LNFDEHGVITNAVDCSSSFLSEIQCKTQSFAPNTGVYDLSGFTVKPVATV Y RRIPNLPDCDIDNWLNNVSVPSPLNWERRIFSNCNFNLSTLLRLVHVDSF S CNNLDKSKIFGSCFNSITVDKFAIPNRRRDDLQLGSSGFLQSSNYKIDIS SSS CQLYYSLPLVNVTINNFNPSSWNRRYGFGSFNLSSYDVVYSDHCFSVNSD FCPCADPSVVNSCAKSKPPSAICPAGTKYRHCDLDTTLYVKNWCRCSCLP DPISTYSPNTCPQKKVVVGIGEHCPGLGINEEKCGTQLNHSSCFCSPDAF L GWSFDSCISNNRCNIFSNFIFNGINSGTTCSNDLLYSNTEISTGVCVNYD LY GITGQGIFKEVSAAYYNNWQNLLYDSNGNIIGFKDFLTNKTYTILPCYSG R VSAAFYQNSSSPALLYRNLKCSYVLNNISFISQPFYFDSYLGCVLNAVNL T SYSVSSCDLRMGSGFCIDYALPSSGGSGSGISSPYRFVTFEPFNVSFVND SV ETVGGLFEIQIPTNFTIAGHEEFIQTSSPKVTIDCSAFVCSNYAACHDLL SEY GTFCDNINSILNEVNDLLDITQLQVANALMQGVTLSSNLNTNLHSDVDNI DFKSLLGCLGSQCGSSSRSLLEDLLFNKVKLSDVGFVEAYNNCTGGSEIR D LLCVQSFNGIKVLPPILSETQISGYTTAATVAAMFPPWSAAAGVPFSLNV Q YRINGLGVTMDVLNKNQKLIANAFNKALLSIQNGFTATNSALAKIQSVVN ANAQALNSLLQQLFNKFGAISSSLQEILSRLDPPEAQVQIDRLINGRLTA LN AYVSQQLSDITLIKAGASRAIEKVNECVKSQSPRINFCGNGNHILSLVQN A PYGLLFIHFSYKPTSFKTVLVSPGLCLSGDRGIAPKQGYFIKQNDSWMFT G SSYYYPEPISDKNVVFMNSCSVNFTKAPFIYLNNLQSNLQDIEAELSQIH K NGSGGSGDIIKLLNEQVNKEMQSSNLYMSMSSWCYTHSLDGAGLFLFDH AAEEYEHAKKLIIFLNENNVPVQLTSISAPEHKFEGLTQIFQKAYEHEQH IS ESINNIVDHAIKSKDHATFNFLQWYVAEQHEEEVLFKDILDKIELIGNEN H GLYLADQYVKGIAKSRKSGS | 270 |
| | | HKU1-1b06v1-natlead-nogly | | VIGDFNCTNSFINDYNKTIPRISEDVVDVSLGLGTYYVLNRVYLNTTLLF T GYFPKSGANFRDLALKGSIYLSTLWYKPPFLSDFNNGIFSKVKNTKLYVN NTLYSEFSTIVIGSVFVNTSYTIVVQPHNGILEITACQYTMCEYPHTVCK SK GSIRNESWHIDSSEPLCLFKKNFTYNVSADWLYFHFYQERGVFYAYYADV GMPTTFLFSLYLGTILSHYYVMPLTCNAISSNTDNETLEYWVTPLSRRQY L LNFDEHGVITNAVDCSSSFLSEIQCKTQSFAPNTGVYDLSGFTVKPVATV Y RRIPNLPDCDIDNWLNNVSVPSPLNWERRIFSNCNFNLSTLLRLVHVDSF S CNNLDKSKIFGSCFNSITVDKFAIPNRRRDDLQLGSSGFLQSSNYKIDIS SSS CQLYYSLPLVNVTINNFNPSSWNRRYGFGSFNLSSYDVVYSDHCFSVNSD FCPCADPSVVNSCAKSKPPSAICPAGTKYRHCDLDTTLYVKNWCRCSCLP DPISTYSPNTCPQKKVVVGIGEHCPGLGINEEKCGTQLNHSSCFCSPDAF L GWSFDSCISNNRCNIFSNFIFNGINSGTTCSNDLLYSNTEISTGVCVNYD LY GITGQGIFKEVSAAYYNNWQNLLYDSNGNIIGFKDFLTNKTYTILPCYSG R VSAAFYQNSSSPALLYRNLKCSYVLNNISFISQPFYFDSYLGCVLNAVNL T SYSVSSCDLRMGSGFCIDYALPSSGGSGSGISSPYRFVTFEPFNVSFVND SV ETVGGLFEIQIPTNFTIAGHEEFIQTSSPKVTIDCSAFVCSNYAACHDLL SEY GTFCDNINSILNEVNDLLDITQLQVANALMQGVTLSSNLNTNLHSDVDNI DFKSLLGCLGSQCGSSSRSLLEDLLFNKVKLSDVGFVEAYNNCTGGSEIR D LLCVQSFNGIKVLPPILSETQISGYTTAATVAAMFPPWSAAAGVPFSLNV Q YRINGLGVTMDVLNKNQKLIANAFNKALLSIQNGFTATNSALAKIQSVVN ANAQALNSLLQQLFNKFGAISSSLQEILSRLDPPEAQVQIDRLINGRLTA LN AYVSQQLSDITLIKAGASRAIEKVNECVKSQSPRINFCGNGNHILSLVQN A PYGLLFIHFSYKPTSFKTVLVSPGLCLSGDRGIAPKQGYFIKQNDSWMFT G SSYYYPEPISDKNVVFMNSCSVNFTKAPFIYLNNLQSNLQDIEAELSQIH K QGSGGSGDIIKLLNEQVNKEMQSSNLYMSMSSWCYTHSLDGAGLFLFDH AAEEYEHAKKLIIFLNENNVPVQLTSISAPEHKFEGLTQIFQKAYEHEQH IS ESINNIVDHAIKSKDHATFNFLQWYVAEQHEEEVLFKDILDKIELIGNEN H GLYLADQYVKGIAKSRKSGS | 271 |
| | | HKU1-1b06v2-natlead | | VIGDFNCTNSFINDYNKTIPRISEDVVDVSLGLGTYYVLNRVYLNTTLLF T GYFPKSGANFRDLALKGSIYLSTLWYKPPFLSDFNNGIFSKVKNTKLYVN NTLYSEFSTIVIGSVFVNTSYTIVVQPHNGILEITACQYTMCEYPHTVCK SK GSIRNESWHIDSSEPLCLFKKNFTYNVSADWLYFHFYQERGVFYAYYADV GMPTTFLFSLYLGTILSHYYVMPLTCNAISSNTDNETLEYWVTPLSRRQY L LNFDEHGVITNAVDCSSSFLSEIQCKTQSFAPNTGVYDLSGFTVKPVATV Y RRIPNLPDCDIDNWLNNVSVPSPLNWERRIFSNCNFNLSTLLRLVHVDSF S CNNLDKSKIFGSCFNSITVDKFAIPNRRRDDLQLGSSGFLQSSNYKIDIS SSS CQLYYSLPLVNVTINNFNPSSWNRRYGFGSFNLSSYDVVYSDHCFSVNSD FCPCADPSVVNSCAKSKPPSAICPAGTKYRHCDLDTTLYVKNWCRCSCLP DPISTYSPNTCPQKKVVVGIGEHCPGLGINEEKCGTQLNHSSCFCSPDAF L GWSFDSCISNNRCNIFSNFIFNGINSGTTCSNDLLYSNTEISTGVCVNYD LY GITGQGIFKEVSAAYYNNWQNLLYDSNGNIIGFKDFLTNKTYTILPCYSG R VSAAFYQNSSSPALLYRNLKCSYVLNNISFISQPFYFDSYLGCVLNAVNL T SYSVSSCDLRMGSGFCIDYALPSSGGSGSGISSPYRFVTFEPFNVSFVND SV ETVGGLFEIQIPTNFTIAGHEEFIQTSSPKVTIDCSAFVCSNYAACHDLL SEY GTFCDNINSILNEVNDLLDITQLQVANALMQGVTLSSNLNTNLHSDVDNI DFKSLLGCLGSQCGSSSRSLLEDLLFNKVKLSDVGFVEAYNNCTGGSEIR D LLCVQSFNGIKVLPPILSETQISGYTTAATVAAMFPPWSAAAGVPFSLNV Q YRINGLGVTMDVLNKNQKLIANAFNKALLSIQNGFTATNSALAKIQSVVN ANAQALNSLLQQLFNKFGAISSSLQEILSRLDPPEAQVQIDRLINGRLTA LN AYVSQQLSDITLIKAGASRAIEKVNECVKSQSPRINFCGNGNHILSLVQN A PYGLLFIHFSYKPTSFKTVLVSPGLCLSGDRGIAPKQGYFIKQNDSWMFT G SSYYYPEPISDKNVVFMNSCSVNFTKAPFIYLNNLQSNLQDLEAELSQLH K NGSGGSGDIIKLLNEQVNKEMQSSNLYMSMSSWCYTHSLDGAGLFLFDH AAEEYEHAKKLIIFLNENNVPVQLTSISAPEHKFEGLTQIFQKAYEHEQH IS ESINNIVDHAIKSKDHATFNFLQWYVAEQHEEEVLFKDILDKIELIGNEN H GLYLADQYVKGIAKSRKSGS | 272 |
| | | HKU1-1b06v2-natlead-nogly | | VIGDFNCTNSFINDYNKTIPRISEDVVDVSLGLGTYYVLNRVYLNTTLLF T GYFPKSGANFRDLALKGSIYLSTLWYKPPFLSDFNNGIFSKVKNTKLYVN NTLYSEFSTIVIGSVFVNTSYTIVVQPHNGILEITACQYTMCEYPHTVCK SK GSIRNESWHIDSSEPLCLFKKNFTYNVSADWLYFHFYQERGVFYAYYADV GMPTTFLFSLYLGTILSHYYVMPLTCNAISSNTDNETLEYWVTPLSRRQY L LNFDEHGVITNAVDCSSSFLSEIQCKTQSFAPNTGVYDLSGFTVKPVATV Y RRIPNLPDCDIDNWLNNVSVPSPLNWERRIFSNCNFNLSTLLRLVHVDSF S CNNLDKSKIFGSCFNSITVDKFAIPNRRRDDLQLGSSGFLQSSNYKIDIS SSS CQLYYSLPLVNVTINNFNPSSWNRRYGFGSFNLSSYDVVYSDHCFSVNSD FCPCADPSVVNSCAKSKPPSAICPAGTKYRHCDLDTTLYVKNWCRCSCLP DPISTYSPNTCPQKKVVVGIGEHCPGLGINEEKCGTQLNHSSCFCSPDAF L GWSFDSCISNNRCNIFSNFIFNGINSGTTCSNDLLYSNTEISTGVCVNYD LY GITGQGIFKEVSAAYYNNWQNLLYDSNGNIIGFKDFLTNKTYTILPCYSG R VSAAFYQNSSSPALLYRNLKCSYVLNNISFISQPFYFDSYLGCVLNAVNL T SYSVSSCDLRMGSGFCIDYALPSSGGSGSGISSPYRFVTFEPFNVSFVND SV ETVGGLFEIQIPTNFTIAGHEEFIQTSSPKVTIDCSAFVCSNYAACHDLL SEY GTFCDNINSILNEVNDLLDITQLQVANALMQGVTLSSNLNTNLHSDVDNI DFKSLLGCLGSQCGSSSRSLLEDLLFNKVKLSDVGFVEAYNNCTGGSEIR D LLCVQSFNGIKVLPPILSETQISGYTTAATVAAMFPPWSAAAGVPFSLNV Q YRINGLGVTMDVLNKNQKLIANAFNKALLSIQNGFTATNSALAKIQSVVN ANAQALNSLLQQLFNKFGAISSSLQEILSRLDPPEAQVQIDRLINGRLTA LN AYVSQQLSDITLIKAGASRAIEKVNECVKSQSPRINFCGNGNHILSLVQN A PYGLLFIHFSYKPTSFKTVLVSPGLCLSGDRGIAPKQGYFIKQNDSWMFT G SSYYYPEPISDKNVVFMNSCSVNFTKAPFIYLNNLQSNLQDLEAELSQLH K QGSGGSGDIIKLLNEQVNKEMQSSNLYMSMSSWCYTHSLDGAGLFLFDH AAEEYEHAKKLIIFLNENNVPVQLTSISAPEHKFEGLTQIFQKAYEHEQH IS ESINNIVDHAIKSKDHATFNFLQWYVAEQHEEEVLFKDILDKIELIGNEN H GLYLADQYVKGIAKSRKSGS | 273 |
| | | HKU1-1b06v3-natlead-nogly | | VIGDFNCTNSFINDYNKTIPRISEDVVDVSLGLGTYYVLNRVYLNTTLLF T GYFPKSGANFRDLALKGSIYLSTLWYKPPFLSDFNNGIFSKVKNTKLYVN NTLYSEFSTIVIGSVFVNTSYTIVVQPHNGILEITACQYTMCEYPHTVCK SK GSIRNESWHIDSSEPLCLFKKNFTYNVSADWLYFHFYQERGVFYAYYADV GMPTTFLFSLYLGTILSHYYVMPLTCNAISSNTDNETLEYWVTPLSRRQY L LNFDEHGVITNAVDCSSSFLSEIQCKTQSFAPNTGVYDLSGFTVKPVATV Y RRIPNLPDCDIDNWLNNVSVPSPLNWERRIFSNCNFNLSTLLRLVHVDSF S CNNLDKSKIFGSCFNSITVDKFAIPNRRRDDLQLGSSGFLQSSNYKIDIS SSS CQLYYSLPLVNVTINNFNPSSWNRRYGFGSFNLSSYDVVYSDHCFSVNSD FCPCADPSVVNSCAKSKPPSAICPAGTKYRHCDLDTTLYVKNWCRCSCLP DPISTYSPNTCPQKKVVVGIGEHCPGLGINEEKCGTQLNHSSCFCSPDAF L GWSFDSCISNNRCNIFSNFIFNGINSGTTCSNDLLYSNTEISTGVCVNYD LY GITGQGIFKEVSAAYYNNWQNLLYDSNGNIIGFKDFLTNKTYTILPCYSG R VSAAFYQNSSSPALLYRNLKCSYVLNNISFISQPFYFDSYLGCVLNAVNL T SYSVSSCDLRMGSGFCIDYALPSSGGSGSGISSPYRFVTFEPFNVSFVND SV ETVGGLFEIQIPTNFTIAGHEEFIQTSSPKVTIDCSAFVCSNYAACHDLL SEY GTFCDNINSILNEVNDLLDITQLQVANALMQGVTLSSNLNTNLHSDVDNI DFKSLLGCLGSQCGSSSRSLLEDLLFNKVKLSDVGFVEAYNNCTGGSEIR D LLCVQSFNGIKVLPPILSETQISGYTTAATVAAMFPPWSAAAGVPFSLNV Q YRINGLGVTMDVLNKNQKLIANAFNKALLSIQNGFTATNSALAKIQSVVN ANAQALNSLLQQLFNKFGAISSSLQEILSRLDPPEAQVQIDRLINGRLTA LN AYVSQQLSDITLIKAGASRAIEKVNECVKSQSPRINFCGNGNHILSLVQN A PYGLLFIHFSYKPTSFKTVLVSPGLCLSGDRGIAPKQGYFIKQNDSWMFT G SSYYYPEPISDKNVVFMNSCSVNFTKAPFIYLNNSISNLQDIEAELSQIH KQ GSGGSGDIIKLLNEQVNKEMQSSNLYMSMSSWCYTHSLDGAGLFLFDHA AEEYEHAKKLIIFLNENNVPVQLTSISAPEHKFEGLTQIFQKAYEHEQHI SE SINNIVDHAIKSKDHATFNFLQWYVAEQHEEEVLFKDILDKIELIGNENH G LYLADQYVKGIAKSRKSGS | 274 |
| | | HKU1-1b06v4-natlead-nogly | | VIGDFNCTNSFINDYNKTIPRISEDVVDVSLGLGTYYVLNRVYLNTTLLF T GYFPKSGANFRDLALKGSIYLSTLWYKPPFLSDFNNGIFSKVKNTKLYVN NTLYSEFSTIVIGSVFVNTSYTIVVQPHNGILEITACQYTMCEYPHTVCK SK GSIRNESWHIDSSEPLCLFKKNFTYNVSADWLYFHFYQERGVFYAYYADV GMPTTFLFSLYLGTILSHYYVMPLTCNAISSNTDNETLEYWVTPLSRRQY L LNFDEHGVITNAVDCSSSFLSEIQCKTQSFAPNTGVYDLSGFTVKPVATV Y RRIPNLPDCDIDNWLNNVSVPSPLNWERRIFSNCNFNLSTLLRLVHVDSF S CNNLDKSKIFGSCFNSITVDKFAIPNRRRDDLQLGSSGFLQSSNYKIDIS SSS CQLYYSLPLVNVTINNFNPSSWNRRYGFGSFNLSSYDVVYSDHCFSVNSD FCPCADPSVVNSCAKSKPPSAICPAGTKYRHCDLDTTLYVKNWCRCSCLP DPISTYSPNTCPQKKVVVGIGEHCPGLGINEEKCGTQLNHSSCFCSPDAF L GWSFDSCISNNRCNIFSNFIFNGINSGTTCSNDLLYSNTEISTGVCVNYD LY GITGQGIFKEVSAAYYNNWQNLLYDSNGNIIGFKDFLTNKTYTILPCYSG R VSAAFYQNSSSPALLYRNLKCSYVLNNISFISQPFYFDSYLGCVLNAVNL T SYSVSSCDLRMGSGFCIDYALPSSGGSGSGISSPYRFVTFEPFNVSFVND SV ETVGGLFEIQIPTNFTIAGHEEFIQTSSPKVTIDCSAFVCSNYAACHDLL SEY GTFCDNINSILNEVNDLLDITQLQVANALMQGVTLSSNLNTNLHSDVDNI DFKSLLGCLGSQCGSSSRSLLEDLLFNKVKLSDVGFVEAYNNCTGGSEIR D LLCVQSFNGIKVLPPILSETQISGYTTAATVAAMFPPWSAAAGVPFSLNV Q YRINGLGVTMDVLNKNQKLIANAFNKALLSIQNGFTATNSALAKIQSVVN ANAQALNSLLQQLFNKFGAISSSLQEILSRLDPPEAQVQIDRLINGRLTA LN AYVSQQLSDITLIKAGASRAIEKVNECVKSQSPRINFCGNGNHILSLVQN A PYGLLFIHFSYKPTSFKTVLVSPGLCLSGDRGIAPKQGYFIKQNDSWMFT G SSYYYPEPISDKNVVFMNSCSVNFTKAPFIYLNNSISNLQDLEAELSQLH K QGSGGSGDIIKLLNEQVNKEMQSSNLYMSMSSWCYTHSLDGAGLFLFDH AAEEYEHAKKLIIFLNENNVPVQLTSISAPEHKFEGLTQIFQKAYEHEQH IS ESINNIVDHAIKSKDHATFNFLQWYVAEQHEEEVLFKDILDKIELIGNEN H GLYLADQYVKGIAKSRKSGS | 275 |
| | | M1-RBD-Ferr | | EGVECDFSPLLSGTPPQVYNFKRLVFTNCNYNLTKLLSLFSVNDFTCSQI SP AAIASNCYSSLILDYFSYPLSMKSDLSVSSAGPISQFNYKQSFSNPTCLI LAT VPHNLTTITKPLKYSYINKCSRLLSDDRTEVPQLVNANQYSPCVSIVPST V WEDGDYYRKQLSPLEGGGWLVASGSTVAMTEQLQMGFGITVQYGTDTN SVCPKGSGGGGESQVRQQFSKDIEKLLNEQVNKEMQSSNLYMSMSSWCY THSLDGAGLFLFDHAAEEYEHAKKLIIFLNENNVPVQLTSISAPEHKFEG L TQIFQKAYEHEQHISESINNIVDHAIKSKDHATFNFLQWYVAEQHEEEVL F KDILDKIELIGNENHGLYLADQYVKGIAKSRKSGS | 276 |
| | | M2-RBD-Ferr | | ECDFSPLLSGTPPQVYNFKRLVFTNCNYNLTKLLSLFSVNDFTCSQISPA AI ASNCYSSLILDYFSYPLSMKSDLSVSSAGPISQFNYKQSFSNPTCLILAT VP HNLTTITKPLKYSYINKCSRLLSDDRTEVPQLVNANQYSPCVSIVPSTVW E DGDYYRKQLSPLEGGGWLVASGSTVAMTEQLQMGFGITVQYGTDTNSV CPK GSGGGGESQVRQQFSKDIEKLLNEQVNKEMQSSNLYMSMSSWCYTHSLD GAGLFLFDHAAEEYEHAKKLIIFLNENNVPVQLTSISAPEHKFEGLTQIF QK AYEHEQHISESINNIVDHAIKSKDHATFNFLQWYVAEQHEEEVLFKDILD K IELIGNENHGLYLADQYVKGIAKSRKSGS | 277 |
| | | M3-RBD-Ferr | | EGVECDFSPLLSGTPPQVYNFKRLVFTNCNYNLTKLLSLFSVNDFTCSQI SP AAIASNCYSSLILDYFSYPLSMKSDLSVSSAGPISQFNYKQSFSNPTCLI LAT VPHNLTTITKPLKYSYINKCSRLLSDDRTEVPQLVNANQYSPCVSIVPST V WEDGDYYRKQLSPLEGGGWLVASGSTVAMTEQLQMGFGITVQNGTDTN SVCPK GSGGGGESQVRQQFSKDIEKLLNEQVNKEMQSSNLYMSMSSWCYTHSLD GAGLFLFDHAAEEYEHAKKLIIFLNENNVPVQLTSISAPEHKFEGLTQIF QK AYEHEQHISESINNIVDHAIKSKDHATFNFLQWYVAEQHEEEVLFKDILD K IELIGNENHGLYLADQYVKGIAKSRKSGS | 278 |
| | | M4-RBD-Ferr | | EGVECDFSPLLSGTPPQVYNFKRLVFTNCNYNLTKLLSLFSVNDFTCSQI SP AAIASNCYSSLILDYFSYPLSMKSDLSVSSAGPISQFNYKQSFSNPTCLI LAT VPHNLTTITKPLKYSYINKCSRLLSDDRTEVPQLVNANQYSPCVSIVPST V WEDGDYYRKQLSPLEGGGWLVASGSTVAMTEQLQMGFGITVQRGTDTN SVCPK GSGGGGESQVRQQFSKDIEKLLNEQVNKEMQSSNLYMSMSSWCYTHSLD GAGLFLFDHAAEEYEHAKKLIIFLNENNVPVQLTSISAPEHKFEGLTQIF QK AYEHEQHISESINNIVDHAIKSKDHATFNFLQWYVAEQHEEEVLFKDILD K IELIGNENHGLYLADQYVKGIAKSRKSGS | 279 |
| | | M5-RBD-Ferr | | ECDFSPLLSGTPPQVYNFKRLVFTNCNYNLTKLLSLFSVNDFTCSQISPA AI ASNCYSSLILDYFSYPLSMKSDLSVSSAGPISQFNYKQSFSNPTCLILAT VP HNLTTITKPLKYSYINKCSRLLSDDRTEVPQLVNANQYSPCVSIVPSTVW E DGDYYRKQLSPLEGGGWLVASGSTVAMTEQLQMGFGITVQNGTDTNSV CPK GSGGGGESQVRQQFSKDIEKLLNEQVNKEMQSSNLYMSMSSWCYTHSLD GAGLFLFDHAAEEYEHAKKLIIFLNENNVPVQLTSISAPEHKFEGLTQIF QK AYEHEQHISESINNIVDHAIKSKDHATFNFLQWYVAEQHEEEVLFKDILD K IELIGNENHGLYLADQYVKGIAKSRKSGS | 280 |
| | | M6-RBD-Ferr | | ECDFSPLLSGTPPQVYNFKRLVFTNCNYNLTKLLSLFSVNDFTCSQISPA AI ASNCYSSLILDYFSYPLSMKSDLSVSSAGPISQFNYKQSFSNPTCLILAT VP HNLTTITKPLKYSYINKCSRLLSDDRTEVPQLVNANQYSPCVSIVPSTVW E DGDYYRKQLSPLEGGGWLVASGSTVAMTEQLQMGFGITVQRGTDTNSV CPKGSGGGGESQVRQQFSKDIEKLLNEQVNKEMQSSNLYMSMSSWCYTH SLDGAGLFLFDHAAEEYEHAKKLIIFLNENNVPVQLTSISAPEHKFEGLT QI FQKAYEHEQHISESINNIVDHAIKSKDHATFNFLQWYVAEQHEEEVLFKD I LDKIELIGNENHGLYLADQYVKGIAKSRKSGS | 281 |
| Intentionally Left Blank | | | | | 282 |
| Intentionally Left Blank | | | | | 283 |
| 1B-05 with DS1 +D614G, native leader | pCoV204 | S2P.1154-DS1-del-Ferritin-D614G | | VNLTTRTQLPPAYTNSFTRGVYYPDKVFRSSVLHSTQDLFLPFFSNVTWF HAIHVSGTNGTKRFDNPVLPFNDGVYFASTEKSNIIRGWIFGTTLDSKTQ SLLIVNNATNVVIKVCEFQFCNDPFLGVYYHKNNKSWMESEFRVYSSANN CTFEYVSQPFLMDLEGKQGNFKNLREFVFKNIDGYFKIYSKHTPINLVRD LPQGFSALEPLVDLPIGINITRFQTLLALHRSYLTPGDSSSGWTAGAAAY YVGYLQPRTFLLKYNENGTITDAVDCALDPLSETKCTLKSFTVEKGIYQT SNFRVQPTESIVRFPNITNLCPFGEVFNATRFASVYAWNRKRISNCVADY SVLYNSASFSTFKCYGVCPTKLNDLCFTNVYADSFVIRGDEVRQIAPGQT GKIADYNYKLPDDFTGCVIAWNSNNLDSKVGGNYNYLYRLFRKSNLKPFE RDISTEIYQAGSTPCNGVEGFNCYFPLQSYGFQPTNGVGYQPYRVVVLSF ELLHAPATVCGPKKSTNLVKNKCVNFNFNGLTGTGVLTESNKKFLPFQQF GRDIADTTDAVRDPQTLEILDITPCSFGGVSVITPGTNTSNQVAVLYQGV NCTEVPVAIHADQLTPTWRVYSTGSNVFQTRAGCLIGAEHVNNSYECDIP IGAGICASYQTQTNSPGSASSVASQSIIAYTMSLGAENSVAYSNNSIAIP TNFTISVTTEILPVSMTKTSVDCTMYICGDSTECSNLLLQYGSFCTQLNR ALTGIAVEQDKNTQEVFAQVKQIYKTPPIKDFGGFNFSQILPDPSKPSKR SFIEDLLFNKVTLADAGFIKQYGDCLGDIAARDLICAQKFNGLTVLPPLL TDEMIAQYTSALLAGTITSGWTFGAGAALQIPFAMQMAYRFNGIGVTQNV LYENQKLIANQFNSAIGKIQDSLSSTASALGKLQDVVNQNAQALNTLVKQ LSSNFGAISSVLNDILSRLCPPEAEVQIDRLITGRLQSLQTYVTQQLIRA AEIRASANLAATKMSECVLGQSKRVDFCGKGYHLMSFPQSAPHGVVFLHV TYVPAQEKNFTTAPAICHDGKAHFPREGVFVSNGTHWFVTQRNFYEPQII TTDNTFVSGNCDVVIGIVNNTVYDPLQPELDSFKEELDKGSGGSGDIIKL LNEQVNKEMQSSNLYMSMSSWCYTHSLDGAGLFLFDHAAEEYEHAKKLII FLNENNVPVQLTSISAPEHKFEGLTQIFQKAYEHEQHISESINNIVDHAI KSKDHATFNFLQWYVAEQHEEEVLFKDILDKIELIGNENHGLYLADQYVK GIAKSRKSGS | 284 |
| 1B-06-PL with DS1 + D614G | pCoV205 | S2P.1158op1-DS1-del-Ferritin-D614G | | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVCPTKLNDLCFTNVYADSFVIRGDEVRQIAPGQTGK I ADYNYKLPDDFTGCVIAWNSNNLDSKVGGNYNYLYRLFRKSNLKPFERDI STEIYQAGSTPCNGVEGFNCYFPLQSYGFQPTNGVGYQPYRVVVLSFELL HAPATVCGPKKSTNLVKNKCVNFNFNGLTGTGVLTESNKKFLPFQQFGRD IADTTDAVRDPQTLEILDITPCSFGGVSVITPGTNTSNQVAVLYQGVNCT EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TISVTTEILPVSMTKTSVDCTMYICGDSTECSNLLLQYGSFCTQLNRALT GIAVEQDKNTQEVFAQVKQIYKTPPIKDFGGFNFSQILPDPSKPSKRSFI EDLLFNKVTLADAGFIKQYGDCLGDIAARDLICAQKFNGLTVLPPLLTDE MIAQYTSALLAGTITSGWTFGAGAALQIPFAMQMAYRFNGIGVTQNVLYE NQKLIANQFNSAIGKIQDSLSSTASALGKLQDVVNQNAQALNTLVKQLSS NFGAISSVLNDILSRLCPPEAEVQIDRLITGRLQSLQTYVTQQLIRAAEI RASANLAATKMSECVLGQSKRVDFCGKGYHLMSFPQSAPHGVVFLHVTYV PAQEKNFTTAPAICHDGKAHFPREGVFVSNGTHWFVTQRNFYEPQIITTD NTFVSGNCDVVIGIVNNTVYDPLQSELDSIKEELDKIHKNGSGGSGDIIK LLNEQVNKEMQSSNLYMSMSSWCYTHSLDGAGLFLFDHAAEEYEHAKKLI IFLNENNVPVQLTSISAPEHKFEGLTQIFQKAYEHEQHISESINNIVDHA IKSKDHATFNFLQWYVAEQHEEEVLFKDILDKIELIGNENHGLYLADQYV KGIAKSRKSGS | 285 |
| 1B-08-PL with DS1 + D614G | pCoV206 | S2P.1158oplx2-DS1-del-Ferritin-D614G | | SSQCVNLTTRTQLPPAYTNSFTRGVYYPDKVFRSSVLHSTQDLFLPFFSN VTWFHAIHVSGTNGTKRFDNPVLPFNDGVYFASTEKSNIIRGWIFGTTLD SKTQSLLIVNNATNVVIKVCEFQFCNDPFLGVYYHKNNKSWMESEFRVYS SANNCTFEYVSQPFLMDLEGKQGNFKNLREFVFKNIDGYFKIYSKHTPIN LVRDLPQGFSALEPLVDLPIGINITRFQTLLALHRSYLTPGDSSSGWTAG AAAYYVGYLQPRTFLLKYNENGTITDAVDCALDPLSETKCTLKSFTVEKG IYQTSNFRVQPTESIVRFPNITNLCPFGEVFNATRFASVYAWNRKRISNC VADYSVLYNSASFSTFKCYGVCPTKLNDLCFTNVYADSFVIRGDEVRQIA PGQTGKIADYNYKLPDDFTGCVIAWNSNNLDSKVGGNYNYLYRLFRKSNL KPFERDISTEIYQAGSTPCNGVEGFNCYFPLQSYGFQPTNGVGYQPYRVV VLSFELLHAPATVCGPKKSTNLVKNKCVNFNFNGLTGTGVLTESNKKFLP FQQFGRDIADTTDAVRDPQTLEILDITPCSFGGVSVITPGTNTSNQVAVL YQGVNCTEVPVAIHADQLTPTWRVYSTGSNVFQTRAGCLIGAEHVNNSYE CDIPIGAGICASYQTQTNSPGSASSVASQSIIAYTMSLGAENSVAYSNNS IAIPTNFTISVTTEILPVSMTKTSVDCTMYICGDSTECSNLLLQYGSFCT QLNRALTGIAVEQDKNTQEVFAQVKQIYKTPPIKDFGGFNFSQILPDPSK PSKRSFIEDLLFNKVTLADAGFIKQYGDCLGDIAARDLICAQKFNGLTVL PPLLTDEMIAQYTSALLAGTITSGWTFGAGAALQIPFAMQMAYRFNGIGV TQNVLYENQKLIANQFNSAIGKIQDSLSSTASALGKLQDVVNQNAQALNT LVKQLSSNFGAISSVLNDILSRLCPPEAEVQIDRLITGRLQSLQTYVTQQ LIRAAEIRASANLAATKMSECVLGQSKRVDFCGKGYHLMSFPQSAPHGVV FLHVTYVPAQEKNFTTAPAICHDGKAHFPREGVFVSNGTHWFVTQRNFYE PQIITTDNTFVSGNCDVVIGIVNNTVYDPLQSELDSIKEELDKIHKNLDS IKEELDKIHKNGSGGSGDIIKLLNEQVNKEMQSSNLYMSMSSWCYTHSLD GAGLFLFDHAAEEYEHAKKLIIFLNENNVPVQLTSISAPEHKFEGLTQIF QKAYEHEQHISESINNIVDHAIKSKDHATFNFLQWYVAEQHEEEVLFKDI LDKIELIGNENHGLYLADQYVKGIAKSRKSGS | 286 |
| 1B-05 with N165Q (more “up” RBD) | pCoV207 | S2P.1154-del-Ferritin-N165Q | | VNLTTRTQLPPAYTNSFTRGVYYPDKVFRSSVLHSTQDLFLPFFSNVTWF HAIHVSGTNGTKRFDNPVLPFNDGVYFASTEKSNIIRGWIFGTTLDSKTQ SLLIVNNATNVVIKVCEFQFCNDPFLGVYYHKNNKSWMESEFRVYSSANQ CTFEYVSQPFLMDLEGKQGNFKNLREFVFKNIDGYFKIYSKHTPINLVRD LPQGFSALEPLVDLPIGINITRFQTLLALHRSYLTPGDSSSGWTAGAAAY YVGYLQPRTFLLKYNENGTITDAVDCALDPLSETKCTLKSFTVEKGIYQT SNFRVQPTESIVRFPNITNLCPFGEVFNATRFASVYAWNRKRISNCVADY SVLYNSASFSTFKCYGVSPTKLNDLCFTNVYADSFVIRGDEVRQIAPGQT GKIADYNYKLPDDFTGCVIAWNSNNLDSKVGGNYNYLYRLFRKSNLKPFE RDISTEIYQAGSTPCNGVEGFNCYFPLQSYGFQPTNGVGYQPYRVVVLSF ELLHAPATVCGPKKSTNLVKNKCVNFNFNGLTGTGVLTESNKKFLPFQQF GRDIADTTDAVRDPQTLEILDITPCSFGGVSVITPGTNTSNQVAVLYQDV NCTEVPVAIHADQLTPTWRVYSTGSNVFQTRAGCLIGAEHVNNSYECDIP IGAGICASYQTQTNSPGSASSVASQSIIAYTMSLGAENSVAYSNNSIAIP TNFTISVTTEILPVSMTKTSVDCTMYICGDSTECSNLLLQYGSFCTQLNR ALTGIAVEQDKNTQEVFAQVKQIYKTPPIKDFGGFNFSQILPDPSKPSKR SFIEDLLFNKVTLADAGFIKQYGDCLGDIAARDLICAQKFNGLTVLPPLL TDEMIAQYTSALLAGTITSGWTFGAGAALQIPFAMQMAYRFNGIGVTQNV LYENQKLIANQFNSAIGKIQDSLSSTASALGKLQDVVNQNAQALNTLVKQ LSSNFGAISSVLNDILSRLDPPEAEVQIDRLITGRLQSLQTYVTQQLIRA AEIRASANLAATKMSECVLGQSKRVDFCGKGYHLMSFPQSAPHGVVFLHV TYVPAQEKNFTTAPAICHDGKAHFPREGVFVSNGTHWFVTQRNFYEPQII TTDNTFVSGNCDVVIGIVNNTVYDPLQPELDSFKEELDKGSGGSGDIIKL LNEQVNKEMQSSNLYMSMSSWCYTHSLDGAGLFLFDHAAEEYEHAKKLII FLNENNVPVQLTSISAPEHKFEGLTQIFQKAYEHEQHISESINNIVDHAI KSKDHATFNFLQWYVAEQHEEEVLFKDILDKIELIGNENHGLYLADQYVK GIAKSRKSGS | 287 |
| 1B-05 with N234Q (more “down” RBD) | pCoV208 | S2P.1154-del-Ferritin-N234Q | | VNLTTRTQLPPAYTNSFTRGVYYPDKVFRSSVLHSTQDLFLPFFSNVTWF HAIHVSGTNGTKRFDNPVLPFNDGVYFASTEKSNIIRGWIFGTTLDSKTQ SLLIVNNATNVVIKVCEFQFCNDPFLGVYYHKNNKSWMESEFRVYSSANN CTFEYVSQPFLMDLEGKQGNFKNLREFVFKNIDGYFKIYSKHTPINLVRD LPQGFSALEPLVDLPIGIQITRFQTLLALHRSYLTPGDSSSGWTAGAAAY YVGYLQPRTFLLKYNENGTITDAVDCALDPLSETKCTLKSFTVEKGIYQT SNFRVQPTESIVRFPNITNLCPFGEVFNATRFASVYAWNRKRISNCVADY SVLYNSASFSTFKCYGVSPTKLNDLCFTNVYADSFVIRGDEVRQIAPGQT GKIADYNYKLPDDFTGCVIAWNSNNLDSKVGGNYNYLYRLFRKSNLKPFE RDISTEIYQAGSTPCNGVEGFNCYFPLQSYGFQPTNGVGYQPYRVVVLSF ELLHAPATVCGPKKSTNLVKNKCVNFNFNGLTGTGVLTESNKKFLPFQQF GRDIADTTDAVRDPQTLEILDITPCSFGGVSVITPGTNTSNQVAVLYQDV NCTEVPVAIHADQLTPTWRVYSTGSNVFQTRAGCLIGAEHVNNSYECDIP IGAGICASYQTQTNSPGSASSVASQSIIAYTMSLGAENSVAYSNNSIAIP TNFTISVTTEILPVSMTKTSVDCTMYICGDSTECSNLLLQYGSFCTQLNR ALTGIAVEQDKNTQEVFAQVKQIYKTPPIKDFGGFNFSQILPDPSKPSKR SFIEDLLFNKVTLADAGFIKQYGDCLGDIAARDLICAQKFNGLTVLPPLL TDEMIAQYTSALLAGTITSGWTFGAGAALQIPFAMQMAYRFNGIGVTQNV LYENQKLIANQFNSAIGKIQDSLSSTASALGKLQDVVNQNAQALNTLVKQ LSSNFGAISSVLNDILSRLDPPEAEVQIDRLITGRLQSLQTYVTQQLIRA AEIRASANLAATKMSECVLGQSKRVDFCGKGYHLMSFPQSAPHGVVFLHV TYVPAQEKNFTTAPAICHDGKAHFPREGVFVSNGTHWFVTQRNFYEPQII TTDNTFVSGNCDVVIGIVNNTVYDPLQPELDSFKEELDKGSGGSGDIIKL LNEQVNKEMQSSNLYMSMSSWCYTHSLDGAGLFLFDHAAEEYEHAKKLII FLNENNVPVQLTSISAPEHKFEGLTQIFQKAYEHEQHISESINNIVDHAI KSKDHATFNFLQWYVAEQHEEEVLFKDILDKIELIGNENHGLYLADQYVK GIAKSRKSGS | 288 |
| 1B-06-PL with N165Q (more “up” RBD) | pCoV209 | S2P.1158op1-del-Ferritin-N165Q | | SSQCVNLTTRTQLPPAYTNSFTRGVYYPDKVFRSSVLHSTQDLFLPFFSN VTWFHAIHVSGTNGTKRFDNPVLPFNDGVYFASTEKSNIIRGWIFGTTLD SKTQSLLIVNNATNVVIKVCEFQFCNDPFLGVYYHKNNKSWMESEFRVYS SANQCTFEYVSQPFLMDLEGKQGNFKNLREFVFKNIDGYFKIYSKHTPIN LVRDLPQGFSALEPLVDLPIGINITRFQTLLALHRSYLTPGDSSSGWTAG AAAYYVGYLQPRTFLLKYNENGTITDAVDCALDPLSETKCTLKSFTVEKG IYQTSNFRVQPTESIVRFPNITNLCPFGEVFNATRFASVYAWNRKRISNC VADYSVLYNSASFSTFKCYGVSPTKLNDLCFTNVYADSFVIRGDEVRQIA PGQTGKIADYNYKLPDDFTGCVIAWNSNNLDSKVGGNYNYLYRLFRKSNL KPFERDISTEIYQAGSTPCNGVEGFNCYFPLQSYGFQPTNGVGYQPYRVV VLSFELLHAPATVCGPKKSTNLVKNKCVNFNFNGLTGTGVLTESNKKFLP FQQFGRDIADTTDAVRDPQTLEILDITPCSFGGVSVITPGTNTSNQVAVL YQDVNCTEVPVAIHADQLTPTWRVYSTGSNVFQTRAGCLIGAEHVNNSYE CDIPIGAGICASYQTQTNSPGSASSVASQSIIAYTMSLGAENSVAYSNNS IAIPTNFTISVTTEILPVSMTKTSVDCTMYICGDSTECSNLLLQYGSFCT QLNRALTGIAVEQDKNTQEVFAQVKQIYKTPPIKDFGGFNFSQILPDPSK PSKRSFIEDLLFNKVTLADAGFIKQYGDCLGDIAARDLICAQKFNGLTVL PPLLTDEMIAQYTSALLAGTITSGWTFGAGAALQIPFAMQMAYRFNGIGV TQNVLYENQKLIANQFNSAIGKIQDSLSSTASALGKLQDVVNQNAQALNT LVKQLSSNFGAISSVLNDILSRLDPPEAEVQIDRLITGRLQSLQTYVTQQ LIRAAEIRASANLAATKMSECVLGQSKRVDFCGKGYHLMSFPQSAPHGVV FLHVTYVPAQEKNFTTAPAICHDGKAHFPREGVFVSNGTHWFVTQRNFYE PQIITTDNTFVSGNCDVVIGIVNNTVYDPLQSELDSIKEELDKIHKNGSG GSGDIIKLLNEQVNKEMQSSNLYMSMSSWCYTHSLDGAGLFLFDHAAEEY EHAKKLIIFLNENNVPVQLTSISAPEHKFEGLTQIFQKAYEHEQHISESI NNIVDHAIKSKDHATFNFLQWYVAEQHEEEVLFKDILDKIELIGNENHGL YLADQYVKGIAKSRKSGS | 289 |
| 1B-06-PL with N234Q (more “down” RBD) | pCoV210 | S2P.1158op1-del-Ferritin-N234Q | | SSQCVNLTTRTQLPPAYTNSFTRGVYYPDKVFRSSVLHSTQDLFLPFFSN VTWFHAIHVSGTNGTKRFDNPVLPFNDGVYFASTEKSNIIRGWIFGTTLD SKTQSLLIVNNATNVVIKVCEFQFCNDPFLGVYYHKNNKSWMESEFRVYS SANNCTFEYVSQPFLMDLEGKQGNFKNLREFVFKNIDGYFKIYSKHTPIN LVRDLPQGFSALEPLVDLPIGIQITRFQTLLALHRSYLTPGDSSSGWTAG AAAYYVGYLQPRTFLLKYNENGTITDAVDCALDPLSETKCTLKSFTVEKG IYQTSNFRVQPTESIVRFPNITNLCPFGEVFNATRFASVYAWNRKRISNC VADYSVLYNSASFSTFKCYGVSPTKLNDLCFTNVYADSFVIRGDEVRQIA PGQTGKIADYNYKLPDDFTGCVIAWNSNNLDSKVGGNYNYLYRLFRKSNL KPFERDISTEIYQAGSTPCNGVEGFNCYFPLQSYGFQPTNGVGYQPYRVV VLSFELLHAPATVCGPKKSTNLVKNKCVNFNFNGLTGTGVLTESNKKFLP FQQFGRDIADTTDAVRDPQTLEILDITPCSFGGVSVITPGTNTSNQVAVL YQDVNCTEVPVAIHADQLTPTWRVYSTGSNVFQTRAGCLIGAEHVNNSYE CDIPIGAGICASYQTQTNSPGSASSVASQSIIAYTMSLGAENSVAYSNNS IAIPTNFTISVTTEILPVSMTKTSVDCTMYICGDSTECSNLLLQYGSFCT QLNRALTGIAVEQDKNTQEVFAQVKQIYKTPPIKDFGGFNFSQILPDPSK PSKRSFIEDLLFNKVTLADAGFIKQYGDCLGDIAARDLICAQKFNGLTVL PPLLTDEMIAQYTSALLAGTITSGWTFGAGAALQIPFAMQMAYRFNGIGV TQNVLYENQKLIANQFNSAIGKIQDSLSSTASALGKLQDVVNQNAQALNT LVKQLSSNFGAISSVLNDILSRLDPPEAEVQIDRLITGRLQSLQTYVTQQ LIRAAEIRASANLAATKMSECVLGQSKRVDFCGKGYHLMSFPQSAPHGVV FLHVTYVPAQEKNFTTAPAICHDGKAHFPREGVFVSNGTHWFVTQRNFYE PQIITTDNTFVSGNCDVVIGIVNNTVYDPLQSELDSIKEELDKIHKNGSG GSGDIIKLLNEQVNKEMQSSNLYMSMSSWCYTHSLDGAGLFLFDHAAEEY EHAKKLIIFLNENNVPVQLTSISAPEHKFEGLTQIFQKAYEHEQHISESI NNIVDHAIKSKDHATFNFLQWYVAEQHEEEVLFKDILDKIELIGNENHGL YLADQYVKGIAKSRKSGS | 290 |
| 1B-06-PL with HexaPro + D614G + N165Q (more “up” RBD) | pCoV211 | S2P-HexaPro.1158op1-del-Ferritin-D614G-N165Q | | SSQCVNLTTRTQLPPAYTNSFTRGVYYPDKVFRSSVLHSTQDLFLPFFSN VTWFHAIHVSGTNGTKRFDNPVLPFNDGVYFASTEKSNIIRGWIFGTTLD SKTQSLLIVNNATNVVIKVCEFQFCNDPFLGVYYHKNNKSWMESEFRVYS SANQCTFEYVSQPFLMDLEGKQGNFKNLREFVFKNIDGYFKIYSKHTPIN LVRDLPQGFSALEPLVDLPIGINITRFQTLLALHRSYLTPGDSSSGWTAG AAAYYVGYLQPRTFLLKYNENGTITDAVDCALDPLSETKCTLKSFTVEKG IYQTSNFRVQPTESIVRFPNITNLCPFGEVFNATRFASVYAWNRKRISNC VADYSVLYNSASFSTFKCYGVSPTKLNDLCFTNVYADSFVIRGDEVRQIA PGQTGKIADYNYKLPDDFTGCVIAWNSNNLDSKVGGNYNYLYRLFRKSNL KPFERDISTEIYQAGSTPCNGVEGFNCYFPLQSYGFQPTNGVGYQPYRVV VLSFELLHAPATVCGPKKSTNLVKNKCVNFNFNGLTGTGVLTESNKKFLP FQQFGRDIADTTDAVRDPQTLEILDITPCSFGGVSVITPGTNTSNQVAVL YQGVNCTEVPVAIHADQLTPTWRVYSTGSNVFQTRAGCLIGAEHVNNSYE CDIPIGAGICASYQTQTNSPGSASSVASQSIIAYTMSLGAENSVAYSNNS IAIPTNFTISVTTEILPVSMTKTSVDCTMYICGDSTECSNLLLQYGSFCT QLNRALTGIAVEQDKNTQEVFAQVKQIYKTPPIKDFGGFNFSQILPDPSK PSKRSPIEDLLFNKVTLADAGFIKQYGDCLGDIAARDLICAQKFNGLTVL PPLLTDEMIAQYTSALLAGTITSGWTFGAGPALQIPFPMQMAYRFNGIGV TQNVLYENQKLIANQFNSAIGKIQDSLSSTPSALGKLQDVVNQNAQALNT LVKQLSSNFGAISSVLNDILSRLDPPEAEVQIDRLITGRLQSLQTYVTQQ LIRAAEIRASANLAATKMSECVLGQSKRVDFCGKGYHLMSFPQSAPHGVV FLHVTYVPAQEKNFTTAPAICHDGKAHFPREGVFVSNGTHWFVTQRNFYE PQIITTDNTFVSGNCDVVIGIVNNTVYDPLQSELDSIKEELDKIHKNGSG GSGDIIKLLNEQVNKEMQSSNLYMSMSSWCYTHSLDGAGLFLFDHAAEEY EHAKKLIIFLNENNVPVQLTSISAPEHKFEGLTQIFQKAYEHEQHISESI NNIVDHAIKSKDHATFNFLQWYVAEQHEEEVLFKDILDKIELIGNENHGL YLADQYVKGIAKSRKSGS | 291 |
| 1B-06-PL with HexaPro + D614G + N234Q (more “down” RBD) | pCoV212 | S2P-HexaPro.1158 op1-del-Ferritin-D614G-N234Q | | SSQCVNLTTRTQLPPAYTNSFTRGVYYPDKVFRSSVLHSTQDLFLPFFSN VTWFHAIHVSGTNGTKRFDNPVLPFNDGVYFASTEKSNIIRGWIFGTTLD SKTQSLLIVNNATNVVIKVCEFQFCNDPFLGVYYHKNNKSWMESEFRVYS SANNCTFEYVSQPFLMDLEGKQGNFKNLREFVFKNIDGYFKIYSKHTPIN LVRDLPQGFSALEPLVDLPIGIQITRFQTLLALHRSYLTPGDSSSGWTAG AAAYYVGYLQPRTFLLKYNENGTITDAVDCALDPLSETKCTLKSFTVEKG IYQTSNFRVQPTESIVRFPNITNLCPFGEVFNATRFASVYAWNRKRISNC VADYSVLYNSASFSTFKCYGVSPTKLNDLCFTNVYADSFVIRGDEVRQIA PGQTGKIADYNYKLPDDFTGCVIAWNSNNLDSKVGGNYNYLYRLFRKSNL KPFERDISTEIYQAGSTPCNGVEGFNCYFPLQSYGFQPTNGVGYQPYRVV VLSFELLHAPATVCGPKKSTNLVKNKCVNFNFNGLTGTGVLTESNKKFLP FQQFGRDIADTTDAVRDPQTLEILDITPCSFGGVSVITPGTNTSNQVAVL YQGVNCTEVPVAIHADQLTPTWRVYSTGSNVFQTRAGCLIGAEHVNNSYE CDIPIGAGICASYQTQTNSPGSASSVASQSIIAYTMSLGAENSVAYSNNS IAIPTNFTISVTTEILPVSMTKTSVDCTMYICGDSTECSNLLLQYGSFCT QLNRALTGIAVEQDKNTQEVFAQVKQIYKTPPIKDFGGFNFSQILPDPSK PSKRSPIEDLLFNKVTLADAGFIKQYGDCLGDIAARDLICAQKFNGLTVL PPLLTDEMIAQYTSALLAGTITSGWTFGAGPALQIPFPMQMAYRFNGIGV TQNVLYENQKLIANQFNSAIGKIQDSLSSTPSALGKLQDVVNQNAQALNT LVKQLSSNFGAISSVLNDILSRLDPPEAEVQIDRLITGRLQSLQTYVTQQ LIRAAEIRASANLAATKMSECVLGQSKRVDFCGKGYHLMSFPQSAPHGVV FLHVTYVPAQEKNFTTAPAICHDGKAHFPREGVFVSNGTHWFVTQRNFYE PQIITTDNTFVSGNCDVVIGIVNNTVYDPLQSELDSIKEELDKIHKNGSG GSGDIIKLLNEQVNKEMQSSNLYMSMSSWCYTHSLDGAGLFLFDHAAEEY EHAKKLIIFLNENNVPVQLTSISAPEHKFEGLTQIFQKAYEHEQHISESI NNIVDHAIKSKDHATFNFLQWYVAEQHEEEVLFKDILDKIELIGNENHGL YLADQYVKGIAKSRKSGS | 292 |
| 1B-08-PL with HexaPro + D614G + N165Q (more “up” RBD) | pCoV213 | S2P-HexaPro.1158 op1x2-del-Ferritin-D614G-N165Q | | SSQCVNLTTRTQLPPAYTNSFTRGVYYPDKVFRSSVLHSTQDLFLPFFSN VTWFHAIHVSGTNGTKRFDNPVLPFNDGVYFASTEKSNIIRGWIFGTTLD SKTQSLLIVNNATNVVIKVCEFQFCNDPFLGVYYHKNNKSWMESEFRVYS SANQCTFEYVSQPFLMDLEGKQGNFKNLREFVFKNIDGYFKIYSKHTPIN LVRDLPQGFSALEPLVDLPIGINITRFQTLLALHRSYLTPGDSSSGWTAG AAAYYVGYLQPRTFLLKYNENGTITDAVDCALDPLSETKCTLKSFTVEKG IYQTSNFRVQPTESIVRFPNITNLCPFGEVFNATRFASVYAWNRKRISNC VADYSVLYNSASFSTFKCYGVSPTKLNDLCFTNVYADSFVIRGDEVRQIA PGQTGKIADYNYKLPDDFTGCVIAWNSNNLDSKVGGNYNYLYRLFRKSNL KPFERDISTEIYQAGSTPCNGVEGFNCYFPLQSYGFQPTNGVGYQPYRVV VLSFELLHAPATVCGPKKSTNLVKNKCVNFNFNGLTGTGVLTESNKKFLP FQQFGRDIADTTDAVRDPQTLEILDITPCSFGGVSVITPGTNTSNQVAVL YQGVNCTEVPVAIHADQLTPTWRVYSTGSNVFQTRAGCLIGAEHVNNSYE CDIPIGAGICASYQTQTNSPGSASSVASQSIIAYTMSLGAENSVAYSNNS IAIPTNFTISVTTEILPVSMTKTSVDCTMYICGDSTECSNLLLQYGSFCT QLNRALTGIAVEQDKNTQEVFAQVKQIYKTPPIKDFGGFNFSQILPDPSK PSKRSPIEDLLFNKVTLADAGFIKQYGDCLGDIAARDLICAQKFNGLTVL PPLLTDEMIAQYTSALLAGTITSGWTFGAGPALQIPFPMQMAYRFNGIGV TQNVLYENQKLIANQFNSAIGKIQDSLSSTPSALGKLQDVVNQNAQALNT LVKQLSSNFGAISSVLNDILSRLDPPEAEVQIDRLITGRLQSLQTYVTQQ LIRAAEIRASANLAATKMSECVLGQSKRVDFCGKGYHLMSFPQSAPHGVV FLHVTYVPAQEKNFTTAPAICHDGKAHFPREGVFVSNGTHWFVTQRNFYE PQIITTDNTFVSGNCDVVIGIVNNTVYDPLQSELDSIKEELDKIHKNLDS IKEELDKIHKNGSGGSGDIIKLLNEQVNKEMQSSNLYMSMSSWCYTHSLD GAGLFLFDHAAEEYEHAKKLIIFLNENNVPVQLTSISAPEHKFEGLTQIF QKAYEHEQHISESINNIVDHAIKSKDHATFNFLQWYVAEQHEEEVLFKDI LDKIELIGNENHGLYLADQYVKGIAKSRKSGS | 293 |
| 1B-08-PL with HexaPro + D614G + N234Q (more “down” RBD) | pCoV214 | S2P-HexaPro.1158 op1x2-del-Ferritin-D614G-N234Q | | SSQCVNLTTRTQLPPAYTNSFTRGVYYPDKVFRSSVLHSTQDLFLPFFSN VTWFHAIHVSGTNGTKRFDNPVLPFNDGVYFASTEKSNIIRGWIFGTTLD SKTQSLLIVNNATNVVIKVCEFQFCNDPFLGVYYHKNNKSWMESEFRVYS SANNCTFEYVSQPFLMDLEGKQGNFKNLREFVFKNIDGYFKIYSKHTPIN LVRDLPQGFSALEPLVDLPIGIQITRFQTLLALHRSYLTPGDSSSGWTAG AAAYYVGYLQPRTFLLKYNENGTITDAVDCALDPLSETKCTLKSFTVEKG IYQTSNFRVQPTESIVRFPNITNLCPFGEVFNATRFASVYAWNRKRISNC VADYSVLYNSASFSTFKCYGVSPTKLNDLCFTNVYADSFVIRGDEVRQIA PGQTGKIADYNYKLPDDFTGCVIAWNSNNLDSKVGGNYNYLYRLFRKSNL KPFERDISTEIYQAGSTPCNGVEGFNCYFPLQSYGFQPTNGVGYQPYRVV VLSFELLHAPATVCGPKKSTNLVKNKCVNFNFNGLTGTGVLTESNKKFLP FQQFGRDIADTTDAVRDPQTLEILDITPCSFGGVSVITPGTNTSNQVAVL YQGVNCTEVPVAIHADQLTPTWRVYSTGSNVFQTRAGCLIGAEHVNNSYE CDIPIGAGICASYQTQTNSPGSASSVASQSIIAYTMSLGAENSVAYSNNS IAIPTNFTISVTTEILPVSMTKTSVDCTMYICGDSTECSNLLLQYGSFCT QLNRALTGIAVEQDKNTQEVFAQVKQIYKTPPIKDFGGFNFSQILPDPSK PSKRSPIEDLLFNKVTLADAGFIKQYGDCLGDIAARDLICAQKFNGLTVL PPLLTDEMIAQYTSALLAGTITSGWTFGAGPALQIPFPMQMAYRFNGIGV TQNVLYENQKLIANQFNSAIGKIQDSLSSTPSALGKLQDVVNQNAQALNT LVKQLSSNFGAISSVLNDILSRLDPPEAEVQIDRLITGRLQSLQTYVTQQ LIRAAEIRASANLAATKMSECVLGQSKRVDFCGKGYHLMSFPQSAPHGVV FLHVTYVPAQEKNFTTAPAICHDGKAHFPREGVFVSNGTHWFVTQRNFYE PQIITTDNTFVSGNCDVVIGIVNNTVYDPLQSELDSIKEELDKIHKNLDS IKEELDKIHKNGSGGSGDIIKLLNEQVNKEMQSSNLYMSMSSWCYTHSLD GAGLFLFDHAAEEYEHAKKLIIFLNENNVPVQLTSISAPEHKFEGLTQIF QKAYEHEQHISESINNIVDHAIKSKDHATFNFLQWYVAEQHEEEVLFKDI LDKIELIGNENHGLYLADQYVKGIAKSRKSGS | 294 |
| 1B-06-PL with glycan at N146 on Ferritin to fill in gap | pCoV215 | S2P.1158op1-del-Ferritin-N146glyc | | SSQCVNLTTRTQLPPAYTNSFTRGVYYPDKVFRSSVLHSTQDLFLPFFSN VTWFHAIHVSGTNGTKRFDNPVLPFNDGVYFASTEKSNIIRGWIFGTTLD SKTQSLLIVNNATNVVIKVCEFQFCNDPFLGVYYHKNNKSWMESEFRVYS SANNCTFEYVSQPFLMDLEGKQGNFKNLREFVFKNIDGYFKIYSKHTPIN LVRDLPQGFSALEPLVDLPIGINITRFQTLLALHRSYLTPGDSSSGWTAG AAAYYVGYLQPRTFLLKYNENGTITDAVDCALDPLSETKCTLKSFTVEKG IYQTSNFRVQPTESIVRFPNITNLCPFGEVFNATRFASVYAWNRKRISNC VADYSVLYNSASFSTFKCYGVSPTKLNDLCFTNVYADSFVIRGDEVRQIA PGQTGKIADYNYKLPDDFTGCVIAWNSNNLDSKVGGNYNYLYRLFRKSNL KPFERDISTEIYQAGSTPCNGVEGFNCYFPLQSYGFQPTNGVGYQPYRVV VLSFELLHAPATVCGPKKSTNLVKNKCVNFNFNGLTGTGVLTESNKKFLP FQQFGRDIADTTDAVRDPQTLEILDITPCSFGGVSVITPGTNTSNQVAVL YQDVNCTEVPVAIHADQLTPTWRVYSTGSNVFQTRAGCLIGAEHVNNSYE CDIPIGAGICASYQTQTNSPGSASSVASQSIIAYTMSLGAENSVAYSNNS IAIPTNFTISVTTEILPVSMTKTSVDCTMYICGDSTECSNLLLQYGSFCT QLNRALTGIAVEQDKNTQEVFAQVKQIYKTPPIKDFGGFNFSQILPDPSK PSKRSFIEDLLFNKVTLADAGFIKQYGDCLGDIAARDLICAQKFNGLTVL PPLLTDEMIAQYTSALLAGTITSGWTFGAGAALQIPFAMQMAYRFNGIGV TQNVLYENQKLIANQFNSAIGKIQDSLSSTASALGKLQDVVNQNAQALNT LVKQLSSNFGAISSVLNDILSRLDPPEAEVQIDRLITGRLQSLQTYVTQQ LIRAAEIRASANLAATKMSECVLGQSKRVDFCGKGYHLMSFPQSAPHGVV FLHVTYVPAQEKNFTTAPAICHDGKAHFPREGVFVSNGTHWFVTQRNFYE PQIITTDNTFVSGNCDVVIGIVNNTVYDPLQSELDSIKEELDKIHKNGSG GSGDIIKLLNEQVNKEMQSSNLYMSMSSWCYTHSLDGAGLFLFDHAAEEY EHAKKLIIFLNENNVPVQLTSISAPEHKFEGLTQIFQKAYEHEQHISESI NNIVDHAIKSKDHATFNFLQWYVAEQHEEEVLFKDILDKIELIGNETHGL YLADQYVKGIAKSRKSGS | 295 |
| 1B-06-PL with glycan at N77on Ferritin to fill in gap | pCoV216 | S2P.1158op1-DS1-del-Ferritin-N77glyc | | SSQCVNLTTRTQLPPAYTNSFTRGVYYPDKVFRSSVLHSTQDLFLPFFSN VTWFHAIHVSGTNGTKRFDNPVLPFNDGVYFASTEKSNIIRGWIFGTTLD SKTQSLLIVNNATNVVIKVCEFQFCNDPFLGVYYHKNNKSWMESEFRVYS SANNCTFEYVSQPFLMDLEGKQGNFKNLREFVFKNIDGYFKIYSKHTPIN LVRDLPQGFSALEPLVDLPIGINITRFQTLLALHRSYLTPGDSSSGWTAG AAAYYVGYLQPRTFLLKYNENGTITDAVDCALDPLSETKCTLKSFTVEKG IYQTSNFRVQPTESIVRFPNITNLCPFGEVFNATRFASVYAWNRKRISNC VADYSVLYNSASFSTFKCYGVSPTKLNDLCFTNVYADSFVIRGDEVRQIA PGQTGKIADYNYKLPDDFTGCVIAWNSNNLDSKVGGNYNYLYRLFRKSNL KPFERDISTEIYQAGSTPCNGVEGFNCYFPLQSYGFQPTNGVGYQPYRVV VLSFELLHAPATVCGPKKSTNLVKNKCVNFNFNGLTGTGVLTESNKKFLP FQQFGRDIADTTDAVRDPQTLEILDITPCSFGGVSVITPGTNTSNQVAVL YQDVNCTEVPVAIHADQLTPTWRVYSTGSNVFQTRAGCLIGAEHVNNSYE CDIPIGAGICASYQTQTNSPGSASSVASQSIIAYTMSLGAENSVAYSNNS IAIPTNFTISVTTEILPVSMTKTSVDCTMYICGDSTECSNLLLQYGSFCT QLNRALTGIAVEQDKNTQEVFAQVKQIYKTPPIKDFGGFNFSQILPDPSK PSKRSFIEDLLFNKVTLADAGFIKQYGDCLGDIAARDLICAQKFNGLTVL PPLLTDEMIAQYTSALLAGTITSGWTFGAGAALQIPFAMQMAYRFNGIGV TQNVLYENQKLIANQFNSAIGKIQDSLSSTASALGKLQDVVNQNAQALNT LVKQLSSNFGAISSVLNDILSRLDPPEAEVQIDRLITGRLQSLQTYVTQQ LIRAAEIRASANLAATKMSECVLGQSKRVDFCGKGYHLMSFPQSAPHGVV FLHVTYVPAQEKNFTTAPAICHDGKAHFPREGVFVSNGTHWFVTQRNFYE PQIITTDNTFVSGNCDVVIGIVNNTVYDPLQSELDSIKEELDKIHKNGSG GSGDIIKLLNEQVNKEMQSSNLYMSMSSWCYTHSLDGAGLFLFDHAAEEY EHAKKLIIFLNENNVPVQLTSISAPNHSFEGLTQIFQKAYEHEQHISESI NNIVDHAIKSKDHATFNFLQWYVAEQHEEEVLFKDILDKIELIGNENHGL YLADQYVKGIAKSRKSGS | 296 |
| 1B-06-PL with HexaPro + D614G with glycan at N146 on Ferritin to fill in gap | pCoV217 | S2P-HexaPro.1158op1-del-Ferritin-D614G-N146glyc | | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQGVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSPIEDLL FN KVTLADAGFIKQYGDCLGDIAARDLICAQKFNGLTVLPPLLTDEMIAQYT SALLAGTITSGWTFGAGPALQIPFPMQMAYRFNGIGVTQNVLYENQKLIA NQFNSAIGKIQDSLSSTPSALGKLQDVVNQNAQALNTLVKQLSSNFGAIS S VLNDILSRLDPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQSELDSIKEELDKIHKNGSGGSGDIIKLLNEQVNK E MQSSNLYMSMSSWCYTHSLDGAGLFLFDHAAEEYEHAKKLIIFLNENNV PVQLTSISAPEHKFEGLTQIFQKAYEHEQHISESINNIVDHAIKSKDHAT FNF LQWYVAEQHEEEVLFKDILDKIELIGNETHGLYLADQYVKGIAKSRKSGS | 297 |
| 1B-06-PL with HexaPro + D614G with glycan at N77on Ferritin to fill in gap | pCoV218 | S2P-HexaPro.1158op1-del-Ferritin-D614G-N77glyc | | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQGVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSPIEDLL FN KVTLADAGFIKQYGDCLGDIAARDLICAQKFNGLTVLPPLLTDEMIAQYT SALLAGTITSGWTFGAGPALQIPFPMQMAYRFNGIGVTQNVLYENQKLIA NQFNSAIGKIQDSLSSTPSALGKLQDVVNQNAQALNTLVKQLSSNFGAIS S VLNDILSRLDPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQSELDSIKEELDKIHKNGSGGSGDIIKLLNEQVNK E MQSSNLYMSMSSWCYTHSLDGAGLFLFDHAAEEYEHAKKLIIFLNENNV PVQLTSISAPNHSFEGLTQIFQKAYEHEQHISESINNIVDHAIKSKDHAT FNF LQWYVAEQHEEEVLFKDILDKIELIGNENHGLYLADQYVKGIAKSRKSGS | 298 |
| 1B-08-PL with HexaPro + D614G with glycan at N146 on Ferritin to fill in gap | pCoV219 | S2P-HexaPro.1158oplx2-del-Ferritin-D614G-N146glyc | | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQGVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSPIEDLL FN KVTLADAGFIKQYGDCLGDIAARDLICAQKFNGLTVLPPLLTDEMIAQYT SALLAGTITSGWTFGAGPALQIPFPMQMAYRFNGIGVTQNVLYENQKLIA NQFNSAIGKIQDSLSSTPSALGKLQDVVNQNAQALNTLVKQLSSNFGAIS S VLNDILSRLDPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQSELDSIKEELDKIHKNLDSIKEELDKIHKNGSGG SG DIIKLLNEQVNKEMQSSNLYMSMSSWCYTHSLDGAGLFLFDHAAEEYEH AKKLIIFLNENNVPVQLTSISAPEHKFEGLTQIFQKAYEHEQHISESINN IVD HAIKSKDHATFNFLQWYVAEQHEEEVLFKDILDKIELIGNETHGLYLADQ YVKGIAKSRKSGS | 299 |
| 1B-08-PL with HexaPro + D614G with glycan at N77on Ferritin to fill in gap | pCoV220 | S2P-HexaPro.1158oplx2-del-Ferritin-D614G-N77glyc | | SSQCVNLTTRTQLPPAYTNSFTRGVYYPDKVFRSSVLHSTQDLFLPFFSN V TWFHAIHVSGTNGTKRFDNPVLPFNDGVYFASTEKSNIIRGWIFGTTLDS K TQSLLIVNNATNVVIKVCEFQFCNDPFLGVYYHKNNKSWMESEFRVYSSA NNCTFEYVSQPFLMDLEGKQGNFKNLREFVFKNIDGYFKIYSKHTPINLV R DLPQGFSALEPLVDLPIGINITRFQTLLALHRSYLTPGDSSSGWTAGAAA Y YVGYLQPRTFLLKYNENGTITDAVDCALDPLSETKCTLKSFTVEKGIYQT S NFRVQPTESIVRFPNITNLCPFGEVFNATRFASVYAWNRKRISNCVADYS V LYNSASFSTFKCYGVSPTKLNDLCFTNVYADSFVIRGDEVRQIAPGQTGK I ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFEL L HAPATVCGPKKSTNLVKNKCVNFNFNGLTGTGVLTESNKKFLPFQQFGR DIADTTDAVRDPQTLEILDITPCSFGGVSVITPGTNTSNQVAVLYQGVNC T EVPVAIHADQLTPTWRVYSTGSNVFQTRAGCLIGAEHVNNSYECDIPIGA GICASYQTQTNSPGSASSVASQSIIAYTMSLGAENSVAYSNNSIAIPTNF TIS VTTEILPVSMTKTSVDCTMYICGDSTECSNLLLQYGSFCTQLNRALTGIA V EQDKNTQEVFAQVKQIYKTPPIKDFGGFNFSQILPDPSKPSKRSPIEDLL FN KVTLADAGFIKQYGDCLGDIAARDLICAQKFNGLTVLPPLLTDEMIAQYT SALLAGTITSGWTFGAGPALQIPFPMQMAYRFNGIGVTQNVLYENQKLIA NQFNSAIGKIQDSLSSTPSALGKLQDVVNQNAQALNTLVKQLSSNFGAIS S VLNDILSRLDPPEAEVQIDRLITGRLQSLQTYVTQQLIRAAEIRASANLA AT KMSECVLGQSKRVDFCGKGYHLMSFPQSAPHGVVFLHVTYVPAQEKNFT TAPAICHDGKAHFPREGVFVSNGTHWFVTQRNFYEPQIITTDNTFVSGNC DVVIGIVNNTVYDPLQSELDSIKEELDKIHKNLDSIKEELDKIHKNGSGG SG DIIKLLNEQVNKEMQSSNLYMSMSSWCYTHSLDGAGLFLFDHAAEEYEH AKKLIIFLNENNVPVQLTSISAPNHSFEGLTQIFQKAYEHEQHISESINN IVD HAIKSKDHATFNFLQWYVAEQHEEEVLFKDILDKIELIGNENHGLYLADQ YVKGIAKSRKSGS | 300 |
| | pCoV223 | PrefLead-His8-3c-S2P.1158op1-del-Ferritin-N1158Q | | HHHHHHHHGPLEVLFQGPSSQCVNLTTRTQLPPAYTNSFTRGVYYPDKVF RSSVLHSTQDLFLPFFSNVTWFHAIHVSGTNGTKRFDNPVLPFNDGVYFA S TEKSNIIRGWIFGTTLDSKTQSLLIVNNATNVVIKVCEFQFCNDPFLGVY Y HKNNKSWMESEFRVYSSANNCTFEYVSQPFLMDLEGKQGNFKNLREFVF KNIDGYFKIYSKHTPINLVRDLPQGFSALEPLVDLPIGINITRFQTLLAL HRS YLTPGDSSSGWTAGAAAYYVGYLQPRTFLLKYNENGTITDAVDCALDPL SETKCTLKSFTVEKGIYQTSNFRVQPTESIVRFPNITNLCPFGEVFNATR FA SVYAWNRKRISNCVADYSVLYNSASFSTFKCYGVSPTKLNDLCFTNVYA DSFVIRGDEVRQIAPGQTGKIADYNYKLPDDFTGCVIAWNSNNLDSKVGG NYNYLYRLFRKSNLKPFERDISTEIYQAGSTPCNGVEGFNCYFPLQSYGF Q PTNGVGYQPYRVVVLSFELLHAPATVCGPKKSTNLVKNKCVNFNFNGLT GTGVLTESNKKFLPFQQFGRDIADTTDAVRDPQTLEILDITPCSFGGVSV IT PGTNTSNQVAVLYQDVNCTEVPVAIHADQLTPTWRVYSTGSNVFQTRAG CLIGAEHVNNSYECDIPIGAGICASYQTQTNSPGSASSVASQSIIAYTMS LG AENSVAYSNNSIAIPTNFTISVTTEILPVSMTKTSVDCTMYICGDSTECS NL LLQYGSFCTQLNRALTGIAVEQDKNTQEVFAQVKQIYKTPPIKDFGGFNF S QILPDPSKPSKRSFIEDLLFNKVTLADAGFIKQYGDCLGDIAARDLICAQ KF NGLTVLPPLLTDEMIAQYTSALLAGTITSGWTFGAGAALQIPFAMQMAYR FNGIGVTQNVLYENQKLIANQFNSAIGKIQDSLSSTASALGKLQDVVNQN AQALNTLVKQLSSNFGAISSVLNDILSRLDPPEAEVQIDRLITGRLQSLQ TY VTQQLIRAAEIRASANLAATKMSECVLGQSKRVDFCGKGYHLMSFPQSAP HGVVFLHVTYVPAQEKNFTTAPAICHDGKAHFPREGVFVSNGTHWFVTQ RNFYEPQIITTDNTFVSGNCDVVIGIVNNTVYDPLQSELDSIKEELDKIH KN GSGGSGDIIKLLNEQVNKEMQSSNLYMSMSSWCYTHSLDGAGLFLFDHA AEEYEHAKKLIIFLNENNVPVQLTSISAPEHKFEGLTQIFQKAYEHEQHI SE SINNIVDHAIKSKDHATFNFLQWYVAEQHEEEVLFKDILDKIELIGNENH G LYLADQYVKGIAKSRKSGS | 301 |
| | pCoV-BoS1 | SARS-CoV-2, SARS-CoV-1 HKU-1, MERS-CoV, 229E, | | NITNLCPFGEVFNATRFASVYAWNRKRISNCVADYSVLYNSASFSTFKCY GVSPTKLNDLCFTNVYADSFVIRGDEVRQIAPGQTGKIADYNYKLPDDFT GCVIAWNSNNLDSKVGGNYNYLYRLFRKSNLKPFERDISTEIYQAGSTPC NGVEGFNCYFPLQSYGFQPTNGVGYQPYRVVVLSFELLHAPATVCGGGG S TNLCPFGEVFNATKFPSVYAWERKKISNCVADYSVLYNSTFFSTFKCYGV SATKLNDLCFSNVYADSFVVKGDDVRQIAPGQTGVIADYNYKLPDDFMG CVLAWNTRNIDATSTGNYNYKYRYLRHGKLRPFERDISNVPFSPDGKPCT PPALNCYWPLNDYGFYTTTGIGYQP CGGGGS DCDIDKWLNNFNVPSPLNWERKIFSNCNFNLSTLLRLVHTDSFSCNNFDE S KIYGSCFKSIVLDKFAIPNSRRSDLQLGSSGFLQSSNYKIDTTSSSCQLY YSL PAINVTINNYNPSSWNRRYGFNNFNLSSHSVVYSRYCFSVNNTFCPCAKP S FASSCKSHKPPSASCPIGTNYRSCESTTVLDHTDWCRCSCLPDPITAYDP RS CSQKKSLVGVGEHCAGFGVDEEKCGVLDGSYNVSCLCSTDAFLGWSYDT CVSNNRCNIFSNFILNG CGGGGS EAKPSGSVVEQAEGVECDFSPLLSGTPPQVYNFKRLVFTNCNYNLTKLLS LFSVNDFTCSQISPAAIASNCYSSLILDYFSYPLSMKSDLSVSSAGPISQ FNY KQSFSNPTCLILATVPHNLTTITKPLKYSYINKCSRFLSDDRTEVPQLVN AN QYSPCVSIVPSTVWEDGDYYRKQLSPLEGGGWLVASGSTVAMTEQLQM GFGITVQYGTDTNSVCPK CGGGGS SIVSLPVYHKHTFIVLYVDFKPQSGGGKCFNCYPAGVNITLANFNETKGP L CVDTSHFTTKYVAVYANVG RWSASINTGNCPFSFGKVNNFVKFGSVCFSLKDIPGGCAMPIVANWAYSK YYTIGSLYVSWSDGDGITGV PQPVEGVSS GSGGGGESQVRQQFSKDIEKLLNEQVNKEMQSSNLYMSMSSWCYTHSLD GAGLFLFDHAAEEYEHAKKLIIFLNENNVPVQLTSISAPEHKFEGLTQIF QK AYEHEQHISESINNIVDHAIKSKDHATFNFLQWYVAEQHEEEVLFKDILD K IELIGNENHGLYLADQYVKGIAKSRKSGS | 302 |
| | pCoV-BoS2 | | | TNLCPFGEVFNATKFPSVYAWERKKISNCVADYSVLYNSTFFSTFKCYGV SATKLNDLCFSNVYADSFVVKGDDVRQIAPGQTGVIADYNYKLPDDFMG CVLAWNTRNIDATSTGNYNYKYRYLRHGKLRPFERDISNVPFSPDGKPCT PPALNCYWPLNDYGFYTTTGIGYQP CGGGGS DCDIDKWLNNFNVPSPLNWERKIFSNCNFNLSTLLRLVHTDSFSCNNFDE S KIYGSCFKSIVLDKFAIPNSRRSDLQLGSSGFLQSSNYKIDTTSSSCQLY YSL PAINVTINNYNPSSWNRRYGFNNFNLSSHSVVYSRYCFSVNNTFCPCAKP S FASSCKSHKPPSASCPIGTNYRSCESTTVLDHTDWCRCSCLPDPITAYDP RS CSQKKSLVGVGEHCAGFGVDEEKCGVLDGSYNVSCLCSTDAFLGWSYDT CVSNNRCNIFSNFILNG CGGGGS EAKPSGSVVEQAEGVECDFSPLLSGTPPQVYNFKRLVFTNCNYNLTKLLS LFSVNDFTCSQISPAAIASNCYSSLILDYFSYPLSMKSDLSVSSAGPISQ FNY KQSFSNPTCLILATVPHNLTTITKPLKYSYINKCSRFLSDDRTEVPQLVN AN QYSPCVSIVPSTVWEDGDYYRKQLSPLEGGGWLVASGSTVAMTEQLQM GFGITVQYGTDTNSVCPK CGGGGS SIVSLPVYHKHTFIVLYVDFKPQSGGGKCFNCYPAGVNITLANFNETKGP L CVDTSHFTTKYVAVYANVG RWSASINTGNCPFSFGKVNNFVKFGSVCFSLKDIPGGCAMPIVANWAYSK YYTIGSLYVSWSDGDGITGV PQPVEGVSS NITNLCPFGEVFNATRFASVYAWNRKRISNCVADYSVLYNSASFSTFKCY GVSPTKLNDLCFTNVYADSFVIRGDEVRQIAPGQTGKIADYNYKLPDDFT GCVIAWNSNNLDSKVGGNYNYLYRLFRKSNLKPFERDISTEIYQAGSTPC NGVEGFNCYFPLQSYGFQPTNGVGYQPYRVVVLSFELLHAPATVCGGGG S GSGGGGESQVRQQFSKDIEKLLNEQVNKEMQSSNLYMSMSSWCYTHSLD GAGLFLFDHAAEEYEHAKKLIIFLNENNVPVQLTSISAPEHKFEGLTQIF QK AYEHEQHISESINNIVDHAIKSKDHATFNFLQWYVAEQHEEEVLFKDILD K IELIGNENHGLYLADQYVKGIAKSRKSGS | 303 |
| | pCoV-BoS3 | | | NITNLCPFGEVFNATRFASVYAWNRKRISNCVADYSVLYNSASFSTFKCY GVSPTKLNDLCFTNVYADSFVIRGDEVRQIAPGQTGKIADYNYKLPDDFT GCVIAWNSNNLDSKVGGNYNYLYRLFRKSNLKPFERDISTEIYQAGSTPC NGVEGFNCYFPLQSYGFQPTNGVGYQPYRVVVLSFELLHAPATVCGGGG S TNLCPFGEVFNATKFPSVYAWERKKISNCVADYSVLYNSTFFSTFKCYGV SATKLNDLCFSNVYADSFVVKGDDVRQIAPGQTGVIADYNYKLPDDFMG CVLAWNTRNIDATSTGNYNYKYRYLRHGKLRPFERDISNVPFSPDGKPCT PPALNCYWPLNDYGFYTTTGIGYQP CGGGGS EAKPSGSVVEQAEGVECDFSPLLSGTPPQVYNFKRLVFTNCNYNLTKLLS LFSVNDFTCSQISPAAIASNCYSSLILDYFSYPLSMKSDLSVSSAGPISQ FNY KQSFSNPTCLILATVPHNLTTITKPLKYSYINKCSRFLSDDRTEVPQLVN AN QYSPCVSIVPSTVWEDGDYYRKQLSPLEGGGWLVASGSTVAMTEQLQM GFGITVQYGTDTNSVCPK CGGGGS SIVSLPVYHKHTFIVLYVDFKPQSGGGKCFNCYPAGVNITLANFNETKGP L CVDTSHFTTKYVAVYANVG RWSASINTGNCPFSFGKVNNFVKFGSVCFSLKDIPGGCAMPIVANWAYSK YYTIGSLYVSWSDGDGITGV PQPVEGVSS GSGGGGESQVRQQFSKDIEKLLNEQVNKEMQSSNLYMSMSSWCYTHSLD GAGLFLFDHAAEEYEHAKKLIIFLNENNVPVQLTSISAPEHKFEGLTQIF QK AYEHEQHISESINNIVDHAIKSKDHATFNFLQWYVAEQHEEEVLFKDILD K IELIGNENHGLYLADQYVKGIAKSRKSGS | 304 |
| | pCoV-BoS4 | | | NITNLCPFGEVFNATRFASVYAWNRKRISNCVADYSVLYNSASFSTFKCY GVSPTKLNDLCFTNVYADSFVIRGDEVRQIAPGQTGKIADYNYKLPDDFT GCVIAWNSNNLDSKVGGNYNYLYRLFRKSNLKPFERDISTEIYQAGSTPC NGVEGFNCYFPLQSYGFQPTNGVGYQPYRVVVLSFELLHAPATVCGGGG S DCDIDKWLNNFNVPSPLNWERKIFSNCNFNLSTLLRLVHTDSFSCNNFDE S KIYGSCFKSIVLDKFAIPNSRRSDLQLGSSGFLQSSNYKIDTTSSSCQLY YSL PAINVTINNYNPSSWNRRYGFNNFNLSSHSVVYSRYCFSVNNTFCPCAKP S FASSCKSHKPPSASCPIGTNYRSCESTTVLDHTDWCRCSCLPDPITAYDP RS CSQKKSLVGVGEHCAGFGVDEEKCGVLDGSYNVSCLCSTDAFLGWSYDT CVSNNRCNIFSNFILNG CGGGGS EAKPSGSVVEQAEGVECDFSPLLSGTPPQVYNFKRLVFTNCNYNLTKLLS LFSVNDFTCSQISPAAIASNCYSSLILDYFSYPLSMKSDLSVSSAGPISQ FNY KQSFSNPTCLILATVPHNLTTITKPLKYSYINKCSRFLSDDRTEVPQLVN AN QYSPCVSIVPSTVWEDGDYYRKQLSPLEGGGWLVASGSTVAMTEQLQM GFGITVQYGTDTNSVCPK CGGGGS SIVSLPVYHKHTFIVLYVDFKPQSGGGKCFNCYPAGVNITLANFNETKGP L CVDTSHFTTKYVAVYANVG RWSASINTGNCPFSFGKVNNFVKFGSVCFSLKDIPGGCAMPIVANWAYSK YYTIGSLYVSWSDGDGITGV PQPVEGVSS GSGGGGESQVRQQFSKDIEKLLNEQVNKEMQSSNLYMSMSSWCYTHSLD GAGLFLFDHAAEEYEHAKKLIIFLNENNVPVQLTSISAPEHKFEGLTQIF QK AYEHEQHISESINNIVDHAIKSKDHATFNFLQWYVAEQHEEEVLFKDILD K IELIGNENHGLYLADQYVKGIAKSRKSGS | 305 |
| | pCoV-BoS5 | | | NITNLCPFGEVFNATRFASVYAWNRKRISNCVADYSVLYNSASFSTFKCY GVSPTKLNDLCFTNVYADSFVIRGDEVRQIAPGQTGKIADYNYKLPDDFT GCVIAWNSNNLDSKVGGNYNYLYRLFRKSNLKPFERDISTEIYQAGSTPC NGVEGFNCYFPLQSYGFQPTNGVGYQPYRVVVLSFELLHAPATVCGGGG S TNLCPFGEVFNATKFPSVYAWERKKISNCVADYSVLYNSTFFSTFKCYGV SATKLNDLCFSNVYADSFVVKGDDVRQIAPGQTGVIADYNYKLPDDFMG CVLAWNTRNIDATSTGNYNYKYRYLRHGKLRPFERDISNVPFSPDGKPCT PPALNCYWPLNDYGFYTTTGIGYQP CGGGGS DCDIDKWLNNFNVPSPLNWERKIFSNCNFNLSTLLRLVHTDSFSCNNFDE S KIYGSCFKSIVLDKFAIPNSRRSDLQLGSSGFLQSSNYKIDTTSSSCQLY YSL PAINVTINNYNPSSWNRRYGFNNFNLSSHSVVYSRYCFSVNNTFCPCAKP S FASSCKSHKPPSASCPIGTNYRSCESTTVLDHTDWCRCSCLPDPITAYDP RS CSQKKSLVGVGEHCAGFGVDEEKCGVLDGSYNVSCLCSTDAFLGWSYDT CVSNNRCNIFSNFILNG CGGGGS SIVSLPVYHKHTFIVLYVDFKPQSGGGKCFNCYPAGVNITLANFNETKGP L CVDTSHFTTKYVAVYANVG RWSASINTGNCPFSFGKVNNFVKFGSVCFSLKDIPGGCAMPIVANWAYSK YYTIGSLYVSWSDGDGITGV PQPVEGVSS GSGGGGESQVRQQFSKDIEKLLNEQVNKEMQSSNLYMSMSSWCYTHSLD GAGLFLFDHAAEEYEHAKKLIIFLNENNVPVQLTSISAPEHKFEGLTQIF QK AYEHEQHISESINNIVDHAIKSKDHATFNFLQWYVAEQHEEEVLFKDILD K IELIGNENHGLYLADQYVKGIAKSRKSGS | 306 |
| | pCoV-BoS6 | | | NITNLCPFGEVFNATRFASVYAWNRKRISNCVADYSVLYNSASFSTFKCY GVSPTKLNDLCFTNVYADSFVIRGDEVRQIAPGQTGKIADYNYKLPDDFT GCVIAWNSNNLDSKVGGNYNYLYRLFRKSNLKPFERDISTEIYQAGSTPC NGVEGFNCYFPLQSYGFQPTNGVGYQPYRVVVLSFELLHAPATVCGGGG S TNLCPFGEVFNATKFPSVYAWERKKISNCVADYSVLYNSTFFSTFKCYGV SATKLNDLCFSNVYADSFVVKGDDVRQIAPGQTGVIADYNYKLPDDFMG CVLAWNTRNIDATSTGNYNYKYRYLRHGKLRPFERDISNVPFSPDGKPCT PPALNCYWPLNDYGFYTTTGIGYQP CGGGGS DCDIDKWLNNFNVPSPLNWERKIFSNCNFNLSTLLRLVHTDSFSCNNFDE S KIYGSCFKSIVLDKFAIPNSRRSDLQLGSSGFLQSSNYKIDTTSSSCQLY YSL PAINVTINNYNPSSWNRRYGFNNFNLSSHSVVYSRYCFSVNNTFCPCAKP S FASSCKSHKPPSASCPIGTNYRSCESTTVLDHTDWCRCSCLPDPITAYDP RS CSQKKSLVGVGEHCAGFGVDEEKCGVLDGSYNVSCLCSTDAFLGWSYDT CVSNNRCNIFSNFILNG CGGGGS EAKPSGSVVEQAEGVECDFSPLLSGTPPQVYNFKRLVFTNCNYNLTKLLS LFSVNDFTCSQISPAAIASNCYSSLILDYFSYPLSMKSDLSVSSAGPISQ FNY KQSFSNPTCLILATVPHNLTTITKPLKYSYINKCSRFLSDDRTEVPQLVN AN QYSPCVSIVPSTVWEDGDYYRKQLSPLEGGGWLVASGSTVAMTEQLQM GFGITVQYGTDTNSVCPK GSGGGGESQVRQQFSKDIEKLLNEQVNKEMQSSNLYMSMSSWCYTHSLD GAGLFLFDHAAEEYEHAKKLIIFLNENNVPVQLTSISAPEHKFEGLTQIF QK AYEHEQHISESINNIVDHAIKSKDHATFNFLQWYVAEQHEEEVLFKDILD K IELIGNENHGLYLADQYVKGIAKSRKSGS | 307 |

TABLE 19

Titers for mouse sera in each immunization group, against up to three SARS-CoV-2 coating antigens, (1) RBD, (2) NTD, and (3) S-2P trimer. Values shown are the Arithmetic Mean End Point
Data is shown for Week 2 (2 weeks following 1^{st} immunization), and Week 5 (2 weeks following 2^{nd} immunization) with the exception of the RBD-His immunization group where week 10 and 12 results are shown

| Table 19 Immunogen | Immunogen Design | AH | C57BL/6 mice | | ALFQ | AH | BALB/c mice | | ALFQ | Coating Antigen |
|---|---|---|---|---|---|---|---|---|---|---|
| | | Week 2 | Week 5 | Week 2 | Week 5 | Week 2 | Week 5 | Week 2 | Week 5 | |
| RBD-His | RBD monomer | 1,140 (wk10) | 489 (wk12) | 46,080 (wk10) | 36,800 (wk12) | 1,040 (wk10) | 578 (wk12) | 91,200 (wk10) | 25,600 (wk12) | RBD |
| S-2P | Prefusion stabilized trimer | 9,920 580 520 | 184,320 3,840 2,680 | 40,960 1,280 2,720 | 2,785,280 256,000 40,960 | 3,200 320 280 | 261,12 60,800 4,840 | 7,680 210 580 | 614,400 194,560 40,960 | S-2P RBD NTD |
| pCoV23 | NTD-Ferritin | 1,680 | 7,040 | 4,400 | 56,320 | 760 | 5,120 | 1,440 | 55,040 | NTD |
| pCoV65 | NTD-Ferritin | | | 6,720 25,600 | 16,640 | | | 560 5,775 | 19,200 | S-2P NTD |
| pCoV58 | RBD-Ferritin | 9,920 2133 | | 32,000 970 | 51,200 21,760 | 6,080 2,000 | | 40,960 450 | 48,640 37,120 | S-2P RBD |
| pCoV50 | RBD-Ferritin | | | | | | | 24,320 380 | 102,400 32,000 | S-2P RBD |
| pCoV59 | RBD-Ferritin | | | | | | | 7,360 240 | 171,520 37,120 | S-2P RBD |
| pCoV1B-05 | S-Trimer-Ferritin | | | 69,689 5,486 4,089 | | | | 39,680 880 1360 | | S-2P RBD NTD |
| pCoV1B-05 (50 ug/dose) | S-Trimer-Ferritin | | | 153,600 30,720 10,880 | | | | ND | | S-2P RBD NTD |
| pCoV71 | NTD-His | | | 26,240 | | | | 2,260 | | NTD |
| pCoV122 | RBD-NTD-Ferritin | | | 4,960 6,240 4,340 | | | | 5,440 | | S-2P RBD NTD |
| pCoV111 | S1-Ferritin | | | 71,600 1,520 4,320 | | | | 33,280 2,000 | | S-2P RBD NTD |
| pCoV50+pCo V65 | Co-express RBD-Ferr & NTD-Ferr | | | 32,640 2,240 3,680 | | | | 21,760 2,440 | | S-2P RBD NTD |
| pCoV56+pCo V65 | Co-express RBD-Ferr & NTD-Ferr | | | 6,400 2,880 8640 | | | | 24,040 920 | | S-2P RBD NTD |
| pCoV58+pCo V65 | Co-express RBD-Ferr & NTD-Ferr | | | 87,040 11,600 2,000 | | | | 38,400 2,560 | | S-2P RBD NTD |
| pCoV59+pCo V65 | Co-express RBD-Ferr & NTD-Ferr | | | 56,960 6,720 4,400 | | | | 12,880 2,400 | | S-2P RBD NTD |
| pCoV129 | RBD-Ferritin | 3,520 1,140 | | 3,600 820 | | 2,400 460 | | 5,760 4,578 | | S-2P RBD |
| pCoV130 | RBD-Ferritin | | | | | | | 2,400 620 | | S-2P RBD |
| pCoV131 | RBD-Ferritin | 5,600 2,260 | | 24,178 3,111 | | 4,720 1,120 | | 8,320 1,800 | | S-2P RBD |
| pCoV127 | RBD-Ferritin | <1,600 200 | | 7,040 2,760 | | 800 578 | | 1,733 1,511 | | S-2P RBD |
| pCoV125 | RBD-NTD-Ferritin | | | | | 2,644 1,822 | | 14,133 >2,400 | | S-2P RBD |
| pCoV146 | RBD-NTD-Ferritin | | | 43,520 18,880 20,800 | | | | 17,280 8,320 | | S-2P RBD NTD |
| pCoV147 | RBD-NTD-Ferritin | | | 19,200 10,880 26,880 | | | | | | S-2P RBD NTD |
| *pCoV1B-06-PL | S-Trimer-Ferritin | | | 409,600 200,000 4,640 | | | | 51,200 14,080 | | S-2P RBD NTD |
| AH alone BALB/c | | | | | | 100 100 | 100 100 | 100 100 | 100 100 | S-2P, RBD |
| ALFQ alone BALB/c | | | | | | | | | | |

TABLE 20

VOC RBDs

| Table 20 Construct Name | Sequence | SEQ ID NO: |
|---|---|---|
| RBD-His6-E484K | NITNLCPFGEVFNATRFASVYAWNRKRISNCVADYSVLYNSASFSTFKCY GVSPTKLNDLCFTNVYADSFVIRGD EVRQIAPGQTGKIADYNYKLPDDFTGCVIAWNSNNLDSKVGGNYNYLYRL FRKSNLKPFERDISTEIYQAGSTPC NGVKGFNCYFPLQSYGFQPTNGVGYQPYRVVVLSFELLHAPATVCGPGSH HHHHH | 308 |
| RBD-His6-N501Y | NITNLCPFGEVFNATRFASVYAWNRKRISNCVADYSVLYNSASFSTFKCY GVSPTKLNDLCFTNVYADSFVIRGD EVRQIAPGQTGKIADYNYKLPDDFTGCVIAWNSNNLDSKVGGNYNYLYRL FRKSNLKPFERDISTEIYQAGSTPC NGVEGFNCYFPLQSYGFQPTYGVGYQPYRVVVLSFELLHAPATVCGPGSH HHHHH | 309 |
| RBD-His6-E484K-N501Y | NITNLCPFGEVFNATRFASVYAWNRKRISNCVADYSVLYNSASFSTFKCY GVSPTKLNDLCFTNVYADSFVIRGD EVRQIAPGQTGKIADYNYKLPDDFTGCVIAWNSNNLDSKVGGNYNYLYRL FRKSNLKPFERDISTEIYQAGSTPC NGVKGFNCYFPLQSYGFQPTYGVGYQPYRVVVLSFELLHAPATVCGPGSH HHHHH | 310 |
| RBD-His6-K417N | NITNLCPFGEVFNATRFASVYAWNRKRISNCVADYSVLYNSASFSTFKCY GVSPTKLNDLCFTNVYADSFVIRGD EVRQIAPGQTGNIADYNYKLPDDFTGCVIAWNSNNLDSKVGGNYNYLYRL FRKSNLKPFERDISTEIYQAGSTPC NGVEGFNCYFPLQSYGFQPTNGVGYQPYRVVVLSFELLHAPATVCGPGSH HHHHH | 311 |
| RBD-His6-E484K-N501Y-K417N | NITNLCPFGEVFNATRFASVYAWNRKRISNCVADYSVLYNSASFSTFKCY GVSPTKLNDLCFTNVYADSFVIRGD EVRQIAPGQTGNIADYNYKLPDDFTGCVIAWNSNNLDSKVGGNYNYLYRL FRKSNLKPFERDISTEIYQAGSTPC NGVKGFNCYFPLQSYGFQPTYGVGYQPYRVVVLSFELLHAPATVCGPGSH HHHHH | 312 |

### REFERENCES

1. Bai, H., Li, Y., Michael, N.L., Robb, M.L., and Rolland, M. (2019). The breadth of HIV-1 neutralizing antibodies depends on the conservation of key sites in their epitopes. PLoS Comput Biol 15, e1007056.
2. Bangaru, S., Lang, S., Schotsaert, M., Vanderven, H.A., Zhu, X., Kose, N., Bombardi, R., Finn, J.A., Kent, S.J., Gilchuk, P., et al. (2019). A Site of Vulnerability on the Influenza Virus Hemagglutinin Head Domain Trimer Interface. Cell 177, 1136-1152 e1118.
3. Barber, C.B., D.P. Dobkin, and H. Huhdanpaa (1996). The quickhull algorithm for convex hulls. ACM Transactions on Mathematical Software 22, 469-483.
4. Chan, Y.P., Yan, L., Feng, Y.R., and Broder, C.C. (2009). Preparation of recombinant viral glycoproteins for novel and therapeutic antibody discovery. Methods in molecular biology 525, 31-58, xiii.
5. Ge, X.Y., Li, J.L., Yang, X.L., Chmura, A.A., Zhu, G., Epstein, J.H., Mazet, J.K., Hu, B., Zhang, W., Peng, C., et al. (2013). Isolation and characterization of a bat SARS-like coronavirus that uses the ACE2 receptor. Nature 503, 535-538.
6. Hamelryck, T., and Manderick, B. (2003). PDB file parser and structure class implemented in Python. Bioinformatics 19, 2308-2310.
7. Hu, B., Zeng, L.P., Yang, X.L., Ge, X.Y., Zhang, W., Li, B., Xie, J.Z., Shen, X.R., Zhang, Y.Z., Wang, N., et al. (2017). Discovery of a rich gene pool of bat SARS-related coronaviruses provides new insights into the origin of SARS coronavirus. PLoS pathogens 13, e1006698.
8. Letko, M., Marzi, A., and Munster, V. (2020). Functional assessment of cell entry and receptor usage for SARS-CoV-2 and other lineage B betacoronaviruses. Nature microbiology.
9. Li, F. (2016). Structure, Function, and Evolution of Coronavirus Spike Proteins. Annual review of virology 3, 237-261.
10. Lu, R., Zhao, X., Li, J., Niu, P., Yang, B., Wu, H., Wang, W., Song, H., Huang, B., Zhu, N., et al. (2020). Genomic characterisation and epidemiology of 2019 novel coronavirus: implications for virus origins and receptor binding. Lancet 395, 565-574.
11. Menachery, V.D., Yount, B.L., Jr., Debbink, K., Agnihothram, S., Gralinski, L.E., Plante, J.A., Graham, R.L., Scobey, T., Ge, X.Y., Donaldson, E.F., et al. (2015). A SARS-like cluster of circulating bat coronaviruses shows potential for human emergence. Nature medicine 21, 1508-1513.
12. Munster, V.J., Koopmans, M., van Doremalen, N., van Riel, D., and de Wit, E. (2020). A Novel Coronavirus Emerging in China - Key Questions for Impact Assessment. The New England journal of medicine 382, 692-694.
13. ter Meulen, J., van den Brink, E.N., Poon, L.L., Marissen, W.E., Leung, C.S., Cox, F., Cheung, C.Y., Bakker, A.Q., Bogaards, J.A., van Deventer, E., et al. (2006). Human monoclonal antibody combination against SARS coronavirus: synergy and coverage of escape mutants. PLoS Med 3, e237.
14. Tian, X., Li, C., Huang, A., Xia, S., Lu, S., Shi, Z., Lu, L., Jiang, S., Yang, Z., Wu, Y., et al. (2020). Potent binding of 2019 novel coronavirus spike protein by a SARS coronavirus-specific human monoclonal antibody. Emerging microbes & infections 9, 382-385.
15. Tripp, R.A., Haynes, L.M., Moore, D., Anderson, B., Tamin, A., Harcourt, B.H., Jones, L.P., Yilla, M., Babcock, G.J., Greenough, T., et al. (2005). Monoclonal antibodies to SARS-associated coronavirus (SARS-CoV): Identification of neutralizing and antibodies reactive to S, N, M and E viral proteins. Journal of Virological Methods.
16. Wang, L., Shi, W., Chappell, J.D., Joyce, M.G., Zhang, Y., Kanekiyo, M., Becker, M.M., van Doremalen, N., Fischer, R., Wang, N., et al. (2018). Importance of Neutralizing Monoclonal Antibodies Targeting Multiple Antigenic Sites on the Middle East Respiratory Syndrome Coronavirus Spike Glycoprotein To Avoid Neutralization Escape. Journal of virology 92.
17. Wang, L., Shi, W., Joyce, M.G., Modjarrad, K., Zhang, Y., Leung, K., Lees, C.R., Zhou, T., Yassine, H.M., Kanekiyo, M., et al. (2015). Evaluation of candidate vaccine approaches for MERS-CoV. Nature communications 6, 7712.
18. West, B.R., Moyer, C.L., King, L.B., Fusco, M.L., Milligan, J.C., Hui, S., and Saphire, E.O. (2018). Structural Basis of Pan-Ebolavirus Neutralization by a Human Antibody against a Conserved, yet Cryptic Epitope. mBio 9.
19. Wrapp, D., Wang, N., Corbett, K.S., Goldsmith, J.A., Hsieh, C.L., Abiona, O., Graham, B.S., and McLellan, J.S. (2020). Cryo-EM structure of the 2019-nCoV spike in the prefusion conformation. Science.
20. Wu, F., Zhao, S., Yu, B., Chen, Y.M., Wang, W., Song, Z.G., Hu, Y., Tao, Z.W., Tian, J.H., Pei, Y.Y., et al. (2020). A new coronavirus associated with human respiratory disease in China. Nature.
21. Yan, R., Zhang, Y., Guo, Y., Xia, L., and Zhou, Q. (2020a). Structural basis for the recognition of the 2019-nCoV by human ACE2. BiorXiv.
22. Yan, R., Zhang, Y., Li, Y., Xia, L., Guo, Y., and Zhou, Q. (2020b). Structural basis for the recognition of the SARS-CoV-2 by full-length human ACE2. Science.
23. Yang, X.L., Hu, B., Wang, B., Wang, M.N., Zhang, Q., Zhang, W., Wu, L.J., Ge, X.Y., Zhang, Y.Z., Daszak, P., et al. (2015). Isolation and Characterization of a Novel Bat Coronavirus Closely Related to the Direct Progenitor of Severe Acute Respiratory Syndrome Coronavirus. Journal of virology 90, 3253-3256.
24. Zhou, P., Yang, X.L., Wang, X.G., Hu, B., Zhang, L., Zhang, W., Si, H.R., Zhu, Y., Li, B., Huang, C.L., et al. (2020). A pneumonia outbreak associated with a new coronavirus of probable bat origin. Nature.

What is claimed is:

1. A nanoparticle comprising a fusion protein comprising a nanoparticle-forming peptide and at least one antigenic coronavirus peptide selected from:
a. a receptor-binding domain (RBD) of a coronavirus, or a fragment or variant thereof,
b. an N-terminal domain (NTD) of a coronavirus, or a fragment or variant thereof,
c. an S1 domain of a coronavirus, or a fragment or variant thereof,
d. a stabilized extracellular spike S-2P domain of a coronavirus, or a fragment or variant thereof,
e. a stabilized extracellular spike S domain of a coronavirus, or a fragment or variant thereof, or
f. a stabilized extracellular spike S-trimer of a coronavirus, or a fragment or variant thereof,.

2. (canceled)

3. The nanoparticle of claim 1, wherein the nanoparticle-forming peptide comprises or is Helicobacter pylori ferritin (Hpf) or a fragment or variant thereof.

4. The nanoparticle of claim 1, wherein the nanoparticle-forming peptide comprises an amino acid sequence selected from:
a. ESQVRQQFSKDIEKLLNEQVNKEMQSSNLYMSMSSWCYTHSLDGAGLFLFDHA AEEYEHAKKLIIFLNENNVPVQLTSISAPEHKFEGLTQIFQKAYEHEQHISESINNIV DHAIKSKDHATFNFLQWYVAEQHEEEVLFKDILDKIELIGNENHGLYLADQYVK GIAKSRKSGS or a fragment or variant thereof,
b. DIIKLLNEQVNKEMQSSNLYMSMSSWCYTHSLDGAGLFLFDHAAEEYEHAKKLI IFLNENNVPVQLTSISAPEHKFEGLTQIFQKAYEHEQHISESINNIVDHAIKSKDHA TFNFLQWYVAEQHEEEVLFKDILDKIELIGNENHGLYLADQYVKGIAKSRKSGS or a fragment or variant thereof, and
c. SKDIIKLLNEQVNKEMQSSNLYMSMSSWCYTHSLDGAGLFLFDHAAEEY EHAKKLIIFLNENNVPVQLTSISAPEHKFEGLTQIFQKAYEHEQHISESINNIVDHAI KSKDHATFNFLQWYVAEQHEEEVLFKDILDKIELIGNENHGLYLADQYVKGIAK SRKSGS or a fragment or variant thereof.

5. The nanoparticle of claim 1, wherein the nanoparticle possesses a 4-fold axis or a 3-fold axis.

6. The nanoparticle of claim 1, wherein the antigenic coronavirus peptide is connected to the nanoparticle-forming peptide via a linker.

7. The nanoparticle of claim 6, wherein the linker comprises an amino acid sequence selected from:
a. GSGGGG,
b. GGGG
c. GSGG
d. GGG, and
e. SGG.

8. The nanoparticle of claim 1, wherein the fusion protein comprises 2-10 antigenic coronavirus peptides connected in series, optionally wherein the antigenic coronavirus peptides are connected via peptide linkers.

9. The nanoparticle of claim 1, wherein the antigenic coronavirus peptide is isolated or derived from a coronavirus selected from SARS-CoV-2, human coronavirus OC43 (hCoV-OC43), Middle East respiratory syndrome-related coronavirus (MERS-CoV), severe acute respiratory syndrome-related coronavirus (SARS-CoV-1), HKU-1, 229E, or NL63.

10. The nanoparticle of claim 1, wherein the nanoparticle comprises one or more of:
a. an Hpf or a fragment or variant thereof connected via a peptide linker to an RBD or a fragment or variant thereof,
b. an Hpf or a fragment or variant thereof connected via a peptide linker to an NTD or a fragment or variant thereof,
c. an Hpf or a fragment or variant thereof connected via a peptide linker to an S1 or a fragment or variant thereof,
d. an Hpf or a fragment or variant thereof connected via a peptide linker to a stabilized extracellular spike domain (S-2P) or a fragment or variant thereof,
e. any fusion protein disclosed in Table 3, and
f. any fusion protein disclosed in Table 18.

11-13. (canceled)

14. A vaccine comprising the nanoparticle of claim 1.

15. The vaccine of claim 14, wherein the vaccine further comprises one or more adjuvants selected from ALFQ, alhydrogel, and combinations thereof.

16. A messenger RNA (mRNA) encoding a nanoparticle according to claim 1.

17. A method of treating or preventing a coronavirus infection in a subject in need thereof, comprising administering to a subject in need thereof the nanoparticle according to claim 1, or a vaccine comprising the nanoparticle, or an mRNA encoding the nanoparticle.

18-22. (canceled)

23. The method of claim 17, wherein prior to administering the nanoparticle or vaccine to the subject, the subject is administered a priming dose of a DNA sequence encoding a receptor-binding domain (RBD) of a coronavirus, or a fragment or variant thereof.

24-32. (canceled)

33. A method of screening for binding molecules capable of binding to coronavirus, comprising contacting a binding molecule with a nanoparticle listed in Table 18 to identify a binding molecule that binds to the nanoparticle.

34. A DNA molecule, comprising a sequence encoding a nanoparticle according to claim 1.

35. A DNA molecule, comprising a sequence encoding a receptor receptor-binding domain (RBD) of a coronavirus, or a fragment or variant thereof.

36-39. (canceled)

40. A method of priming an immune response in a subject, comprising administering to a subject a DNA molecule comprising a sequence encoding a receptor receptor-binding domain (RBD) of a coronavirus or a fragment or variant thereof, or a plasmin comprising the DNA molecule that can express the DNA molecule in vivo, prior to administering to the subject the nanoparticle according to claim 1, or a vaccine comprising the nanoparticle, or an mRNA encoding the nanoparticle.

41. (canceled)

42. (canceled)

43. A method of treating or preventing a coronavirus infection in a subject in need thereof, comprising administering to the subject an anti-coronavirus antibody obtained from or cloned from an immunized subject that was administered a nanoparticle according to claim 1, or a vaccine comprising the nanoparticle, or an mRNA encoding the nanoparticle.

44. (canceled)

45. (canceled)
